SEQUENCE LISTING (172 sequences)

SEQ ID NO: 1 - protein, 8 aa, Artificial sequence
  Synthetic peptide
WSHPQFEK

SEQ ID NO: 2 - protein, 8 aa, Artificial sequence
  Synthetic peptide
WSHPQFEK

SEQ ID NO: 3 - protein, 8 aa, Artificial sequence
  Synthetic peptide
WSHPQFEK

SEQ ID NO: 4 - protein, 8 aa, Artificial sequence
  Synthetic peptide
WSHPQFEK

SEQ ID NO: 5 - protein, 8 aa, Artificial sequence
  Synthetic peptide
WSHPQFED

SEQ ID NO: 6 - protein, 8 aa, Artificial sequence
  Synthetic peptide
WSHPQFED

SEQ ID NO: 7 - protein, 8 aa, Artificial sequence
  Synthetic peptide
WSHPQFED

SEQ ID NO: 8 - protein, 8 aa, Artificial sequence
  Synthetic peptide
WSHPQFEK

SEQ ID NO: 9 - protein, 8 aa, Artificial sequence
  Synthetic peptide
WSHPQFEK

SEQ ID NO: 10 - protein, 46 aa, Artificial sequence
  Synthetic peptide
KAGVCPPKKSAQCLRYKKPECQSDWQCPGKKRCCPDTCGIKCLDPV

SEQ ID NO: 11 - protein, 46 aa, Artificial sequence
  Synthetic peptide
KPGKCPVTYGQCLMLNPPNFCEMDGQCKRDLKCCMGMCGKSCVSPV

SEQ ID NO: 12 - protein, 46 aa, Artificial sequence
  Synthetic peptide
KPGSCPIILIRCAMLNPPNRCLKDTDCPGIKKCCEGSCGMACFVPQ

SEQ ID NO: 13 - protein, 45 aa, Artificial sequence
  Synthetic peptide
FPRRCPKIREECEFQERDVCTKDRQCQDNKKCCVFSCGKKCLDLK

SEQ ID NO: 14 - protein, 58 aa, Artificial sequence
  Synthetic peptide
GYRDKKRMQKTQLSPEIKVCQQQPKLYLCKHLCESHRDCQANNICCSTYCGNVCMSIL

SEQ ID NO: 15 - protein, 38 aa, Artificial sequence
  Synthetic peptide
EIKVCQQQPKLYLCKHLCESHRDCQANNICCSTYCGNV

SEQ ID NO: 16 - protein, 52 aa, Artificial sequence
  Synthetic peptide
GYRDKMRMQRIKVCEKRPSIDLCIHHCSYFQKCETNKICCSAFCGNICMSIL

SEQ ID NO: 17 - protein, 46 aa, Artificial sequence
  Synthetic peptide
RADRCPPPPTLPPGACQAARCQADSECPRHRRCCYNGCAYACLEAV

SEQ ID NO: 18 - protein, 43 aa, Artificial sequence
  Synthetic peptide
KTGVCPELQADQNCTQECVSDSECADNLKCCSAGCATFCSLPN

SEQ ID NO: 19 - protein, 54 aa, Artificial sequence
  Synthetic peptide
SLPNDKEGSCPQVNINFPQLGLCRDQCQVDSQCPGQMKCCRNGCGKVSCVTPNF

SEQ ID NO: 20 - protein, 41 aa, Artificial sequence
  Synthetic peptide
KEGECPPHKNPCKELCQGDELCPAEQKCCTTGCGRICRDIP

SEQ ID NO: 21 - protein, 44 aa, Artificial sequence
  Synthetic peptide
RKRDCPRVIRKQSCLKRCITDETCPGVKKCCTLGCNKSCVVPIS

SEQ ID NO: 22 - protein, 41 aa, Artificial sequence
  Synthetic peptide
FGGECPADPLPCEELCDGDASCPQGHKCCSTGCGRTCLGDI

SEQ ID NO: 23 - protein, 47 aa, Artificial sequence
  Synthetic peptide
DIEGGRGGDCPKVLVGLCIVGCVMDENCQAGEKCCKSGCGRFCVPPV

SEQ ID NO: 24 - protein, 45 aa, Artificial sequence
  Synthetic peptide
KSGGCPPDDGPCLLSVPDQCVEDSQCPLTRKCCYRACFRQCVPRV

SEQ ID NO: 25 - protein, 45 aa, Artificial sequence
  Synthetic peptide
KLGSCPEDQLRCLSPMNHLCHKDSDCSGKKRCCHSACGRDCRDPA

SEQ ID NO: 26 - protein, 39 aa, Artificial sequence
  Synthetic peptide
KPCPKIKVECEVEEIDQCTKPRDCPENMKCCPFSRGKKC

SEQ ID NO: 27 - protein, 44 aa, Artificial sequence
  Synthetic peptide
KPGLCPKERLTCTTELPDSCNTDFDCKEYQKCCFFACQKKCMDP

SEQ ID NO: 28 - protein, 53 aa, Artificial sequence
  Synthetic peptide
CRTACMLIVKDGQCPLFPFTERKECPPSCHSDIDCPQTDKCCESRCGFVCARA

SEQ ID NO: 29 - protein, 43 aa, Artificial sequence
  Synthetic peptide
KKGFCPRKPLLCTKIDKPKCLQDEECPLVEKCCSHCGLKCMDP

SEQ ID NO: 30 - protein, 65 aa, Artificial sequence
  Synthetic peptide
SFWNKDPFLDMIRETECWVQPPYKYCEKRCTKIMTCVRPNHTCCWTYCGNICLDNEEPLKSMLNP

SEQ ID NO: 31 - protein, 62 aa, Artificial sequence
  Synthetic peptide
EMRKKRYDRKELLLEECWGKPNVKECTNKCSKAFRCKDKNYTCCWTYCGNICWINVETSGDY

SEQ ID NO: 32 - protein, 45 aa, Artificial sequence
  Synthetic peptide
KAGVCPADNVRCFKSDPPQCHTDQDCLGERKCCYLKCHFKVCIPV

SEQ ID NO: 33 - protein, 71 aa, Artificial sequence
  Synthetic peptide
SPKQRVLKYILEPPPCISAPENCTHLCTMQEDCEKGHQCCSSFCGIVCSSETFQKRNRIKHKGSEVIMPAN

SEQ ID NO: 34 - protein, 242 aa, Artificial sequence [sequence omitted]

SEQ ID NO: 35 - protein, 131 aa, Artificial sequence
  Synthetic peptide
MKSSGLFPFLVLLALGTLAPWAVEGSGKSFKAGVCPPKKSAQCLRYKKPECQSDWQCPGKKRCCPDTCGIKCLDPVMVEGSGKSFKAGVCPPKKSAQCLRYKKPECQSDWQCPGKKRCCPDTCGIKCLDPV

SEQ ID NO: 36 - protein, 56 aa, Artificial sequence
  Synthetic peptide
DTPNPTRRKPGKCPVTYGQCLMLNPPNFCEMDGQCKRDLKCCMGMCGKSCVSPVKA

SEQ ID NO: 37 - protein, 123 aa, Artificial sequence
  Synthetic peptide
MRTQSLLLLGALLAVGSQLPAVFGRKKGEKSGGCPPDDGPCLLSVPDQCVEDSQCPLTRKCCYRACFRQCVPRVSVKLGSCPEDQLRCLSPMNHLCHKDSDCSGKKRCCHSACGRDCRDPARG

SEQ ID NO: 38 - protein, 149 aa, Artificial sequence
  Synthetic peptide
MWPNSILVLMTLLISSTLVTGGGVKGEEKRVCPPDYVRCIRQDDPQCYSDNDCGDQEICCFWQCGFKCVLPVKDNSEEQIPQSKVGGVKGEEKRVCPPDYVRCIRQDDPQCYSDNDCGDQEICCFWQCGFKCVLPVKDNSEEQIPQSKV

SEQ ID NO: 39 - protein, 141 aa, Artificial sequence
  Synthetic peptide
MKLLGLSLLAVTILLCCNMARPEIKKKNVFSKPGYCPEYRVPCPFVLIPKCRRDKGCKDALKCCFFYCQMRCVDPWESPEARPEIKKKNVFSKPGYCPEYRVPCPFVLIPKCRRDKGCKDALKCCFFYCQMRCVDPWESPE

SEQ ID NO: 40 - DNA, 69 bp, Artificial sequence
  Oligonucleotide
aagcttatggattttcaagtgcagattttcagcttcctgctaatcagtgcttcagtcataatgtccaga

SEQ ID NO: 41 - DNA, 69 bp, Artificial sequence
  Oligonucleotide
tctttggctgtgtctctaggtcagagagccaccatctcctgcagagccagtgaaagtgttgaatattat

SEQ ID NO: 42 - DNA, 69 bp, Artificial sequence
  Oligonucleotide
ccaggacagccacccaaactcctcatctctgctgctagcaacgtagaatctggggtccctgccaggttt

SEQ ID NO: 43 - DNA, 69 bp, Artificial sequence
  Oligonucleotide
aacatccatcctgtggaggaggatgatattgcaatgtatttctgtcagcaaagtaggaaggttccatgg

SEQ ID NO: 44 - DNA, 69 bp, Artificial sequence
  Oligonucleotide
tagagacacagccaaagaagctggagattgggtgagcacaatgtcgactcctctggacattatgactga

SEQ ID NO: 45 - DNA, 69 bp, Artificial sequence
  Oligonucleotide
tttgggtggctgtcctggtttctgttggtaccactgcattaaacttgtgacataatattcaacactttc

SEQ ID NO: 46 - DNA, 69 bp, Artificial sequence
  Oligonucleotide
ctccacaggatggatgttgaggctgaagtctgtcccagacccactgccactaaacctggcagggacccc

SEQ ID NO: 47 - DNA, 69 bp, Artificial sequence
  Oligonucleotide
ggatccaccgccaccccgtttgatttccagcttggtgcctccaccgaacgtccatggaaccttcctact

SEQ ID NO: 48 - DNA, 66 bp, Artificial sequence
  Oligonucleotide
ggatccggcggaggtgggtcgggtggcggcggatctcaggtgcagctgaaggagtcaggacctggc

SEQ ID NO: 49 - DNA, 66 bp, Artificial sequence
  Oligonucleotide
acatgcaccgtctcagggttctcattaaccggctatggtgtaaactgggttcgccagcctccagga

SEQ ID NO: 50 - DNA, 66 bp, Artificial sequence
  Oligonucleotide
ggtgatggaagcacagactataattcagctctcaaatccagactatcgatcaccaaggacaactcc

SEQ ID NO: 51 - DNA, 67 bp, Artificial sequence
  Oligonucleotide
ctgcaaactgatgacacagccagatactactgtgctcgagatggttatagtaactttcattactatg

SEQ ID NO: 52 - DNA, 66 bp, Artificial sequence
  Oligonucleotide
ccctgagacggtgcatgtgatggacaggctctgtgagggcgccaccaggccaggtcctgactcctt

SEQ ID NO: 53 - DNA, 66 bp, Artificial sequence
  Oligonucleotide
gtctgtgcttccatcaccccatatcattcccagccactctagaccctttcctggaggctggcgaac

SEQ ID NO: 54 - DNA, 66 bp, Artificial sequence
  Oligonucleotide
tgtgtcatcagtttgcagactgttcatttttaagaaaacttggctcttggagttgtccttggtgat

SEQ ID NO: 55 - DNA, 67 bp, Artificial sequence
  Oligonucleotide
tgatcagaggagacggtgactgaggttccttgaccccagtagtccataacatagtaatgaaagttac

SEQ ID NO: 56 - DNA, 824 bp, Artificial sequence
  Synthetic
aagcttatggattttcaagtgcagattttcagcttcctgctaatcagtgcttcagtcataatgtccagaggagtcgacattgtgctcacccaatctccagcttctttggctgtgtctctaggtcagagagccaccatctcctgcagagccagtgaaagtgttgaatattatgtcacaagtttaatgcagtggtaccaacagaaaccaggacagccacccaaactcctcatctctgctgctagcaacgtagaatctggggtccctgccaggtttagtggcagtgggtctgggacagactttagcctcaacatccatcctgtggaggaggatgatattgcaatgtatttctgtcagcaaagtaggaaggttccatggacgttcggtggaggcaccaagctggaaatcaaacggggtggcggtggatccggcggaggtgggtcgggtggcggcggatctcaggtgcagctgaaggagtcaggacctggcctggtggcgccctcacagagcctgtccatcacatgcaccgtctcagggttctcattaaccggctatggtgtaaactgggttcgccagcctccaggaaagggtctagagtggctgggaatgatatggggtgatggaagcacagactataattcagctctcaaatccagactatcgatcaccaaggacaactccaagagccaagttttcttaaaaatgaacagtctgcaaactgatgacacagccagatactactgtgctcgagatggttatagtaactttcattactatgttatggactactggggtcaaggaacctcagtcaccgtctcctctgatca

SEQ ID NO: 57 - protein, 248 aa, Artificial sequence
  2E12 VL-VH
DIVLTQSPASLAVSLGQRATISCRASESVEYYVTSLMQWYQQKPGQPPKLLISAASNVESGVPARFSGSGSGTDFSLNIHPVEEDDIAMYFCQQSRKVPWTFGGGTKLEIKRGGGGSGGGGSGGGGSQVQLKESGPGLVAPSQSLSITCTVSGFSLTGYGVNWVRQPPGKGLEWLGMIWGDGSTDYNSALKSRLSITKDNSKSQVFLKMNSLQTDDTARYYCARDGYSNFHYYVMDYWGQGTSVTVSS

SEQ ID NO: 58 - DNA, 62 bp, Artificial sequence
  Synthetic primer
atagtctaggtcgacattgtgctgacacagtttcctgctagccttagcgtatttttggggca

SEQ ID NO: 59 - DNA, 62 bp, Artificial sequence
  Synthetic primer
gccaaagtgtcagtacatatggctatagttatatgcactggaaccaacagaaaccaggacag

SEQ ID NO: 60 - DNA, 62 bp, Artificial sequence
  Synthetic primer
atccaatctagaatttggggtccctgccaggttcagtggcagtgggtctgggacagacttca

SEQ ID NO: 61 - DNA, 62 bp, Artificial sequence
  Synthetic primer
gaggatgctgcaacctattattgtcagcacattagggagcttccttacacgttcggaggggg

SEQ ID NO: 62 - DNA, 62 bp, Artificial sequence
  Synthetic reverse primer
atgtactgacactttggctggccctgcatgaaatggtggccctctgccccaaaaatacgcta

SEQ ID NO: 63 - DNA, 62 bp, Artificial sequence
  Synthetic reverse primer
ccaaattctagattggatacaagataaatgaggagtctgggtggctgtcctggtttctgttg

SEQ ID NO: 64 - DNA, 62 bp, Artificial sequence
  Synthetic reverse primer
ataggttgcagcatcctcctcctccacaggatggatgttgagggtgaagtctgtcccagacc

SEQ ID NO: 65 - DNA, 61 bp, Artificial sequence
  Synthetic reverse primer
acctccgccggatccaccgccacctttgatttccagcttggtcccccctccgaacgtgtaa

SEQ ID NO: 66 - DNA, 24 bp, Artificial sequence
  Synthetic primer
atagtctaggtcgacattgtgctg

SEQ ID NO: 67 - DNA, 23 bp, Artificial sequence
  Synthetic primer
acctccgccggatccaccgccac

SEQ ID NO: 68 - DNA, 69 bp, Artificial sequence
  Synthetic primer
ggtggcggtggatccggcggaggtgggtcgggtggcggcggatcggaggtacagcttctggagtctggg

SEQ ID NO: 69 - DNA, 69 bp, Artificial sequence
  Synthetic primer
ttgtcctgcacagcttctggcttcaacattaaagacacctatatgcactgggtgaagcagaggcctgaa

SEQ ID NO: 70 - DNA, 69 bp, Artificial sequence
  Synthetic primer
gcgaatggtaatactaaatatgacccgaagttccagggcaaggccactataacagcagacacatcctcc

SEQ ID NO: 71 - DNA, 69 bp, Artificial sequence
  Synthetic primer
tctgaggacaccgcggtctattactgtgctaggccatctatttactacggtagtaaccactggtacttc

SEQ ID NO: 72 - DNA, 69 bp, Artificial sequence
  Synthetic primer
agaagctgtgcaggacaacttgactgaggcccctggcttcacaagctctgccccagactccagaagctg

SEQ ID NO: 73 - DNA, 69 bp, Artificial sequence
  Synthetic primer
tttagtattaccattcgcaggatcgatccttccgatccactccaggccctgttcaggcctctgcttcac

SEQ ID NO: 74 - DNA, 69 bp, Artificial sequence
  Synthetic primer
gaccgcggtgtcctcagatgtcaggctgctgagctgcaggtaggctgtgttggaggatgtgtctgctgt

SEQ ID NO: 75 - DNA, 72 bp, Artificial sequence
  Synthetic primer
tgatactatcagatctgaggagacggtgactgaggttcctgcgccccagacatcgaagtaccagtggttact

SEQ ID NO: 76 - DNA, 24 bp, Artificial sequence
  Synthetic primer
ggtggcggtggatccggcggaggt

SEQ ID NO: 77 - DNA, 23 bp, Artificial sequence
  Synthetic primer
tgatactatcagatctgaggaga

SEQ ID NO: 78 - DNA, 819 bp, Mus musculus
aagcttgccgccatggattttcaagtgcagattttcagcttcctgctaatcagtgcttcagtcataattgccagaggagtcgactctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggataccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtggcggtggctcgggcggtggtgggtcgggtggcggcgggagctctcaggtgcagctggtgcagtctggggctgagtcgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatccgaagacacggccgtgtattactgtgcaaggttgacgcggaataagtttaagtcgcgtggtcattggggccaaggtaccctggtcaccgtgtcgagagatctg

SEQ ID NO: 79 - protein, 246 aa, Mus musculus
DSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGGSGGGGSSQVQLVQSGAESKKPGASVKVSCKASGYTFTSYAMHWVRQAPGQRLEWMGWINAGNGNTKYSQKFQGRVTITRDTSASTAYMELSSLRSEDTAVYYCARLTRNKFKSRGHWHQHTLVTVSRDL

SEQ ID NO: 80 - DNA, 47 bp, Artificial sequence
  Synthetic primer
acgcgtaagcttgccgccccatggcctggacccctctctggctcact

SEQ ID NO: 81 - DNA, 46 bp, Artificial sequence
  Synthetic primer
agagctcccgccgccacccgacccaccaccgcccgagccaccgcca

SEQ ID NO: 82 - DNA, 71 bp, Artificial sequence
  Synthetic primer
ggacccctctctggctcactctcctcactctttgcataggttccgtggtttcttctgagctgactcaggac

SEQ ID NO: 83 - DNA, 70 bp, Artificial sequence
  Synthetic primer
caccgcccgagccaccgccacctaggacggtcagcttggtccctccgccgaataccacatggttaccact

SEQ ID NO: 84 - DNA, 65 bp, Artificial sequence
  Synthetic primer
tcttctgagctgactcaggaccctgctgtgtctgtggccttgggacagacagtcaggatcacatg

SEQ ID NO: 85 - DNA, 65 bp, Artificial sequence
  Synthetic primer
aataccacatggttaccactgctgtcccgggagttacagtaatagtcagcctcatcttccgcctg

SEQ ID NO: 86 - DNA, 66 bp, Artificial sequence
  Synthetic primer
cagacagtcaggatcacatgccaaggagacagcctcagaagctattatgcaagctggtaccagcag

SEQ ID NO: 87 - DNA, 65 bp, Artificial sequence
  Synthetic primer
tcagcctcatcttccgcctgagccccagtgatggtcaaggaagctgtgtttcctgagctggagcc

SEQ ID NO: 88 - DNA, 68 bp, Artificial sequence
  Synthetic primer
tatgcaagctggtaccagcagaagccaggacaggcccctgtacttgtcatctatggtaaaaacaaccg

SEQ ID NO: 89 - DNA, 68 bp, Artificial sequence
  Synthetic primer
gtgtttcctgagctggagccagagaatcggtctggtatccctgagggccggttgtttttaccatagat

SEQ ID NO: 90 - DNA, 30 bp, Artificial sequence
  Synthetic primer
gggagctcttctgagctgactcaggaccct

SEQ ID NO: 91 - DNA, 52 bp, Artificial sequence
  Synthetic primer
cagatctaggacggtcagcttggtccctccgccgaataccacatggttacca

SEQ ID NO: 92 - DNA, 65 bp, Artificial sequence
  Synthetic primer
acgcgtaagcttgccgccatggactggacctggagaatcctcttcttggtggcagcagccacagg

SEQ ID NO: 93 - DNA, 43 bp, Artificial sequence
  Synthetic primer
agagctcccgccgccacccgacccaccaccgcccgagccaccg

SEQ ID NO: 94 - DNA, 70 bp, Artificial sequence
  Synthetic primer
tggtggcagcagccacaggagcccactcccaggtgcagctggtgcagtctggggctgagtcgaagaagcc

SEQ ID NO: 95 - DNA, 48 bp, Artificial sequence
  Synthetic primer
caccaccgcccgagccaccgccacctctcgacacggtgaccagggtac

SEQ ID NO: 96 - DNA, 68 bp, Artificial sequence
  Synthetic primer
ggctgagtcgaagaagcctggggcctcagtgaaggtttcctgcaaggcttctggatacaccttcacta

SEQ ID NO: 97 - DNA, 70 bp, Artificial sequence
  Synthetic primer
cttggccccaatgaccacgcgacttaaacttattccgcgtcaaccttgcacagtaatacacggccctgtc

SEQ ID NO: 98 - DNA, 70 bp, Artificial sequence
  Synthetic primer
ttctggatacaccttcactagctatgctatgcattgggtgcgccaggcccccggacaaaggcttgagtgg

SEQ ID NO: 99 - DNA, 70 bp, Artificial sequence
  Synthetic primer
acagtaatacacggccgtgtcttcggatctcaggctgctcagctccatgtaggctgtgctcgcggatgtg

SEQ ID NO: 100 - DNA, 68 bp, Artificial sequence
  Synthetic primer
ccggacaaaggcttgagtggatgggatggatcaacgctggcaatggtaacacaaaatattcacagaag

SEQ ID NO: 101 - DNA, 68 bp, Artificial sequence
  Synthetic primer
ggctgtgctccgccatgtgtccctggtaatggtgactctgccctggaacttctgtgaatattttgtgt

SEQ ID NO: 102 - DNA, 50 bp, Artificial sequence
  Synthetic primer
gggagctctcaggtgcagctggtgcagtctggggctgagtcgaagaagcc

SEQ ID NO: 103 - DNA, 49 bp, Artificial sequence
  Synthetic primer
cagatctctcgacacggtgaccagggtaccttggccccaatgaccacgc

SEQ ID NO: 104 - DNA, 815 bp, Homo sapiens
acgcgtaagcttgccgccccatggcctggacccctctctggctcactctcctcactctttgcataggttccgtggtttcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggataccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtggcggtggctcgggcggtggtgggtcgggtggcggcgggagctctcaggtgcagctggtgcagtctggggctgagtcgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatccgaagacacggccgtgtattactgtgcaaggttgacgcggaataagtttaagtcgcgtggtcattggggccaaggtaccctggtcaccgtgtcgagagatctg

SEQ ID NO: 105 - protein, 265 aa, Homo sapiens
MAWTPLWLTLLTLCIGSVVSSELTQDPAVSVALGQTVRITCQGDSLRSYYASWYQQKPGQAPVLVIYGKNNRPSGIPDRFSGSSSGBTASLTITGAQAEDEADYYCNSRDSSGNHVVFGGGTKLTVLGGGGSGGGGSGGGGSSQVQLVQSGAESKKPGASVKVSCKASGYTFTSYAMHWVRQAPGQRLEWMGWINAGNGNTKYSQKFQGRVTITRDTSASTAYMELKSLRSEDTAVYYCARLTRNKFKSRGHWGQGTLVTVSRDL

SEQ ID NO: 106 - protein, 264 aa, Homo sapiens
MAWTPLWLTLLTLCIGSVVSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGGSGGGGSSQVQLVQSGAESKKPGASVKVSCKASGYTFTSYAMHWVRQAPGQRLEWMGWINAGNGNTKYSQKFQGRVTITRDTSASTAYMELSSLRSEDTAVYYCARLTRNKFKSRGHWGQGTLVTVSRD

SEQ ID NO: 107 - DNA, 810 bp, Homo sapiens
acgcgtaagcttgccgccatggactggacctggagaatcctcttcttggtggcagcagccacaggagcccactcccaggtgcagctggtgcagtctggggctgagtcgaagaagcctggggcctcagtgaaggtttcctgcaaggcttctggatacaccttcactagctatgctatgcattgggtgcgccaggcccccggacaaaggcttgagtggatgggatggatcaacgctggcaatggtaagagaaaatattcacagaagttccagggcagagtcaccattaccagggacacatccgcgagcacagcctacatggagctgagcagcctgagatccgaagacacggccgtgtattactgtgcaaggttgacgcggaataagtttaagtcgcgtggtcattggggccaaggtaccctggtcaccgtgtcgagaggtggcggtggctcgggcggtggtgggtcgggtggcggcgggagctcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggataccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctagatctg

SEQ ID NO: 108 - protein, 263 aa, Homo sapiens
MDWTWRILFLVAAATGAHSQVQLVQSGAESKKPGASVKVSCKASGYTFTSYAMHWVRQAPGQRLEWMGWINAGNGNTKYSQKFQGRVTITRDTSASTAYMELSSLRSEDTAVYYCARLTRNKFKSRGHWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLDL

SEQ ID NO: 109 - DNA, 483 bp, Artificial sequence
  Synthetic oligonucleotide
gtggtcctaaaggtcaaggccaagatcctgttgagggtcaagaccaagacgaaggtccaggtccagtcaaagttgaaattctagacataggacaagatccagtcaagggtcaagatccagtcaagggtcaagatccagtcaagggtcaagatccagtcaagggtcaagatctagtcaaaagtcaagatccagtcaaagctgaacttccagacataggacaggatgtagtcaaaggtcatgaaccagtcgagggtcaagatccagtcaatgcccaacttccagacaaagtacaagatccagtcaaagcccaacctgcagtcccaggtcgattccttctctctaagcgtggccactgccctaggattctttttcgttgcccgctgagcaatccctctaacaagtgttggagagattatgactgtccaggggtcaagaagtgctgtgaaggcttttgcgggaaggattgtttgtatcccaagtgag

SEQ ID NO: 110 - protein, 159 aa, Artificial sequence
  Synthetic peptide
GPKGQGQDPVEGQDQDEGPGPVKVEILDIGQDPVKGQDPVKGQDPVKGQDPVKGQDLVKSQDPVKAELPDIGQDVVKGHEPVEGQDPVNAQLPDKVQDPVKAQPAVPGRFLLSKRGHCPRILFRCPLSNPSNKCWRDYDCPGVKKCCEGFCGKDCLYPK

SEQ ID NO: 111 - protein, 76 aa, Artificial sequence
  Synthetic peptide
MKSSGLFPFLVLLALGTLAPWAVEGSGKSFKAGVCPPKKSAQCLRYKKPECQSDWQCPGKKRCCPDTCGIKCLDPV

SEQ ID NO: 112 - protein, 56 aa, Artificial sequence; identical to SEQ ID NO: 36
  Synthetic peptide

SEQ ID NO: 113 - protein, 46 aa, Artificial sequence; identical to SEQ ID NO: 10
  Synthetic peptide

SEQ ID NO: 114 - protein, 45 aa, Artificial sequence; identical to SEQ ID NO: 25
  Synthetic peptide

SEQ ID NO: 115 - protein, 45 aa, Artificial sequence
  Synthetic peptide
KAGVCPADNVRCFKSDPPQCHTDQDCLGERKCCYLHCGFKCVIPV

SEQ ID NO: 116 - protein, 44 aa, Artificial sequence; identical to SEQ ID NO: 27
  Synthetic peptide

SEQ ID NO: 117 - protein, 45 aa, Artificial sequence; identical to SEQ ID NO: 24
  Synthetic peptide

SEQ ID NO: 118 - protein, 43 aa, Artificial sequence; identical to SEQ ID NO: 29
  Synthetic peptide

SEQ ID NO: 119 - protein, 46 aa, Artificial sequence; identical to SEQ ID NO: 11
  Synthetic peptide

SEQ ID NO: 120 - protein, 46 aa, Artificial sequence; identical to SEQ ID NO: 12
  Synthetic peptide

SEQ ID NO: 121 - protein, 53 aa, Artificial sequence; identical to SEQ ID NO: 28
  Synthetic peptide

SEQ ID NO: 122 - protein, 46 aa, Artificial sequence; identical to SEQ ID NO: 17
  Synthetic peptide

SEQ ID NO: 123 - protein, 45 aa, Artificial sequence; identical to SEQ ID NO: 13
  Synthetic peptide

SEQ ID NO: 124 - protein, 39 aa, Artificial sequence; identical to SEQ ID NO: 26
  Synthetic peptide

SEQ ID NO: 125 - protein, 43 aa, Artificial sequence
  Synthetic peptide
KTGVCPELQADQNCTQECVSDSECRDNLKCCSAGCATFCSLPN

SEQ ID NO: 126 - protein, 44 aa, Artificial sequence; identical to SEQ ID NO: 21
  Synthetic peptide

SEQ ID NO: 127 - protein, 54 aa, Artificial sequence; identical to SEQ ID NO: 19
  Synthetic peptide

SEQ ID NO: 128 - protein, 47 aa, Artificial sequence; identical to SEQ ID NO: 23
  Synthetic peptide

SEQ ID NO: 129 - protein, 41 aa, Artificial sequence; identical to SEQ ID NO: 20
  Synthetic peptide

SEQ ID NO: 130 - protein, 41 aa, Artificial sequence; identical to SEQ ID NO: 22
  Synthetic peptide

SEQ ID NO: 131 - protein, 58 aa, Artificial sequence; identical to SEQ ID NO: 14
  Synthetic peptide

SEQ ID NO: 132 - protein, 52 aa, Artificial sequence; identical to SEQ ID NO: 16
  Synthetic peptide

SEQ ID NO: 133 - protein, 38 aa, Artificial sequence; identical to SEQ ID NO: 15
  Synthetic peptide

SEQ ID NO: 134 - protein, 65 aa, Artificial sequence; identical to SEQ ID NO: 30
  Synthetic peptide

SEQ ID NO: 135 - protein, 62 aa, Artificial sequence; identical to SEQ ID NO: 31
  Synthetic peptide

SEQ ID NO: 136 - protein, 71 aa, Artificial sequence
  Synthetic peptide
SPKQRVLKYILEPPPCISAPENCTHLCTMQEDCEKGFQCCSSFCGIVCSSETFQKRNRIKHKGSEVIMPAN

SEQ ID NO: 137 - protein, 58 aa, Artificial sequence
  Synthetic peptide
SPKQRVLKYGLCPPPCISAPENCTHLCTMDEDCEKGFKCCSSFCGIVCSSETFQKRNR

SEQ ID NO: 138 - protein, 184 aa, Homo sapiens
CTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 139 - protein, 194 aa, Homo sapiens
CSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 140 - protein, 198 aa, Homo sapiens
EACSCAPAHPQQHICHSALVIRAKISSEKVVPASADPADTEKMLRYEIKQIKMFKGFEKVKDVQYIYTPFDSSLCCGVKLEANSQKQYLLTGQVLSDGKVFIHLCNYIEPWEDLSLVQRESLNHHYHLNCGCQITTCYTVPCTISAPNECLWTDWLLERKLYGYQAQHYVCMKHVDGTCSWYRGHLPLRKEFVDIVQP

SEQ ID NO: 141 - protein, 188 aa, Homo sapiens
CTCSPSHPQDAFCNSDIVIRAKVVGKKLVKEGPFGTLVYTIKQMKMYRGFTKMPHVQYIHTEASESLCGLKLEVNKYQYLLTGRVYDGKMYTGLCNFVERWDQLTLSQRKGLNYRYHLGCNCKIKSCYYLPCFVTSKNECLWTDMLSNFGYPGYQSKHYACIRQKGGYCSWYRGWAPPDKSIINATDP

SEQ ID NO: 142 - protein, 187 aa, Homo sapiens
CTCSPSHPQDAFCNSDLVIRAKVVGKKEVKEGTFGTLRYEIKQMKMYKGFTKMPDVQYIYTPASWSVCGLKLEVNKYQYLLTGRVLDGKMHITLCNFVEPWDQLSLSQRKGLNYRYHLGCECKIKSCYYIPCFVTSKNECLWTDMLLNFGYPGYQSKHACIRRKGGYCSWYRGWAPPDKSITNATDP

SEQ ID NO: 143 - protein, 112 aa, Artificial sequence
  Synthetic peptide
RKKTFLSVHEVMAVENYAKDSLQWITDQYNKESDDKYHFRIFRVLKVQRQVTDHLEYHLNVEMQWTTCQKPETTNCVPQERELHKQVNCFFSVFAVPWFEQYKILNKSCSSD

SEQ ID NO: 144 - protein, 121 aa, Artificial sequence
  Synthetic peptide
KDPKKNETGVLRKLKPVNASNANVKQCLWFAMQEYNKESEDKYVFLVVKTLQADLQVTNLLEYLIDVEIARSDCRKPLSTNEICAIQENSKLKRKLSCSFLVGALPWNGEFTVMEKKCEDA

SEQ ID NO: 145 - protein, 120 aa, Artificial sequence
  Synthetic peptide
SSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 146 - protein, 122 aa, Artificial sequence
  Synthetic peptide
GSASAQSRTLAGGIHATDLNDKSVQCALDFAISEYNKVINKDEYYSRPLQVMAAYQQIVGGVNYYFNVKFGRTTCTKSQPNLDNCPFHDQPHLKRKAFCSFQINEVPWEDKISILNYKCRKV

SEQ ID NO: 147 - protein, 101 aa, Artificial sequence
  Synthetic peptide
DEWVQRALHFAISEYNKATKDDYYRRPLRVLRARQQTVGGVNYFFDVEVGRTICTKSQPNLDTCAFHEQPELQKKQLCSFEIYEVPWENRRSLVKSRCQES

SEQ ID NO: 148 - protein, 121 aa, Artificial sequence
  Synthetic peptide
SSSKEENRIIPGGIYDADLNDEWVQRALHFAISEYNKATEDEYYRRPLQVLRAREQTFGGVNYFFDVEVGRTICTKSQPNLDTCAFHEQPELQKKQLCSFEIYEVPWEDRMSLVNSRCQEA

SEQ ID NO: 149 - protein, 121 aa, Artificial sequence
  Synthetic peptide
WSPQEEDRIIEGGIYDADLNDERVQRALHFVISEYNKATEDEYYRRLLRVLRAREQIVGGVNYFFDIEVGRTICTKSQPNLDTCAFHEQPELQKKQLCSFQIYEVPWEDRMSLVNSRCQEA

SEQ ID NO: 150 - protein, 119 aa, Artificial sequence
  Synthetic peptide
WHFHEQRDCDEHNVMARYLPATVEFAVHTFNQQSKDYYAYRLGHILNSWKEQVESKTVFSMELLLGRTRCGKFEDDIDNCHFQESTELNNTFTCFFTISTRPWMTQFSLLNKTCLEGFH

SEQ ID NO: 151 - protein, 146 aa, Artificial sequence
  Synthetic peptide
AKLGHFQRWEGFQQKLMSKKNMNSTLNFFIQSYNNASNDTYLYRVQRLIRSQMQERVSHWMLGVHTNSTTDSRQLTTGVEYIVTVKIGWTKCKRNDTSNSSCPLQSKKLRKSLICESLIYTMPWINYFQLWNNSCLEAEHVGRNLR

SEQ ID NO: 152 - protein, 98 aa, Artificial sequence
  Synthetic peptide
MIPGGLSEAKPATPEIQEIVDKVKPQLEEKTNETYGKLEAVQYKTQVVAGTNYYIKVRAGDNKYMHLKVFKSLPGQNEDLVLTGYQVDKNKDDELTGF

SEQ ID NO: 153 - protein, 29 aa, Artificial sequence
  Synthetic peptide
GLFAYKSYRLQVGVGRTCKCQELCFIPNC

SEQ ID NO: 154 - DNA, 1238 bp, Artificial sequence
  2E12 scFv SCC SLPI
aagcttatggattttcaagtgcagattttcagcttcctgctaatcagtgcttcagtcataatgtccagaggagtcgacattgtgctcacccaatctccagcttctttggctgtgtctctaggtcagagagccaccatctcctgcagagccagtgaaagtgttgaatattatgtcacaagtttaatgcagtggtaccaacagaaaccaggacagccacccaaactcctcatctctgctgctagcaacgtagaatctggggtccctgccaggtttagtggcagtgggtctgggacagactttagcctcaacatccatcctgtggaggaggatgatattgcaatgtatttctgtcagcaaagtaggaaggttccatggacgttcggtggaggcaccaagctggaaatcaaacggggtggcggtggatccggcggaggtgggtcgggtggcggcggatctcaggtgcagctgaaggagtcaggacctggcctggtggcgccctcacagagcctgtccatcacatgcaccgtctcagggttctcattaaccggctatggtgtaaactgggttcgccagcctccaggaaagggtctagagtggctgggaatgatatggggtgatggaagcacagactataattcagctctcaaatccagactatcgatcaccaaggacaactccaagagccaagttttcttaaaaatgaacagtctgcaaactgatgacacagccagatactactgtgctcgagatggttatagtaactttcattactatgttatggactactggggtcaaggaacctcagtcaccgtctcctctgatcaggagcccaaatcttctgacaaaactcacacatgtccaccgtgcccatctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagctagcgcttggagccacccgcagttcgaaaaataagcggccgctctaga

SEQ ID NO: 155 - protein, 382 aa, Artificial sequence
  2E12 svFc SCC SLPI
DIVLTQSPASLAVSLGQRATISCRASESVEYYVTSLMQWYQQKPGQPPKLLISAASNVESGVPARFSGSGSGTDFSLNIHPVEEDDIAMYFCQQSRKVPWTFGGGTKLEIKRGGGGSGGGGSGGGGSQVQLKESGPGLVAPSQSLSITCTVSPFSLTGYGVNWVRQPPGKGLEWLGMIWGDGSTDYNSALKSRLSITKDNSKSQVFLKMNSLQTDDTARYYCARDGYSNFHYYVMDYWGQGTSVTVSSDQEPKSSDKTHTCPPCPSGKSFKAGVCPPKKSAQCLRYKKPECQSDWQCPGKKRCCPDTCGIKCLDPVDTPNPTRRKPGKCPCTYGQCLMLNPPNFCEMDGQCKRDLKCCMGMCGKSCVSPVKASAWSHPQFEK

SEQ ID NO: 156 - DNA, 1208 bp, Artificial sequence
  2E12 scFv (5 amino acid linker) SSS SLPI
aagcttatggattttcaagtgcagattttcagcttcctgctaatcagtgcttcagtcataatgtccagaggagtcgacattgtgctcacccaatctccagcttctttggctgtgtctctaggtcagagagccaccatctcctgcagagccagtgaaagtgttgaatattatgtcacaagtttaatgcagtggtaccaacagaaaccaggacagccacccaaactcctcatctctgctgctagcaacgtagaatctggggtccctgccaggtttagtggcagtgggtctgggacagactttagcctcaacatccatcctgtggaggaggatgatattgcaatgtatttctgtcagcaaagtaggaaggttccatggacgttcggtggaggcaccaagctggaaatcaaacggggtggcggtggatcccaggtgcagctgaaggagtcaggacctggcctggtggcgccctcacagagcctgtccatcacatgcaccgtctcagggttctcattaaccggctatggtgtaaactgggttcgccagcctccaggaaagggtctagagtggctgggaatgatatggggtgatggaagcacagactataattcagctctcaaatccagactatcgatcaccaaggacaactccaagagccaagttttcttaaaaatgaacagtctgcaaactgatgacacagccagatactactgtgctcgagatggttatagtaactttcattactatgttatggactactggggtcaaggaacctcagtcaccgtctcctctgatcaggagcccaaatcttctgacaaaactcacacatccccaccgtccccatctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaacaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagctagcgcttggagccacccgcagttcgaaaaataagcggccgctctaga

SEQ ID NO: 157 - protein, 340 aa, Artificial sequence
  2E12 scFv (5 amino acid linker) SSS SLPI
DIVLTQSPASLAVSLGQRATISCRASESVEYYVTSLMQWYQQKPGQPPKLLISAASNVESGVPARFSGSGSGTDFSLNIHPVEEDDIAMYFCQQSRKVPWTFGGGTKLEIKRGGGGSQVQLKESGPGLVAPSQSLSITCTVSGFSLTGYGVNWVRQPPGKGLEWLGMIWGDGSTDYNSALKSRLSITKDNSKSQVFLKMNSLQTDDTARYYCARDGYSNFHYYVMDYWGQGTSVTVSSDQEPKSSDKTHTSPPSPSGKSFKAGVCPPKKSAQCLRYKKPECQSDWQCPGKKRCCPDTCGIKCLDPVDTPNPTRRKPGKCPVTYGQCLMLNPPNFCEMDGQ

SEQ ID NO: 158 - DNA, 1606 bp, Artificial sequence
  2E12 scFv SSS SLPI CH3
aagcttatggattttcaagtgcagattttcagcttcctgctaatcagtgcttcagtcataatgtccagaggagtcgacattgtgctcacccaatctccagcttctttggctgtgtctctaggtcagagagccaccatctcctgcagagccagtgaaagtgttgaatattatgtcacaagtttaatgcagtggtaccaacagaaaccaggacagccacccaaactcctcatctctgctgctagcaacgtagaatctggggtccctgccaggtttagtggcagtgggtctgggacagactttagcctcaacatccatcctgtggaggaggatgatattgcaatgtatttctgtcagcaaagtaggaaggttccatggacgttcggtggaggcaccaagctggaaatcaaacggggtggcggtggatccggcggaggtgggtcgggtggcggcggatctcaggtgcagctgaaggagtcaggacctggcctggtggcgccctcacagagcctgtccatcacatgcaccgtctcagggttctcattaaccggctatggtgtaaactgggttcgccagcctccaggaaagggtctagagtggctgggaatgatatggggtgatggaagcacagactataattcagctctcaaatccagactatcgatcaccaaggacaactccaagagccaagttttcttaaaaatgaacagtctgcaaactgatgacacagccagatactactgtgctcgagatggttatagtaactttcattactatgttatggactactggggtcaaggaacctcagtcaccgtctcctctgatcaggagcccaaatcttctgacaaaactcacacatccccaccgtccccatctggaaagtccttcaaagctggagtctgtcctcctaagaaatctgcccagtgccttagatacaagaaacctgagtgccagagtgactggcagtgtccagggaagaagagatgttgtcctgacacttgtggcatcaaatgcctggatcctgttgacaccccaaacccaaaaggaggaagcctgggaagtgcccagtgacttatggccaatgtttgatgcttaacccccccaatttctgtgagatggatggccagtgcaagcgtgacttgaagtgttgcatgggcatgtgtgggaaatcctgcgtttcccctgtgaaagctggtggtggcggctctgggggtggcggctccggaggcggtgggtctgggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctagcgcttggagccacccgcagttcgaaaaataagcggccgctctaga

SEQ ID NO: 159 - protein, 505 aa, Artificial sequence
  2E12 scFv SSS SLPI CH3
DIVLTQSPASLAVSLGQRATISCRASESVEYYVTSLMQWYQQKPGQPPKLLISAASNVESGVPARFSGSGSGTDFSLNIHPVEEDDIAMYFCQQSRKVPWTFGGGTKLEIKRGGGGSGGGGSGGGGSQVQLKESGPGLVAPSQSLSITCTVSGFSLTGYGVNWVRQPPGKGLEWLGMIWGDGSTDYNSALKSRLSITKDNSKSQVFLKMNSLQTDDTARYYCARDGYSNFHYYVMDYWGQGTSVTVSSDQEPKSSDKTHTSPPSPSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKAGGGGSGGGGSGGGGSGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKASAWSHPQFEK

SEQ ID NO: 160 - protein, 6 aa, Artificial sequence
  Synthetic peptide
GQDPVK

SEQ ID NO: 161 - protein, 5 aa, Artificial sequence
  Synthetic peptide
GGGGS

SEQ ID NO: 162 - protein, 18 aa, Artificial sequence
  Synthetic peptide
DQEPKSCDKTHTCPPCPA

SEQ ID NO: 163 - protein, 18 aa, Artificial sequence
  Synthetic peptide
DQEPKSSDKTHTSPPSPA

SEQ ID NO: 164 - protein, 18 aa, Artificial sequence
  Synthetic peptide
DLEPKSCDKTHTCPPCPA

SEQ ID NO: 165 - protein, 4 aa, Artificial sequence
  Synthetic peptide
CPPC

SEQ ID NO: 166 - protein, 4 aa, Artificial sequence
  Synthetic peptide
CPXC

SEQ ID NO: 167 - protein, 5 aa, Artificial sequence
  Synthetic peptide
CPPCP

SEQ ID NO: 168 - protein, 5 aa, Artificial sequence
  Synthetic peptide
CPXCP

SEQ ID NO: 169 - protein, 9 aa, Artificial sequence
  Synthetic peptide
AWRHPQFGG

SEQ ID NO: 170 - protein, 9 aa, Artificial sequence
  Synthetic peptide
AQRHPQFGG

SEQ ID NO: 171 - protein, 9 aa, Artificial sequence
  Synthetic peptide
SWSHPQFEK

SEQ ID NO: 172 - protein, 242 aa, Artificial sequence
  Synthetic peptide
MKSSGLFPFLVLLALGTLAPWAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKAVEGSGKSFKAGVCPPKKSAQCLRYKKPECQSDWQCPGKKRCCPDTCGIKCLDPVDTPNPTRRKPGKCPVTYGQCLMLNPPNFCEMDGQCKRDLKCCMGMCGKSCVSPVKA

(19) United States
(12) Patent Application Publication
(10) Pub. No.: US 2008/0181892 A1
(43) Pub. Date: Jul. 31, 2008

(54) Binding Domain Fusion Protein

(75) Inventors: Jeffrey A. Ledbetter, Shoreline, WA (US); Martha Susan Hayden-Ledbetter, Shoreline, WA (US); Peter Armstrong Thompson, Bellevue, WA (US); Philip H. Tan, Lynnwood, WA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Trubion Pharmaceuticals, Seattle, WA (US)

(21) Appl. No.: 11/659,904

(22) PCT Filed: Aug. 10, 2005
(86) PCT No.: PCT/US05/28496

§ 371 Date: Sep. 24, 2007

Related U.S. Application Data
(60) Provisional application No. 60/600,755, filed on Aug. 11, 2004.

(51) Int. Cl. A61K 39/00; C07K 16/00; C07H 21/04; C12N 5/10; C12N 15/00

(52) U.S. Cl. 424/134.1; 530/387.3; 536/23.4; 435/326; 435/320.1

(57) ABSTRACT

The present invention relates to constructs and methods for the treatment of diseases, disorders and conditions, including those relating to or involving autoimmune disorders, inflammation, bacterial, fungal, and viral infections, and diseases caused by or involving uncontrolled or abnormal proliferation of cells, including cancer.

### FIELD

The field includes peptides and proteins, the preparation and use of such peptides and proteins and nucleic acids encoding the same, and methods for the prevention and treatment of conditions, diseases and disorders that would be improved, eased, or lessened by the administration of, for example, polypeptide and/or nucleic acid constructs of the invention, including, by way of example, conditions, diseases and disorders including inflammation, infection, and cancer and other conditions, diseases and disorders of uncontrolled cell proliferation.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Many diseases, disorders, and conditions involve inflammation. Inflammation is a complex multifactoral process. Uncontrolled inflammation is damaging to tissues and exacerbates disease symptoms. Diseases, disorders, and conditions that lead to inflammation include, for example, bacterial infections, fungal infections, and viral infections. Diseases, disorders, and conditions that involve unwanted or uncontrolled proliferation of cells often have an inflammation component as well. These include, for example, diseases, disorders, and conditions that involve neovascularization and vascularization, including cancer and other proliferative diseases.

Cytokines form one of the major classes of chemical mediators responsible for initiating, regulating and terminating the inflammatory response. Their synthesis, switch-on, and switch-off mechanisms and their modes of action are tightly regulated in what is referred to as a cytokine network. Early cytokines, such as interleukin-1 (IL-1) and tumor necrosis factor (TNF), are synthesized very quickly, within one hour of the onset of inflammation, or in response to stimuli such as bacterial lipopolysaccharides (LPS). Cytokines set in motion a migration of inflammatory cells such as neutrophils and monocytes, whose function is to eliminate injurious agents and restore homeostasis.

It is thought that inflammatory cells use a variety of proteases/proteinases, including neutrophil and monocyte/macrophage metalloproteases and elastases, to migrate from the vascular space to gain access to an inflammatory site through the interstitium. Sallenave J. R., 2000 Resp. Res., 1:87-92. Studies of arthritis and tumor progression have reported that proteases, in specific circumstances metalloproteinases (MMPs), play a central role extracellular matrix turnover. Hayashi, T. et at., 1997 Hum. Pathol. 28, 1071-1078. While these proteinases play an important function in inflammation, and in numerous other biological processes, there can be undesirable proteinase-associated effects and disorders associated with the proteinases. For example, when the serine protease elastase is secreted or abnormally released at sites of inflammation it can cause severe tissue damage and can damage a broad range of connective tissue components such as elastin, proteoglycans, and collagen. Wolters, P. J., and Chapman, H. A., 2000 Respir. Res. 1, 170-177.

Proteinases are also linked with many diseases and disorders. For example, cysteine proteases have been reported to be implicated in rheumatoid arthritis. Duffy, J. M. et al., 1994 Eur. J Clin. Chem. Clin. Biochem. 32, 429-434. Proteinases have been linked to proliferating diseases such as cancer by playing a role in tissue remodeling and cancer cell invasion. As a specific example, cysteine proteases have been described to play roles in physiological and pathological processes and have been implicated in cancer invasion and metastasis. Mignatti, P., and Rifkin, D. B., 1993 Phsyiol. Rev. 73:161-195. Cysteine proteases have also been reported to be implicated in pulmonary emphysema (Chapman, H. A., et al., 1994 Am. J Respir. Crit. Care Maed. 150:155-159), muscular dystrophy (Takeda, A., et al., 1992 Biochem. J. 288:643-648), and Alzheimer's disease (Marks, N., et al., 1995 Int. J. Peptide Protein Res. 46:306, 313). Lysosomal cysteine protease released from cells degrade extracellular collagen, fibronectin, and laminin, resulting in deleterious effects to the matrix, as reviewed by Wolters, P. J., and Chapman, H. A., 2000 Respir. Res. 1:170-177. Proteinase-3 is a neutral serine proteinase in human polymorphonuclear leukocytes and is found in high levels in Wegnener's granulomatosis, a severe vasculitis in organs such as kidney, nose, and lung. Proteinase-3 upregulation can lead to lung damage and controlling its activity could provide therapy against diseases involving antineutrophil cytoplasmic autoantibodies. Brockman, H. et al., 2002 Arthritis Res. 4:L220-225.

Protein inhibitors of proteinases that function to regulate proteinases and negate their potential injurious effects have also been identified. These inhibitors of proteinases are called “antiproteinases” and have been classified into different families. Based upon their regulation and biological role, antiproteinases have been grouped as “alarm” inhibitors and “systemic” inhibitors. Sallenave J. R., 2000 Resp. Res., 1:87-92.

The alarm type group of proteases have been reported to include two low-molecular-mass proteinase inhibitors of the antileukoprotease (ALP) family, including (i) ALP, also sometimes referred to as secretory leukocyte proteinase inhibitor or SLPI (Stolk, J. and Hiemstra, P., 1998 Molecular Biology of the Lung, vol I: In Emphysema and Infection. Edited by Stockley R A. Basel: Birkhauser, 55-68), and (ii) elastase-specific inhibitor (ESI, also sometimes referred to as elafin or SKALP; Schalkwijk J, et al., 1999 Biochem J 340:569-577).

More recently, the name “trappin” gene family has been proposed to refer to this group of proteins collectively. “Trappin” is an acronym for TRansglutaminase substrate and wAP domain containing ProteIN and denotes the ability of this protein to become trapped in tissues and act as an anchored protein. Schalkwijk, J. et al., supra. Trappin family members have been defined by an N-terminal transglutaminase substrate domain which contains hexapeptide repeats with a consensus sequence of glycine-glutamine-asparagine-proline-valine-lysine (GQDPVK) and a C-terminal inhibitor (WAP) domain that folds into a four-disulphide core. ALP/SLPI is known as trappin-1, while SKALP/elafin is known as trappin-2 because it was the second protein that was identified as a trappin family protein. Schalkwijk, J. et al., supra.

The C-terminal WAP domain, reported to be the active proteinase inhibitory domain of trappins, is characterized as having eight cysteine residues that form four disulfide bonds that function to stabilize the domain. Schalkwijk, J. et al., supra. The three-dimensional structures for trappin-1 and trappin-2 have been solved by X-ray crystallography, and the WAP domains from these proteins share the same distribution of disulphide bridges and a common methionine residue in the inhibitory active center. Grutter, M. G. et al., 1988 EMBO J. 7:345-351; Tsunemi, M., et al., 1996 Biochemistry 35:11570-11576. The polypeptide chain in each WAP domain was reportedly arranged in the form of a stretched spiral, with a regular β-hairpin loop formed by two internal strands of the domain accompanied by two external strands linked by the proteinase binding segment, and each domain included four disulfide bonds. A second domain was said to include a reactive loop having elastase and chymotrypsin binding property, with a scissile bond between Leu72 and Met73. This peptide segment reportedly appeared to have a conformation somewhat similar to other serine protease inhibitors. Grutter, M. G. et al., supra.

The N-terminal transglutaminase substrate domain has been reported to have repeating sequences rich in glutamine and lysine residues. These residues are said to function as acyl donor and acceptor sites, respectively, in the formation of an isopeptide bond. The overall length of this domain varies among species, with human trappins reported to have five repeats of this domain sequence.

Trappin-1 has been said to be the same as SLPI. Thompson, R. C., and Ohlsson, K., 1986 Proc. Natl. Acad. Sci. USA 82:6692-6696; Saheki T., et al., 1992 Biochem. Res. Commun. 185:240-245. Trappin-1/SPL1 has been described as a 11.7 kDa protein, comprising 107 amino acid residues that include 16 cysteine residues that form eight disulfide bridges. SLPI is described as having the shape of a boomerang. According to Grutter et al., supra, the domain boundary of the two halves of SLPI is aspartic acid 52 with residues 1 to 51 (Val) forming the first domain arid residues 53 (Thr) to 107 (Ala) forming the second domain, with few noncovalent contacts between the two domains. The core contains a small number of hydrophobic amino acid residues and contains several polar residues, some of which form hydrogen bonds in electrostatic interactions within the core. Grutter et al., supra. Molecules that may be the same as or similar to trappin-1 have also previously been referred to as sodium/potassium ATPase inhibitor-1 or SPAI-2 (Araki, K., et al., 1989 Biochem. Biophys. Res. Commun. 164:496-502), anti-leucoprotease or ALP (Klasen, E. E., and Kramps, J. A., 1986 Biochem. Biophys. Res. Commun. 128:285-289), human seminal fluid inhibitor or HUSI-I (Schiessler, H., et al., 1976 Hoppe-Seyler's Z. Physiol. Chem. 357:1251-1260; Seemuller, U., et al., 1986 FEBS Lett. 199:43-48), whey acidic protein family member (Drenth, J., et al., 1980 J. Biol. Chem. 255:2652-2655), ALP/HUSI-I (Wingens, M., et al., 1998 J. Invest. Dermatol. 111:996-1002), BSI-ATE (Hochstrasser. K., et al., 1981 Hoppe-Seyer's Z. Physiol. Chem. 362:1369-1375), bronchial mucus inhibitor (BMI), mucus proteinase inhibitor (MPI), and trappin-4 and trappin-5 (Zeeuwen et al., 1997 Biol Chem. 272(33):20471-8).422

The primary structure of porcine SPAI-2 was reported by Kuroki et al., 1995 J. Biol. Chem. 270:22428-22433. It was described as a 187 amino acid protein, including a hydrophobic presequence of 21 amino acids, followed by a sequence of 166 amino acids of which the first 105 amino acids (ending at aspartic acid 126) was designated a prosequence that is removed to yield the mature SPAI-2 sequence containing 61 amino acids (beginning with proline 127). The protein was described as having 16 hexapeptide repeats that are highly homologous to the repetitive sequence in the elafin TGase domain that the authors concluded serves as an anchor to localize elafin covalently to specific sites on extracellular matrix proteins. Kuroki et al., 1995, supra.

SLPI muteins with altered proteinase inhibitory (PI) activity through mutation of the conserved Leu72 residue in hSLPI have been reported to diminish PI activity towards elastase, chymotrypsin and/or trypsin. See Eisenberg, et al., 1990 JBC 265(14): 7976-7981. SLPI muteins with diminished PI activity have been reported to not have pro-metastatic or tumorigenic potential, as opposed to the parent molecule (Devoogt, et al., 2003 Proc Natl Acad Sci. 100(10):5778-82), perhaps related to a deficient ability to protect progranulin from proteolytic digestion (He, Z. and Bateman, A., 2003 J Mol Med. 81(10):600-12), or the analogous ability to protect proepithelin from conversion to epitlielin (Zhu, et al 2002 Cell. 111(6):867-78). Additionally, PI-deficient SLPI variants lose the ability to inhibit NFκB production by pulmonary cells (Mulligan, M., et al., 2000. Am J Pathol. 156(3):1033-9), or by LPS-induced monocytes (Taggart, C., et al., 2002. J Biol Chem. 277(37):33648-53). SLPI muteins retain other properties however, including the inhibition of monocyte prostaglandin H synthase-2, responsible for lower PGE2 and MMP production by LPS or ConA stimulated macrophages (Zhang, et al., 1997 JCI 5:894-900), and inhibition of viral infection of monocytes (McNeely, T., et al., 1997 Blood 90:1141-1149). SLPI thus has multiple functions, some of which are dependent on its PI activity, and others that are not.

Trappin-2, or skin-derived antileukoproteinase, was apparently discovered independently by two different groups. Wiedow, O., et al., 1990 J. Biol. Chem. 265:14791-14795, Schalkwijk, J., et al., 1990 Br. J. Dermatol. 122:631-641. Of the groups working with the protein, one named it “elafin” because it can inhibit elastase and the other named it SKin-derived AntiLeukoProteinase (or “SKALP”) because it was structurally similar to antileucoprotease of the Whey Acid Protein, or “WAP”, family known for its four-disulphide core protein. One difference in SKALP was an N-terminal domain that had no similarity to then-known members of the WAP family. This domain was recognized as a transglutaminase substrate and termed “cementoin”. The TGase substrate sequence in elafin is homologous to a guinea pig seminal vesicle protein that forms part of the vaginal plug. Nara, K., et al., J. Biochem. (Tokyo) 115:441-448, 1994. Mature trappin-1 (SLPI) does not have an amino terminal TGase substrate domain.

Trappin-2 contains a highly variable region that is believed to be the region that interacts with elastase. The three residues comprising this highly variable region are Ala-24, Met-25, and Leu-26. This region in other members of the trappin family may also act as a specificity determinant for proteinases. It has been reported that ten residues of elafin are involved in its interaction with elastase, including the three already identified as the highly variable region of elafin. Trappin-2 was originally identified as a non-SLPI, low-molecular-mass, anti-elastase. Hochstrasser K, et al., 1981 Hoppe-Seyler's Z Physiol Chem 362:1369-1375; Kramps J A, and Klasen E C, 1985 Exp Lung Res 9:151-165; reviewed in Sallenave J-M, et al., supra. Trappin-2 has been reported to be the same as elafin. Wiedow, O., et al., 1990 supra. Molecules that may the same as or similar to trappin-2 have also sometimes been called skin-derived anti-leucoproteinase or SKALP (Schalkwijk, J., et al., 1991 supra), elafin/SKALP, and elastase specific inhibitor (ESI) (Sallenave, J.-M., et al., 1992 Biol. Chem. Hoppe Seyler 373:27-33).

The elafin molecule shows about a 38% homology with the C-terminal half of SLPI, and the active sites of both inhibitors are reportedly similar. Francart, et al., 1997 J. Mol. Biol. 268:666-677. Like SLPI, elafin has a high content of cysteine residues, which are arranged in four disulfide bonds in the C-terminal proteinase-inhibiting region. NMR spectroscopy studies of recombinant elafin (r-elafin) indicated that the protein has a flat core and a flexible amino terminal extremity, with a central twisted hairpin reportedly flanked by two external units. Francart, et al., 1997 supra. According to Francart et al., residues 14 to 57 reportedly assume a disc-like segment said to result in a two-dimensional aspect, with r-elafin assuming a double-stranded, twisted β-sheet formation stabilized by main chain hydrogen bonds. The core B-sheet was said to be linked to an external binding loop by the cysteine 23 to cysteine 49 disulfide bridge. The flexibility and mobility of this loop in solution is said to be similar to that observed in other protease inhibitors, such as bovine pancreatic trypsin inhibitor. Kraumsoe, J. A., et al., 1996 Biochem. 35:9090-9096. Residues 47 to 50 reportedly form a β-turn, and two disulfides (cysteine 14-cysteine 21 and cysteine 28-cysteine 31) reportedly connect the central β-hairpin of r-elafin to both external segments and produce a spiral motif where each external strand runs parallel to its corresponding strand in the central β-sheet. Both external segments were said to be connected by a loop (residues 22-27) that comprise the site with which, for example, elastase interacts. The scissile peptide bond is predicted to be between Ala24 and Met25 according to Francart et al., and this was reportedly confirmed by in a crystallographic structure of elafin complexed with porcine pancreatic elastase. Tsunemi, et al., 1996 Biochem. 35:11570-11576.

Elafin, a low molecular weight inhibitor of elastase, possess a WAP motif and an amino acid sequence substrate for TGase and purified SKALP/elafin is a substrate for transglutaminase crosslinking, Molhuizen, H. O. F., et al., 1992 J. Biol. Chem. 268:12028-12032, which assists in immobilizing the protease inhibitor to epidermal proteins and to cornified envelopes. Elafin is synthesized as a large precursor molecule with distinct biological features including the ability to covalently attach to epidermal proteins. Elafin, is found in the epidermis of several inflammatory skin diseases, but not in normal human epidermis. SKALP/elafin is found in the suprabasal differentiated keratinocytes of psoriatic epidermis. Schwalkjik, J., et al., 1993 J. Invest. Dermatol. 100:390-293.

Trappins are encoded by 2 kilobase single-copy genes that contain three exons. The first exon codes for the 5′ untranslated region, the signal peptide and the first couple of amino acid residues of the protein. The second exon contains the sequence for most of the protein, and the third exon codes for the 3′ untranslated region. There is the a high degree of conservation of intron sequences among the members of the trappin family. The human trappin-2 gene (SKALP) is located on chromosome 20. The two domain structure of those genes in the trappin family is thought to have evolved by exon shuffling because many proteins have the same motif as these trappin genes. Researchers believe that exon shuffling and gene multiplication of the SLPI gene and a group of genes called REST genes created the trapppin genes.

At the genetic level, a locus containing fourteen genes that encode proteins that exhibit homology with WAP domains has been identified on human chromosome 20q12-13.1. Clauss, A., et al., 2002 Biochem. J. 368:233-242. Included among the fourteen genes are elafin, SLPI, human epididymis gene product 4, eppin, and huWAP2. huWAP2 (Accession No. AY037803), with four disulfide core proteins, has also been described as a putative proteinase inhibitor. Lundwall, A., and Clauss, A., 2002 Biochem. Biophys. Res. Commun. 290:452-456. At least three closely related members of the elafin subfamily have been identified and it has been proposed that their genes arose by accelerated evolution. Tamechika et al., 1996 J. Biol Chem. 271:7012-7018. Primary sequence identity between elafin and the carboxyl terminal domain of SLPI has been reported to be about 38 percent. Tamechika et al., supra. Several genes from different species have been discovered that are homologous to SLPI and elafin. Schalkwijk, J., et al., 1999 Biochem. J. 340:569-577; Zeeuwen, P. L. J. M., et al., 1997 J. Biol. Chem. 272, 20471-20478; Furutani, Y., et al., 1998 J. Biochem (Tokyo) 124:491-502.

Proteases inhibited by trappin family proteins have been at least partially characterized. SLPI has been reported to inhibit chymotrypsin and trypsin (Smith, C. E., and Johnson, D. A., 1985 Biochem. J. 225:463-472), human mast cell chymase (Walter, M., et al., 1996 Arch. Biochem. Biophys. 327:81-88), stratum corneum chymotryptic enzyme (Franzke, C. W., et al., 1996 J. Biol. Chem. 271, 21886-21890), human leukocyte elastase (Ying, Q. L., et al., 1994 Biochemistry 33: 5445-5450), human cathepsin G (Smith, C. E., et al., 1985 Biochem. J. 225:463-472), bovine chymotrypsin (Wiedow, O., et al., 1993 Adv. Exp. Med. Biol. 336:61-64), pig chymotrypsin (Smith, C. E., et al., 1985) and tryptase (Beppu, Y., et al., J. Biochem. 121:309-216, 1997). Trappin-2 has been described to inhibit human leukocyte elastase (Ying, Q. L. and Simon, S. R., 1993 Biochemistry 32:1866-1874), pig pancreatic elastase (Wiedow, O., et al., 1993 Adv. Exp. Med. Biol. 336:61-64), and stratum corneum chymotryptic enzyme (Franzke, C. W., et al., 1997 in Proteolysis in Cell Functions, pp. 438-446, IOS Press, Amsterdam).

Expression of trappin-2/elafin, originally found in the skin of patients with psoriasis, seems to be low in normal skin, but can be induced with trauma or irritation. The presence of a signal sequence in elafin indicated that the protein is secreted. During wound healing, expression of elafin is said to be increased because keratinocytes are migrating though an environment of increased activated neutrophils that are secreting proteinases such as elastase and proteinase. See Alkemade, Hans, A. C, et al., (1994) “Demonstration of Skin-Derived Antileukoproteinase (SKALP) and Its Target Enzyme Human Leukocyte Elastase in Squamous Cell Carcinoma,” Journal of Pathology 174:121-129; Alkemade, J. A. C., et al., (1994) “Skalp/elafin is an inducible proteinase inhibitor in human epidermal keratinocytes,” Journal of Cell Science. 107: 2335-42.; Boelsma, Esther, et al. (1998) “Expression of Skin-Derived Antileukoproteinase (SKALP) in Reconstructed Human Epidermis and Its Value as a Marker for Skin Irritation,” Acta Derm. Venereol. 78:107-113; Goselink, Henriette M., et al., (1996) “Colony Growth of Human Hematopoietic progenitor Cells in the Absence of Serum Is Supported by a Proteinase Inhibitor Identified as Antileukoproteinase,” The Journal of Experimental Medicine. 184:1305-12; Kuijpers, Astrid, L., (1996) “SKALP is decreased in pustualr forms of psoriasis. A clue to the pathogenesis of pustule formation?” Archives of Dermatological Research 288:641-7; Molhuizen, Henri O. F., (1995); “Structural, Biochemical, and Cell Biological Aspects of the Serine Proteinase Inhibitor SKALP/Elafin/ESI,” Biological Chemistry Hoppe-Seyler 376:(1)1-7; Schalkwijk, Joost, et al., (1999) “The trappin gene family: proteins defined by an N-terminal transglutaminase substrate domain and C-terminal four-disulphide core,” Biochem. J. 340: 569-577.

The alarm-type protease inhibitors SLPI and elafin may be part of a first wave of local, inducible antiproteinase defense network, i.e., potent locally produced elastase inhibitors having characteristics that allow them to be present first at the onset of inflammation. They have been described to be synthesized and secreted locally at the site of injury in response to primary cytokines such as IL-1 and TNF. Sallenave J. M., 1994 et al., Am J Respir Cell Mol Biol, 11:733-741. Alarm signals such as bacterial LPS, IL-1, TNF, neutrophil elastase and defensins are reported to be able to switch on production of protease inhibitors. Schalkwijk J. et al. 1994, supra; Van Wetering S, et al., 2000 Am J Physiol Lung Cell Mol Physiol 278:L51-L58; Jin F Y, et al., 1998 Infect Immun 66:2447-245. Conversely, anti-inflammatory and remodeling cytokines such as transforming growth factor-β may switch them off (see, e.g., for SLPI, Jaumann F, et al., 2000 Eur Respir J 15:1052-1057).

The systemic-type protease inhibitors, which include al-proteinase inhibitor (Al-Pi) and antichymotrypsin, are reportedly upregulated mainly by a later wave of cytokines such as those of the IL-6 family (e.g., IL-6 and oncostatin M). Sallenave J. M., 2000 Resp. Res, 1:87-92. Interferon-γ was described to inhibit LPS-induced SLPI up-regulation in macrophages, perhaps indicating regulation of SLPI at the interface between innate and adaptive immunity. Jin F. Y., et al., 1997 Cell 88:417-426.

Antiproteinases are also thought to be implicated in modulating immune system functions. For example, SLPI has been described to possibly play a role in signal transduction pathways in monocytes. SLPI has also been reported to suppress the production of monocyte prostaglandin H synthase-2 (PGHS-2) and matrix metalloproteinase (Zhang, Y., et al., J. Clin. Invest. 99:894-900, 1997). The inhibitory effect of SLPI did not necessarily depend on its ability to inhibit protease activity, as muteins of SLPI with significantly lower antiprotease activity were also described to suppress induction of PGHS-2 and matrix metalloptoteinases. The authors concluded that SLPI functions as a potent anti-inflammatory agent by interfering with the signal transduction pathway leading to monocyte matrix metalloproteinase production. Zhang, Y., supra. The addition of recombinant SLPI to human monocytes or the transfection of macrophages with SLPI or elafin has been described to downregulate pro-inflammatory mediators such as TNF and matrix metalloproteinases on stimulation with LPS, for example. Id.; Zhang Y, et al., 1997 J Clin Invest 99:894-900. It may also function to interfere directly (by binding to LPS) or indirectly (with LPS in a feedback fashion) by downregulating nuclear factor-κB function, for example. Lentsch A B, et al., 1999 Am J Pathol 154:239-247.

One potential mode of action for antiproteinases in modulating immune system function has been postulated to be through proteinase 3. Proteinase 3 is present in high concentrations in the human neutrophil (Campbell, E. J., 1999 et al., J. Immunol. 165:3366-3374, 1999), and proteinase 3 activity has been reported to be inhibited by elafin. Sallenave, J.-M., et al., Biol Chem Hoppe Seyler. 373:27-33, 1992. It has also been suggested that SLPI, in addition to its role as a protease inhibitor, has additional modes of action including the upregulation of regenerative genes in lung tissue. Positive regulation of hepatocyte growth factor production in human lung fibroblasts by SLPI has also been reported. Kikuchi et al., 2000 Am. J Respir. Cell Mol. Biol. 23:364-370.

In lung, SLPI has been reported to be produced in vitro by tracheal, bronchial, bronchiolar and type II alveolar cells, and by monocytes, alveolar macrophages and neutrophils. Id.; Sallenave J. M., et al., 1997 J Leukoc Biol 61:695-702. SLPI has also been described to be produced in vivo by tracheal serous glands and bronchiolar Clara cells, and to be associated with elastin fibers in the alveolar interstitium. Stolk and Hiemstra, supra. Outside the lung, it is reportedly secreted in a variety of mucosal sites (leading to its alternative name, mucosal proteinase inhibitor). Stolk and Hiemstra, supra. SLPI has also been reported to be secreted in bronchial and cervical mucus (Ohlsson, K., et al., 1977 Hoppe-Seyler's Z. Physiol. Chem 357(5):583-589), seminal plasma (Schiessler, H., et al., 1976 Hoppe-Seyler's Z. Physiol. Chem. 357:1252-1260), and parotid and submandibular salivary glands (Ohlsson, M., et al., 1983 Hoppe-Seyler's Z. Physiol. 364:1323-1328; Thompson, R. C., and Ohlsson, K., 1986 Proc. Natl. Acad. Sci. USA 83:6692-6696).

As indicated above, proteases and protease inhibitors have a role in disease. Serine proteases have been implicated in respiratory and immune disorders, including the pathophysiology of asthma. Leukocyte serine proteases and mast cells are elevated in the airways of asthmatic patients. Wenzel et al. 1988, Am Rev Respir Dis 137: 1002-1008, Fahy et al., 1995 J Allergy Clin Immunol 95:843-852. SLPI is reportedly increased in patients with acute respiratory distress syndrome (ARDS) (Sallenave, J.-M., et al., 1999 Eur. Respir. J. 14:1029-1036), in patients with pneumonia (Asano, S., et al., 1995 Am. J Respir. Crit. Care Med. 151:1576-1581), and in febrile patients (Duits, L. A., et al., 2003 Clin. Microbiol. Infect. 9:605-613), consistent with the function of SLPI as an alarm inhibitor in an inflammatory response. In ARDS, elafin expression is also reportedly markedly increased in bronchoalveolar lavage fluid compared with control subjects. Sallenave J. M, et al., Eur Respir J 1999, supra.

The presence of elafin has also been described at mucosal sites in many other tissues. Trappin-2 has been reported to be present in bronchial secretions (Sallenave J M, and Ryle A P, 1991 Biol Chem Hoppe-Seyler 372:13-21), and in the skin (Wiedow et al. (1990), supra; Molhuizen et al., 1993 J Biol Chem 268:12028-12032). Elafin is expressed lung cell lines and has been implicated as having a role during inflammation in peripheral lung. One elafin-immunoreactive species (12-14 kD) of elafin was reported to be secreted by A549 lung carcinoma cells, and two elafin-immunoreactive species (12-14 kD, and 6 kD) were reported to be secreted by NCI-H322 lung carcinoma cells. Sallenave, J. M, et al., 1993 Am. J. Respir. Cell Mol. Biol. 8:126-133. The cell lines have features of type II alveolar cells and Clara cells, respectively, and there may be a role during inflammation in peripheral lung for type II alveolar cells in the defense against neutrophil elastase.

Protease inhibitors have been reported to reduce antigen-induced responses in vivo, Clark et al.; 1995 Am J Respir Crit Care Med 152:2076-2083, Fujimoto et al., 1995 Respir Physiol 100:91-100, and SPLI is thought to play a role in several respiratory diseases, such as acute respiratory distress syndrome, asthma, cystic fibrosis, pneumonia (Reid, P. T., and Sallenave, J. M., 2001 Curr. Opin. Invest. Drugs 2, 59-67), and emphysema (Knight, K. R., et al., Respirol. 2, 91-95, 1997). Administration of SLPI was described to be beneficial in an animal model of asthma as determined by (i) an inhibition of leukocyte influx into airways after chronic allergen exposure, (ii) prevention of antigen-induced decrease of tracheal mucus velocity, and (iii) an inhibition of late-phase bronchoconstriction and development of hyperresponsiveness. Wright, C. D., et al., 1999 J. Pharmacol. Exp. Ther. 289:1007-1014.

rSLPI has been reported as protective against inflammatory stimulation. Lucey, E. C., et al., J. Lab. Clin. Med 115:224-232; Vogelmeier, C., et al., J. Clin. Invest. 87:482-488, 1991. Inflammatory stimulation can be induced by intratracheal treatment with human leukocyte elastase. In these studies, intratracheal administration of 3 mg of recombinant SLPI eight hours before administration by intratracheal instillation of 0.25 mg of human neutrophil elastase was described to result in protection against induction of emphysema and secretory cell metaplasia. It was also reported that 59 percent of rSLPI and 44 percent of rSLPI could be recovered by lung lavage one and four hours after administration, respectively, indicating a half-life of approximately two hours. Lucey, E. supra; Vogelmeier et al., supra. SLPI deficient mice (SLPI−/−) have been described to be more susceptible to LPS-induced endotoxin shock than the parent strain (SLPI+/+), Nakamura, A., 2003 et al., J. Exp. Med. 5:669-674, consistent with the notion that SLPI may attenuate excessive inflammatory response and assist in a balanced functioning of innate immunity.

Inflammation is a major factor in many diseases, disorders and conditions. Inflammation plays a major role, for example, in the pathogenesis of cystic fibrosis lung disease. It has been suggested that an underlying cause of cystic fibrosis is that antiproteases are outnumbered by proteases, and that restoring the balance of these two classes of enzymes could prove beneficial. Birrer P., 1995 Respiration 62(Suppl.1):25-8. Unfortunately, studies administering SLPI to CF patients have been hampered by a short half-life and poor accessibility of the recombinant product to diseased areas in cystic fibrosis patients (reviewed in Stolk and Hiemstra, supra). Authors have commented that additional new anti-inflammatory therapies for cystic fibrosis would be beneficial. Oermann, C. M., Curr. Opin. Invest. Drugs 2, 900-996, 2001; Reid, P. T., and Sallenave, J.-M., Curr. Opin. Invest. Drugs 2, 59-67, 2001.

It is reported that bacterial LPS is able to upregulate SLPI production in vitro in macrophages (Jin F Y, et al., 1998 Infect Immun 66:2447-2452), and SLPI and elafin have been reported to have antimicrobial properties in vitro. Schalkwijk J (1999), et al., supra; Simpson A J, et al., 1999 FEBS Lett 452:309-313. SLPI and elafin are reportedly effective against both Gram positive and Gram negative bacteria. For reviews, see Schalkwijk, J., Wiedow, O., and Hirose, S., 1999 Biochem. J. 340:569-577; Sallenave, J.-M., 2000 Respir. Res. 1:87-92; Sallemave. J.-M., 2002 Biochem. Soc. Trans. 30:111-115; Hiemstra, P. S., Biochem. Soc. Trans. 30:116-120, 2002; Tomee, J. F., C., et al., 1998 Thorax 53:114-116; and Rogers, D. F., and Laurent, G. J., 1998 Thorax 53:200-203.

Two novel antibacterial WAP motif proteins SWAM1 and SWAM2 were reportedly cloned from mice. Hagiwara et al., 2003 J. Immunol. 170:1973-1979. Both were described to have a single WAP domain homologous with SLPI and elafin. At a concentration of 10 μM, both SWAM1 and SWAM2 were reported to inhibit growth of E. coli and Staphylococcus aureus by 90 percent. Hagiwara et al., supra. rSLPI has also been reported to be capable of inhibiting pathogenic fungi (at micromolar range concentrations), for example, Aspergillus fumigatus and Candida albicans. Quiescent A. fumigatus was reported to be resistant to rSLPI but to become sensitive to rSLPI when cells were induced to become metabolically active. The amino terminal domain of SLPI is reported to possess antifungal activity. Tomee, J. F. C. et al., 1997 J Infect Dis 176:740-747.

SLPI in oral secretions may also protect against viral infection by, for example, HIV-1. Shine, N., et al., J. Dent. Res. 76:634-640, 1997; Wahl et al., Oral. Dis. 3(Suppl 1):S64-S69, 1997; Shugars, D. E., J. Infect. Dis. 179(Suppl. 3):S431-S435, 1999; Shugars, D. C., et al., Arch. Oral Biol. 44:445-453, 1999. But see Shine et al., Bioorg. Chem. 30:249-263, 2002. There are several reports that rSLPI is capable of inhibiting HIV-1 infection of macrophages and primary T-cells. Wahl, S. M., et al. (1997), supra; Shugars, D. E., J. Infect. Dis. 179(Suppl. 3):S431-S435, 1999; Shugars, D. C., et al. (1999), supra; McNeely, T. B., et al., J. Clin. Invest. 96:456-464, 1995; McNeely, T. B., et al., Blood 90:1141-1149, 1997; Shugars, D. C., et al., Oral. Dis. 3(Suppl.1):70-S72, 1997. It has been reported SLPI did not prevent HIV-1 infection on human T-cell lines, peripheral blood lymphocytes, and primary macrophages (Turpin, J. A., et al., 1996 Antivral Res. 29, 2269-277), and variable protection of macrophages has also been reported (Konopka, K., et al., J. Dent. Res. 78:1773-1776, 1999). In addition to HIV-1, SLPI is capable of inhibiting Sendai and influenza A viruses. Shugars, D. E., 1999 J. Infect. Dis. 179(Suppl.3):S431-S435.

It has been suggested that SLPI might inhibit HIV-1 infection at some step of infection after viral binding to cell surfaces but before reverse transcription. McNeely, T. B., et al., J. Clin. Invest. 96:456-464, 1995; McNeely, T. B., et al., Blood 90:1141-1149, 1997. SLPI may affect molecules on the cell surface that participate in virus entry. It has been reported that rSLPI may interact with another cell surface molecule other than CD4 (the primary receptor for HIV-1) (Wahl, S. M., et al., Oral. Dis. 3(Suppl 1):S64-S69, 1997; McNeely, T. B., et al., J. Clin. Invest. 96:456-464, 1995; McNeely, T. B., et al. (1997), supra; Shugars, D. C., et al., 1997 Oral. Dis. 3(Suppl. 1):S70-S72), consistent with lack of correlation of rSLPI binding to human T-cells that express CD4, CD26, and CCR5 (Konopka, K., et al., 1999 J. Dent. Res. 78:341). In addition to CD4, the primary receptor for HIV-1 entry, other molecules and cofactors also play important roles in the fusion between virus and cell membrane. J. P. Moore, et al., 1999 Curr. Opinion Immunol. 17:551-562, 1997; Berger, E. A., et al., Annu. Rev. Immunol. 17:657-700. Recombinant, renatured SLPI from E. coli reduced infection of differentiated THP-1 cells by HIV-1_{Ba-L}, and it possessed antiviral activity that was not observed with commercially prepared recombinant SLPI. Both sSLPI (SLPI produced from a synthetic gene) and rSLPI (commercially available rSLPI) are reported to have comparable antiprotease activity. Kohno, T., et al., 1990 Met. Enzymol. 185:187-195.

Tissue inhibitors of metalloproteinases (TIMPs) are a family of closely related proteins that were originally described as inhibitors of metalloproteinase enzymes. Stetler-Stevenson, W. G., et al., 1999 Ann Rev Cell Biol. 9:541-573; Stetler-Stevenson, W. G., et al., 1990 J. Biol. Chem. 265:13933-13938. The TIMP gene family comprises TIMP1, TIMP2, TIMP3, and TIMP4. Each exhibits a TIMP motif and possesses a TIMP domain. Tissue inhibitor of metalloproteinase 2 (TIMP2) is a constitutive 24,000 molecular weight protein that contains twelve cysteines that form six disulfide bonds. Nine isoforms of the TIMP motif have been identified. TIMP proteins bind to and irreversibly inactivate extracellular matrix metalloproteinases (MMP), including MMP-1, MMP-2, MMP-3, MMP-7, MMP-8, MMP-9, MMP-10, MMP-13, MMP-14, MMP-15, MMP-16 and MMP-19, and collagenases. For example, TIMP-2 inhibits MMP-2, which digests extracellular matrix and allows the growth and invasion of cancer cells into tissue. Musso, O., et al., 1997 J. Hepatol. 26:593-605. TIMP-4 appears to play a role in tissue remodeling and extracellular matrix hemostasis. Gomez, D. E., et al., 1997 Eur. J Cell. Biol. 74:111-122.

In addition to blocking MMP activity, TIMPs have also been reported to exert growth factor activities that are independent of their MMP inhibitory function. Montgomery A. M., et al. 1994 Cancer Res. 54:5467-5473; Hayakawa T., et al. 1992 FEBS Lett. 298(1):29-32; Stetler-Stevenson, W. G., et al., 1992 FEBS Left. 296:231-234. TIMP-1 and TIMP-2 have also been reported to inhibit tumor growth, invasion by tumor cells, and metastatic spread of tumors, and to inhibit angiogenesis. Valente, P., et al., 1998 Int. J Cancer 75:246-253. TIPM-1 has also been reported to affect germinal center B-cell differentiation by upregulating CD40 and CD23 and downregulating CD77. Guedez et al. 1998 Blood 92:1342-1349. TIMP-1 expression was also reported to regulate IL-10 levels in B cells. Guedez, et al., 2001 Blood 97:1796-1802. Other antimicrobial peptides include defensins, cathelicidins, and histatins. Mammalian antimicrobial peptides are an important component of host defenses at mucosal surfaces. In the lungs, airway surfaces are covered by an airway surface liquid (asl) that is a thin layer of fluid that covers the lumenal surface of the airway. Various components and factors present within the surface liquids that line the airway provide a first line of defense against viruses and bacteria. In addition to SLPI, several antimicrobial factors also present in this fluid are lysozyme, lactoferrin, defensins, and cathelicidins. Niyonsaba, F., et al., 2003 Curr. Drug Targets Inflamm. Allergy 2:224-231; Oppenheim, J. J., et al., 2003 Ann. Rheum. Dis. 62(suppl.2)ii:17-21.

Mammalian defensins have been characterized as being cationic, nonglycosylated, arginine-rich peptides having an approximate molecular mass of 3.5 to 4.5 kDa. Bals R., 2000 Respir Res. 1(3):141-50. They contain six cysteine residues that form three intramolecular disulfide bridges. Leher R., 1991 Cell 64:229-230. Defensins can be divided into three classes: α-defensins, β-defensins, and θ-defensins. Bals R., supra. α-Defensins are about 29-35 amino acid residues in length and contain three disulfide bridges and have a triple-stranded β-sheet structure with a β-hairpin that contains cationic amino acids. β-Defensins are about 36-42 amino acid residues in length. θ-Defensins have a circular structure produced by the post-translational ligation of two truncated α-defensins. Bals R., supra. The α-defensins human neutrophil peptides 1-4 (HNP-1 to HNP-4) are localized in azurophilic granules of neutrophil granulocytes and contribute to the oxygen-independent killing of phagocytized microorganisms. Ganz T., et al., 1985 J Clin Invest 76:1427-1435, Selsted M. E., et al. 1985 J Clin Invest 76:1436-1439.

In addition to their role as antimicrobial agents, these antimicrobial peptide have functions in inflammation, wound repair, and in the regulation of the immune system. Bals R. supra. Human neutrophil defensins have been reported as being cytotoxic to various cell types (Van Wetering S., et al., 1997 Leukoc Biol 62:217-226) as inducing cytokine synthesis in airway epithelial cells (Van Wetering S., et al. 1997 Am J Physiol Lung Cell Mol Physiol 272:L888-L896), monocytes (Territo M. C. et al., 1989 J Clin Invest 84:2017-2020), and T lymphocytes (Chertov O. et al., 1996 J Biol Chem 271:2935-2940), as increasing the release of SLPI from respiratory epithelial cells (Van Wetering S., et al., 2000 Am J Physiol Lung Cell Mol Physiol 278:L51-L58). Also, α-defensins have been reported as being involved in the chemotaxis of monocytes and T cells (Yang D., et al., 2000 J Leukoc Biol. 68:9-14), the modulation of cell proliferation and an antibody response, and the inhibition of complement activation, of fibrinolysis and of the activity of serpin family members (reviewed in Van Wetering S., et al., 1999 J Allergy Clin Immunol 104:1131-1138). Human β-defensins have been identified as potent ligands for the chemokine receptor CCR6, providing a link between innate and acquired immunity. Yang D., et al., 1999 Science 286:525-528. Defensins, in conjunction with antigens, have also been reported to stimulate and enhance Th1-dependent cellular and Th2-dependent humoral cytokine produce and immune responses. Oppenheim, J. J., et al., 2003 Ann. Rheum. Dis. 62(Suppl 2)ii: 17-21.

Antimicrobial peptides of the cathelicidin family have a highly conserved signal sequence and propeptide region, but have substantial divergence in the C-terminal domain containing the mature peptide, which can range in size from about 12 to 80 or more amino acids. Zanetti M., et al., 1995 FEBS Lett 374:1-5. The human cathelicidin LL-37/hCAP-18 is present in myeloid cells as granules, but is also found in inflamed skin tissue where it has been reported to be regulated by inflammatory stimuli. Frohm M., et al., 1997 J Biol Chem 272:15258-15263. CAP 18, in addition to being found in the peroxidase-negative granules of neutrophils, is also present to a lesser extent in subpopulations of lymphocytes and monocytes, in squamous epithelia of the mouth, tongue, esophagus, cervix, vagina, and pulmonary epithelium, in keratinocytes in inflammatory skin diseases, and in the epididymus. Oppenheim, J. J., et al., (2003) supra.

Cystatins, small proteins that reversibly inhibit cysteine proteinases, including papain-like cysteine proteinases, are widely distributed in tissues and body fluids. Abhrahamson, M., 1994 Methods Enzymol. 244:685-700. Normally, cysteine proteinase activity can not be measured in body fluids except in pathogenic conditions exemplified by endotoxin-induced sepsis, metastasizing cancer, rheumatoid arthritis, purelent bronchiectasis, and periodontitis, which indicates a tight enzyme regulation by cystatins is a necessity in the normal state. Ni, J., et al., 1998 J. Biol. Chem. 273:24797-24804.

The cystatin family of proteins is structurally and functionally similar and probably constitutes a single evolutionary protein superfamily. There are at least three different subclassifications or families of cystatins. Cystatins in family I (cystatin family) do not have disulfide bridges and contain approximately 100 amino acid residues. Cystatins in family II (stefin family) have two disulfide bonds and contain approximately 120 amino acid residues. Examples of family II members include without limitation cystatin C, D, S, SN, and SA, all of which are secreted proteins. Cystatins in family III (kininogen family) contain three cystatin-like domains, each of which contain two disulfide bonds and may be glycosylated. The structures of the cystatin-like domains in the family III are homologous with family II cystatin. For Review see Abrahamson M. et al., 2003 Biochem Soc Symp. 70:179-99.

Members of the cystatin superfamily serve a protective function to regulate the activities of proteinases, which otherwise may cause uncontrolled proteolysis and tissue damage. These peptidases play key roles in physiological processes, such as intracellular protein degradation (cathepsins B, H and L), are pivotal in the remodeling of bone (cathepsin K), and may be important in the control of antigen presentation (cathepsin S, mammalian legumain). Moreover, the activities of such peptidases are increased in pathophysiological conditions, such as cancer metastasis and inflammation. Additionally, such peptidases are essential for several pathogenic parasites and bacteria. Thus cystatins not only have capacity to regulate normal body processes and perhaps cause disease when down-regulated, but may also participate in the defence against microbial infections. Abrahamson M. et al., supra.

Cathepsin D is a prognostic indicator of breast cancer, and cathepsin B is a diagnostic marker for the transition from premalignant to malignant in breast cancer. MMP-9 may predict the pathological stage and grade of bladder cancer. MT1-MMP and MMP-2 may predict the pathological stage and grade of prostate cancer. Mutations in cystatins are also associated with disease. For example, mutations in cystatin C, a member of family II, are associated with cerebrovascular amyloidosis. Mutations in cystatin B segregate with a form of progressive myoclonus epilepsy. Pennacchio, L. A., et al., 1996 Science 271:731-1734. There are also data on the ability of cystatins to inhibit viral replication. For example, human cystatin C inhibits replication of herpes simplex virus (Bjoreck, L., et al., 1990 J. Virol. 64:941-943), human cystatin D inhibits replication of corona virsues (Collins, A. R., and Grubb, A., 1998 Oral. Microbiol. Immunol. 13:59-61), and human cystatin A inhibits rhabdovirus-induced apoptosis (Bjorklund, H. V., et al., 1997 J. Virol. 71:5658-5662). Cystatin D may play a protective role against proteinases present in the oral cavity. Freije, J. P., et al., 1993 J. Biol. Chem. 268:15737-15744.

The potential for use of protease inhibitors for therapy yet to be realized. One protease inhibitor, Zemaira™, an alpha-proteinase inhibitor (A₁-PI),was approved by the FDA in 2003, but its indicated use is very specific. Zemaira™ is an intravenous therapy for individuals with A₁-PI deficiency and clinical evidence of emphysema. See, for example, Schalkwijk, J., et al., 1999 Biochem. J. 340:569-577; Reid, P. T., and Sallenave, J. M., 2001 Curr. Opin. Invest. Drugs 2:59-67; Oermann, C. M., 2001 Curr. Opin. Invest. Drugs 2:900-996; Ward, P. A., and Lentsch, A. B., 2002 Mol. Cell. Biochem. 234/235:225-228; Somerville, R. P. T., et al., 2003 Genome Biol. 4:216, 2003.

There are a vast number of diseases and disorders in which the therapeutic agents available for treatment and methods of treatment are unavailable, limited, or inadequate. These diseases and disorders include diseases and disorders of the immune system (e.g., inflammation), infections and infectious diseases, proliferating diseases (e.g., cancers), respiratory disorders (e.g., ARDS), vascular disorders, and other disorders described herein. The opportunities for improvement of the treatment of these diseases are vast, and the stakes are high.

For example, autoimmune disorders are a prevalent and costly type of immune system malfunction. Any one of at least 80 different autoimmune diseases can result when the immune system becomes unregulated and attacks healthy tissue. Autoimmune diseases are on the rise and reportedly affect more than 50 million people in the U.S. In many autoimmune diseases, cell, tissue, joint and organ damage results from the uncontrolled activation of a immense array of inflammatory pathways. Inflammatory disease, including rheumatoid arthritis, lupus, psoriasis, multiple sclerosis and asthma remain a major cause of mortality and morbidity worldwide. Rheumatoid arthritis (RA) is one such chronic inflammatory disease characterized by inflammation of the joints, leading to swelling, pain, and loss of function. RA affects at least an estimated 2.5 million people in the United States. RA is caused by a combination of events including an initial infection or injury, an abnormal immune response, and genetic factors. While autoreactive T cells and B cells are present in RA, the detection of high levels of antibodies that collect in the joints, called rheumatoid factor, is used in the diagnosis of RA.

Current therapy for RA includes many medications for managing pain and slowing the progression of the disease. No therapy has been found that can cure the disease. Medications include nonsteroidal antiInflammatory drugs (NSAIDS) and disease modifying antirheumatic drugs (DMARDS). NSAIDS are useful in benign disease, but fail to prevent the progression to joint destruction and debility in severe RA. Both NSAIDS and DMARDS are, furthermore, associated with significant unwanted side effects. Only one new DMARD, Leflunomide, has been approved in over 10 years. Leflunomide blocks production of autoantibodies, reduces inflammation, and slows progression of RA. However, this drug also causes severe side effects including nausea, diarrhea, hair loss, rash, and liver injury.

Other important diseases without adequate therapy include ocular diseases, and disorders. Age-related macular degeneration (AMD) is a major cause of blindness that affects the central portion of the retina (the macula). Wet AMD is one form of two forms of the condition that involves the formation of neovascular membranes. It is through the leakage and bleeding of these blood vessels that vision loss, which is usually irreversible, occurs. Wet AMD can be further subdivided into classic and occult and it is the classic form that is more threatening to sight. The prevalence of wet AMD has been estimated at 3 per 1000 at age 60-64 years and 117 per 1000 at 90 years and over. Meads C., et al., 2003 Health Technol Assess. 7(9):v-vi, 1-98. In addition to AMD, other ocular diseases, including retinitis pigmentosa, glaucoma, retinal detachment, diabetic retinopathy, and pathological myopia result in apoptotic death of retinal cells. It has been speculated that inhibition of processes that participate in retinal cell apoptosis has been speculated to decrease the number of dead cells and prevent the irreversible loss of visual function associated with some pathologies such as glaucoma. Garcia M, and Vecino E., 2003 Arch Soc Esp Oftalmol. 78(7):351-64.

There is also a clear need for novel agents useful for the treatment cancer. Cancer includes a broad range of diseases, affecting approximately one in four individuals worldwide. Rapid and unregulated proliferation of malignant cells is a hallmark of many types of cancer, including hematological malignancies. The onset of many cancers may be associated with immune system problems. The increase in the incidence of many types of cancer (tumors) in humans with advancement of age may be correlated with a decline in the peak efficiency of the immune system that occurs about 25 years of age. Although patients with a hematologic malignant condition have benefited from advances in cancer therapy in the past two decades, Multani et al., 1998 J. Clin. Oncology 16:3691-3710, and remission times have increased, most patients still relapse and succumb to their disease. Barriers to cure with cytotoxic drugs include, for example, tumor cell resistance and the high toxicity of chemotherapy, which prevents optimal dosing in many patients.

There is a clear need for novel and efficacious molecules for the treatment of the above-noted diseases and disorders. There is also a need for therapeutic molecules for the treatment of these diseases and disorders that have reduced side effects and a higher efficacy. The compositions and methods of the present invention described and claimed herein provide such improved compositions and methods as well as other advantages.

### SUMMARY

The inventions described and claimed herein have many attributes and embodiments including, but not limited to, those set forth or described or referenced in this Summary. The inventions described and claimed herein are not limited to or by the features or embodiments identified in this Summary, which is included for purposes of illustration only and not restriction.

The invention relates, in one aspect, to therapeutic agents and compositions capable of treating, preventing, or suppressing diseases, disorders and conditions relating to the activity or activation of proteases.

The invention relates, in another aspect, to therapeutic agents and compositions capable of treating, preventing, or suppressing diseases, disorders and conditions that would be benefited or ameliorated by anti-proteinase action.

The invention relates, in yet another aspect, to therapeutic agents and compositions capable of treating, preventing, or suppressing diseases and disorders of the immune system.

The invention also relates to therapeutic agents and compositions capable of treating, preventing, or suppressing infections.

The invention further relates to therapeutic agents and compositions capable of treating, preventing, or suppressing proliferating diseases (e.g., tumors and cancers).

The invention also relates to therapeutic agents and compositions capable of treating, preventing, or suppressing respiratory diseases, disorders and conditions, including ARDS.

The invention also relates to therapeutic agents and compositions capable of treating, preventing, or suppressing vascular diseases, disorders and conditions.

The invention also relates to therapeutic agents and compositions capable of treating, preventing, or suppressing inflammation, as well as inflammatory diseases, disorders and conditions.

The invention provides binding domain fusion proteins, compositions comprising, consisting essentially of, or consisting of binding domain fusion proteins, and methods of use of binding domain fusion proteins, including therapeutic methods of treating a patient in need thereof.

In one aspect, the binding domain fusion protein comprises a polypeptide or other agent having a desired biological activity against a protease. The binding domain fusion protein also comprises a binding domain polypeptide capable of binding to a protease-associated molecule, in other words, non-protease molecules to which the binding domain fusion protein can bind and exert activity against a target protease. The desired biological activity can be, for example, any biological activity associated with modulation of the target protease. Protease-associated molecules include but are not limited to enzymes; ligands and receptors involved in signal transduction; molecules involved in inflammation; proteins involved in immune system functions; regulatory proteins including protein kinases and phosphatases; structural proteins; and the like, as well as targets associated with or involved in any one or more of the diseases, disorders, and/or conditions noted herein. Target-associated molecules are generally molecules that are or will be sufficiently close to or otherwise physically associated with protease activity, for example, local protease activity, such that the binding domain fusion protein can inhibit or modulate the target protease.

The protease-associated molecule is generally an protease-associated target for delivery of the binding domain fusion protein to a site of protease activity or expression, and the desired biological activity is modulation of one or more of the activity, expression, or other properties of the protease to be modulated.

In certain embodiments, the protease is one or more of those proteinases described herein or otherwise now known in the art or later discovered. Accordingly, it is an aspect to provide a binding domain fusion protein that comprises, consists essentially of, or consists of, a polypeptide having a binding domain polypeptide capable of binding to a proteinase-associated molecule and a polypeptide having a proteinase inhibition activity. Polypeptides with proteinase inhibition activity include, for example, anti-proteinases and proteinase inhibitor domains having activity against a proteinase.

Proteinase inhibitors and proteinase inhibitor domains, including but not limited to those described herein, may be included in, and used to prepare, the binding domain fusion proteins. Other proteases and other proteinase inhibitor domains now known or later discovered may also be used.

Proteinase inhibitors and protease inhibitor domains may comprise, consist essentially of, or consist of, a polypeptide. Non-polypeptide proteinase inhibitor molecules are also contemplated.

In some embodiments, the invention provides a compound comprising a protease inhibitor molecule connected to one or more immunoglobulin domains. The immunoglobulin domain may be selected, for example, from the group consisting of a CH2CH3, a CH3, a hinge-CH2CH3, a hinge-CH3, a CH1-hinge-CH2CH3, a CH1-hinge-CH3, and C_{L} (constant region of a light chain). In certain embodiments, the immunoglobulin domain is a primate immunoglobulin domain. In still other embodiments, the immunoglobulin domain is a human immunoglobulin domain. In other embodiments, the immunoglobulin domain is an immunoglobulin domain that has been humanized (in whole or in part). In other embodiments the protease inhibitor is a protein.

In certain embodiments, the binding domain fusion protein comprises, consists essentially of, or consists of i) a first polypeptide having or constituting a binding domain polypeptide capable of binding to a protease-associated molecule, and ii) a second polypeptide comprising or constituting a polypeptide (including proteinase inhibitors and protease inhibitor domains) capable of inhibiting said protease.

In another embodiment, the binding domain fusion protein optionally comprises, consists essentially of, or consists of a third polypeptide comprising a connecting region joining the said first and second polypeptides. The connecting region is preferably a polypeptide but need not be.

In certain embodiments, the binding domain polypeptide comprises an immunoglobulin or a portion or variant thereof. For example, the binding domain polypeptide may be a monoclonal antibody or binding portion thereof, including but not limited to, Fab, Fab′, F(ab′)₂ and Fv fragments, a single chain binding protein, single chain Fv (scFv), polypeptides comprising, consisting essentially of, or consisting of an immunoglobulin light chain variable region polypeptide and an immunoglobulin heavy chain variable region polypeptide. Such immunoglobulins or immunoglobulin portions or variants include not only native molecules, but also those that are chimeric or that are humanized (in whole or in part), or otherwise made less immunogenic for human or other use.

In another aspect, the binding domain polypeptide enables the binding domain fusion protein described herein to bind to a selected protease-associated molecule. For example, the binding domain polypeptide can bind to a molecule selected from CD45, CD45 RA, CD45 RO, VEGF. The binding domain polypeptide can bind to a molecule on one or more particular cell types, such as a leukocyte, a T lymphocyte (e.g., CD2, CD3, CD4, CD5, CD6, CD7, CD8, CD25, CD28, CD69, CD154, CD152 (CTLA-4), and ICOS antigens), a helper T cell, a monocyte, a dendritic cell, an immune effector cell, a B cell (e.g., MHC class II, CD19, CD20, CD21, CD22, CD23, CD37 and CD40 antigens). Other binding domain targets include cell surface markers from normal or malignant cells; cytokines (including growth factors and mediators of signal transduction); proteins in the blood or tissues; infectious targets including viral, bacterial, fungal and parasite targets; and intracellular targets, including intracellular protein targets.—Other protease-associated molecules for binding by constructs of the invention include tumor antigens. Examples of tumor antigens that may be targeted by constructs of the invention include Squamous Cell Carcinoma Antigen 1 (SCCA-1; Protein T4-A); Squamous Cell Carcinoma Antigen 2 (SCCA-2); Ovarian carcinoma antigen CA125 (1A1-3B; KIAA0049); Mucin 1 (Tumor-Associated Mucin; Carcinoma-Associated Mucin; Polymorphic Epithelial Mucin; Pem; Pemt; Episialin; Tumor-Associated Epithelial Membrane Antigen; Ema; H23AG; Peanut-Reactive Urinary Mucin; Pum; Breast Carcinoma-Associated Antigen DF3); CTCL tumor antigen se1-1; CTCL tumor antigen se14-3; CTCL tumor antigen se20-4; CTCL tumor antigen se20-9; CTCL tumor antigen se33-1; CTCL tumor antigen se37-2; CTCL tumor antigen se57-1; CTCL tumor antigen se89-1; Prostate-specific membrane antigen; 5T4 oncofetal trophoblast glycoprotein; Orf73 Kaposi's sarcoma-associated herpesvirus; MAGE-C1 (cancer/testis antigen CT7); MAGE-B1 antigen (MAGE-XP antigen; DAM10); MAGE-B2 antigen (DAM6); MAGE-2 antigen; MAGE-4a antigen; MAGE-4b antigen; Colon cancer antigen NY-CO-45; Lung cancer antigen NY-LU-12 variant A; Cancer associated surface antigen; Adenocarcinoma antigen ART1; Paraneoplastic associated brain-testis-cancer antigen (onconeuronal antigen MA2; paraneoplastic neuronal antigen); Neuro-oncological ventral antigen 2 (NOVA2); Hepatocellular carcinoma antigen gene 520; Tumor-associated antigen CO-029; Tumor-associated antigen MAGE-X2; Synovial sarcoma, X breakpoint 2; Squamous cell carcinoma antigen recognized by T cell; Serologically defined colon cancer antigen 1; Serologically defined breast cancer antigen NY-BR-15; Serologically defined breast cancer antigen NY-BR-16; Chromogranin A; parathyroid secretory protein 1; DUPAN-2; CA 19-9; CA 72-4; CA 195; and, L6.

Connecting regions, such as connecting region polypeptides, for example, are those molecules that permit both ends of the molecule to perform their desired functions including, for example, those that permit the targeting or binding domain to interact with its target and permit the inhibitory domain to perform its desired function.

Connecting region polypeptides include, for example, immunoglobulin hinge regions from IgG, IgA, IgE, IgM and IgD. They also include variants of these sequences, including variants that include substitutions or deletions of one or more of the cysteine residues normally found in these immunoglobulin hinge regions.

Proteinase inhibitors and protease inhibitor domains of the binding domain fusion protein include, but are not limited to, for example, trappin polypeptides or portions thereof having proteinase inhibitor activity. Proteinase inhibitors include naturally and non-naturally occurring analogs of trappin polypeptides that have proteinase inhibitor activity.

Proteinase inhibitors also include but are not limited to, for example, SLPI polypeptides, naturally and non-naturally occurring analogs of SLPI polypeptides that have proteinase inhibitor activity, elafin polypeptides, and naturally and non-naturally occurring analogs of elafin polypeptides that have proteinase inhibitor activity.

Additionally, for example, proteinase inhibitors also include, but are not limited to, WAP motif polypeptides having proteinase inhibitor activity, and naturally and non-naturally occurring analogs of such WAP motif polypeptides that have proteinase inhibitor activity.

Proteinase inhibitors also include but are not limited to, for example, TIMP polypeptides having proteinase inhibitor activity, and naturally and non-naturally occurring analogs of TIMP polypeptides that have proteinase inhibitor activity.

Further proteinase inhibitors also include but are not limited to, for example, cystatin polypeptides having proteinase inhibitor activity, naturally and non-naturally occurring analogs of cystatin polypeptides that have proteinase inhibitor activity.

Proteinase inhibitors also include but are not limited to, for example, defensin polypeptides having proteinase inhibitor activity, and naturally and non-naturally occurring analogs of defensin polypeptides that have proteinase inhibitor activity.

Proteases for inhibition by binding domain fusion proteins of the invention include any desired protease. Such proteases include, but are not limited to, for example, intracellular proteases, including caspases; proteases involved in the regulation of complement activation; proteases involved in the regulation of coagulation; proteases involved in the regulation of signal transduction; and, proteases involved in the expression or activity of prostaglandins (e.g., PGHS-2). Other proteases include, but are not limited to, matrix metalloproteinases, elastase, alpha₁-proteinase, proteinase 3, chymotrypsin, trypsin, human mast cell chymase, stratum corneum chymotryptic enzyme, human cathepsin G, bovine chymotrypsin, pig chymotrypsin, tryptase, human leukocyte elastase, pig pancreatic elastase, stratum corneum chymotryptic enzyme. Protease targets also include, but are not limited to, proteinases that have as substrates, for example, elastin, proteoglycans, and collagen.

In another aspect, binding domain fusion proteins may comprise, consist essentially or, or consist of, analogs of a proteinase inhibitor polypeptide or domain. Thus, it is another aspect to provide binding domain fusion proteins that comprise, consist essentially of, or consist of i) a first polypeptide having a binding domain polypeptide capable of binding to a protease-associated molecule; ii) a second polypeptide comprising a connecting region attached to said first polypeptide; and iii) a third polypeptide comprising an analog of a proteinase inhibitor capable of inhibiting said protease. In certain other embodiments, the proteinase inhibitor analog has a proteinase inhibition activity that is reduced. Such proteinase inhibitor analogs, for example, may have selective amino acid deletions, insertions, or substitutions in comparison to a proteinase inhibitor polypeptide described or referenced herein or otherwise now known or later discovered.

Certain embodiments of binding domain fusion proteins have one or more than one TGase motif, for example between about three and about fifteen TGase motifs (e.g. Gly-Gln-Asp-Pro-Val-Lys), between about four and about ten TGase motifs, or one, two, three, four, five, one or two, one to five, or more than five TGase motifs. An exemplary TGase motif comprises, consists essentially of, or consists of, for example, the amino acid sequence Gly-Gln-Asp-Pro-Val-Lys. Accordingly, it is another aspect to provide binding domain fusion proteins that comprises, consists essentially of, or consists of i) a first polypeptide having a binding domain polypeptide capable of binding to a protease or a protease-associated molecule; ii) a second polypeptide comprising, consisting essentially of, or consisting of, a proteinase inhibitor domain; and iii) a third polypeptide comprising, consisting essentially of, or consisting of, one or more TGase motifs. Optionally, the binding domain fusion protein may include one or more additional molecules comprising, consisting essentially of, or consisting of, a connecting region linking one or more of these polypeptides, for example, linking the first polypeptide and second polypeptides, linking the second and third polypeptides, and/or linking the first and third polypeptides. These polypeptides may be in any desired order that retains the desired functional activity or activities of the binding domain fusion protein and/or any of its components. Connecting regions include those described herein.

In another aspect, the TGase motif acts as a substrate for transglutaminase and, by promoting transglutaminase cross-linking, acts as an anchoring motif for the binding domain fusion protein.

Certain embodiments of binding domain fusion proteins may also comprise one or more dimerization domains, for example one, two, three, one to five, or five or more dimerization domains. These include any sequence or molecule that allows two or more binding domain fusion proteins to associate, either covalently or noncovalently.

In exemplary embodiments, a dimerization domain comprises an immunoglobulin hinge domain or variant or analog, including, for example, those described herein. In other embodiments, a dimerization domain comprises an immunoglobulin CH2CH3 domain or an immunoglobulin CH3 domain or analog, including those described herein. Such regions include IgG CH2CH3 domains or CH3 domains or analogs thereof. Other immunoglobulins, including but not limited to IgA immunoglobulins, may be used to construct CH2CH3 domains or CH3 domains or analogs thereof. In preferred embodiments the dimerization domains are primate dimerization domains. In other embodiments in which the primate dimerization domain is not a wild-type or naturally occurring molecule, the dimerization domains are prepared from or derived from primate dimerization domains. In more preferred embodiments the dimerization domains are human (or humanized, in whole or in part) dimerization domains. In other embodiments in which the human dimerization domain is not a wild-type or naturally occurring molecule, the dimerization domains are prepared from or derived from human dimerization domains.

Certain embodiments of binding domain fusion proteins, for example, comprise a connecting region polypeptide. Human polypeptides and polypeptides derived or prepared from human polypeptides are preferred as connecting region polypeptides. Connecting region polypeptides may include, for example, polypeptides comprising, consisting essentially of, or consisting of, a peptide or polypeptide spacer from about 15 to about 115 amino acids in length; from about 10 to about 50 amino acids in length; from about 15 to about 35 amino acids in length; from about 18 to about 32 amino acids in length; from about 5 to about 15 amino acids in length; or any other desired number of amino acids. In other certain embodiments, the connecting region comprises, consisting essentially of, or consisting of, a dimerization domain. In certain embodiments, the connecting region comprises a naturally-occurring or altered immunoglobulin hinge or hinge-acting polypeptide. An immunoglobulin hinge region polypeptide may comprise, consist essentially or, or consist of, for example, any hinge or hinge-acting peptide or polypeptide that occurs naturally, for example, a naturally occurring hinge region selected from a human hinge or portion thereof; human IgG hinge or a portion thereof; human IgA hinge or a portion thereof; human IgE hinge or a portion thereof; camelid hinge region or a portion thereof; IgG1, IgG2 or IgG3 llama hinge region or portion thereof; nurse shark hinge region or portion thereof; and spotted ratfish hinge region or a portion thereof. In other certain embodiments, the connecting region comprises, consist essentially of, or consist of, by way of example and not limitation, an IgG1, IgG2, IgG3 or IgG4 hinge region having less cysteine residues than occurring naturally in these hinge regions, for example, those hinge regions that normally have three cysteine residues that have been altered to have zero, one, or two cysteine amino acid residues; a human IgG hinge region having zero, one, or two cysteine amino acid residues; a wild type human IgG1 immunoglobulin hinge region; a hinge region, including a immunoglobulin hinge region, comprising a glycosylation site; a hinge region, including a immunoglobulin hinge region, having no cysteine residues that are capable of forming disulfide bonds; a hinge region, including a immunoglobulin hinge region, comprising one cysteine residue; a hinge region comprising a mutated or otherwise altered wild-type immunoglobulin hinge region polypeptide comprising no more than one cysteine residue; a hinge region, including a immunoglobulin hinge region, that is altered so that said protein has a reduced ability to dimerize; a hinge region, including a immunoglobulin hinge region, with three cysteine residues and one proline residue wherein one or more of said cysteine residues has been deleted or substituted and said proline reside has been substituted or deleted; a hinge region that comprises a mutated or otherwise altered wild-type immunoglobulin hinge region polypeptide comprising first, second, and third cysteine residues wherein the first cysteine reside is N-terminal to the second cysteine and the second cysteine is N-terminal to the third cysteine and the first cysteine residue is substituted or deleted. This is not an exhaustive list.

Certain embodiments of the binding domain fusion proteins comprise a naturally occurring or altered immunoglobulin constant region domain. Accordingly, it is another aspect to provide binding domain fusion proteins that comprise, consists essentially of, or consist of: i) a first polypeptide comprising, consisting essentially of, or consisting of a binding domain polypeptide capable of binding to a protease or protease-associated molecule; ii) a second polypeptide comprising a connecting region attached to said first polypeptide; iii) a third polypeptide comprising, consisting essentially of, or consisting of a proteinase inhibitor domain; and iv) a fourth polypeptide comprising, consisting essentially of, or consisting of, an immunoglobulin constant region or portion thereof. In certain embodiments, the immunoglobulin constant region embodiments comprises, consists essentially of, or consists of, an immunoglobulin CH3 region, including CH3 analogs. In other embodiments, the binding domain fusion proteins comprise other immunoglobulin constant regions or analogs, including those described herein. In another aspect, the immunoglobulin constant region domain of the binding domain fusion proteins is capable of mediating immunological effector functions including, for example, one or more of complement dependent cytotoxicity, antibody dependent cellular cytotoxicity, FcR binding, protein A binding, and decreasing a number of target cells.

In certain other further embodiments a binding domain fusion protein is provided that comprises, consists essentially of, or consists of: i) a first polypeptide having a binding domain polypeptide capable of binding to a protease or protease-associated molecule; ii) a second polypeptide comprising a connecting region attached to said first polypeptide; iii) a third polypeptide comprising, consisting essentially of, or consisting of, a proteinase inhibitor or proteinase inhibitor domain; and iv) one or more dimerization domains, wherein said first polypeptide is N-terminal to said second polypeptide and said second polypeptide is N-terminal to said proteinase inhibitor domain, wherein said proteinase inhibitor domain comprises one or more WAP domains, and wherein said one or more WAP domains is N-terminal to said one or more dimerization domains.

In certain other further embodiments a binding domain fusion protein is provided that comprises, consists essentially of, or consists of: i) a first polypeptide having a binding domain polypeptide capable of binding to a protease or protease-associated molecule; ii) a second polypeptide comprising a connecting region attached to said first polypeptide; iii) a third polypeptide comprising, consisting essentially of, or consisting of, a proteinase inhibitor or proteinase inhibitor domain; and iv) one or more dimerization domains, wherein said first polypeptide is N-terminal to said second polypeptide and said second polypeptide is N-terminal to said one or more dimerization domains, and wherein said one or more dimerization domains is N-terminal to said proteinase inhibitor or proteinase inhibitor domain, and wherein said proteinase inhibitor domain comprises one or more WAP domains.

In certain other further embodiments a binding domain fusion protein is provided that comprises, consists essentially of, or consists of: i) a first polypeptide having a binding domain polypeptide capable of binding to a protease or protease-associated molecule; ii) a second polypeptide comprising a connecting region attached to said first polypeptide; iii) a third polypeptide comprising, consisting essentially of, or consisting of, a proteinase inhibitor or proteinase inhibitor domain; iv) one or more dimerization domains, and v) one or more TGase domains, wherein said first polypeptide is N-terminal to said second polypeptide and said second polypeptide is N-terminal to said proteinase inhibitor or proteinase inhibitor domain, wherein said proteinase inhibitor or proteinase inhibitor domain comprises one or more WAP domains, and wherein said one or more WAP domains is N-terminal to said one or more dimerization domains, and wherein said one or more dimerization domains is N-terminal to said one or more TGase domains.

In certain other further embodiments a binding domain fusion protein is provided that comprises, consists essentially of, or consists of: i) a first polypeptide having a binding domain polypeptide capable of binding to a protease or protease-associated molecule; ii) a second polypeptide comprising a connecting region attached to said first polypeptide; iii) a third polypeptide comprising a proteinase inhibitor or proteinase inhibitor domain; iv) one or more dimerization domains; and v) one or more TGase domains, wherein said first polypeptide is N-terminal to said second polypeptide and said second polypeptide is N-terminal to said proteinase inhibitor domain, wherein said proteinase inhibitor domain comprises one or more WAP domains, and wherein said one or more WAP domains is N-terminal to said one or more TGase domains, and wherein said TGase domain is N-terminal to said one or more dimerization domains.

In another aspect, certain constructs are provided that preferably do not have binding domains (e.g. immunoglobulin variable regions). These embodiments may, for example, comprise, consist essentially or, or consist of, two domains, such as a proteinase inhibitor domain and an immunoglobulin constant region domain. An example of this type of construct is SLPI-CH2CH3 or a SLPI analog —CH2CH3. In certain further embodiments, other members of the trappin family may be fused or otherwise joined to an immunoglobulin constant region domain. These constructs are useful in various methods of treatment described herein.

Also provided are binding domain fusion proteins and compositions thereof having antibacterial activity. Methods of treating a patient having a bacterial infection comprising administering an effective antibacterial amount of a binding domain fusion protein are also provided.

Also provided are binding domain fusion proteins and compositions thereof having anti-inflammatory activity. Methods of treating a patient having an inflammatory disorder comprising administering an effective anti-inflammatory amount of a binding domain fusion protein are also provided.

Also provided are binding domain fusion proteins and compositions thereof having anti-viral activity, including, for example, binding domain fusion proteins and compositions thereof that are effective for the treatment of an HIV infection in a patient. Methods of treating a patient having a viral infection, for example, an HIV infection, comprising administering an effective anti-viral or anti-HIV amount of a binding domain fusion protein are also provided.

Also provided are binding domain fusion proteins and compositions thereof that are effective for the treatment of a pulmonary or lung disorder in a patient. Methods of treating a pulmonary or lung disorder in a patient comprising administering an effective amount of a binding domain fusion protein are also provided. Further provided are binding domain fusion proteins and compositions thereof that are effective for the treatment of a pulmonary or lung inflammation in a patient. Methods of treating pulmonary or lung inflammation in a patient comprising administering an effective amount of a binding domain fusion protein are also provided.

Also provided are binding domain fusion proteins and compositions thereof that are effective for the treatment of a vascular disorder in a patient. Methods of treating vascular disorders in a patient comprising administering an effective amount of a binding domain fusion protein are also provided.

Also provided are binding domain fusion proteins and compositions thereof that are effective for the treatment of an ophthalmic disease or disorder in a patient. Methods of treating an ophthalmic disease or disorder in a patient comprising administering an effective amount of a binding domain fusion protein are also provided. In another aspect binding domain fusion proteins and compositions thereof are provided that are effective for the treatment of age related macular degenerative disease in a patient. Methods of treating of age related macular degenerative disease in a patient comprising administering an effective amount of a binding domain fusion protein are also provided.

These and other aspects and embodiments of the inventions described and claimed herein will be apparent from and throughout the application and claims, all of which shall be considered to be a part of the written description thereof.

### BRIEF DESCRIPTION OF THE DRAWINGS

FIG. 1. Pig SPAI-2, exon 1. The cDNA of Sus scrofa (pig) for SPAI-2, exon 1 (ACCESSION D17754) was translated into amino acids. The arrow pointing upwards indicates the site of prosequence cleavage between aspartic acid and proline. Alternating stretches of six amino acids are underlined in the amino terminal region of the protein. These homologous repeating units are substrates for TGase crosslinking.

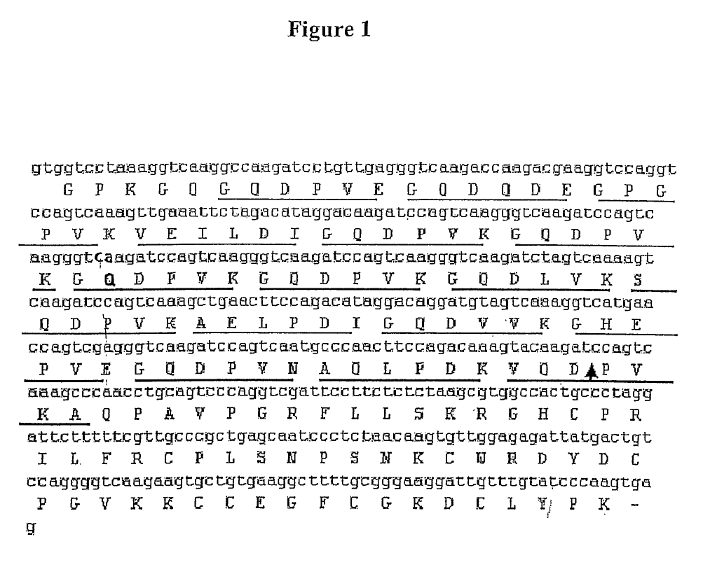

FIG. 2. Homology of amino and carboxy terminal domains of human SLPI. The amino acid sequence for human secretory leukocyte protease inhibitor (ATCC Accession No. AAH20708) is aligned to show the location of the amino and carboxy terminal domains and the position of the conserved cysteine residues.

FIG. 3. provides a schematic illustration of one type of binding domain fusion protein, showing an organization of a synthetic gene encoding functional units of one binding domain and one protease inhibition domain. Spacers (illustrated by black bars) are units that may not have a function other than to separate functional units and allow full biological activity of functional units to be achieved. Each spacer is an optional feature of the binding domain fusion protein.

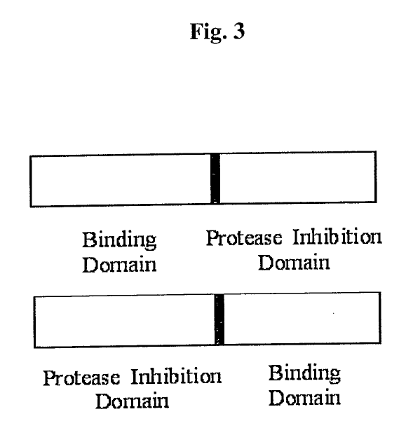

FIG. 4. provides a schematic illustration of another of the various types of binding domain fusion protein, showing an organization of a binding domain fusion protein with combinations of one binding domain, one proteinase inhibition domain, and one dimerization domain.

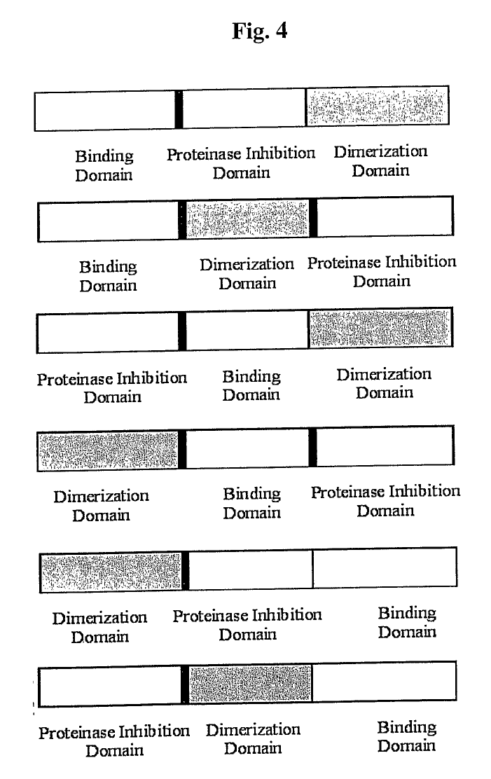

FIG. 5. provides a schematic illustration of another of the various types of binding domain fusion proteins, showing the organization of a binding domain fusion protein comprised of WAP, TIMP, or cystatin domains and one dimerization domain.

FIG. 6. indicates homology among human WAP domains. ATCC Accession Numbers of the proteins from which the WAP domains were extracted are provided at the left side of the Figure. The connected arrows at the top of the figure show the disulfide-bonding pattern.

FIG. 7. indicates homology among human TIMP domains. The TIMP domains from TIM1_HUMAN (ATCC Accession Number P01033), TIM2_HUMAN (ATCC Accession Number P16035), TIM3_HUMAN (ATCC Accession Number P35625), and TIM4_HUMAN (ATCC Accession Number PQ9937) are aligned to illustrate the overall homology and cysteine homology.

FIG. 8. indicates homology among human cystatin domains. The ATCC Accession Numbers of the proteins from which the cystatin domains were extracted are reported at the left side of the Figure.

FIG. 9. provides a schematic illustration of another of the various types of binding domain fusion proteins, showing an organization of functional units of a synthetic gene that encodes a binding domain fusion protein comprised of domains of variable H and L chain in both orientations, whey acid proteins(s), and a CH3-type dimerization domain.

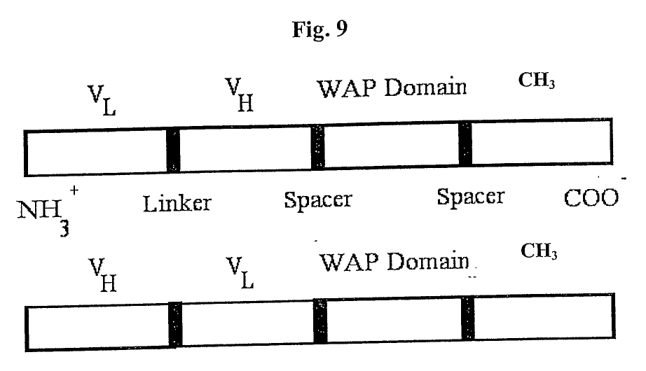

FIG. 10. provides a schematic illustration of another of the various types of binding domain fusion proteins, showing three classes of a binding domain fusion protein comprised of both orientations of variable H and L chains in a binding domain, immunoglobulin hinge dimerization domain, WAP motif from a SLPI proteinase inhibition domain, and a CH3 dimerization domain. Strep tags are included the illustrated embodiments.

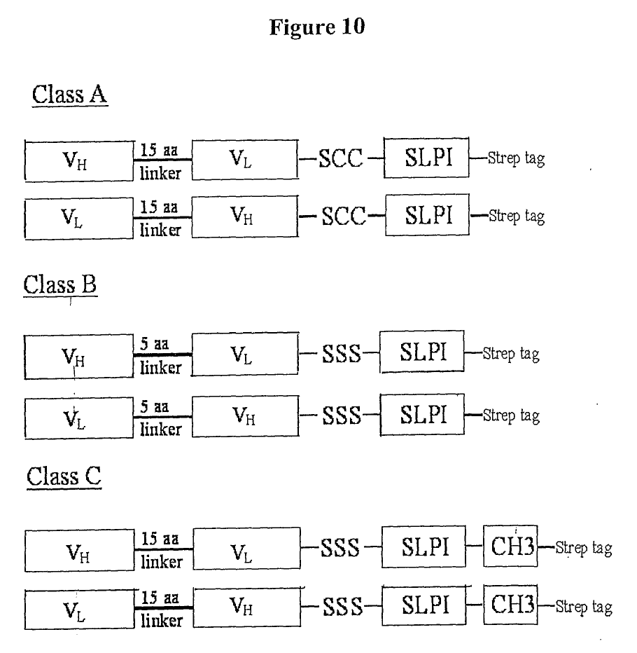

FIG. 11 illustrates the nucleic acid and protein sequence of an anti-CD28 (2E12) construct CD28 scFv-SCC-SLPI. The SCC hinge SLPI strep tag was generated by PCR by adding N-terminal to C-terminal the SLPI gene, the SCC hinge, and the strep tag. The 2E12 scFv with 15 amino acid linker was assembled with the SCC hinge SPLI tag using the BciI restriction site to give the 2E12 scFv-SCC-SPLI. The sequence was confirmed by DNA sequencing.

FIG. 12. illustrates the nucleic acid and protein sequence of an anti-CD28 (2E12) construct CD28 scFv-(5aa linker)-SSS-SLPI. The SSS hinge SLPI strep tag was generated by PCR by adding N-terminal to C-terminal the SLPI gene, the SSS hinge, and the strep tag. The 2E12 scFv with 5 amino acid linker was assembled with the SSS hinge SPLI tag to give the 2E12 scFv-(5aa linker)-SSS-SLPI. The sequence was confirmed by DNA sequencing.

FIG. 13. illustrates the nucleic acid and protein sequence of an anti-CD28 (2E12) construct CD28 scFv-SSS-SLPI-CH3. The SSS hinge SLPI CH3 strep tag was generated by creating SSS hinge SPLI and CH3 strep tag fragments through PCR and fusing them by overlap extension of the two fragments. The 2E12 scFv with 15 amino acid linker was assembled with SSS SLPI CH3 to give 2E12 CD28 scFv-SSS-SLPI-CH3. The sequence was confirmed by DNA sequencing.

FIG. 14. illustrates the binding activity of 2E12-SLPI conjugates. The mammalian vectors harboring different conjugate genes (scFv-SCC-SLPI, scFv(5aa linker)-SSS-SLPI and scFv-SSS-SLPI-CH3) were transfected into COS cells and the supernatants collected. The supernatants were then incubated with CD28-CHO cells, washed, and then probed with goat anti-SLPI followed by rabbit anti-goat FITC. FACS analysis shows that we have made anti CD28 scFv-SLPI conjugates that have a binding domain capable of binding the CD28-CHO cells and a C-terminal domain that can be detected by anti-SLPI antibody.

FIG. 15. illustrates the effect of SLPI Ig and 2E12 scFv-SLPI conjugates on elastase protease activity. Both the 2E12-SLPI conjugates (scFv-SLPI and scFv-SLPI-CH3) and SLPI Ig inhibited elastase proteolytic activity in a dose dependent fashion.

FIG. 16. illustrates the effect of 2E12 SMIP on CD3 blast PBMC proliferation.

### DETAILED DESCRIPTION

The practice of the present invention may employ various conventional techniques of molecular biology (including recombinant techniques), microbiology, cell biology, biochemistry, nucleic acid chemistry, and immunology, which are within the skill of the art. Such techniques are explained fully in the literature, and include but are not limited to, by way of example only, MOLECULAR CLONING: A LABORATORY MANUAL, second edition (Sambrook et al., 1989) and MOLECULAR CLONING: A LABORATORY MANUAL, third edition (Sambrook and Russel, 2001), jointly and individually referred to herein as “Sambrook”; OLIGONUCLEOTIDE SYNTHESIS (M. J. Gait, ed., 1984); ANIMAL CELL CULTURE (R. I. Freshney, ed., 1987); HANDBOOK OF EXPERIMENTAL IMMUNOLOGY (D. M. Weir & C. C. Blackwell, eds.); GENE TRANSFER VECTORS FOR MAMMALIAN CELLS (J. M. Miller & M. P. Calos, eds., 1987); CURRENT PROTOCOLS IN MOLECULAR BIOLOGY (F. M. Ausubel et al., eds., 1987, including supplements through 2001); PCR: THE POLYMERASE CHAIN REACTION, (Mullis et al., eds., 1994); CURRENT PROTOCOLS IN IMMUNOLOGY (J. E. Coligan et al., eds., 1991); THE IMMUNOASSAY HANDBOOK (D. Wild, ed., Stockton Press NY, 1994); BIOCONJUGATE TECHNIQUES (Greg T. Hermanson, ed., Academic Press, 1996); METHODS OF IMMUNOLOGICAL ANALYSIS (R. Masseyeff, W. H. Albert, and N. A. Staines, eds., Weinheim: VCH Verlags gesellschaft mbH, 1993), Harlow and Lane (1988) ANTIBODIES, A LABORATORY MANUAL, Cold Spring Harbor Publications, New York, and Harlow and Lane (1999) USING ANTIBODIES: A LABORATORY MANUAL Cold Spring Harbor Laboratory Press, Cold Spring Harbor, N.Y. (jointly and individually referred to herein as Harlow and Lane), Beaucage et al. eds., CURRENT PROTOCOLS IN NUCLEIC ACID CHEMISTRY John Wiley & Sons, Inc., New York, 2000); and Agrawal, ed., PROTOCOLS FOR OLIGONUCLEOTIDES AND ANALOGS, SYNTHESIS AND PROPERTIES Humana Press Inc., New Jersey, 1993).

### Definitions

Before further describing the inventions in general and in terms of various nonlimiting specific embodiments, certain terms used in the context of the describing the invention are set forth. Unless indicated otherwise, the following terms have the following meanings when used herein and in the appended claims. Those terms that are not defined below or elsewhere in the specification shall have their art-recognized meaning.

The terms “allele” or “allelic sequence,” as used herein, refer to a naturally-occurring alternative form of a gene encoding a polypeptide (i.e., a polynucleotide encoding an binding domain fusion protein). Alleles often result from mutations (i.e., changes in the nucleic acid sequence), and sometimes produce altered and/or differently regulated mRNAs or polypeptides whose structure and/or function may or may not be altered. Common mutational changes that give rise to alleles are generally ascribed to natural deletions, additions, or substitutions of nucleotides that may or may not affect the encoded amino acids. Each of these types of changes may occur alone, in combination with the others, or one or more times within a given gene, chromosome or other cellular polynucleotide. Any given gene may have no, one or many allelic forms. As used herein, the term “allele” refers to either or both a gene or an mRNA transcribed from the gene.

An “amino acid” is a molecule having the structure wherein a central carbon atom (the “alpha (α)-carbon atom”) is linked to a hydrogen atom, a carboxylic acid group (the carbon atom of which is referred to as a “carboxyl carbon atom”), an amino group (the nitrogen atom of which is referred to as an “amino nitrogen atom”), and a side chain group, R. In the process of being incorporated into a protein, an amino acid loses one or more atoms of its amino and carboxylic groups in a dehydration reaction that links one amino acid to another. As a result, when incorporated into a protein, an amino acid is often referred to as an “amino acid residue.” An amino acid may be derivatized or modified before or after incorporation into a protein (for example, by glycosylation, by formation of cysteine through the oxidation of the thiol side chains of two non-contiguous cysteine amino acid residues, resulting in a disulfide covalent bond that frequently plays an important role in stabilizing the folded conformation of a protein, etc.). An amino acid may be one that occurs in nature in proteins, or it may be non-naturally occurring (i.e., is produced by synthetic methods such as solid state and other automated synthesis methods). Examples of non-naturally occurring amino acids include α-amino isobutyric acid, 4-amino butyric acid, L-amino butyric acid, 6-amino hexanoic acid, 2-amino isobutyric acid, 3-amino propionic acid, ornithine, norleusine, norvaline, hydroxproline, sarcosine, citralline, cysteic acid, t-butylglyine, t-butylalanine, phenylylycine, cyclohexylalanine, β-alanine, fluoro-amino acids, including beta and gamma amino acids, designer amino acids (for example, β-methyl amino acids, α-methyl amino acids, Nα-methyl amino acids), and amino acid analogs in general. Amino acid analogs refer to compounds that have the same basic chemical structure as a naturally occurring amino acid, i.e., an alpha-carbon that is bound to a hydrogen, a carboxyl group, an amino group, and an R group, for example, but have modified R groups (for example, norleucine) or modified peptide backbones, while retaining the same basic chemical structure as a naturally occurring amino acid. Amino acid mimetics refers to chemical compounds that have a structure that is different from the general chemical structure of an amino acid, but that generally function in a manner similar to a naturally occurring amino acid.

In addition to its substituent groups, two different enantiomeric forms of each amino acid exist, designated D and L. In mammals, only L-amino acids are incorporated into naturally occurring proteins, although the invention contemplates proteins incorporating one or more D- and L-amino acids, as well as proteins comprised of just D- or just L-amino acid residues.

Herein, the following abbreviations may be used for the following amino acids (and residues thereof): alanine (Ala, A); arginine (Arg, R); asparagine (Asn, N); aspartic acid (Asp, D); cysteine (Cys, C); glycine (Gly, G); glutamic acid (Glu, E); glutamine (Gln, Q); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val, V).

The term “amino acid sequence” refers to an oligopeptide, peptide, polypeptide, or protein sequence, a fragment of any of these, and to naturally occurring or synthetic molecules, as well as to electronic or other representations of foregoing suitable for use in conjunction with a computer, for example.

The term “antibody” is used in the broadest sense, and specifically covers monoclonal antibodies (including full length monoclonal antibodies), polyclonal antibodies, multispecific antibodies (e.g., bispecific antibodies), antibody fragments (e.g., Fab, F(ab′)₂ and Fv), and antibody derivatives (e.g. recombinant or synthetic) so long as they exhibit the desired biological activity. Antibodies (Abs) and immunoglobulins (Igs) are glycoproteins having the same structural characteristics. While antibodies exhibit binding specificity to a specific antigen, immunoglobulins include both antibodies and other antibody-like molecules that lack antigen specificity. Polypeptides of the latter kind are, for example, produced at low levels by the lymph system and at increased levels by myelomas.

Native antibodies and immunoglobulins are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82, 4592-4596 (1985)). Five human immunoglobulin classes are defined on the basis of their heavy chain composition, and are named IgG, IgM, IgA, IgE, and IgD. The IgG-class and IgA-class antibodies are further divided into subclasses, namely, IgG1, IgG2, IgG3, and IgG4, and IgA1 and IgA2. The heavy chains in IgG, IgA, and IgD antibodies have three constant region domains, which are designated CH1, CH2, and CH3, and the heavy chains in IgM and IgE antibodies have four constant region domains, CH1, CH2, CH3 and CH4. Thus, heavy chains have one variable region and three or four constant regions. Immunoglobulin structure and function are reviewed, for example, in Harlow et al., Eds., Antibodies: A Laboratory Manual, Chapter 14, Cold Spring Harbor Laboratory, Cold Spring Harbor (1988).

The heavy chains of immunoglobulins can also be divided into three functional regions: the Fd region (a fragment comprising V_{H} and CH1, i.e., the two N-terminal domains of the heavy chain), the hinge region, and the Fc region (the “fragment crystallizable” region, derived from constant regions and formed after pepsin digestion). The Fd region in combination with the light chain forms an Fab (the “fragment antigen-binding”). Because an antigen will react stereochemically with the antigen-binding region at the amino terminus of each Fab the IgG molecule is divalent, i.e., it can bind to two antigen molecules. The Fc contains the domains that interact with immunoglobulin receptors on cells and with the initial elements of the complement cascade. Thus, the Fc fragment is generally considered responsible for the effector functions of an immunoglobulin, such as complement fixation and binding to Fc receptors. Pepsin sometimes also cleaves before the third constant domain (CH3) of the heavy chain to give a large fragment F(abc) and a small fragment pFcb. These terms are also used for analogous regions of the other immunoglobulins. The hinge region, found in IgG, IgA, and IgD class antibodies, acts as a flexible spacer allowing the Fab portion to move freely in space. In contrast to the constant regions, the hinge domains are structurally diverse, varying in both sequence and length among immunoglobulin classes and subclasses.

For example, the length and flexibility of the hinge region varies among the IgG subclasses. The hinge region of IgG1 reportedly encompasses amino acids 216-231 and because it is freely flexible, the Fab fragments can rotate about their axes of symmetry and move within a sphere centered at the first of two inter-heavy chain disulfide bridges. IgG2 has a shorter hinge than IgG1, reportedly 12 amino acid residues and four disulfide bridges. The hinge region of IgG2 lacks a glycine residue, it is relatively short and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), and is reported to contain 62 amino acids (including 21 prolines and 11 cysteines), forming an inflexible poly-proline double helix. In IgG3 the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge region reportedly decreases in the order IgG3>IgG1>IgG4>IgG2. The four IgG subclasses also differ from each other with respect to their effector functions. This difference is related to differences in structure, including with respect to the interaction between the variable region, Fab fragments, and the constant Fc fragment.

According to crystallographic studies, the immunoglobulin hinge region can be further subdivided functionally into three regions: the upper hinge region, the core region, and the lower hinge region. Shin et al., 1992 Immunological Reviews 130:87. The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the CH2 domain and includes residues in CH2. Id. The core hinge region of human IgG1 contains the sequence Cys-Pro-Pro-Cys that, when dimerized by disulfide bond formation, results in a cyclic octapeptide believed to act as a pivot, thus conferring flexibility. The hinge region may also contain one or more glycosylation sites, which include a number of structurally distinct types of sites for carbohydrate attachment. For example, IgA1 normally contains five glycosylation sites within a 17 amino acid segment of the hinge region, conferring resistance of the hinge region polypeptide to intestinal proteases, considered an advantageous property for a secretory immunoglobulin.

Conformational changes permitted by the structure and flexibility of the immunoglobulin hinge region polypeptide sequence may also affect the effector functions of the Fe portion of the antibody. Three general categories of effector functions associated with the Fc region include (1) activation of the classical complement cascade, (2) interaction with effector cells, and (3) compartmentalization of immunoglobulins. The different human IgG subclasses vary in the relative efficacies with which they fix complement, or activate and amplify the steps of the complement cascade. See, e.g., Kirschfink, 2001 Immunol. Rev. 180:177; Chakraborti et al., 2000 Cell Signal 12:607; Kohl et al., 1999 Mol. Immunol. 36:893; Marsh et al., 1999 Curr. Opin. Nephrol. Hypertens. 8:557; Speth et al., 1999 Wien Klin. Wochenschr. 111:378.

Complement-dependent cytotoxicity (CDC) is believed to be a significant mechanism for clearance of specific target cells such as tumor cells. CDC is a stream of events that consists of a series of enzymes that become activated by each other in a cascade fashion. Complement has an important role in clearing antigen, accomplished by its four major functions: (1) local vasodilation; (2) attraction of immune cells, especially phagocytes (chemotaxis); (3) tagging of foreign organisms for phagocytosis (opsonization); and (4) destruction of invading organisms by the membrane attack complex (MAC attack). The central molecule is the C3 protein. It is an enzyme that is split into two fragments by components of either the classical pathway or the alternative pathway. Antibodies, especially IgG and IgM, induce the classical pathway while the alternative pathway is nonspecifically stimulated by bacterial products like lipopolysaccharide (LPS). Briefly, the products of the C3 split include a small peptide C3a that is chemotactic for phagocytic immune cells and results in local vasodilation by causing the release of C5a fragment from C5. The other part of C3, C3b coats antigens on the surface of foreign organisms and acts to opsonize the organism for destruction. C3b also reacts with other components of the complement system to form an MAC consisting of C5b, C6, C7, C8 and C9.

In general, IgG1 and IgG3 most effectively fix complement, IgG2 is less effective, and IgG4 does not activate complement. Complement activation is initiated by binding of C1q, a subunit of the first component C1 in the cascade, to an antigen-antibody complex. Even though the binding site for C1q is located in the CH2 domain of the antibody, the hinge region influences the ability of the antibody to activate the cascade. For example, recombinant immunoglobulins lacking a hinge region are reportedly unable to activate complement. Shin et al., 1992. Without the flexibility conferred by the hinge region, the Fab portion of the antibody bound to the antigen may not be able to adopt the conformation required to permit C1q to bind to CH2. See id. Hinge length and segmental flexibility have been reported to correlate with complement activation; however, the correlation is not absolute. Human IgG3 molecules with altered hinge regions that are as rigid as IgG4, for example, can still effectively activate the cascade.

These antibodies, binding portions or fragments thereof, hinge portions or fragments thereof, and effector regions or portions thereof, are all useful in the constructs of the invention.

The term “variable” in the context of variable domain of antibodies refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877). With respect to Applicants' anti-IgE antibody, certain CDRs were defined by combining the Kabat et al. and Chothia et al. approaches. The more highly conserved portions of variable domains are called the framework (FR). The variable domains of native heavy and light chains each comprise four FR regions, largely adopting a B-sheet configuration, connected by three CDRs, which form loops connecting, and in some cases forming part of, the B-sheet structure. The CDRs in each chain are held together in close proximity by the FR regions and, with the CDRs from the other chain, contribute to the formation of the antigen binding site of antibodies (see Kabat et al.) The constant domains are not involved directly in binding an antibody to an antigen, but exhibit various effector functions, such as participation of the antibody in antibody-dependent cellular toxicity.

The term “antibody fragment” refers to a portion of a full-length antibody, and includes the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments which are capable of cross-linking antigen, and a residual other fragment (which is termed pFc′). Additional fragments can include diabodies, linear antibodies, single-chain antibody molecules, and multispecific antibodies formed from antibody fragments. As used herein, “binding fragment” with respect to antibodies, refers to Fv, F(ab) and F(ab′)₂ fragments and functional mutants and analogs thereof.

The Fab fragment, also designated as F(ab)′, also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

The light chains of antibodies (immunoglobulin) from any vertebrate species can be assigned to one of two clearly distinct types, called kappa (κ) and lambda (λ), based on the amino sequences of their constant domain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies may be made, for example, by the hybridoma method first described by Kohler and Milstein, Nature 256: 495 (1975), or may be made by recombinant methods, e.g., as described in U.S. Pat. No. 4,816,567. Monoclonal antibodies may also be isolated from phage antibody libraries using the techniques described in Clackson et al., Nature 352: 624-628 (1991), as well as in Marks et al., J. Mol. Biol. 222: 581-597 (1991).

The monoclonal antibodies herein specifically include monoclonal or recombinant antibodies or fragments thereof that have been altered by any means to be less immunogenic in humans.

Thus, for example, the monoclonal antibodies/fragments herein specifically include “chimeric” antibodies and “humanized” antibodies. Generally, in chimeric antibodies, a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567); Morrison et al. Proc. Natl Acad. Sci. 81: 6851-6855 (1984).

“Humanized” forms of non-human (e.g., murine) antibodies or fragments are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, corresponding non-human residues replace Fv framework residues of the human immunoglobulin. Furthermore, humanized antibody may comprise residues that are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. For further details, see, e.g.: Jones et al., Nature 321: 522-525 (1986); Reichmann et al., Nature 332: 323-329 (1988) and Presta, Curr. Op. Struct. Biol. 2: 593-596 (1992).

“Single-chain Fv” or “scFv” antibody fragments may comprise the VH and VL domains of an antibody present in a single polypeptide chain. The scFv polypeptide may further comprise a polypeptide linker between the VH and VL domains that enables the scFv to form the desired structure for antigen binding.

The term “diabodies” includes small antibody fragments with two antigen-binding sites, which fragments comprise a heavy chain variable domain (VH) connected to a light chain variable domain (VL) in the same polypeptide chain (VH-VL). For example, by using no linker or a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad Sci. USA 90:6444-6448 (1993).

As used herein, all numbering of immunoglobulin amino acid residues that appears herein is done according to the immunoglobulin amino acid residue numbering system of Kabat et al., Sequences of Proteins of Immunological Interest (National Institute of Health, Bethesda, Md. 1987).

In general, the term “biologically active” refers to a protein having the function, for example, the structural, regulatory, or biochemical function, of a naturally occurring molecule. The functional activity may be less than, greater than, or about the same as, the naturally occurring molecule. In the therapeutic application of the inventions, the term “biologically active” indicates that a molecule has an activity that impacts an animal suffering from a disease or disorder in a positive sense and/or impacts a pathogen or parasite in a negative sense. Thus, a biologically active molecule may cause or promote biological or biochemical activity within an animal that is detrimental to the growth and/or maintenance of a pathogen or parasite, or of cells, tissues, or organs of an animal that have abnormal growth or biochemical characteristics, such as cancer cells or inflammation, for example.

In the context of prophylactic applications of the invention, the term “biologically active” indicates that a molecule can be used to induce or stimulate an immunoreactive or other desired response, such as an anti-protease response. In some preferred embodiments, the immunoreactive or other response is designed to be prophylactic. In other preferred embodiments, the immunoreactive or other response is designed to cause the immune or other system, such as the protease system, of an animal to react to the detriment of cells of an animal, such as cancer cells, that have abnormal growth or biochemical characteristics.

The terms “binding construct” and “binding domain fusion protein construct” as used herein may refer to, for example, engineered constructs including polypeptides, recombinant polypeptides, synthetic, semi-synthetic or other fusion proteins that are capable of binding a target, for example, an antigen. One or more non-peptide sequences may also be included, for example, as connecting regions.

A “cell” means any living suitable cell for the purposes of the invention, including but not limited to, the manufacture of binding domain fusion proteins. Cells include eukaryotic and prokaryotic cells. Preferred eukaryotic cells include vertebrate cells such as mammalian cells (for example, human, murine, ovine, porcine, equine, canine, and feline cells), avian cells, fish cells, and invertebrate cells such as insect cells and yeast cells. Preferred prokaryotic cells are bacterial cells.

The term “composition” as used herein is intended to encompass a product comprising one or more specified ingredients in specified or other amounts, as well as any product which results, directly or indirectly, from combination of the specified ingredients in such specified or other amounts.

A “compound” is a molecule, and includes, for example, small molecules, proteins, carbohydrates, and lipids.

A “compound known to interact” with a protein means a compound that has been identified as interacting with a protein or other target.

The term “conservative substitution,” when describing a polypeptide, refers to a change in the amino acid composition of the polypeptide that does not substantially alter the activity of the polypeptide, i.e., substitution of amino acids with other amino acids having similar properties. Conservative substitution tables providing functionally similar amino acids are well known in the art. The following six groups each contain amino acids that are generally understood to represent conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W) (see also, Creighton, 1984, Proteins, W.H. Freeman and Company).

In addition to the above-defined conservative substitutions, other modifications of amino acid residues can also result in “conservatively modified variants.” For example, one may regard all charged amino acids as substitutions for each other whether they are positive or negative. In addition, conservatively modified variants can also result from individual substitutions, deletions or additions which alter, add or delete a single amino acid or a small percentage of amino acids, for example, often less than 5%, in an encoded sequence. Further, a conservatively modified variant can be made from a recombinant polypeptide by substituting a codon for an amino acid employed by the native or wild-type gene with a different codon for the same amino acid.

The terms “control elements” or “regulatory sequences” include enhancers, promoters, transcription terminators, origins of replication, chromosomal integration sequences, 5′ and 3′ untranslated regions, with which polypeptides or other biomolecules interact to carry out transcription and translation. For eukaryotic cells, the control sequences will generally include a promoter and preferably an enhancer, for example, derived from immunoglobulin genes, SV40, cytomegalovirus, and a polyadenylation sequence, and may include splice donor and acceptor sequences. Depending on the vector system and host utilized, any number of suitable transcription and translation elements, including constitutive and inducible promoters, may be used. When referring to binding domain fusion protein, a promoter other than that naturally associated with the binding domain fusion protein coding sequence can be referred to as a “heterologous” promoter.

A “deletion” refers to a change in an amino acid or nucleotide sequence due to the absence of one or more amino acid residues or nucleotides. The terms “insertion” or “addition” refer to changes in an amino acid or nucleotide sequence resulting in the addition of one or more amino acid residues or nucleotides, respectively, to a molecule or representation thereof, as compared to a reference sequence, for example, the sequence found in the naturally occurring molecule. A “substitution” refers to the replacement of one or more amino acids or nucleotides by different amino acids or nucleotides, respectively.

As used herein, the term “derivative” includes a chemical modification of a polypeptide, polynucleotide, or other molecule. In the context of this invention, a “derivative polypeptide”, for example, one modified by glycosylation, pegylation, or any similar process, retains binding domain fusion protein activity. For example, the term “derivative” of binding domain fusion protein includes binding domain fusion proteins, variants, or fragments that have been chemically modified, as, for example, by addition of one or more polyethylene glycol molecules, sugars, phosphates, and/or other such molecules, where the molecule or molecules are not naturally attached to wild-type binding domain fusion proteins. A “derivative” of a polypeptide further includes those polypeptides that are “derived” from a reference polypeptide by having, for example, amino acid substitutions, deletions, or insertions relative to a reference polypeptide. Thus, a polypeptide may be “derived” from a wild-type polypeptide or from any other polypeptide. As used herein, a compound, including polypeptides, may also be “derived” from a particular source, for example from a particular organism, tissue type, or from a particular polypeptide, nucleic acid, or other compound that is present in a particular organism or a particular tissue type.

As used herein, a “detectable label” has the ordinary meaning in the art and refers to an atom (for example, radionuclide), molecule (for example, fluorescein), or complex, that is or can be used to detect (for example, due to a physical, chemical or optical property), indicate the presence of a molecule or to enable binding of another molecule to which it is covalently bound or otherwise associated. The term “label” also refers to covalently bound or otherwise associated molecules (for example, a biomolecule such as an enzyme) that act on a substrate to produce a detectable atom, molecule or complex. Detectable labels suitable for use in the present invention include, for example, any composition detectable by spectroscopic, photochemical, biochemical, immunochemical, electrical, optical, chemical, or other means.

A “disorder” is any condition that would benefit from treatment with a molecule or composition described herein. This includes chronic and acute disorders or diseases including those pathological conditions that predispose the mammal to the disorder in question.

The term “epitope” has its ordinary meaning of a site on an antigen or antigenic molecule recognized by an antibody or a binding portion thereof or other binding molecule, such as, for example, an scFv. Epitopes may be molecules or segments of amino acids, including segments that represent a small portion of a whole protein or polypeptide. Epitopes may be conformational (i.e., discontinuous). That is, they may be formed from amino acids encoded by noncontiguous parts of a primary sequence that have been juxtaposed by protein folding.

The term “fusion protein,” refers to a composite polypeptide, i.e., a single contiguous amino acid sequence, made up of two (or more) distinct, polypeptides that fused or otherwise linked together, directly or indirectly, in a single amino acid sequence. Generally, the molecules are linked directly. However, the molecules may be linked indirectly, e.g., by another sequence or molecule, if the overall function and/or activity of the fusion is not undesirably adversely affected. Thus, for example, a fusion protein may include a single amino acid sequence that contains two entirely distinct amino acid sequences or two similar or identical polypeptide sequences that are not normally found together in the same configuration in a single amino acid sequence found in nature. Fusion proteins may be prepared using either recombinant nucleic acid methods, i.e., as a result of transcription and translation of a recombinant gene fusion product, which fusion comprises a segment encoding a polypeptide of the invention and a segment encoding a heterologous polypeptide, or by chemical synthesis methods well known in the art.

The term “high affinity” for binding domain polypeptides described herein refers to an association constant (Ka) of at least about 10⁶M⁻¹, preferably at least about 10⁸M⁻¹, more preferably at least about 10⁹M⁻¹ or greater, more preferably at least about 10¹⁰M⁻¹ or greater, for example, up to 10¹²M⁻¹ or greater. However, “high affinity” binding can vary for other binding domain polypeptides.

“Hybridization” refers to any process by which a single-stranded nucleic acid molecule, portion thereof, or single-stranded region of an otherwise double-stranded nucleic acid molecule binds through base pairing with a complementary single-stranded nucleic acid molecule, portion thereof, or single-stranded region of an otherwise double-stranded nucleic acid molecule. Hybridization may be performed where both nucleic acid molecules are in solution, or between one nucleic acid molecule in solution and another nucleic acid molecule immobilized on a solid support (for example, paper, membranes, filters, chips, pins, glass slides, or any other appropriate substrate to nucleic acids can be fixed).

The terms “immunogen” and “immunogenic” have their ordinary meaning in the art, i.e., an immunogen is a molecule, such as a polypeptide or other antigen, that can elicit an adaptive immune response upon introduction into a person or an animal.

An “isolated” molecule (for example, a polypeptide or polynucleotide) refers to a molecule that is present outside of from its original environment or has been removed from its original environment (for example, the natural environment if it is naturally-occurring). For example, a naturally-occurring polynucleotide or polypeptide present in a living animal is not isolated, but the same polynucleotide or polypeptide, separated from some or all of the coexisting materials in the natural system (for example, proteins, lipids, carbohydrates, nucleic acids), is isolated. Such polynucleotides could be part of a vector and/or such polynucleotides or polypeptides could be part of a composition, and still be isolated in that such vector or composition is not part of its natural environment.

“Mammal” for purposes of treatment refers to any animal classified as a mammal, including human, domestic and farm animals, nonhuman primates, and zoo, sports, or pet animals, such as dogs, horses, cats, cows, etc.

The term “modulate” refers to a change in the biochemical activity. For example, modulation may involve an increase or a decrease in catalytic rate, substrate binding characteristics, an increase or decrease in expression, etc. Modulation may occur, for example, by covalent or non-covalent interaction with the protein, and can involve an increase or decrease in biochemical activity. A “modulator” includes a compound that causes a change, i.e., an increase or decrease, in activity of a protein, and, for example, is typically a ligand, either peptidic, polypeptidic, or a small molecule (for example, an agonist or antagonist). A modulator may act directly, for example, by interacting with a protein to cause an increase or decrease in activity. A modulator may also act indirectly, for example, by interfering with, i.e., antagonizing or blocking, the action of another molecule that causes an increase or decrease in activity of the protein. The terms “modulator” and “modulation” of a molecule of interest, as used herein in its various forms, is intended to encompass antagonism, agonism, partial antagonism and/or partial agonism of an activity associated the protease of interest. In various embodiments, “modulators” may inhibit or stimulate protease expression or activity. Such modulators include small molecules agonists and antagonists of a protease molecule, antisense molecules, ribozymes, triplex molecules, and RNAi polynucleotides, and others.

The phrases “nucleic acid”, “nucleic acid molecule”, and the like refer to a nucleotide, oligonucleotide, polynucleotide, or any fragment thereof. These phrases also refer to single-stranded or double-stranded DNA and/or RNA of cellular or synthetic origin. In this context, “fragments” refer to those nucleic acid molecules that, when translated, produce polypeptides retaining some functional characteristic, for example, antigenicity or a structural domain of a naturally occurring polypeptide. Unless specifically limited, the disclosure of a polynucleotide sequence is also intended to refer to the complementary sequence. As used herein, the term “polynucleotide” includes oligonucleotides.

The terms “operably associated” and “operably linked” refer to functionally related nucleic acid molecules. For example, a promoter is operably associated with or operably linked to a coding sequence if the promoter assists in control of transcription and/or translation of the encoded polypeptide in an appropriate host cell or other expression system. While operably associated or operably linked nucleic acid molecules can be contiguous and in the same reading frame, certain genetic elements need not be contiguously linked to the nucleic acid encoding the polypeptide(s) to be expressed. For example, enhancers need not be located in close proximity to the coding sequences whose transcription they enhance.

The phrase “percent (%) identity” refers to the percentage of sequence similarity found in a comparison of two or more amino acid sequences. Percent identity can be determined electronically using any suitable software. Likewise, “similarity” between two polypeptides (or one or more portions of either or both of them) is determined by comparing the amino acid sequence of one polypeptide to the amino acid sequence of a second polypeptide. Any suitable algorithm useful for such comparisons can be adapted for application in the context of the invention.

By “pharmaceutically acceptable” it is meant, for example, a carrier, diluent or excipient that is compatible with the other ingredients of the formulation and generally safe for administration to a recipient thereof.

The term “polypeptide” is used interchangeably herein with the term “protein,” and refers to a polymer composed of amino acid residues linked by amide linkages, including synthetic, naturally-occurring and non-naturally occurring analogs thereof (amino acids and linkages). Peptides are examples of polypeptides.

A “polynucleotide” means a plurality of nucleotides. Thus, the terms “nucleotide sequence” or “nucleic acid” or “polynucleotide” or “oligonucleotide” are used interchangeably and refer to a heteropolymer of nucleotides or the sequence of these nucleotides. These phrases also refer to DNA or RNA of genomic or synthetic origin which may be single-stranded or double-stranded and may represent the sense or the antisense strand, to peptide nucleic acid (PNA) or to any DNA-like or RNA-like material. It is contemplated that where the polynucleotide is RNA, the T (thymine) in the sequences provided herein is substituted with U (uracil). A polynucleotide that encodes a polypeptide, a polypeptide fragment, or a polypeptide variant refers to a polynucleotide encoding: the mature form of the polypeptide found in nature; the mature form of the polypeptide found in nature and additional coding sequence, for example, a leader or signal sequence or a proprotein sequence; either of the foregoing and non-coding sequences (for example, introns or non-coding sequence 5′ and/or 3′ of the coding sequence for the mature form of the polypeptide found in nature); fragments of the mature form of the polypeptide found in nature; and variants of the mature form of the polypeptide found in nature. Thus, the phrase “binding domain fusion protein-encoding polynucleotide” and the like encompass polynucleotides that include only a coding sequence for a desired binding domain fusion protein, fragment, or variant, as well as a polynucleotide that includes additional coding and/or non-coding sequences.

In general, the term “protein” refers to any polymer of two or more individual amino acids (whether or not naturally occurring) linked via peptide bonds, as occur when the carboxyl carbon atom of the carboxylic acid group bonded to the α-carbon of one amino acid (or amino acid residue) becomes covalently bound to the amino nitrogen atom of the amino group bonded to the a-carbon of an adjacent amino acid. These peptide bond linkages, and the atoms comprising them (i.e., α-carbon atoms, carboxyl carbon atoms (and their substituent oxygen atoms), and amino nitrogen atoms (and their substituent hydrogen atoms)) form the “polypeptide backbone” of the protein. In addition, as used herein, the term “protein” is understood to include the terms “polypeptide” and “peptide” (which, at times, may be used interchangeably herein). Similarly, protein fragments, analogs, derivatives, and variants are may be referred to herein as “proteins,” and shall be deemed to be a “protein” unless otherwise indicated. The term “fragment” of a protein refers to a polypeptide comprising fewer than all of the amino acid residues of the protein. As will be appreciated, a “fragment” of a protein may be a form of the protein truncated at the amino terminus, the carboxy terminus, and/or internally (such as by natural splicing), and may also be variant and/or derivative. A “domain” of a protein is also a fragment, and comprises the amino acid residues of the protein required to confer biochemical activity corresponding to naturally occurring protein.

A “variant” or “analog” refers to a protein altered by one or more amino acids in relation to a reference protein (for example, a naturally occurring form of the protein), for example, by one or more amino acid sequence substitutions, deletions, and/or insertions. A variant or analog may have “conservative” changes, wherein a substituted amino acid has similar structural or chemical properties (for example, replacement of leucine with isoleucine). Alternatively, a variant or analog may one or more have “non-conservative” changes (for example, replacement of glycine with tryptophan). Other variations include amino acid deletions or insertions, or both. Such variants and analogs can be prepared from corresponding nucleic acid molecule variants, which have a nucleotide sequence that varies accordingly from the nucleotide sequences, for example, for binding domain fusion protein constructs.

The term “analog” as used herein generally refers to compounds that are generally structurally similar to the compound of which they are an analog, or “parent” compound. Generally analogs will retain certain characteristics of the parent compound, e.g., a biological or pharmacological activity. An analog may lack other, less desirable characteristics, e.g., antigenicity, proteolytic instability, toxicity, and the like. An analog includes compounds in which a particular biological activity of the parent is reduced, while one or more distinct biological activities of the parent are unaffected in the “analog.” As applied to polypeptides, the term “analog” may have varying ranges of amino acid sequence identity to the parent compound, for example at least about 70%, more preferably at least about 80%-85% or about 86%-89%, and still more preferably at least about 90%, about 92%, about 94%, about 96%, about 98% or about 99% of the amino acids in a given amino acid sequence the parent or a selected portion or domain of the parent. As applied to polypeptides, the term “analog” generally refers to polypeptides which are comprised of a segment of about at least 3 amino acids that has substantial identity to at least a portion of a binding domain fusion protein. Analogs typically are at least 5 amino acids long, at least 20 amino acids long or longer, at least 50 amino acids long or longer, at least 100 amino acids long or longer, at least 150 amino acids long or longer, at least 200 amino acids long or longer, and more typically at least 250 amino acids long or longer. Some analogs may lack substantial biological activity but may still be employed for various uses, such as for raising antibodies to predetermined epitopes, as an immunological reagent to detect and/or purify reactive antibodies by affinity chromatography, or as a competitive or noncompetitive agonist, antagonist, or partial agonist of a binding domain fusion protein function.

Unless otherwise indicated, a protein's amino acid sequence (i.e., its “primary structure” or “primary sequence”) will be written from amino-terminus to carboxy-terminus. In non-biological systems (for example, those employing solid state synthesis), the primary structure of a protein (which also includes disulfide (cysteine) bond locations) can be determined by the user.

The terms “proteinase” and “protease” are used interchangeably herein. Proteinases are able to degrade a target protein sequence, such as through the breaking of one or more amide linkages of a polypeptide or any other mode that removes one or more amino acids from the target protein.

The terms “proteinase inhibitor” and “protease inhibitor” are used interchangeably herein, and include any agent, including proteinaceous or non-proteinaceous agents, that affects or modulates the activity of a proteinase or protease.

The term “recombinant” refers to a polynucleotide synthesized or otherwise manipulated in vitro (for example, “recombinant polynucleotide”), to methods of using recombinant polynucleotides to produce gene products in cells or other biological systems, or to a polypeptide (“recombinant protein”) encoded by a recombinant polynucleotide. Thus, a “recombinant” polynucleotide is defined either by its method of production or its structure. In reference to its method of production, the process refers to use of recombinant nucleic acid techniques, for example, involving human intervention in the nucleotide sequence, typically selection or production. Alternatively, it can be a polynucleotide made by generating a sequence comprising a fusion of two or more fragments that are not naturally contiguous to each other. Thus, for example, products made by transforming cells with any non-naturally occurring vector is encompassed, as are polynucleotides comprising sequence derived using any synthetic oligonucleotide process. Similarly, a “recombinant” polypeptide is one expressed from a recombinant polynucleotide.

A “recombinant host cell” is a cell that contains a vector, for example, a cloning vector or an expression vector, or a cell that has otherwise been manipulated by recombinant techniques to express a protein of interest.

A “small molecule” includes an organic molecule generally having a molecular weight of less than about 5000 daltons. A small molecule may be naturally occurring or synthetic. Small molecules include, for example, organic protease inhibitors.

The phrase “specifically immunoreactive,” or “specifically binds” when referring to the interaction between an antibody or other binding molecule and a protein or polypeptide or eptiope, refers to an antibody or other binding molecule that recognizes and detectably binds with high affinity to the target of interest. Preferably, under designated or desired conditions, the specified antibodies or binding molecules bind to a particular polypeptide, protein or eptiope and do not bind in a significant or undesirable amount to other molecules present in a sample, i.e., that are not undesirably cross-reactive with non-target antigens and/or epitopes. A variety of immunoassay formats may be used to select antibodies or other binding molecule that are immunoreactive with a particular polypeptide and have a desired specificity. For example, solid-phase ELISA immunoassays are routinely used to select monoclonal antibodies having a desired immunoreactivity and specificity. See, Harlow, 1988, ANTIBODIES, A LABORATORY MANUAL, Cold Spring Harbor Publications, New York (hereinafter, “Harlow”), for a description of immunoassay formats and conditions that may be used to determine or assess immunoreactivity and specificity. Thus, for example, the terms “specific binding”, “specifically binding”, “specificity”, and the like refer to an interaction between a protein and a modulator (for example, an agonist or an antagonist), an antibody, etc., that is not random. “Selective binding”, “selectivity”, and the like refer the preference of a compound to interact with one molecule as compared to another. Preferably, interactions between compounds, particularly modulators, and proteins are both specific and selective.

The term “stably transformed” refers to a nucleic acid molecule that has been inserted into a host cell and exists in the host cell, either as a part of the host cell genomic DNA or as an independent molecule (for example, extra-chromosomally), and that is maintained and replicated in the parent host cell so that it is passed down through successive generations of the host cell.

The term “stringent conditions” refers to conditions that permit hybridization between polynucleotides. Stringent conditions can be defined by salt concentration, the concentration of organic solvent (for example, formamide), temperature, and other conditions well known in the art. In particular, stringency can be increased by reducing the concentration of salt, increasing the concentration of organic solvents, (for example, formamide), or raising the hybridization temperature. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, for example, formamide, while high stringency hybridization can be obtained in the presence of an organic solvent (for example, at least about 35% formamide, most preferably at least about 50% formamide). Stringent temperature conditions will ordinarily include temperatures of at least about 30° C., more preferably of at least about 37° C., and most preferably of at least about 42° C. Varying additional parameters, for example, hybridization time, the concentration of detergent, for example, sodium dodecyl sulfate (SDS), and the inclusion or exclusion of carrier DNA, are well known to those skilled in the art. Various levels of stringency are accomplished by combining these various conditions as needed, and are within the skill in the art. Stringent hybridization conditions may also be defined by conditions in a range from about 5° C. to about 20° C. or 25° C. below the melting temperature (Tm) of the target sequence and a probe with exact or nearly exact complementarity to the target. As used herein, the melting temperature is the temperature at which a population of double-stranded nucleic acid molecules becomes half-dissociated into single strands. Methods for calculating the Tm of nucleic acids are well known in the art (see, for example, Berger and Kimmel, 1987, METHODS IN ENZYMOLOGY, Vol. 152: Guide To Molecular Cloning Techniques, San Diego: Academic Press, Inc., and Sambrook et al. (1989) MOLECULAR CLONING: A LABORATORY MANUAL, 2nd Ed., Vols. 1-3, Cold Spring Harbor Laboratory). As indicated by standard references, a simple estimate of the Tm value may be calculated by the equation: Tm=81.5+0.41(% G+C), when a nucleic acid is in aqueous solution at 1 M NaCl (see for example, Anderson and Young, “Quantitative Filter Hybridization” in NUCLEIC ACID HYBRIDIZATION (1985)). Other references include more sophisticated computations which take structural as well as sequence characteristics into account for the calculation of Tm. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (for example, the presence or absence of formamide, dextran sulfate, polyethylene glycol). The effects of these factors are well known and are discussed in standard references in the art, see for example, Sambrook, supra, and Ausubel, supra. Typically, stringent hybridization conditions are salt concentrations less than about 1.0 M sodium ion, typically about 0.01 to 1.0 M sodium ion at pH 7.0 to 8.3, and temperatures at least about 30° C. for short probes (for example, 10 to 50 nucleotides) and at least about 60° C. for long probes (for example, greater than 50 nucleotides). As noted, stringent conditions may also be achieved with the addition of destabilizing agents such as formamide, in which case lower temperatures may be employed.

The term “substantially purified” or “isolated” refers to nucleic acids or polypeptides that are removed from their natural environment and are isolated or separated, and are at least about 50% free, preferably 60% free, more preferably at least about 75% free, and most preferably at least about 90% free or more, from other components with which they are naturally associated. Thus, a protein or polypeptide is considered substantially pure when that protein makes up greater than about 50% of the total protein content of the composition containing that protein, and typically, greater than about 60% of the total protein content. More typically, a substantially pure or isolated protein or polypeptide will make up at least 75%, more preferably, at least 90%, of the total protein. Preferably, the protein will make up greater than about 90%, and more preferably, greater than about 95% of the total protein in the composition. When referring to polynucleotides, the terms “substantially pure” or “isolated” generally refer to the polynucleotide separated from contaminants with which it is generally associated, for example, lipids, proteins and other polynucleotides. The substantially pure or isolated constructs, including polynucleotides, of the present invention will be greater than about 50% pure. Typically, these constructs will be more than about 60% pure, more typically, from about 75% to about 90% pure and preferably from about 95% to about 98% pure.

“Substitutional” variants are those that have at least one amino acid residue in a native sequence removed and a different amino acid inserted in its place at the same position. The substitutions may be single, where only one amino acid in the molecule as been substituted, or they may be multiple, where two or more amino acids have been substituted in the same molecule. “Insertional” variants are those with one or more amino acids inserted immediately adjacent to an amino acid at a particular position in a native sequence. Immediately adjacent to an amino acid means connected to either the a-carboxyl or a-amino functional group of the amino acid. “Deletional” variants are those with one or more amino acids in the native amino acid sequence removed. Ordinarily, deletional variants will have one or two amino acids deleted in a particular region of the molecule.

A molecule (for example, a nucleic acid, protein, or small molecule) that is “synthetic” is one that is produced in whole or in part by chemical synthesis methods.

A “target proteinase” includes proteinases that are modulated directly or indirectly by a binding domain fusion protein described herein. A “proteinase-associated molecule” includes molecules that are present in proximity to a particular target proteinase or are otherwise associated with a particular target proteinase such that binding of the binding domain fusion protein can inhibit or modulate the target proteinase. Various non-limiting example include proteinase-associated molecules that are expressed in or on the surface of a particular cell type that expresses a particular target proteinase. Proteinase-associated molecules also include but are not limited to cell surface antigens on cell types known to express a particular target proteinase.

The term “therapeutically effective amount” means the amount of the subject compound that will elicit a desired response, for example, a biological or medical response of a tissue, system, animal, or human that is sought, for example, by a researcher, veterinarian, medical doctor, or other clinician.

“Treatment” refers to both therapeutic treatment and prophylactic or preventative measures. Those in need of treatment include those already with the disorder as well as those in which the disorder is to be prevented or it progress stopped or slowed.

“Transformation” describes a process by which an exogenous nucleic acid molecule enters a recipient cell. Transformation may occur under natural or artificial conditions according to various methods well known in the art, and may rely on any known method for the insertion of exogenous nucleic acid molecules into a prokaryotic or eukaryotic host cell. The method for transformation is selected based on the type of host cell being transformed and may include viral infection, calcium phosphate precipitation, electroporation, heat shock, lipofection, and particle bombardment. A “transformed” cell includes stably transformed cells in which the inserted DNA is capable of replication either as an autonomously replicating plasmid or as part of the host chromosome, as well as transiently transformed cells in which the inserted nucleic acid molecule may not replicate or segregate.

The term “vector” refers to a nucleic acid molecule amplification, replication, and/or expression vehicle in the form of a plasmid, phage, viral, or other system (be it naturally occurring or synthetic) for the delivery of nucleic acids to cells where the plasmid, phage, or virus may be functional with bacterial, yeast, invertebrate, and/or mammalian host cells. The vector may remain independent of host cell genomic DNA or may integrate in whole or in part with the genomic DNA. The vector will generally but need not contain all necessary elements so as to be functional in any host cell it is compatible with. An “expression vector” is a vector capable of directing the expression of an exogenous polynucleotide, for example, a polynucleotide encoding a binding domain fusion protein, under appropriate conditions.

Constructs of the Invention

Novel molecules and compositions that are useful as therapeutics, as well as for other purposes including diagnostic and research purposes, are provided herein. The compounds can have binding function(s) and one or more target proteinase modulating activities.

The invention provides compositions that include, and methods for obtaining, constructs and binding domain fusion proteins that, for example, control or prevent inflammation, inflammatory reactions, and bacterial, fungal, and viral infections, and diseases involving abnormal cell proliferation, including cancer, and inhibit proteinases that participate in or are associated with one or more conditions, reactions, or disease processes.

Constructs and binding domain fusion proteins provided herein can have, for example, between two and seven polypeptide domains, and several embodiments comprise between two and five polypeptide domains. Each polypeptide domain generally has a desired functional or structural characteristic, and is modular in that each domain may be in any order that retains the desired functional activity or activities of the binding domain fusion protein and/or any of its components.

In some embodiments, it is preferred that constructs do not have binding domains such as those comprising immunoglobulin variable regions. These constructs are useful in various methods of treatment described herein. These embodiments may, for example, comprise, consist essentially or, or consist of, two domains, such as a proteinase inhibitor domain and an immunoglobulin constant region domain. Examples of these type of constructs include SLPI-CH2CH3 or SLPI analog-Ig. The immunoglobulin constant regions may include any of those referenced herein, for example one or more of CH1, CH2, CH3, and CH4 domains from IgE. Such construct may, for example, comprise, consist essentially or, or consist of, a trappin family member protein domain or analog thereof and an immunoglobulin constant region domain. Non-limiting examples of these types of constructs include SLPI-CH2CH3 and SLPI-CH1CH2CH3. In certain embodiments, amino acid substitutions may be introduced into one or more constant region domain. For example, in certain further embodiments a disulfide bond is introduced by the addition of cysteine residues in the CH2 domain. In another aspect, fusion proteins that do not have a binding domain are provided. Other embodiments comprise, consist essentially or, or consist of, three domains, such as a proteinase inhibitor domain, a connecting region domain, and an immunoglobulin constant region domain. Non-limiting examples of these embodiments include SLPI-CH1-HINGE-CH2CH3 and SLPI-HINGE-CH2CH3. In certain further embodiments, for example a construct comprising SLPI-CH1-HINGE-CH2CH3, are co-expressed with a binding domain-light chain constant region to provide a bispecific-like molecule. Certain proteins and protein domains related to trappins have been described, such as the HE4a fusion proteins described in U.S. Ser. No. 10/233,150 to Schummer et al. entitled “Diagnosis of Carcinomas”, and published as US 2003/0108965A1, the contents of which are both are hereby incorporated by reference herein in their entirety, and which may included or excluded from certain embodiments of the invention.

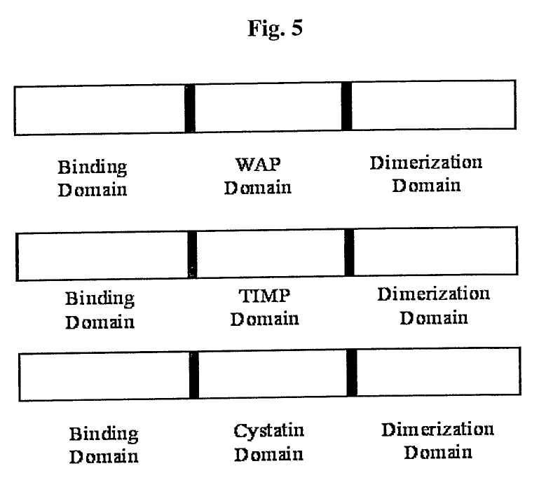

Some embodiments of binding domain fusion proteins have two polypeptide domains, comprising, consisting essentially or, or consisting of, i) anti-proteinase-associated target binding domain and ii) a proteinase inhibitor. In certain embodiments, the binding domain fusion protein comprises, consists essentially of, or consists of a first polypeptide having a binding domain polypeptide capable of binding to a proteinase-associated molecule and a second polypeptide domain capable of inhibiting said proteinase (including protease inhibitors and proteinase inhibitor domains). An example of this type of molecule is anti-CD28scFv-HSLPI (SEQ ID NO: ). See FIG. 5.

In certain embodiments, the binding domain fusion proteins have three polypeptide domains, comprising, consisting essentially or, or consisting of, i) a first polypeptide having a binding domain polypeptide capable of binding to a proteinase or a proteinase-associated molecule; ii) a second polypeptide comprising, consisting essentially of, or consisting of, a proteinase inhibitor domain; and iii) a third polypeptide comprising, consisting essentially of, or consisting of, one or more TGase motifs.

In other embodiments, the binding domain fusion proteins have four polypeptide domains, for example, and comprise, consist essentially or, or consist of, i) a first polypeptide having a binding domain polypeptide capable of binding to a proteinase or a proteinase-associated molecule; ii) a second polypeptide comprising a proteinase inhibitor domain; iii) a third polypeptide comprising, consisting essentially or, or consisting of, one or more TGase motifs, and optionally iv) a connecting region linking one or more of these polypeptides (e.g., linking the first polypeptide and second polypeptides, linking the second and third polypeptides, and/or linking the first and third polypeptides).

Other binding domain fusion proteins may include one or more dimerization domains and have four or five polypeptide domains. For example, in certain embodiments, the binding domain fusion protein has four polypeptide domains and comprises, consists essentially or, or consists of, i) a first polypeptide having a binding domain polypeptide capable of binding to a proteinase or a proteinase-associated molecule; ii) a second polypeptide comprising a proteinase inhibitor domain; iii) a third polypeptide comprising one or more TGase motifs, and iv) one or more dimerization domains (e.g., 1-5 or more). Suitable dimerization domains for these embodiments include a immunoglobulin hinge domain or variant or analog, for example a dimerization domain may be an immunoglobulin CH2CH3 domain or an immunoglobulin CH3 domain or analog (e.g., IgG CH2CH3 or CH3, IgA CH2CH3 or CH3).

Other binding domain fusion proteins have five polypeptide domains. Exemplary embodiments comprise, consist essentially or, or consist of, i) a first polypeptide having a binding domain polypeptide capable of binding to a proteinase or a proteinase-associated molecule; ii) a second polypeptide comprising, consisting essentially or, or consisting of, a proteinase inhibitor domain; iii) a third polypeptide comprising, consisting essentially or, or consisting of, one or more TGase motifs, iv) a connecting region linking one or more of these polypeptides, and v) one or more dimerization domains (e.g., 1-5 or more).

Other embodiments of the binding domain fusion protein that have four polypeptide domains include, for example, binding domain fusion proteins that comprise, consist essentially or, or consist of, i) a first polypeptide comprising, consisting essentially or, or consisting of, a binding domain polypeptide capable of binding to a proteinase or proteinase-associated molecule; ii) a second polypeptide comprising, consisting essentially or, or consisting of, a connecting region attached to said first polypeptide; iii) a third polypeptide comprising, consisting essentially or, or consisting of, a proteinase inhibitor domain; and iv) a fourth polypeptide comprising, consisting essentially or, or consisting of, constant region or portion thereof. In certain embodiments, the immunoglobulin constant region comprises, consists essentially or, or consists of, an immunoglobulin CH3 region, including CH3 analogs. In other embodiments, the binding domain fusion proteins comprise, consist essentially or, or consist of, other immunoglobulin constant regions or analogs, including those described herein.

Other embodiments of the binding domain fusion protein having four domains may comprise, consist essentially or, or consist of, i) a polypeptide binding domain polypeptide capable of binding to a target or a target-associated molecule, ii) a connecting region, iii) a polypeptide proteinase inhibitor domain comprising, consisting essentially or, or consisting of, one or more WAP domains, which is N-terminal to, iv) one or more dimerization domains. Certain other embodiments comprise i) a first polypeptide having a binding domain polypeptide capable of binding to a proteinase or proteinase-associated molecule; ii) a second polypeptide comprising a connecting region attached to said first polypeptide; iii) a third polypeptide comprising, consisting essentially of, or consisting of, a proteinase inhibitor or proteinase inhibitor domain; and iv) one or more dimerization domains, wherein said first polypeptide is N-terminal to said second polypeptide and said second polypeptide is N-terminal to said proteinase inhibitor domain, wherein said proteinase inhibitor domain comprises one or more WAP domains, and wherein said one or more WAP domains is N-terminal to said one or more dimerization domains.

Other embodiments of the binding domain fusion protein having four domains comprising, consisting essentially or, or consisting of, i) a polypeptide binding domain polypeptide capable of binding to a target or a target-associated molecule, ii) a connecting region, iii) one or more dimerization domains which is N-terminal to, iv) a polypeptide proteinase inhibitor domain comprising one or more WAP domains.

Certain other embodiments of the binding domain fusion protein having five domains may also comprise, consist essentially or, or consist of, i) a polypeptide binding domain polypeptide capable of binding to a target or a target-associated molecule, ii) a connecting region, iii) a polypeptide proteinase inhibitor domain comprising, consisting essentially or, or consisting of, one or more WAP domains, which is N-terminal to, iv) one or more TGase domains, which is N-terminal to, v) one or more dimerization domains.

Additional exemplary embodiments of a binding domain fusion protein include without limitation NH₃⁺—{V_{L}—V_{H} or V_{H}—V_{L}}spacer{WAPx}spacer{IgG1 C_{H}³}_{y}—COO—, NH₃⁺—{V_{L}—V_{H} or V_{H}—V_{L}}spacer{TIMP-2x}spacer{IgG1 C_{H}3}_{y}—COO—, NH₃⁺—{V_{L}—V_{H} or V_{H}—V_{L}}spacer{cystatin x}spacer{IgG1 C_{H}3}_{y}—COO—, where x is 0 to 5 and is most preferably 1 or 2 and y is 0 to 5 and preferably 1 or 2. A second preferred embodiment of the invention is without limitation NH₃⁺—{V_{L}—V_{H} or V_{H}—V_{L}}spacer{WAPx}spacer{IgGA C_{H}3}_{y}—COO^{-,} NH₃⁺—{V_{L}—V_{H} or V_{H}—V_{L}}spacer{TIMP-2x}spacer{IgGA C_{H}3}_{y}—COO^{-,} and NH₃⁺—{V_{L}—V_{H} or V_{H}—V_{L}}spacer{cystatin x}spacer{IgGA C_{H}3}_{y}—COO⁻. Within the scope of the present invention are all combinations of protein inhibition domains in the binding domain fusion protein including without limitation NH₃⁺—{V_{L}—V_{H} or V_{H}—V_{L}}spacer{WAPₐ-TIMP-2_{b}-cystatin_{c}}spacer{IgG1 C_{H}3}_{y}—COO—, where a+b+c is equal to 1 to 5 and a, b, and c are 0 to 5, and the individual protein inhibition domains are in any order with or without spacers separating them.

These embodiments are non-limiting examples of specific combinations of polypeptide domains, and numerous other binding domain fusion proteins can be obtained by the desired positioning of any of the modular polypeptide domains described in greater detail below.

### Proteinase Inhibitor Domains

Suitable proteinase inhibition domains of the binding domain proteins include molecules comprising, consisting essentially of, or consisting of, WAP motifs, including for example trappins, which have WAP motifs. One or more WAP motifs, or portion(s) thereof, may by used, including but not limited to domains with protein sequences related to WAP motifs, and domains comprising, consisting essentially of, or consisting of, fragments and variants of WAP motifs having proteinase inhibiting activity. WAP domains may be of mammalian origin, including for example human, non-human primates, camelids, wallaby, etc. or originating from other animals, including for example reptilian, avian (e.g. chicken).

In general, proteinase inhibitor domains of the binding domain fusion proteins may, for example, comprise, consist essentially of, or consist of any of the following: trappin polypeptides having proteinase inhibitor activity, naturally and non-naturally occurring analogs of trappin polypeptides that have proteinase inhibitor activity, SLPI polypeptides, naturally and non-naturally occurring analogs of SLPI polypeptides that have proteinase inhibitor activity, elafin polypeptides, and naturally and non-naturally occurring analogs of elafin polypeptides that have proteinase inhibitor activity, WAP motif polypeptides having proteinase inhibitor activity, and naturally and non-naturally occurring analogs of such WAP motif polypeptides that have proteinase inhibitor activity, TIMP polypeptides having proteinase inhibitor activity, and naturally and non-naturally occurring analogs of TIMP polypeptides that have proteinase inhibitor activity, cystatin polypeptides having proteinase inhibitor activity, naturally and non-naturally occurring analogs of cystatin polypeptides that have proteinase inhibitor activity, defensin polypeptides having proteinase inhibitor activity, and naturally and non-naturally occurring analogs of defensin polypeptides that have proteinase inhibitor activity.

Specific examples of WAP domains or proteins containing WAP domains that can be used in the binding domain fusion proteins include, but are not limited to, Human antileukoproteinase 1 precursor (ALP) (protease inhibitor WAP4, Swiss-Prot ALK1_HUMAN P03973); elafin precursor (elastase-specific inhibitor, human skin-derived antileukoproteinase (SKALP), protease inhibitor WAP3, Swiss-Prot ELAF_HUMAN P19957); pig elafin precursor (WAP-1 protein, Swiss-Prot ELAF_PIG Q29125); human eppin precursor (epididymal protease inhibitor) (serine protease inhibitor-like with Kunitz and WAP domains I (protease inhibitor WAP7, Swiss-Prot EPPI_HUMAN 095925, TrEMBL Q86TP9, Entrez protein NP_{—}852479, AAH44829, NP_{—}065131); rhesus macaque eppin precursor (Epididymal protease inhibitor) (serine protease inhibitor-like with Kunitz and WAP domains 1, Swiss-Prot EPPI_MACMU Q9BDL1); mouse eppin precursor (epididymal protease inhibitor, serine protease inhibitor-like with Kunitz and WAP domains 1, Swiss-Prot EPPI_MOUSE Q9DA01, Entrez protein AAH48637); black-necked spitting cobra nawaprin (similar to elafin) (Swiss-Prot NWAP_NAJNG P60589); pig sodium/potassium ATPase inhibitor SPAI-2 precursor (WAP-2 protein, Swiss-Prot SPAI_PIG P16225); mouse single WAP motif protein 1 precursor (elafin-like protein I, Swiss-Prot SWM1_MOUSE Q9JHY4, Entrez protein NP_{—}067020); mouse single WAP motif protein 2 precursor (elafin-like protein II, Swiss-Prot SWM2_MOUSE Q9JHY3); pig WAP-3 protein precursor (Swiss-Prot WAP3_PIG Q29126); human WAP four-disulfide core domain protein 10A precursor (putative protease inhibitor WAP 10A, Swiss-Prot WFAA_HUMAN Q9H1F0, Entrez protein: NP_{—}542791, XP_{—}215918); rat WAP 10A precursor (Entrez protein XP_{—}215918); mouse WAP 10A precursor (Entrez protein XP_{—}130649); human protein WFDC10B precursor (Swiss-Prot WFAB_HUMAN Q8IUB3); chicken WAP four-disulfide core domain protein 1 precursor (ps20 protein, Swiss-Prot WFDI_CHICK Q8JG33, Entrez protein NP_{—}542181); human WAP four-disulfide core domain protein 1 precursor (prostate stromal protein ps20) (ps20 growth inhibitor, Swiss-Prot WFDI_HUMAN Q9HC57); mouse WAP four-disulfide core domain protein 1 precursor (prostate stromal protein ps20, ps20 growth inhibitor, Swiss-Prot WFD1_MOUSE Q9ESH5); rat WAP four-disulfide core domain protein 1 precursor (prostate stromal protein ps20) (ps20 growth inhibitor, Swiss-Prot WFD1_RAT 070280); dog WAP four-disulfide core domain protein 2 precursor (major epididymis-specific protein E4(CE4), epididymal secretory protein E4, Swiss-Prot WFD2_CANFA Q28894); human WAP four-disulfide core domain protein 2 precursor (Major epididymis-specific protein E4, epididymal secretory protein E4, putative protease inhibitor WAP5 Swiss-Prot WFD2_HUMAN Q14508, Entrez protein NP_{—}542771, NP_{—}542772, NP_{—}542773, NP_{—}542774, NP006094, NP_{—}003055); mouse WAP four-disulfide core domain protein 2 precursor (WAP domain protein HE4, Swiss-Prot WFD2_MOUSE Q9DAU7); pig WAP four-disulfide core domain protein 2 precursor (epididymal secretory protein E4, Swiss-Prot WFD2_PIG Q8MI69); rabbit WAP four-disulfide core domain protein 2 precursor (Major epididymis-specific protein E4, epididymal protein BE-20, Swiss-Prot WFD2_RABIT Q28631); rat WAP four-disulfide core domain protein 2 precursor (epididymal secretory protein 4 (RE4), Swiss-Prot WFD2_RAT Q8CHN3); human WAP four-disulfide core domain protein 3 precursor (putative protease inhibitor WAP14, Swiss-Prot WFD3_HUMAN Q8IUB2, Entrez protein: NP_{—}852666, NP_{—}852782, NP_{—}852663); human WAP four-disulfide core domain protein 5 precursor (putative protease inhibitor WAP1, Swiss-Prot WFD5_HUMAN Q8TCV5, Entrez protein: NP_{—}663627, AAH39173, AAK72468); human WAP four-disulfide core domain protein 6 precursor (putative protease inhibitor WAP6, Swiss-Prot WFD6_HUMAN Q9BQY6, Entrez protein NP_{—}543017); human WAP four-disulfide core domain protein 8 precursor (putative protease inhibitor WAP8, Swiss-Prot WFD8_HUMAN Q8IUA0, Entrez protein: NP_{—}570966, NP_{—}852611); punitive mouse WAP8 (Entrez protein XP_{—}204940); human protein WFDC9 precursor (Swiss-Prot WFD9_HUMAN Q8NEX5); human protein WFDC11 precursor (Swiss-Prot WFDB_HUMAN Q8NEX6, Entrez protein NP_{—}671730); WAP four-disulfide core domain protein 12 precursor (pPutative protease inhibitor WAP12, Swiss-Prot WFDC_HUMAN Q8WWY7, Entrez protein:NP_{—}742143, NP_{—}543145, NP_{—}742003); Protein WFDC13 precursor (Swiss-Prot WFDD_HUMAN Q8IUB5); human protein with a kunitz/bovine pancreatic trypsin inhibitor domain and WAP-type (Whey acidic protein) ‘four-disulfide core’ domains, TrEMBL Q9H3Y3); human ps20 WAP-type four-disulfide core domain protein (Entrez nucleotide AAG15263.1); arabian camel whey acidic protein (WAP, Swiss-Prot WAP_CAMDR P09837); tammar wallaby whey acidic protein (WAP, Swiss-Prot WAP_MACEU Q9N0L8, Entrez protein CAB90357); mouse whey acidic protein (WAP, Swiss-Prot WAP_MOUSE P01173, Entrez protein AAH26780); pig whey acidic protein (WAP, Swiss-Prot WAP_PIG 046655); rabbit whey acidic protein (WAP, Swiss-Prot WAP_RABIT P09412); rat whey acidic protein (WAP, Swiss-Prot WAP_RAT P01174, Entrez protein NP_{—}446203); mouse whey acidic protein precursor (TrEMBL Q7M748); mouse similar to whey acidic protein (TrEMBL Q8R0J0); whey acidic protein (WAP, Swiss-Prot ); brush-tailed possum (TrEMBL Q95JH3, Entrez protein AAK69407); rat protein with WAP-type four-disulfide core domains (Entrez protein XP_{—}215938); human multivalent protease inhibitor protein (Swiss-Prot Q8TEU8, Entrez protein AAL77058); mouse serpina 3 g protein (Entrez protein AAH57144); mouse secretory leukocyte protease inhibitor (Entrez protein AAH28509, NP_{—}035544); human multivalent protease inhibitor (Entrez protein NP_{—}783165); rat secretory leukocyte protease inhibitor (Entrez protein NP_{—}445824, XP_{—}215940); mouse eppin (Entrez protein NP_{—}083601); rat, similar to eppin (Entrez protein XP_{—}345469); human-similar to elafin-like protein from mouse and WAP-type protease inhibitors (Entrez protein CAC36291); Human putative protease inhibitor WAP2 precursor (Entrez protein AAK68848); human putative multivalent protease inhibitor (Entrez protein AAL18839). Other WAP domains contemplated within the scope of the invention are readily identified on various databases, such as Medline (National Library of Medicine), EPASy (Expert Protein Analysis System, which includes Swiss-Prot/TrEMBL Swiss Institute of Bioinformatics, and Protein Data Base (Brookhaven, PDB)). Additional proteinase inhibitor domains include for example eppin, huWAP2, SWAM1, SWAM2, and the proteins specified in humans by LOC 149709.

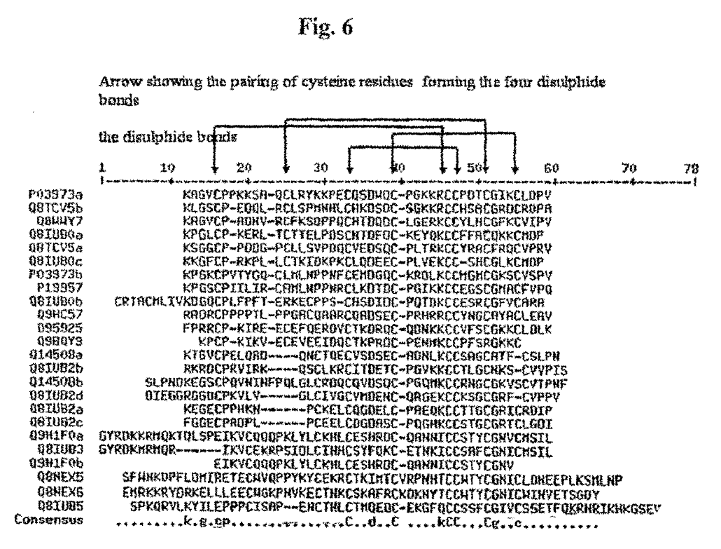

The alignment for several human WAP domains is illustrated in FIG. 6 (ACCESSION NUMBERS are indicated on the left side of the Figure). The connected arrows at the top of the figure illustrate the disulfide bond pairing that is a central feature of the WAP motif. WAP domains are aligned to illustrate homology of amino acid residues and particularly of cysteine residues and their spacing throughout the polypeptide sequence.

Suitable trappin family members that can be utilized in the binding domain fusion proteins include, for example: BTrappin-2 protein (Bovine, TrEMBL 046625); BTrappin-4 protein precursor (Bovine, TrEMBL 046626), BTrappin-5 protein precursor (Bovine, TrEMBL O46627); Trappin-6 (Bovine, TrEMBL 062652, Entrez protein JE0252, BAA28148); STrappin-2 protein precursor (Rhesus macaque, TrEMBL 046643); Trappin (Guinea pig, TrEMBL Q8VID9); Elafin (Trappin-2) (Warthog, TrEMBL Q9XS42, Entrez protein JE0251, BAA77825); SPAI (Trappin-1) (Warthog, TrEMBL # Q9XS43, Entrez protein # JE0250, BAA77826); Trappin (Collared peccary, TrEMBL # Q9XS44, BAA77827); Trappin-11 (Hippopotamus, TrEMBL # Q9XS45, Entrez protein # JE0257); similar to trappin (Norway rat, Entrez protein # XP_{—}345873); elafin (sheep, Entrez protein # AAQ21594); trappin-7 (pig TrEMBL # P79389, Entrez protein # JE0253); trappin-8 (pig, Entrez protein # JE0254); trappin-9 (pig, TrEMBL, # Q9XS46, Entrez protein # JE0255); trappin-10 (collared peccary Entrez protein # JE0256); trappin (domestic guinea pig, Entrez protein # BAB79626); elafin family member protein (pig, Entrez protein # BAA08858, BAA08857); elafin homolog (pig, Entrez protein # BAA12038, BAA77829); trappin (Hippopotamus Entrez protein # BAA77828). {Databases searched: Swiss-Prot, TrEMBL, Entrez protein, PIR/Georgetown}. Other trappin family members, including those described herein, and those now known or later discovered are contemplated as within the scope of the invention.

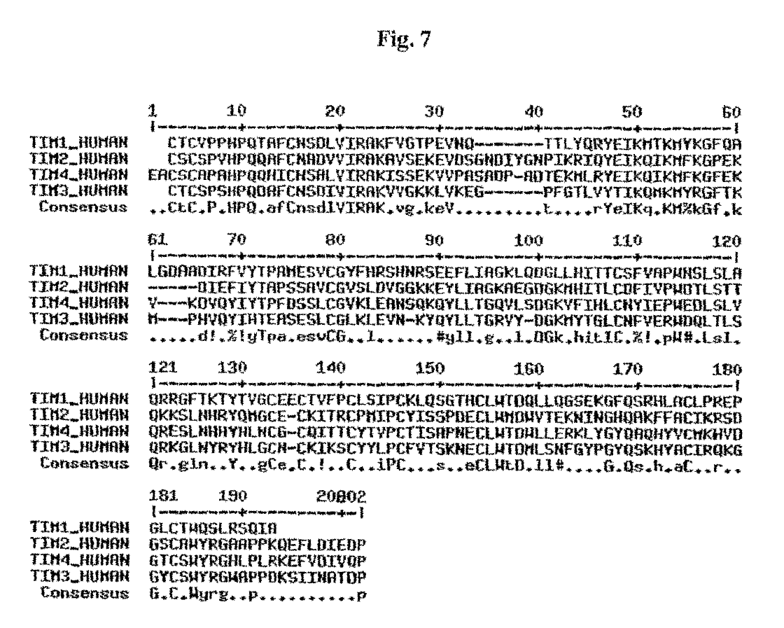

In other embodiments, the binding domain fusion proteins have proteinase inhibition domains comprising a TIMP domain, such as for example a TIMP domain from TIMP1, TIMP2, TIMP3, TIMP4, or a TIMP domain from any other TIMP family member or an isoform of any TIMP motif. Such TIMP domains include for example any polypeptide or portion or variant thereof that inhibits MMP-1, MMP-2, MMP-3, MMP-7, MMP-8, MMP-9, MMP-10, MMP-13, MMP-14, MMP-15, MMP-16 and MMP-19, and collagenases. See for example Bode W., Maskos K., 2003, Biol Chem. 384(6):863-72, Beaudeux J. L., et al., 2004 Clin. Chem. Lab Med. 42(2):121-131, Nagase H., Brew K., 2003 Biochem. Soc. Symp., 70:201-12, Visse R., Nagase H., 2003, Circ. Res., 92(8):827-39, Baker A. H. et al., 2002 J. Cell Sci., 115(19):3719-27, Skiles J. W. et al., 2001 Curr. Med. Chem. 8(4):425-74, and Nagase H., Brew K., 2002 Arthritis Res. 4(Supp. 3)S51-61 the contents of which are all hereby incorporated by reference herein. FIG. 7 illustrates an alignment for four human TIMP domains in which the alignment and spacing of 12 cysteines is observed. In still other embodiments, the binding domain fusion proteins comprise variants of TIMP domains, including substitutional variants, insertional variants, and deletional variants. Preferably, a variant of a TIMP domain has protease/proteinase inhibitor activity.

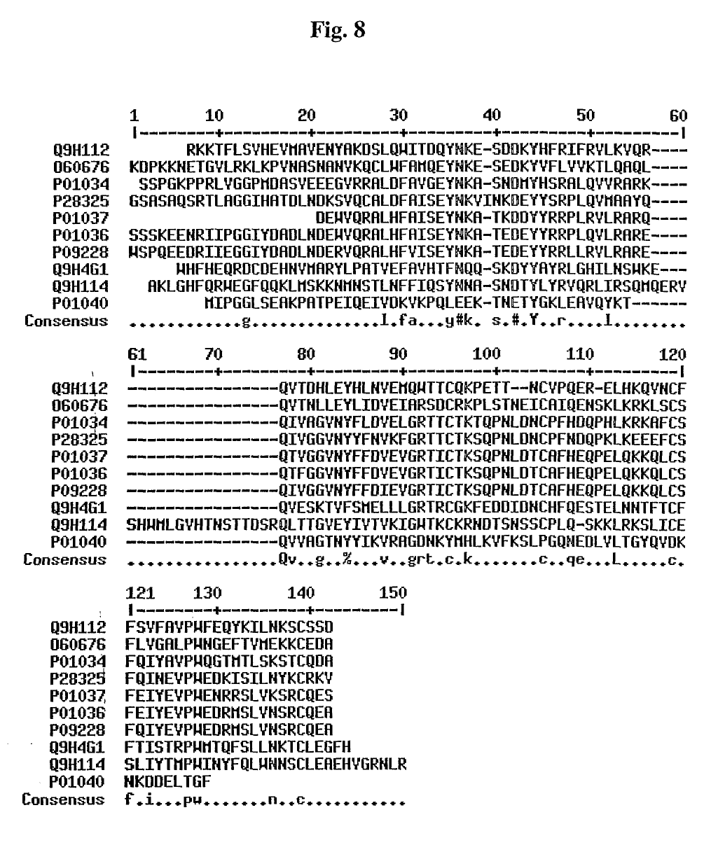

In other embodiments, the binding domain fusion proteins have proteinase inhibition domains comprising, consisting essentially of, or consisting of, a cystatin domain. Exemplary cystatin domains include, for example, those from cystatins in family I (cystatin family), cystatins in family II (stefin family, e.g. cystatin C, D, S, SN, and SA), and cystatins in family III (kininogen family). Such cystatin domains include for example any polypeptide or portion or variant thereof that inhibits a cathepsin (e.g., cathepsins B, H, K, L, and S). In still other embodiments, the binding domain fusion proteins comprise, consist essentially of, or consist of, variants of cystatin domains, including substitutional variants, insertional variants, and deletional variants. Preferably, a variant of a cystatin domain has protease/proteinase inhibitor activity. FIG. 8 illustrates yet another such alignment for several human cystatin domains.

In other embodiments, the binding domain fusion proteins have proteinase inhibition domains that are a Kunitz-type inhibitor, which comprise, consist essentially of, or consist of, an active “Kunitz domain”. Protease inhibitor domains of the binding domain fusion protein may comprising, consisting essentially of, or consisting of, for example, one or more Kunitz domains,. Each Kunitz domain typically contains approximately 50-60 amino acids, and comprises six cysteine residues that form three disulfide bonds that result in a double-loop structure. Representative Kuntz inhibitors that may be used in the protease inhibitor domain of the binding domain fusion proteins include Bovine Pancreatic Trypsin Inhibitor (BPTI, also known as Aprotinin) (Trapnell, J. E., et al, 1974 Brit. J Surg. 61: 177-182; Sher, G, 1977 Am. J. Obstet. Gynecol. 129: 164-170; Auer, L. M., et al., 1979 Acta Neurochir. 49: 207-217; McMichan, J., et al., 1982 Circulatory Shock 9: 107-116; Kobayashi, H., et al., 2004 J Biol Chem 279: 6371-9), Bikunin (Kobayashi, 1995 Br J Cancer, 72:1131-1137; Kobayashi et al., 2001 J. Biol. Chem., 276: 2015-2022), Penthalaris (Francischetti I M, et al., 2004 Thromb. Haemost. 91: 886-98), Ecotin (Dennis, M S et al., 1995 J Biol Chem., 270: 25411-17), amyloid precursor protein (APP) (Preece, P., et al., 2004 Brain Res Mol Brain Res. 122: 1-9), WFIKKN (Hill, J J, et al., 2003 Mol. Endocrinol. 17: 1144-54), tissue factor pathway inhibitor (TFPI) (Hiraishi S., et al., 2002 Biochem Biophys Res Commun. 298: 468-73), urinary trypsin inhibitor (UTI) (Suzuki M., et al., 2001 Biochim Biophys Acta. 1547: 26-36), Hepatocyte growth factor activator inhibitor type 2 (HAI-2) (Itoh H., et al., 1999 Biochem Biophys Res Commun. 255: 740-8). In certain other embodiments, the binding domain fusion proteins comprise, consist essentially of, or consist of, more than one proteinase inhibition domain wherein at least one proteinase inhibitor domain comprises a Kunitz-type inhibitor and one or more additional protease inhibitor domains comprise, for example, a WAP motif.

In another aspect, proteinase inhibitor polypeptide domains comprising a conserved disulfide core are contemplated, for example a proteinase inhibitor domain in which the number of cysteine residues is different than a naturally occurring WAP motif. For example, proteinase inhibitor domains comprising a different number of cysteines that the eight cysteines (that form four disulfide bonds) reported in WAP motifs are contemplated. For example, proteinase inhibitor domains comprising four, six, ten, twelve, fourteen, sixteen, twenty, or more cysteine residues are within the scope of the invention. Preferably, the number of cysteine residues is an even number, and two or more cysteine residues are capable of forming disulfide bonds that stabilize the proteinase inhibitor domain.

In certain other embodiments, the proteinase inhibitor analog has a proteinase inhibition activity that is reduced or that is substantially inactivated. Such proteinase inhibitor analogs, for example, may have selective amino acid deletions, insertions, or substitutions in comparison to a proteinase inhibitor polypeptide described or referenced herein or otherwise now known or later discovered. It is thus another aspect to provide analogs proteinase inhibitor domains of the binding domain fusion protein comprise, consist essentially or, or consist of, naturally and non-naturally occurring analogs of trappin polypeptides with a proteinase inhibitor activity that is reduced or inactive, naturally and non-naturally occurring analogs of SLPI polypeptides with a proteinase inhibitor activity that is reduced or inactive, naturally and non-naturally occurring analogs of elafin polypeptides with a proteinase inhibitor activity that is reduced or inactive, naturally and non-naturally occurring analogs of such WAP motif polypeptides with a proteinase inhibitor activity that is reduced or inactive, naturally and non-naturally occurring analogs of TIMP polypeptides with a proteinase inhibitor activity that is reduced or inactive, naturally and non-naturally occurring analogs of cystatin polypeptides with a proteinase inhibitor activity that is reduced or inactive, naturally and non-naturally occurring analogs of defensin polypeptides with a proteinase inhibitor activity that is reduced or inactive.

It is another aspect of the invention to provide analogs of proteinase inhibitors that have a reduced susceptibility to degradation by a proteinase. In one embodiment, the binding domain fusion proteins comprise an analog of SLPI that has a reduced susceptibility to degradation by a proteinase, or for example an analog of SLPI which is resistant to degradation by one or more particular proteinase. In certain embodiments, the binding domain fusion proteins comprise, consist of, or consist essentially of an analog of SLPI comprising amino acid substitutions or deletions at one or both of amino acids Thr(67) and Tyr(68). In other embodiments, an analog of SLPI comprising amino acid substitutions or deletions at one or more of amino acids Leu(72), Met(73), and Leu(74). In one particular embodiment, Leu at position 72 of SLPI is substituted, for example, with glycine. In preferred embodiments, the analog of SLPI comprises amino acid substitutions or deletions that make the SLPI domain of the binding domain fusion protein at least partially resistant to degradation by a proteinase (e.g. cathepsin). Various analogs and muteins of SLPI are described, for example, in US2002/0010318 to Niven et al., U.S. Pat. No. 6,291,662 to Bandyopadhyay et al., U.S. Pat. No. 5,851,983 to Sugiyama et al., U.S. Pat. No. 5,633,227 to Muller et al., and Eisenberg, S. P. et al., 1990 J. Biol. Chem., 265(14):7976-7981, the contents of which are both are hereby incorporated by reference herein in their entirety, and which may included or excluded from certain embodiments of the invention.

In another aspect, the specificity and potency of inhibition of proteinases by a proteinase inhibitor domain may be altered (e.g., optimized). For the purposes of illustration only, the binding loop of a WAP domain (e.g., a WAP domain from SLPI or elafin) of a binding domain fusion protein is modified to increase a biological activity of the binding domain fusion protein. Phage display of a WAP domain on gene III or gene VIII has been reported to be used to select WAP domains that have improved ability to inhibit or bind to a proteinase. (see Nixon, A. E., 2002 Curr. Pharm. Biotechnol. 3: 1-12). Thus, in certain other embodiments one or more modification (e.g., amino acid insertion, substitution, or deletion) of a proteinase inhibitor is made to change one or more preselected attributes, including for example the absolute or relative molecular weight, complete or partial sequences (amino acids or nucleotide) of polypeptides and nucleic acids, enzymatic activity, ligand binding activity, proteinase resistance, serum stability, pI, antigenicity, the ability to be formulated into various pharmaceutical compositions, ease of production, yield during production, cost of production, associated side effects, specificity, and the like.

In another aspect, any desired naturally occurring and chemically synthesized proteinase inhibitors may be used in the binding domain fusion proteins. These include organic molecules, which may for example be used alone, or as a conjugate to a biological molecule. For example, small molecule protease inhibitors may be attached, conjugated, or otherwise associated with the constructs and binding domain fusion proteins provided herein. See for example, Curci J. A., et al., 2000 J Vasc Surg., 31(2):325-42) describing doxycycline; Moore G. et al., 1999 J Vasc Surg. 29(3):522-32 describing hydroxamate; Supuran et al., 2003 Med. Res. Rev. 23(5):535-58 describing sulfonamide-type protease inhibitors; Tamamura H., Fujii N., 2004 Curr. Drug Targets Infect Disord., 4(2):103-10 describing CXCR4 antagonist; Schirmeister T., Kaeppler U., 2003 Mini Rev. Med. Chem., 3(4):361-73 describing non-peptidic cysteine protease inhibitors; Hernandez A. A., Roush W. R., 2003 Curr. Opin. Chem. Biol. 8(4):459-65 describing synthetic cysteine protease inhibitors; Donkor I. O., 2000 Curr. Med. Chem., 7(12):1171-88 describing calpain inhibitors; and Schimmoller F., et al., 2002 Curr. Pharm. Des. 8(28):2521-31 describing amyloid forming proteases; the contents of which are all hereby incorporated by reference herein in their entirety, and which may included or excluded from certain embodiments of the invention.

Other proteinases and other proteinase inhibitor domains now known or later discovered are contemplated as within the scope of the invention.

In another aspect, the binding domain fusion proteins comprise, consist essentially of, or consist of, proteinase inhibition domains from different sources are linked together in combinations that do not occur in nature. Rotation and translation of the crystal coordinates of the amino and carboxy terminal WAP domains of SLPI allows the two domains to be superimposed. This indicates that the binding loops for the proteinases in the domains are geometrically situated so as to allow two molecules of a proteinase to bind to a single molecule of SLPI at the same time. Spacers are inserted between domains to allow additional flexibility to facilitate more than one proteinase to bind simultaneously to a combination of proteinase inhibition domains, including without limitation WAP domains, TIMP-2 domains, and cystatin domains.

The proteinase inhibitor domains of the binding domain fusion protein inhibit the proteinase activity of any desired target proteinase/proteases, including those known or described herein, or later discovered. Exemplary target proteinases/proteases include intracellular proteases, including caspases including but not limited to caspase 1 to caspase 14, proteases involved in the regulation of complement activation, proteases involved in the regulation of coagulation, proteases involved in the regulation of signal transduction, proteases involved in processing of various precursors of VEGF, proteases involved in the expression or activity of prostaglandins (e.g., PGHS-2), matrix metalloproteinases (e.g. metalloproteinases-2), elastase, alpha1-proteinase, proteinase 3, chymotrypsin, trypsin, human mast cell chymase, stratum corneum chymotryptic enzyme, tryptase, human leukocyte elastase, stratum corneum chymotryptic enzyme, and proteinases that have, for example, elastin, proteoglycans, and collagen as substrates, human neutrophil elastase; other elastases; polymorphonuclear granulocyte; proteinase 3; subtlisin; achrombacter protease; alpha-lytic protease; protease A; glutamic acid specific protease; protease B; epidemolytic (exfoliative) toxin A; exfoliative toxin B; protease Do (DegP, HtrA); mitochondrial serine protease HtrA2; prostate specific antigen; thrombin; neuropsin; heat shock protein 31; family members of trypsin-like proteases including, but not limited to, prokaryotic, eukaryotic, and viral (viral capsid protein, TEV protease, NS3 protease, NSP4 protease) proteases; members of the superfamily of cysteine proteases, including but not limited to, members of papain-like proteases (cathepsin exopeptidases, cathepsin endopeptidases, procathepsin B, cathepsins B, C, F, G, H, K, J, L, L2. M, O, Q, R, S, W, Z, V, and X, cathepsins including but not limited to cathepsin-1, cathepsin-2, cathepsin-3, and cathepsin-6, Trypansoma cruzi cuzain, human bleomycin hydrolase, staphopain, and Staphlococcal pyrogenic exotoxin B), FMDV leader protease (foot-and-mouth disease virus), calpains including but not limited to μ-calpain, m-calpain, calpain1 to calpain 14, calpastatin, calpain large subunit, catalytic domain (domain II), transglutaminase catalytic domain, paracaspase, arylamine-N-acetyltransferase, ubiquitin carboxyl-terminal hydrolase 7, adenoviral protease-like, microbial transglutaminase, otubain families, furin and furin motif-variants, PC5, PC7. For a general description of proteases and inhibitors, see for example Barrett, A. J., 1994, “Classification of peptidases”, Methods Enzymol., 244, 1-15; Otto, H.-H. & Schirmeister, T. 1997 “Cysteine Proteases and their inhibitors”, Chem. Rev. 97, 133-171, the contents of which are hereby incorporated by reference in their entirety.

Binding Domains

The binding domain fusion protein includes a binding domain polypeptide capable of binding to a protease-associated molecule. A binding domain fusion protein, including a binding domain polypeptide, according to the present disclosure includes any polypeptide that possesses the ability to specifically recognize and bind to a cognate biological molecule or complex of more than one molecule or assembly or aggregate, whether stable or transient, of such a molecule. Such molecules include, for example, proteins, polypeptides, peptides, amino acids, or derivatives thereof; lipids, fatty acids or the like, or derivatives thereof; carbohydrates, saccharides or the like or derivatives thereof; nucleic acids, nucleotides, nucleosides, purines, pyrimidines or related molecules, or derivatives thereof, or the like; or any combination thereof such as, for example, glycoproteins, glycopeptides, glycolipids, lipoproteins, proteolipids; or any other biological molecule.

A binding region, including a binding domain polypeptide, for example, may be any naturally occurring, synthetic, semi-synthetic, and/or recombinantly produced binding partner for a biological or other molecule that is a protease-associated structure or molecule of interest, herein sometimes referred to as an “antigen” or an “eptiope” but intended according to the present disclosure to encompass any biological or other molecule to which it is desirable to have the subject fusion protein, bind or specifically bind. Constructs of the invention are defined to be “specific”, “immunospecific” or capable of binding to a desired degree, including specifically binding, if they bind a desired target molecule such as an antigen as provided herein, at a desired level, for example, with a Kₐ of greater than or equal to about 10⁶ M⁻¹, more preferably of greater than or equal to about 10⁷ M⁻¹ and still more preferably of greater than or equal to about 10⁸ M⁻¹. Affinities of even greater than about 10⁸ M⁻¹ are still more preferred, such as affinities equal to or greater than about 10 M-⁻¹, about 10¹⁰ M⁻¹, about 10¹¹ M⁻¹, and about 10¹² M⁻¹. Affinities of binding domain fusion proteins according to the present invention can be readily determined using conventional techniques, for example those described by Scatchard et al., 1949 Ann. N. Y. Acad. Sci. 51: 660. Determination of fusion protein binding can also be performed using any of a number of known methods for identifying and obtaining proteins that specifically interact with other proteins or polypeptides, for example, a yeast two-hybrid screening system such as that described in U.S. Pat. No. 5,283,173 and U.S. Pat. No. 5,468,614, or the equivalent.

In certain preferred embodiments, the binding domain fusion protein comprise, consist essentially of, or consist of, binding domains that are derived from antibodies and immunoglobulins. As such, the binding domains may comprise, for example, monoclonal antibodies (including full length monoclonal antibodies), polyclonal antibodies, multispecific antibodies (e.g., bispecific antibodies), diabodies, triabodies, modified antibodies (e.g., humanized), single chain Fvs (scFvs), and any antibody fragment that exhibits the desired binding activity. Immunoglobulin molecules useful in the invention include proteins of the five human immunoglobulin classes, IgG, IgM, IgA, IgE, and IgD. These include IgG1, IgG2, IgG3, IgG4, IgA1, and IgA2.

Antibody fragments useful in the invention include, for example, light chain variable regions (V_{L} and V_{H}), Fv regions, Fd regions (a fragment comprising V_{H} and CH1, i.e., the two N-terminal domains of the heavy chain), hinge regions (including partial hinge regions, the upper hinge region, the core region, and/or the lower hinge region, with or without glycosylation sites), Fc regions (the “fragment crystallizable” region, derived from constant regions and formed after pepsin digestion), and Fab (“fragment antigen-binding”) regions. Any or all of the above fragments included within constructs of the invention may be naturally occurring. Any or all of the above fragments included within constructs of the invention may be non-naturally occurring, for example, mutated or otherwise altered by amino acid deletion, substitution, or insertion. Antibody fragments, for example immunoglobulin light chain variable region polypeptides and/or heavy chain variable region polypeptides, may be synthetically prepared by techniques well known to those in the art, for example by using different sets of oligonucleotide primers in the polymerase chain reaction. These synthetically prepared antibody fragments may comprise amino acids sequences identical to, or variants of, known or naturally occurring immunoglobulin sequences.

In one aspect of the invention, the binding domain comprises, consists essentially of, or consists of, antibodies, variable regions of H and L chains in either orientation, extracellular domains of cell surface receptors, variable regions of H chains, variable regions of L chains, complementarity determining regions (CDR) to such as CDR3 of H and L chains, humanized camelid H chains, polypeptides selected from phage display libraries, and cytokines that react specifically with receptors displayed on the surface of cells.

Constructs of the invention may or may not include or make use of nonconventional immunoglobulins such as those found in camelids (camels, dromedaries and llamas; Hamers-Casterman et al., 1993 Nature 363: 446; Nguyen et al., 1998 J. Mol. Biol 275: 413), nurse sharks (Roux et al., 1998 Proc. Nat. Acad. Sci. USA 95: 11804), and spotted ratfish (Nguyen, et al., “Heavy-chain antibodies in Camelidae; a case of evolutionary innovation,” 2002 Immunogenetics 54(1):39-47). These antibodies can form antigen-binding regions using only heavy chain variable region, i.e., these functional antibodies are homodimers of heavy chains only (referred to as “heavy-chain antibodies” or “HCAbs”).

Constructs of the invention may or may not include mutations or alterations in immunoglobulin variable region sequences or sequence fragments.

Constructs of the invention may or may not include mutations or alterations in immunoglobulin constant region sequences or sequence fragments.

In an embodiment of the invention, the binding domain fusion protein comprise binding domains that may include at least one native or engineered immunoglobulin variable region polypeptide, such as all or a portion or fragment of a native or engineered heavy chain and/or a native or engineered light chain V-region, provided it is capable of binding or specifically binding an antigen or other desired target structure of interest at a desired level of binding and selectivity.

In other preferred embodiments the binding region or binding domain comprises, consists essentially of, or consists of, a single chain immunoglobulin-derived Fv product, for example, an scFv, which may include all or a portion of at least one native or engineered immunoglobulin light chain V-region and all or a portion of at least one native or engineered immunoglobulin heavy chain V-region, and a linker fused or otherwise connected to the V-regions. ScFvs including naturally occurring variable regions, as well as variable regions that have been mutated or otherwise altered. Other preparation and testing methods are well known in the art. The preparation of single polypeptide chain binding molecules of the Fv region, single-chain Fv molecules, is known in the art. See, e.g., U.S. Pat. No. 4,946,778. In the present invention, single-chain Fv-like molecules that may be included in constructs of the invention may be synthesized by encoding a first variable region of the heavy or light chain, followed by one or more linkers to the variable region of the corresponding light or heavy chain, respectively.

The selection of various appropriate linker(s) between the two variable regions is described in U.S. Pat. No. 4,946,778 (see also, e.g., Huston et al., 1993 Int. Rev. Immunol. 10: 195). An exemplary linker described herein is (Gly-Gly-Gly-Gly-Ser)₃, but may be of any desired length. The linker is used to convert the naturally aggregated but chemically separate heavy and light chains into the amino terminal antigen binding portion of a single polypeptide chain, for example, wherein this antigen binding portion will fold into a structure similar to the original structure made of two polypeptide chains, or that otherwise has the ability to bind to a target, for example a target antigen. In one aspect, the binding domain is comprised of H and L chain variable regions assembled in a binding domain fusion protein so as to recognize and bind to a target molecule. In one aspect of the invention, the linker between H and L variable region or L and H variable region is short and contains less than 10 amino acids, preferably 3 to 10 amino acids, more preferably 2 to 7 amino acids, and most preferably 4 to 6 amino acids, so as to prevent the H and L variable regions (in any orientation) on one polypeptide chain from forming a combining site that recognizes the target and require that H and L regions on two polypeptide chains to form the combining site. In another aspect of the invention, the linker between H and L variable region or L and H variable region is long and contains more than 12 amino acids, preferably 12 to 30 amino acids, more preferably 12 to 20 amino acids, and most preferably 14 to 16 amino acids, so as to allow H and L regions (in any orientation) on one polypeptide chain to form a combining site that recognizes the target. In yet another aspect, the invention includes a construct wherein the binding region binds to an antigen on an immune effector cell. A partial list of particular scFv's that are utilized as the binding domain in specific embodiments is described herein below.

According to certain embodiments of the present invention, the binding domain polypeptide comprises, consists essentially of, or consists of, (a) at least one native or engineered immunoglobulin light chain variable region polypeptide; (b) at least one native or engineered immunoglobulin heavy chain variable region polypeptide; and (c) at least one linker polypeptide that is fused or otherwise connected to the polypeptide of (a) and to the polypeptide of (b). In certain further embodiments the native or engineered immunoglobulin light chain variable region and heavy chain variable region polypeptides are constructed from human immunoglobulins, and in certain other further embodiments the linker polypeptide comprises at least one polypeptide including or having as an amino acid sequence Gly-Gly-Gly-Gly-Ser (SEQ ID NO:______. In other embodiments the linker polypeptide comprises at least two or three repeats of a polypeptide having as an amino acid sequence Gly-Gly-Gly-Gly-Ser (SEQ ID NO:______. In other embodiments the linker comprises a glycosylation site, which in certain further embodiments is an asparagine-linked glycosylation site, an O-linked glycosylation site, a C-mannosylation site, a glypiation site or a phosphoglycation site.

Portions, for example, of a particular immunoglobulin reference sequence and of any one or more additional immunoglobulin sequences of interest that may be compared to a reference sequence. “Corresponding” sequences, regions, fragments or the like, may be identified based on the convention for numbering immunoglobulin amino acid positions according to Kabat, Sequences of Proteins of Immunological Interest, (5^{th} ed. Bethesda, Md.: Public Health Service, National Institutes of Health (1991)). As described herein and known in the art, immunoglobulins comprise products of a gene family the members of which exhibit a high degree of sequence conservation. Amino acid sequences of two or more immunoglobulins or immunoglobulin domains or regions or portions thereof (e.g., V_{H} domains, V_{L} domains, hinge regions, CH2 constant regions, CH3 constant regions) may be aligned and analyzed. Portions of sequences that correspond to one another may be identified, for instance, by sequence homology. Determination of sequence homology may be determined with any of a number of sequence alignment and analysis tools, including computer algorithms well known to those of ordinary skill in the art, such as Align or the BLAST algorithm (Altschul, 1991 J. Mol. Biol. 219: 555-565; Henikoff and Henikoff, 1992 Proc. Natl. Acad. Sci. USA 89: 10915-10919), which is available at the NCBI website (http://www/ncbi.nlm.nih.gov/cgi-bin/BLAST). Default parameters may be used. In certain preferred embodiments, an immunoglobulin sequence of interest or a region, portion, derivative or fragment thereof is greater than about 95% identical to a corresponding reference sequence, and in certain preferred embodiments such a sequence of interest may differ from a corresponding reference at no more than about 1, 2, 3, 4, 5, 6, 7, 8, 9 or 10 amino acid positions.

For example, in certain embodiments the present invention is directed to a construct, comprising in pertinent part a human or other species immunoglobulin heavy chain variable region polypeptide comprising a mutation, alteration or deletion at an amino acid at a location or locations corresponding to one or more of amino acid positions 9, 10, 11, 12, 108, 110, 111, and 112 in, for example, a murine V_{H}-derived sequence. In certain embodiments, for example, the present invention is also directed to a construct, comprising in pertinent part a human immunoglobulin light chain variable region polypeptide, or an immunoglobulin light chain variable region polypeptide from another species, comprising a mutation, alteration or deletion at an amino acid at a location or locations corresponding to one or more of amino acid positions 12, 80, 81, 82, 83, 105, 106, 107 and 108. In still other certain embodiments, for example, the present invention is directed to a construct, comprising in pertinent part (1) a human immunoglobulin heavy chain variable region polypeptide, or an immunoglobulin light chain variable region polypeptide from another species, comprising, consisting essentially of, or consisting of, said heavy chain sequence having a mutation, alteration or deletion at a location or locations corresponding to one or more of amino acid positions 9, 10, 11, 12, 108, 110, 111, and 112, and (2) a human immunoglobulin light chain variable region polypeptide, or an immunoglobulin light chain variable region polypeptide from another species, comprising, consisting essentially of, or consisting of, said light chain sequence having a mutation, alteration or deletion at a location or locations corresponding to one or more of amino acid positions 12, 80, 81, 82, 83, 105, 106, 107 and 108.

As another example, by reference to immunoglobulin sequence compendia and databases such as those cited above, for example, the relatedness of two or more immunoglobulin sequences to each other can readily and without undue experimentation be established in a manner that permits identification of the animal species of origin, the class and subclass (e.g., isotype) of a particular immunoglobulin or immunoglobulin region polypeptide sequence. Any immunoglobulin variable region polypeptide sequence, including native or engineered V_{H} and/or V_{L} and/or single-chain variable region (sFv) sequences or other native or engineered V region-derived sequences or the like, may be used as a binding region or binding domain. Engineered sequences includes immunoglobulin sequences from any species, preferably human or mouse, for example, that include, for example, a mutation, alteration or deletion at an amino acid at a location or locations corresponding to one or more of amino acid positions 9, 10, 11, 12, 108, 110, 111, and 112 in a heavy chain variable region sequence or an scFv, and/or a mutation, alteration or deletion at a location or locations corresponding to one or more of amino acid positions 12, 80, 81, 82, 83, 105, 106, 107 and 108 in a light chain variable region sequence or an scFv.

Various embodiments include, for example, native or engineered immunoglobulin V region polypeptide sequences derived, for example, from antibodies including monoclonal antibodies such as murine or other rodent antibodies, or antibodies or monoclonal antibodies derived from other sources such as goat, rabbit, equine, bovine, camelid or other species, including transgenic animals, and also including human or humanized antibodies or monoclonal antibodies. Non-limiting examples include variable region polypeptide sequences derived from monoclonal antibodies such as those referenced herein and/or described in greater detail in pending applications U.S. Pat. No. 10/627,556 filed Jul. 26, 2003 by Ledbetter et al. entitled “BINDING CONSTRUCTS AND METHODS OF USE THEREOF”, and PCT/US03/41600 filed Dec. 24, 2003 by Ledbetter et al. entitled “BINDING CONSTRUCTS AND METHODS OF USE THEREOF.”

Other binding regions, including binding domain polypeptides, may comprise any protein or portion thereof that retains the ability to bind or specifically bind to an antigen as provided herein, including non-immunoglobulins. Accordingly the invention contemplates constructs, including fusion proteins, comprising binding region or binding domain polypeptides that are derived from polypeptide ligands such as hormones, cytokines, chemokines, and the like; cell surface or soluble receptors for such polypeptide ligands; lectins; intercellular adhesion receptors such as specific leukocyte integrins, selecting, immunoglobulin gene superfamily members, intercellular adhesion molecules (ICAM-1, -2, -3) and the like; histocompatibility antigens; etc.

Other binding regions within the molecules of the invention may include domains that comprise sites for glycosylation, for example, covalent attachment of carbohydrate moieties such as monosaccharides or oligosaccharides.

Still other binding regions within molecules of the invention include polypeptides that may comprise proteins or portions thereof that retain the ability to specifically bind another molecule, including an antigen. Thus, binding regions may comprise or be derived from hormones, cytokines, chemokines, and the like; cell surface or soluble receptors for such polypeptide ligands; lectins; intercellular adhesion receptors such as specific leukocyte integrins, selectins, immunoglobulin gene superfamily members, intercellular adhesion molecules (ICAM-1, -2, -3) and the like; histocompatibility antigens; and so on. Binding regions derived from such molecules generally will include those portions of the molecules necessary or desired for binding to a target.

Generally, target-associated molecules, and in particular proteinase-associated targets, include any protein, carbohydrate, nucleic acid, or other organic molecule. Target-associated molecules may be expressed on the cell surface or in particular cell types, in particular tissues, or at particular locations in an animal or subject. For example, target-associated molecules may be expressed on leukocytes, T lymphocytes (e.g., CD2, CD3, CD4, CD5, CD6, CD7, CD8, CD25, CD28, CD69, CD154, CD152 (CTLA-4), and ICOS antigens), helper T cells, monocytes, dendritic cells, immune effector cells, B cells (e.g., MHC class II, CD19, CD20, CD21, CD22, CD23, CD37 and CD40 antigens). Cell surface markers are another type of target-associated molecules, including cell surface markers from normal or malignant cells. Other binding domain targets include cell surface markers from normal or malignant cells; cytokines (including growth factors and mediators of signal transduction); proteins in the blood or tissues; infectious targets including viral, bacterial, fungal and parasite targets; and intracellular targets, including intracellular protein targets.

Cell surface antigens/receptors that may be proteinase-associated targets for the binding domain polypeptides, or alternatively as suitable sources of binding region or binding domain polypeptides or portions thereof, include the following, or the like: CD2 (e.g., GenBank Ace. Nos. Y00023, SEG_HUMCD2, M16336, M16445, SEG_MUSCD2, M14362), 4-1BB (CDw137, Kwon et al., 1989 Proc. Nat. Acad. Sci. USA 86:1963, 4-IBB ligand (Goodwin et al., 1993 Eur. J. Immunol. 23:2361; Melero et al., 1998 Eur. J. Immunol. 3:116), CD5 (e.g., GenBank Acc. Nos. X78985, X89405), CD10 (e.g., GenBank Acc. Nos. M81591, X76732), CD27 (e.g., GenBank Acc. Nos. M63928, L24495, L08096), CD28 (June et al., 1990 Immunol. Today 11:211; see also, e.g., GenBank Acc. Nos. J02988, SEG_HUMCD28, M34563), CD152/CTLA-4 (e.g., GenBank Acc. Nos. L15006, X05719, SEG_HUMIGCTL), CD40 (e.g., GenBank Acc. Nos. M83312, SEG_MUSC040A0, Y10507, X67878, X96710, U15637, L07414), interferon-γ (IFN-γ; see, e.g., Farrar et al. 1993 Ann. Rev. Immunol. 11:571 and references cited therein, Gray et al. 1982 Nature 295:503, Rinderknecht et al. 1984 J. Biol. Chem. 259:6790, DeGrado et al. 1982 Nature 300:379), interleukin-4 (IL-4; see, e.g., 53^{rd} Forum in Immunology, 1993 Research in Immunol. 144:553-643; Banchereau et al., 1994 in The Cytokine Handbook, 2^{nd} ed., A. Thomson, ed., Academic Press, NY, p. 99; Keegan et al., 1994 J Leukocyt. Biol. 55:272, and references cited therein), interleukin-17 (IL-17) (e.g., GenBank Acc. Nos. U32659, U43088) and interleukin-17 receptor (IL-17R) (e.g., GenBank Acc. Nos. U31993, U58917).

Additional cell surface antigens/receptors that may be proteinase-associated targets for the binding domain polypeptides, or alternatively as suitable sources of binding region or binding domain polypeptides or portions thereof, include the following, or the like: CD59 (e.g., GenBank Acc. Nos. SEG_HUMCD590, M95708, M34671), CD48 (e.g., GenBank Acc. Nos. M59904), CD58/LFA-3 (e.g., GenBank Acc. No. A25933, Y00636, E12817; see also JP 1997075090-A), CD72 (e.g., GenBank Acc. Nos. AA311036, S40777, L35772), CD70 (e.g., GenBank Acc. Nos. Y13636, S69339), CD80/B7.1 (Freeman et al., 1989 J. Immunol. 43:2714; Freeman et al., 1991 J. Exp. Med. 174: 625; see also e.g., GenBank Acc. Nos. U33208, I683379), CD86/B7.2 (Freeman et al., 1993 J. Exp. Med. 178: 2185, Boriello et al., 1995 J. Immunol. 155: 5490; see also, e.g., GenBank Acc. Nos. AF099105, SEG_MMB72G, U39466, U04343, SEG_HSB725, L25606, L25259), B7-H1/B7-DC (e.g., Genbank Acc. Nos. NM_{—}014143, AF177937, AF317088; Dong et al., 2002 Nat. Med. June 24 (epub ahead of print), PMID 12091876; Tseng et al., 2001 J. Exp. Med. 193: 839; Tamura et al., 2001 Blood 97: 1809; Dong et al., 1999 Nat. Med. 5: 1365), CD40 ligand (e.g., GenBank Acc. Nos. SEG_HUMCD40L, X67878, X65453, L07414), IL-17 (e.g., GenBank Acc. Nos. U32659, U43088), CD43 (e.g., GenBank Acc. Nos. X52075, J04536), ICOS (e.g., Genbank Acc. No. AH011568), CD3 (e.g., Genbank Acc. Nos. NM_{—}000073 (gamma subunit), NM_{—}000733 (epsilon subunit), X73617 (delta subunit)), CD4 (e.g., Genbank Acc. No. NM_{—}000616), CD25 (e.g., Genbank Acc. No. NM_{—}000417), CD8 (e.g., Genbank Acc. No. M12828), CD8a (T-cell surface glycoprotein CD8 alpha chain, also referred to as T-lymphocyte differentiation antigen, T8/Leu-2, and Lyt-2); CD11b (e.g., Genbank Acc. No. J03925), CD14 (e.g., Genbank Acc. No. XM_{—}039364), CD56 (e.g., Genbank Acc. No. U63041), CD69 (e.g., Genbank Acc. No. NM_{—}001781) and VLA-4 (α₄β7) (e.g., GenBank Acc. Nos. L12002, X16983, L20788, U97031, L24913, M68892, M95632). The following cell surface receptors are typically associated with B cells: CD19 (e.g., GenBank Acc. Nos. SEG_HUMCD19W0, M84371, SEG_MUSCD19W, M62542), CD20 (e.g., GenBank Acc. Nos. SEG_HUMCD20, M62541), CD22 (e.g., GenBank Acc. Nos. I680629, Y10210, X59350, U62631, X52782, L16928), CD30 (e.g., Genbank Acc. Nos. M83554, D86042), CD153 (CD30 ligand, e.g., GenBank Acc. Nos. L09753, M83554), CD37 (e.g., GenBank Acc. Nos. SEG_MMCD37X, X14046, X53517), CD50 (ICAM-3, e.g., GenBank Acc. No. NM_{—}002162), CD106 (VCAM-1) (e.g., GenBank Acc. Nos. X53051, X67783, SEG_MMVCAM1C, see also U.S. Pat. No. 5,596,090), CD54 (ICAM-1) (e.g., GenBank Acc. Nos. X84737, S82847, X06990, J03132, SEG-_MUSICAM0), interleukin-12 (see, e.g., Reiter et al, 1993 Crit. Rev. Immunol. 13: 1, and references cited therein), CD134 (OX40, e.g., GenBank Acc. No. AJ277151), CD137 (41BB, e.g., GenBank Acc. No. L12964, NM_{—}001561), CD83 (e.g., GenBank Acc. Nos. AF001036, AL021918), DEC-205 (e.g., GenBank Acc. Nos. AF011333, U19271), CD6, CD7 (Entrez protein AAH24376 [Mus musculus], Entrez nucleotide AY407406 [Homo sapiens]), CD21 (Entrez protein CAA66910 [Homo sapiens], Entrez nucleotide AF298224, X98257 [Homo sapiens], Entrez nucleotide AF168683 [Mus musculus]), CD23 (Entrez protein AAL84004, CAA51981, Entrez nucleotide AF381978, X73579 [Rattus norvegicus], Entrez protein AAB28793, AAB28792, AAB28791 Entrez nucleotide AI449163[Mus musculus], Entrez nucleotide E04250 [Homo sapiens], CD45 (Entrez protein AAS46962, AAS46954, AAS46946, AAS46938, AAS46930, AAS46922, Entrez nucleotide AY539659, AY539707, AY539699, AY539691, AY539683, AY539675, AY539667, AJ006102 [Homo sapiens], Entrez protein AAB34268, AAB34274, AAB34272, AAB34270, AAB34268 [Mus musculus], CD45 RA, CD45 RO, CD154 (Entrez nucleotide AY333790 [Canis familiaris], MHC class II [Entrez protein CAD62436, CAD62435, AAB08109 [Homo sapiens], Entrez protein I56028 [Mus musculus]), VEGF (Entrez protein NP_{—}003368, AAD03710, AAC63143, CAA44447 [Homo sapiens], Entrez nucleotide NM_{—}003376, AY047581 [Homo sapiens]).

Other proteinase-associated molecules include tumor antigens. Examples of tumor antigens that may be targeted by constructs of the invention include Squamous Cell Carcinoma Antigen 1 (SCCA-1) (Protein T4-A); Squamous Cell Carcinoma Antigen 2 (SCCA-2); Ovarian carcinoma antigen CA125 (1A1-3B) (KIAA0049); Mucin 1 (Tumor-Associated Mucin) (Carcinoma-Associated Mucin) (Polymorphic Epithelial Mucin) (Pem) (Pemt) (Episialin) (Tumor-Associated Epithelial Membrane Antigen) (Ema) (H23AG) (Peanut-Reactive Urinary Mucin) (Pum) (Breast Carcinoma-Associated Antigen DF3); CTCL tumor antigen se1-1; CTCL tumor antigen se14-3; CTCL tumor antigen se20-4; CTCL tumor antigen se20-9; CTCL tumor antigen se33-1; CTCL tumor antigen se37-2; CTCL tumor antigen se57-1; CTCL tumor antigen se89-1; Prostate-specific membrane antigen; 5T4 oncofetal trophoblast glycoprotein; Orf73 Kaposi's sarcoma-associated herpesvirus; MAGE-C1 (cancer/testis antigen CT7); MAGE-B1 antigen (MAGE-XP antigen) (DAM10); MAGE-B2 antigen (DAM6); MAGE-2 antigen; MAGE-4a antigen; MAGE-4b antigen; Colon cancer antigen NY-CO-45; Lung cancer antigen NY-LU-12 variant A; Cancer associated surface antigen; Adenocarcinoma antigen ART1; Paraneoplastic associated brain-testis-cancer antigen (onconeuronal antigen MA2; paraneoplastic neuronal antigen); Neuro-oncological ventral antigen 2 (NOVA2); Hepatocellular carcinoma antigen gene 520; Tumor-associated antigen CO-029; Tumor-associated antigen MAGE-X2; Synovial sarcoma, X breakpoint 2; Squamous cell carcinoma antigen recognized by T cell; Serologically defined colon cancer antigen 1; Serologically defined breast cancer antigen NY-BR-15; Serologically defined breast cancer antigen NY-BR-16; Chromogranin A; parathyroid secretory protein 1; DUPAN-2; CA 19-9; CA 72-4; CA 195; and, L6 (Tumor-associated antigen L6, also referred to as Transmembrane 4 superfamily member 1, Membrane component surface marker 1, or M3S1).

Examples of cell surface receptors that may be proteinase-associated targets for the binding domain polypeptides, or alternatively as suitable sources of binding region or binding domain polypeptides or portions thereof, include the following, or the like: HER1 (e.g., GenBank Accession Nos. U48722, SEG_HEGFREXS, K03193), HER2 (Yoshino et al., 1994 J. Immunol. 152: 2393; Disis et al., 1994 Canc. Res. 54: 16; see also, e.g., GenBank Acc. Nos. X03363, M17730, SEG_HUMHER20), HER3 (e.g., GenBank Acc. Nos. U29339, M34309), HER4 (Plowman et al., 1993 Nature 366: 473; see also e.g., GenBank Acc. Nos. L07868, T64105), epidermal growth factor receptor (EGFR) (e.g., GenBank Acc. Nos. U48722, SEG_HEGFREXS, K03193), vascular endothelial cell growth factor (e.g., GenBank No. M32977), vascular endothelial cell growth factor receptor (e.g., GenBank Acc. Nos. AF022375, I680143,. U48801, X62568), insulin-like growth factor-I (e.g., GenBank Acc. Nos. X00173, X56774, X56773, X06043, see also European Patent No. GB 2241703), insulin-like growth factor-II (e.g., GenBank Acc. Nos. X03562, X00910, SEG_HUMGFIA, SEG_HUMGFI2, M17863, M17862), transferrin receptor (Trowbridge and Omary, 1981 Proc. Nat. Acad. USA 78:3039; see also e.g., GenBank Acc. Nos. X01060, M11507), estrogen receptor (e.g., GenBank Acc. Nos. M38651, X03635, X99101, U47678, M12674), progesterone receptor (e.g., GenBank Acc. Nos. X51730, X69068, M15716), follicle stimulating hormone receptor (FSH-R) (e.g., GenBank Acc. Nos. Z34260, M65085), retinoic acid receptor (e.g., GenBank Acc. Nos. L12060, M60909, X77664, X57280, X07282, X06538), MUC-1 (Barnes et al., 1989 Proc. Nat. Acad. Sci. USA 86: 7159; see also e.g., GenBank Acc. Nos. SEG_MUSMUCIO, M65132, M64928), NY-ESO-1 (e.g., GenBank Acc. Nos. AJ003149, U87459), NA 17-A (e.g., European Patent No. WO 96/40039), Melan-A/MART-1 (Kawakami et al., 1994 Proc. Nat. Acad. Sci. USA 91:3515; see also e.g., GenBank Acc. Nos. U06654, U06452), tyrosinase (Topalian et al., 1994 Proc. Nat. Acad. Sci. USA 91:9461; see also e.g., GenBank Acc. Nos. M26729, SEG_HUMTYR0, see also Weber et al., J. Clin. Invest (1998) 102: 1258), Gp-100 (Kawakami et al., 1994 Proc. Nat. Acad Sci. USA 91: 3515; see also e.g., GenBank Acc. No. S73003, see also European Patent No. EP 668350; Adema et al., 1994 J. Biol. Chem. 269: 20126), MAGE (van den Bruggen et al., 1991 Science 254: 1643; see also e.g, GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735, M77481), BAGE (e.g., GenBank Acc. No. U19180; see also U.S. Pat. Nos. 5,683,886 and 5,571,711), GAGE (e.g., GenBank Acc. Nos. AF055475, AF055474, AF055473, U19147, U19146, U19145, U19144, U19143, U19142), any of the CTA class of receptors including in particular HOM-MEL-40 antigen encoded by the SSX2 gene (e.g., GenBank Acc. Nos. X86175, U90842, U90841, X86174), carcinoembyonic antigen (CEA, Gold and Freedman, 1985 J. Exp. Med. 121: 439; see also e.g., GenBank Acc. Nos. SEG_HUMCEA, M59710, M59255, M29540), and PyLT (e.g., GenBank Acc. Nos. J02289, J02038).

Binding regions within the molecules of the invention may comprise, for example, binding domains for desired protease-associated molecules, including antigen-binding targets. Binding domains may preferably comprise single chain Fvs and scFv domains. In certain embodiments, molecules of the invention may comprise a binding region having at least one immunoglobulin variable region polypeptide, which may be a light chain or a heavy chain variable region polypeptide. In certain embodiments, molecules of the invention may comprise at least one such light chain V-region and one such heavy chain V-region and at least one linker peptide that connects the V-regions. ScFvs useful in the invention also include those with chimeric binding or other domains or sequences. Other ScFvs useful in the invention also include those with humanized (in whole or in part) binding or other domains or sequences. In such embodiments, all or a portion of an immunoglobulin binding or other sequence that is derived from a non-human source may be “humanized” in whole or in part according to recognized procedures for generating humanized antibodies, i.e., immunoglobulin sequences into which human Ig sequences are introduced to reduce the degree to which a human immune system would perceive such proteins as foreign.

In certain embodiments, the binding domain of the binding domain fusion proteins comprise scFv's that are capable of binding to particular proteinase-associated molecules. Example of scFvs useful in the invention, whether included as murine or other scFvs (including human scFvs), chimeric scFvs, or humanized scFvs, in whole or in part, include but are not limited to anti-human CD28 scFvs (for example, “2E12” scFvs), VEGF scFvs ((for example “LL4” scFvs (Peregrine Pharmaceuticals, Inc), anti-human-VEGF scFvs (see U.S. Pat. No. 6,703,020 to Thorpe et al. and ATCC PTA 1595), and anti-human-VEGF “JH1” scFvs.

In certain preferred embodiments, the polynucleotides having sequence homology or sequence identity immunoglobulin variable region sequences, including those that are known and/or publicly available, are synthetically produced, such as for example by oligonucleotide synthesis, PCR, and other techniques known in the art.

In another aspect of the invention, preferred inhibited proteinases are matrix metalloproteinases, including, but not limited to, matrix metalloproteinases that release fragments of vascular endothelial growth factor (VEGF) from a precursor. Other proteases and modulators of the expression of various isoforms of VEGF are targets of certain embodiments of the binding domain fusion proteins and constructs described herein, including for example proprotein convertases such as furin, furin-motif containing proteins, furin-motif variants, PC5, and PC7. See for example, Siegfried et al., 2003 J. Clin. Invest., 111(11):1723-1732, Joukov V., et al., 1997 EMBO J 16:3898-3911; Khatib A. M., et al., 2002 Am. J Pathol. 160:1921-1935; Zhong, M. et al., 1999 J. Biol. Chem. 274:33913-33920; L Nakayama, K., 1997 Biochem. J. 327:625-635; Salven P., et al. 1998 Am. J. Pathol. 153:103-108 and Seidah, N. G. et al., 1999 Brain Res. 848:45-62; the contents of which are all incorporated by reference herein, and which may included or excluded from certain embodiments of the invention. In another aspect, binding domain fusion proteins inhibit the expression of vascular endothelial growth factor (VEGF) or reduce the amount of VEGF. In exemplary embodiments, binding domain fusion proteins inhibit the expression or reduce the amount of one or more of the three major isoforms (VEGF 121, VEGF 165, and VEGF 189). In certain embodiments, the expression or amount of one or more isoforms of VEGF (e.g. VEGF 121, VEGF 165, and VEGF 189) is reduced at a predetermined desired site.

Certain constructs may comprise binding domains that bind to a VEGF, including for example, anti-human-VEGF scFVs (for example, “LL4” scFvs from Peregrine Pharmaceuticals, Inc), chimeric anti-VEGF antibody (for example, “vmD11”, as described in Ran Y et al., “Construction of anti-human VEGF165 chimeric antibodies and expression in eukaryotic cells” Zhonghua Zhong Liu Za Zhi. November 1999; 21(6): 412-5), anti-human-VEGF scFvs (for example, as described in U.S. Pat. No. 6,703,020 to Thorpe et al. and ATCC PTA 1595), and anti-human-VEGF scFvs (for example, “JH1” scFv, as described in Kulawiec M. et al., “Characterization of a novel bispecific fusion protein incorporating anti-VEGF single chain antibody fragment JH1 and anti-HER2/neu peptide AHNP” Abstracts submitted to the 2003 Annual Meeting of the Regional Cancer Center Consortium for the Biological Therapy of Cancer; page 17).

scFvs useful in the invention also include scFvs, including chimeric and humanized scFvs, having one or more amino acid substitutions. A preferred amino acid substitution is at amino acid position 11 in the variable heavy chain (the V_{H}). Such a substitution may be referred to herein as “XxxV_{H}11Zxx”. Thus, for example, where the normally occurring amino acid at position V_{H}11 is a Leucine, and a Serine amino acid residue is substituted therefore, the substitution is identified as “L V_{H}11S” or “Leu V_{H}11Ser.” Other preferred embodiments of the invention include molecules containing scFvs wherein the amino acid residue normally found at position V_{H}11 is deleted. Still other preferred embodiments embodiments of the invention include molecules containing scFvs wherein the amino acid residues normally found at positions V_{H}10 and/or V_{H}11 and/or V_{H}12 are substituted or deleted.

In other embodiments, the binding domain fusion proteins comprise one or more transglutaminase domain (TGase domain or TGase substrate domain). An exemplary TGase motif comprises, consists essentially of, or consists of, for example, the amino acid sequence Gly-Gln-Asp-Pro-Val-Lys; however, any peptide or polypeptide comprising another amino acid sequence may be used here as a TGase motif so long as it is a functionally active substrate for a transglutaminase. Certain embodiments of binding domain fusion proteins have one or more than one TGase motif, for example one, two, three, four, five, one or two, one to five, or more than five TGase motifs (e.g., Gly-Gln-Asp-Pro-Val-Lys). Certain other exemplary embodiments have between about three and about fifteen TGase motifs (e.g., Gly-Gln-Asp-Pro-Val-Lys), and certain other embodiments have between about four and about ten TGase motifs (e.g., Gly-Gln-Asp-Pro-Val-Lys). In certain embodiments, a TGase domain of a binding domain fusion protein or other construct described herein can allow the binding domain fusion proteins to be anchored at a particular site, for example a site that provides additional biological activity and improves treatment efficiency. Transglutaminase enzymes are described in U.S. Pat. No. 5,428,014 entitled “Transglustaminase cross-linkable polypeptides and methods relating thereto”, and U.S. Pat. No. 5,952,011 entitled “Human Transglutaminases”, the contents of which are both are hereby incorporated by reference herein in their entirety, and which may included or excluded from certain embodiments of the invention.

Various molecules of the invention described and claimed herein include a connecting region joining one domain of the molecule to another domain. In other certain embodiments, the binding domain polypeptide fusion protein does not have a connecting region or spacer region.

The connecting regions may comprise, for example, immunoglobulin hinge region polypeptides, including any hinge peptide or polypeptide that occurs naturally. A connecting region may also include, for example, any artificial peptide or other molecule (including, for example, non-peptide molecules, partial peptide molecules, and peptidomimetics, etc.) useful for joining the tail region and the binding region. These may include, for example, alterations of molecules situated in an immunoglobulin heavy chain polypeptide between the amino acid residues responsible for forming intrachain immunoglobulin-domain disulfide bonds in CH1 and CH2 regions. Naturally occurring hinge regions include those located between the constant region domains, CH1 and CH2, of an immunoglobulin. Useful immunoglobulin hinge region polypeptides include, for example, human immunoglobulin hinge region polypeptides and llama or other camelid immunoglobulin hinge region polypeptides. Other useful immunoglobulin hinge region polypeptides include, for example, nurse shark and spotted ratfish immunoglobulin hinge region polypeptides. Human immunoglobulin hinge region polypeptides include, for example, wild type IgG hinges including wild-type human IgG1 hinges, human IgG-derived immunoglobulin hinge region polypeptides, a portion of a human IgG hinge or IgG-derived immunoglobulin hinge region, wild-type human IgA hinge region polypeptides, human IgA-derived immunoglobulin hinge region polypeptides, a portion of a human IgA hinge region polypeptide or IgA-derived immunoglobulin hinge region polypeptide, wild-type human IgD hinge region polypeptides, human Ig-D derived immunoglobulin hinge region polypeptides, a portion of a human IgD hinge region polypeptide or IgD-derived immunoglobulin hinge region polypeptide, wild-type human IgE hinge-acting region, i.e., IgE CH2 region polypeptides (which generally have 5 cysteine residues), human IgE-derived immunoglobulin hinge region polypeptides, a portion of a human IgE hinge-acting region, i.e., IgE CH2 region polypeptide or IgE-derived immunoglobulin hinge region polypeptide, and so on. A polypeptide “derived from” or that is “a portion or fragment of” an immunoglobulin polypeptide chain region regarded as having hinge function has one or more amino acids in peptide linkage, for example 15-115 amino acids, preferably 95-110, 5-15, 80-94, 60-80, or 5-65 amino acids, preferably 10-50, more preferably 15-35, still more preferably 18-32, still more preferably 20-30, still more preferably 21, 22, 23, 24, 25, 26, 27, 28 or 29 amino acids. Llama immunoglobulin hinge region polypeptides include, for example, an IgG1 llama hinge.

Such connecting regions also include, for example, mutated or otherwise altered or engineered immunoglobulin hinge region polypeptides. A mutated or otherwise altered or engineered immunoglobulin hinge region polypeptide may comprise, consist essentially of, or consist of, a hinge region that has its origin in an immunoglobulin of a species, of an immunoglobulin isotype or class, or of an immunoglobulin subclass that is the same or different from that of any included native or engineered CH2 and CH3 domains. Mutated or otherwise altered or engineered immunoglobulin hinge region polypeptides include those derived or constructed from, for example, a wild-type immunoglobulin hinge region that contains one or more cysteine residues, for example, a wild-type human IgG or IgA hinge region that naturally comprises three cysteines. In such polypeptides the number of cysteine residues may be reduced by amino acid substitution or deletion or truncation, for example. These polypeptides include, for example, mutated human or other IgG1 or IgG3 hinge region polypeptides containing zero, one, or two cysteine residues, and mutated human or other IgA1 or IgA2 hinge region polypeptides that contain zero, one, or two cysteine residues. Mutated or otherwise altered or engineered immunoglobulin hinge region polypeptides include those derived or constructed from, for example, a wild-type immunoglobulin hinge region that contains three or more cysteine residues, for example, a wild-type human IgG2 hinge region (which has 4 cysteines) or IgG3 hinge region which has 11 cysteines). Mutated or otherwise altered or engineered immunoglobulin hinge region polypeptides include those derived or constructed from, for example, an IgE CH2 wild-type immunoglobulin region that generally contains five cysteine residues. In such polypeptides the number of cysteine residues may be reduced by one or more cysteine residues by amino acid substitution or deletion or truncation, for example. Also included are an altered hinge region polypeptides in which cysteine residues in the hinge region are substituted with serine or one or more other amino acids that are less polar, less hydrophobic, more hydrophilic and/or neutral. Such mutated immunoglobulin hinge region polypeptides include, for example, mutated hinge region polypeptides that contain one cysteine residue and that are derived from a wild-type immunoglobulin hinge region polypeptide having two or more cysteine residues, such as a mutated human IgG or IgA hinge region polypeptide that contains one cysteine residue and that is derived from a wild-type human IgG or IgA region polypeptide. Connecting region polypeptides include immunoglobulin hinge region polypeptides that are compromised in their ability to form interchain, homodimeric disulfide bonds.

Mutated immunoglobulin hinge region polypeptides also include mutated hinge region polypeptides that exhibit a reduced ability to dimerize, relative to a wild-type human immunoglobulin G hinge region polypeptide, and mutated hinge region polypeptides that allow expression of a mixture of monomeric and dimeric molecules. Mutated immunoglobulin hinge region polypeptides also include hinge region polypeptides engineered to contain a glycosylation site. Glycosylation sites include, for example, an asparagine-linked glycosylation site, an O-linked glycosylation site, a C-mannosylation site, a glypiation site, and a phosphoglycation site.

Specific connecting regions useful in molecules of the invention described and claimed herein include, for example, the following 18 amino acid sequences, DQEPKSCDKTHTCPPCPA, DQEPKSSDKTHTSPPSPA, and DLEPKSCDKTHTCPPCPA.

An immunoglobulin hinge region polypeptide may comprise, consist essentially or, or consist of, for example, any of (1) any hinge or hinge-acting peptide or polypeptide that occurs naturally for example, a human immunoglobulin hinge region polypeptide including, for example, a wild-type human IgG hinge or a portion thereof, a wild-type human IgA hinge or a portion thereof, a wild-type human IgD hinge or a portion thereof, or a wild-type human IgE hinge-acting region, i.e., IgE CH2, or a portion thereof, a wild-type camelid hinge region or a portion thereof (including a IgG1 llama hinge region or portion thereof, a IgG2 llama hinge region or portion thereof, and a IgG3 llama hinge region or portion thereof), a nurse shark hinge region or portion thereof, and/or a spotted ratfish hinge region or a portion thereof; (2) a mutated or otherwise altered or engineered hinge region polypeptide that contains no cysteine residues and that is derived or constructed from a wild-type immunoglobulin hinge region polypeptide having one or more cysteine residues; (3) a mutated or otherwise altered or engineered hinge region polypeptide that contains one cysteine residue and that is derived from a wild-type immunoglobulin hinge region polypeptide having one or more cysteine residues; (4) a hinge region polypeptide that has been mutated or otherwise altered or engineered to contain or add one or more glycosylation sites, for example, an asparagine-linked glycosylation site, an O-linked glycosylation site, a C-mannosylation site, a glypiation site or a phosphoglycation site; (5) a mutated or otherwise altered or engineered hinge region polypeptide in which the number of cysteine residues is reduced by amino acid substitution or deletion, for example, a mutated or otherwise altered or engineered IgG1 hinge region containing for example zero, one, or two cysteine residues, a mutated or otherwise altered or engineered IgG2 hinge region containing for example zero, one, two or three cysteine residues, a mutated or otherwise altered or engineered IgG3 hinge region containing for example zero, one, two, three, or from four to ten cysteine residues, a mutated or otherwise altered or engineered IgG4 hinge region containing for example zero or one cysteine residues, or a mutated or otherwise altered or engineered human IgA1 or IgA2 hinge region polypeptide that contains zero or only one or two cysteine residues (e.g., an “SCC” hinge), a mutated or otherwise altered or engineered IgD hinge region containing no cysteine residues, or a mutated or otherwise altered or engineered human IgE hinge-acting region, i.e., IgE CH2 region polypeptide that contains zero or only one, two, three or four cysteine residues; or (6) any other connecting region molecule described or referenced herein or otherwise known or later discovered as useful for connecting adjoining immunoglobulin domains such as, for example, a CH1 domain and a CH2 domain. For example, a hinge region polypeptide may be selected from the group consisting of (i) a wild-type human IgG1 immunoglobulin hinge region polypeptide, for example, (ii) a mutated or otherwise altered or engineered human IgG1 or other immunoglobulin hinge region polypeptide that is derived or constructed from a wild-type immunoglobulin hinge region polypeptide having three or more cysteine residues, wherein said mutated human IgG1 or other immunoglobulin hinge region polypeptide contains two cysteine residues and wherein a first cysteine of the wild-type hinge region is not mutated, (iii) a mutated or otherwise altered or engineered human IgG1 or other immunoglobulin hinge region polypeptide that is derived from a wild-type immunoglobulin hinge region polypeptide having three or more cysteine residues, wherein said mutated human IgG1 or other immunoglobulin hinge region polypeptide contains no more than one cysteine residue, and (iv) a mutated or otherwise altered or engineered human IgG1 or other immunoglobulin hinge region polypeptide that is derived from a wild-type immunoglobulin hinge region polypeptide having three or more cysteine residues, wherein said mutated or otherwise altered or engineered human IgG1 or other immunoglobulin hinge region polypeptide contains no cysteine residues. In certain embodiments, for example, the immunoglobulin hinge region polypeptide is a mutated or otherwise altered or engineered hinge region polypeptide and exhibits a reduced ability to dimerize, relative to a wild-type human immunoglobulin G or other wild type hinge region or hinge-acting polypeptide.

An immunoglobulin hinge region polypeptide includes any hinge peptide or polypeptide that occurs naturally, as an artificial peptide or as the result of genetic engineering and that is situated, for example, in an immunoglobulin heavy chain polypeptide between the amino acid residues responsible for forming intrachain immunoglobulin-domain disulfide bonds in CH1 and CH2 regions. Hinge region polypeptides for use in the present invention may also include a mutated or otherwise altered hinge region polypeptide. Accordingly, for example, an immunoglobulin hinge region polypeptide may be derived from, or may be a portion or fragment of (i.e., one or more amino acids in peptide linkage, typically about 15-115 amino acids, preferably about 95-110, about 80-94, about 60-80, or about 5-65 amino acids, preferably about 10-50, more preferably about 15-35, still more preferably about 18-32, still more preferably about 20-30, still more preferably about 21, 22, 23, 24, 25, 26, 27, 28 or 29 amino acids) an immunoglobulin polypeptide chain region classically regarded as having hinge function, including those described herein, but a hinge region polypeptide for use in the instant invention need not be so restricted and may include one or more amino acids situated (according to structural criteria for assigning a particular residue to a particular domain that may vary, as known in the art) in an adjoining immunoglobulin domain such as a CH1 domain and/or a CH2 domain in the cases of IgG, IgA and IgD (or in an adjoining immunoglobulin domain such as a CH1 domain and/or a CH3 domain in the case of IgE), or in the case of certain artificially engineered immunoglobulin constructs, an immunoglobulin variable region domain.

Wild-type immunoglobulin hinge region polypeptides include any known or later-discovered naturally occurring hinge region that is located between the constant region domains, CH1 and CH2, of an immunoglobulin, for example, a human immunoglobulin (or between the CH1 and CH3 regions of certain types of immunoglobulins, such as IgE). For use in constructing one type of connecting region, the wild-type immunoglobulin hinge region polypeptide is preferably a human immunoglobulin hinge region polypeptide, preferably comprising a hinge region from a human IgG, IgA, or IgD immunoglobulin (or the CH2 region of an IgE immunoglobulin), and more preferably, for example, a hinge region polypeptide from a wild-type or mutated human IgG1 isotype as described herein.

As is known to the art, despite the tremendous overall diversity in immunoglobulin amino acid sequences, immunoglobulin primary structure exhibits a high degree of sequence conservation in particular portions of immunoglobulin polypeptide chains, notably with regard to the occurrence of cysteine residues which, by virtue of their sulfyhydryl groups, offer the potential for disulfide bond formation with other available sulfydryl groups. Accordingly, in the context of the present invention wild-type immunoglobulin hinge region polypeptides for use as connecting regions include those that feature one or more highly conserved (e.g., prevalent in a population in a statistically significant manner) cysteine residues, and in certain preferred embodiments a connecting region may comprise, or consist essentially of, or consist of, a mutated hinge region polypeptide may be selected that contains less than the number of naturally-occurring cysteines, for example, zero or one or two cysteine residue(s) in the case of IgG1 hinge regions, or zero or one cysteine residue(s) in the case of IgG4, and that is derived or constructed from (or using) such a wild-type hinge region sequence.

In certain preferred embodiments wherein the connecting region is a hinge region polypeptide and the hinge region polypeptide is a mutated, engineered or otherwise altered human IgG1 immunoglobulin hinge region polypeptide that is derived or constructed from (or using) a wild-type hinge region sequence, it is noted that the wild-type human IgG1 hinge region polypeptide sequence comprises three non-adjacent cysteine residues, referred to as a first cysteine of the wild-type hinge region, a second cysteine of the wild-type hinge region and a third cysteine of the wild-type hinge region, respectively, proceeding along the hinge region sequence from the polypeptide N-terminus toward the C-terminus. This can be referred to herein as a “CCC” hinge (or a “WTH”, i.e., a wild-type hinge). Examples of mutated or engineered hinge regions include those with no cysteines, which may be referred to herein as an “XXX” hinge (or, for example, as “MH-XXX,” referring to a mutant or engineered hinge with three amino acids or other molecules in place of naturally occurring cysteines, such as, for example, “MH-SSS”, which refers to a mutant hinge with three serine residues in place of the naturally occurring cysteine residues. It will be understood that the term “mutant” refers only to the fact that a different molecule or molecules is present, or no molecule, at the position of a naturally occurring residue and does not refer to any particular method by which such substitution, alteration, or deletion has been carried out. Accordingly, in certain embodiments of the present invention, the connecting region may be a hinge region polypeptide and the hinge region polypeptide is a mutated human IgG1 immunoglobulin hinge region polypeptide that contains two cysteine residues and in which the first cysteine of the wild-type hinge region has not changed or deleted, for example. This can be referred to as a “MH-CXX” hinge, for example, a “MH-CSC” hinge, in which case the cysteine residue has been replaced with a serine residue. In certain other embodiments of the present invention the mutated human IgG1 immunoglobulin hinge region polypeptide contains no more than one cysteine residue and include, for example, a “MH-CSS” hinge or a “MH-SSC” hinge or a “MH-SCS” hinge, and in certain other embodiments the mutated human IgG1 immunoglobulin hinge region polypeptide contains no cysteine residues such as, for example, a “MH-SSS” hinge.

A connecting region may comprise a mutated or otherwise altered immunoglobulin hinge region polypeptide, which itself may comprise a hinge region that has its origin in an immunoglobulin of a species, of an immunoglobulin isotype or class, or of an immunoglobulin subclass that is different from that of the tail region, for example, a tail region comprising, or consisting essentially or, or consisting of, CH2 and CH3 domains (or IgE CH3 and CH4 domains). For instance, in certain embodiments of the invention, a construct, for example, a binding domain immunoglobulin fusion protein, may comprise a binding region such as a binding domain polypeptide that is fused or otherwise connected to an immunoglobulin hinge region polypeptide comprising, or consisting essentially of, or consisting of, a wild-type human IgA hinge region polypeptide, or a mutated or otherwise altered human IgA hinge region polypeptide that contains zero or only one or more cysteine residues (but less than the wild-type number of cysteines), as described herein, or a wild-type human IgG hinge, such as an IgG1 hinge, region polypeptide, or a wild-type human IgE hinge-acting region, i.e., IgE CH2 region polypeptide, or a mutated or otherwise altered human IgG hinge, such as an IgG1 hinge, region polypeptide that is or has been mutated or otherwise altered to contain zero, one or two cysteine residues wherein the first cysteine of the wild-type hinge region is not mutated or altered or deleted, as also described herein. Such a hinge region polypeptide may be fused or otherwise connected to, for example, a tail region comprising, or consisting essentially of, or consisting of, an immunoglobulin heavy chain CH2 region polypeptide from a different Ig isotype or class, for example an IgA or an IgD or an IgG subclass (or a CH3 region from an IgE subclass), which in certain preferred embodiments will be the IgG1 or IgA or IgE subclass and in certain other preferred embodiments may be any one of the IgG2, IgG3 or IgG4 subclasses.

For example, and as described in greater detail herein, in certain embodiments of the present invention a connecting region may be selected to be an immunoglobulin hinge region polypeptide, which is or has been derived from a wild-type human IgA hinge region that naturally comprises three cysteines, where the selected hinge region polypeptide is truncated or otherwise altered or substituted relative to the complete and/or naturally-occurring hinge region such that only one or two of the cysteine residues remain (e.g., SEQ ID NOS:35-36).

Similarly, in certain other embodiments of the invention, the construct may be binding domain immunoglobulin fusion protein comprising a binding domain polypeptide that is fused or otherwise connected to an immunoglobulin hinge region polypeptide comprising a mutated or otherwise altered hinge region polypeptide in which the number of cysteine residues is reduced by amino acid substitution or deletion, for example a mutated or otherwise altered IgG1 hinge region containing zero, one or two cysteine residues as described herein, or an IgD hinge region containing zero cysteine residues.

A mutated or otherwise altered hinge region polypeptide may thus be derived or constructed from (or using) a wild-type immunoglobulin hinge region that contains one or more cysteine residues. In certain embodiments, a mutated or otherwise altered hinge region polypeptide may contain zero or only one cysteine residue, wherein the mutated or otherwise altered hinge region polypeptide is or has been derived from a wild type immunoglobulin hinge region that contains, respectively, one or more or two or more cysteine residues. In the mutated or otherwise altered hinge region polypeptide, the cysteine residues of the wild-type immunoglobulin hinge region are preferably deleted or substituted with amino acids that are incapable of forming a disulfide bond. In one embodiment of the invention, a mutated or otherwise altered hinge region polypeptide is or has been derived from a human IgG wild-type hinge region polypeptide, which may include any of the four human IgG isotype subclasses, IgG1, IgG2, IgG3 or IgG4. In certain preferred embodiments, the mutated or otherwise altered hinge region polypeptide is or has been derived from (or using) a human IgA or IgD wild-type hinge region polypeptide. By way of example, a mutated or otherwise altered hinge region polypeptide that is or has been derived from a human IgG1 or IgA wild-type hinge region polypeptide may comprise mutations, alterations, or deletions at two of the three cysteine residues in the wild-type immunoglobulin hinge region, or mutations, alterations, or deletions at all three cysteine residues.

The cysteine residues that are present in a wild-type immunoglobulin hinge region and that are removed or altered by mutagenesis or any other techniques according to certain embodiments of the present invention include cysteine residues that form, or that are capable of forming, interchain disulfide bonds.

In certain embodiments of the binding domain fusion protein, a protein having one or more desired effector functions can be prepared. Without wishing to be bound by particular theory or mechanism of action, the present invention contemplates that mutation, deletion, or other alteration of such hinge region cysteine residues, which are believed to be involved in formation of interchain disulfide bridges, reduces the ability of the subject invention binding domain immunoglobulin fusion protein to dimerize (or form higher oligomers) via interchain disulfide bond formation, while surprisingly not ablating or undesirably compromising the ability of a fusion protein or other construct to promote ADCC, and/or CDC and/or to fix complement. In particular, the Fc receptors that mediate ADCC (e.g., FcRIII, CD16) exhibit low affinity for immunoglobulin Fc domains, supporting the idea that functional binding of Fc to FcR requires avidity stabilization of the Fc-FcR complex by virtue of the dimeric structure of heavy chains in a conventional antibody, and/or FcR aggregation and cross-linking by a conventional antibody Fc structure. Sonderman et al., 2000 Nature 406: 267; Radaev et al., 2001 J. Biol. Chem. 276: 16469; Radaev et al., 2001 J. Biol. Chem. 276: 16478; Koolwijk et al., 1989 J. Immunol. 143: 1656; Kato et al., 2000 Immunol. Today 21: 310. Hence, the constructs, including for example binding domain immunoglobulin fusion proteins, of the present invention provide the advantages associated with single-chain constructs including singe-chain immunoglobulin fusion proteins while also unexpectedly retaining one or more immunological activities. Similarly, the ability to fix complement is typically associated with immunoglobulins that are dimeric with respect to heavy chain constant regions such as those that comprise Fc, while various constructs, including binding domain immunoglobulin fusion proteins, of the present invention, which may, due to the replacement or deletion of hinge region cysteine residues or due to other structural modifications as described herein, for example, have compromised or ablated abilities to form interchain disulfide bonds, exhibit the unexpected ability to fix complement. Additionally, according to certain embodiments of the present invention wherein a construct, including, for example, a binding domain immunoglobulin fusion protein, may comprise a connecting region and tail region comprising, or consisting essentially of, or consisting of, one or more of a human IgE hinge-acting region, i.e., a IgE CH2 region polypeptide, a human IgE CH3 constant region polypeptide, and a human IgE CH4 constant region polypeptide, the invention constructs including fusion proteins unexpectedly retain the immunological activity of mediating ADCC and/or of inducing an allergic response mechanism.

Selection of an immunoglobulin hinge region polypeptide as a connecting region according to certain embodiments of the subject invention constructs, such as binding domain immunoglobulin fusion proteins, may relate to the use of an “alternative hinge region” polypeptide sequence, which includes a polypeptide sequence that is not necessarily derived from any immunoglobulin hinge region sequence per se. Instead, an alternative hinge region refers to a hinge region polypeptide that comprises an amino acid sequence, or other molecular sequence, of at least about ten consecutive amino acids or molecules, and in certain embodiments at least about 11, 12, 13, 14, 15, 16, 17, 18, 19, 20, 21-25, 26-30, 31-50, 51-60, 71-80, 81-90, or 91-110 amino acids or molecules that is present in a sequence described in pending U.S. Pat. No. 10/627,556 or PCT/US03/41600 or, for example a polypeptide sequence that is or has been derived from a region located between intrachain disulfide-generated immunoglobulin-like loop domains of immunoglobulin gene superfamily members such as CD2 (e.g., Genbank Acc. No. NM_{—}001767), CD4 (e.g., Genbank Acc. No. NM_{—}000616), CD5 (e.g., Genbank Acc. No. BC027901), CD6 (e.g., Genbank Acc. No. NM_{—}006725), CD7 (e.g., Genbank Acc. Nos. XM_{—}046782, BC009293, NM_{—}006137) or CD8 (e.g., Genbank Acc. No. M12828), or other Ig superfamily members. By way of non-limiting example, an alternative hinge region used as a connecting region, for example, may provide a glycosylation site as provided herein, or may provide a human gene-derived polypeptide sequence for purposes of enhancing the degree of “humanization” of a fusion protein, or may comprise, or consist essentially of, or consist of, an amino acid sequence that eliminates or reduces the ability of a construct of the invention, such as a fusion protein, to form multimers or oligomers or aggregates or the like. Certain alternative hinge region polypeptide sequences, including those described herein, may be derived or constructed from (or using) the polypeptide sequences of immunoglobulin gene superfamily members that are not actual immunoglobulins per se. For instance and according to non-limiting theory, certain polypeptide sequences that are situated between intrachain disulfide-generated immunoglobulin loop domain of immunoglobulin gene super-family member proteins may be used in whole or in part as alternative hinge region polypeptides as provided herein, or may be further modified for such use. In certain embodiments, the connecting region may function in itself as a dimerization domain. However, in certain other embodiments comprising a dimerization domain, the dimerization domain is separate and distinct from the connecting region.

In another aspect of the invention the binding domain fusion protein may further include a region that comprises, consists essentially of, or consists of, a dimerization domain.

Suitable dimerization domains include those that facilitate the formation of covalent and non-covalent bonds between proteins or domains of proteins. The dimerization domains may, for example, promote the formation of homodimers or heterodimers of constructs and binding domain fusion proteins provided herein. In certain embodiments, dimerization domains promote an absolute shift to a dimeric state for all molecules or substantially all molecules of a population comprising the dimerization domain. In alternative embodiments, the dimerization domain will not promote an absolute shift to a dimeric state and will instead affect the monomer/dimer equilibrium state for a particular construct. This effect may be a function of physiological conditions, such as pH, salt concentrations, protein concentration, and the like. Particular dimerization domains may confer different effects on a monomer vs. dimer equilibrium for particular constructs and binding domain proteins, and these effects may, for example, be dependent on the site of placement within the protein.

Suitable dimerization domains include any of the connecting regions described herein. A connecting region will generally function as a dimerization domain by promoting the formation of a disulfide bond. One or more dimerization domains which are not connecting regions may optionally be present in which the connecting region may or may not function in itself as a dimerization domain. Another suitable dimerization domain may comprise a subportion of a connecting region or a conserved sequence motif of a connecting region, such as for example a conserved sequence in a immunoglobulin hinge region. Exemplary conserved sequences that may be used as dimerization motifs include the four amino acids motifs CPPC and CPXC, where x is selected from the amino acids lysine and glutamine. Suitable five amino acid dimerization motifs include CPPCP and CPXCP, where x is selected from the amino acids lysine and glutamine. While not wishing to be bound to any theory, it is believed that the dimerization motifs comprising cysteine residues facilitate the formation of disulfide bonds, including intrachain disulfide bonds which are effective to promote dimerization. The dimerization domains are another modular component of the constructs that can be placed at any desired location within the constructs and binding domains described herein.

Dimerization domains may comprise sequences that facilitate ionic interactions, hydrophobic interactions, hydrogen bonding, or other non covalent interactions that promote dimer formation. For example, binding domain immunoglobulin variable region interactions may function as a dimerization domain.

Suitable dimerization domains further include immunoglobulin constant regions, such as an immunoglobulin CH2CH3 domain or an immunoglobulin CH3 domain or analog (e.g., IgG CH2CH3 or CH3, IgA CH2CH3 or CH3). The sequence of the immunoglobulin domain in the dimerization domain of a binding domain fusion protein can be of animal origin, for example, mammalian, and preferably is of human origin. However, a dimerization may comprise any immunoglobulin constant region polypeptide sequence. Antibody constant regions may be used in the binding domain fusion proteins other than as a dimerization domain, for example for effector functions associated with the constant region domain.

Immunoglobulin constant regions may be incorporated into the constructs, for example, to facilitate purification of the constructs, to increase half life, or to confer effector functions. Antibody constant regions useful in the invention include the heavy chains in IgG, IgA, and IgD antibodies, which are designated CH1, CH2, and CH3, and the heavy chains in IgM and IgE antibodies, CH1, CH2, CH3 and CH4, and constant regions in immunoglobulin light chains C_{L} (constant region of a light chain). IgG, IgA, and IgD CH2 and/or CH3 regions are preferred. IgM and IgE CH2, CH3 and/or CH4 are preferred. Constructs of the invention may or may not have or mediate antibody dependent-mediated cytotoxicity (ADCC) and/or complement-dependent cytotoxicity (CDC). Constructs of the invention may or may not bind to Fc receptors, including but not limited to, for example, FcγRI (CD64), FcγRII (CD32), FcαRI (CD89), and FcγRIII (CD16) receptors.

In another aspect of the invention the binding domain fusion protein may further include a region that comprises, consists essentially of, or consists of, a native or engineered constant regions from an immunoglobulin heavy chain, other than CH1, for example, the CH2 and CH3 regions of IgG, the CH2 and CH3 regions of IgA, or the CH3 and CH4 regions of IgE.

In another aspect of the invention the binding domain polypeptide modulator fusion protein may further include a region that comprises, consists essentially of, or consists of, a native or engineered immunoglobulin heavy chain CH2-type region, for example, the CH2 region of IgG, the CH2 region of IgA, or the CH3 region of IgE.

In another aspect of the invention the binding domain polypeptide fusion protein may further include a region that comprises, consists essentially of, or consists of, a native or engineered C-terminal constant region from an immunoglobulin heavy chain, for example, the CH3 region of IgG, the CH3 region of IgA, or the CH4 region of IgE. Also within the scope of the present invention are CH3 domains from IgA including J chain tails that are capable of crosslinking polypeptide chains by J chain.

While not wishing to be bound to any particular theory or mechanism, it is believed that in the absence of a dimerization domain a binding domain fusion protein will be present in a monomeric state unless some particular characteristic of the binding domain allows the binding domain fusion protein to form dimers or multimers.

In certain embodiments the present invention provides polynucleotides or vectors (including cloning vectors and expression vectors) or transformed or transfected cells, including isolated or purified or pure polynucleotides, vectors, and isolated transformed or transfected cells, encoding or containing any one of the above or herein described polypeptide or protein constructs of the invention, for example, including binding domain fusion proteins. Thus, in various embodiments the invention provides a recombinant cloning or expression construct comprising any such polynucleotide that is operably linked to a promoter.

A DNA construct encoding a desired construct of the invention, for example, a binding domain-immunoglobulin fusion protein is introduced into a vector, for example, a plasmid, for expression in an appropriate host. In preferred embodiments, the host is a mammalian host, for example, a mammalian cell line. The sequence encoding the ligand or nucleic acid binding domain is preferably codon-optimized for expression in the particular host. Thus, for example, if a construct, for example, is a human binding domain-immunoglobulin fusion and is expressed in bacteria, the codons may be optimized for bacterial usage. For small coding regions, the gene can be synthesized as a single oligonucleotide. For larger proteins, splicing of multiple oligonucleotides, mutagenesis, or other techniques known to those in the art may be used. The sequences of nucleotides in plasmids or other vectors that are regulatory regions, such as promoters and operators, are operationally associated with one another for transcription. The sequence of nucleotides encoding a binding domain-immunoglobulin fusion protein may also include DNA encoding a secretion signal, whereby the resulting peptide is a precursor protein. The resulting processed protein may be recovered from the periplasmic space or the fermentation medium.

In preferred embodiments, the DNA plasmids may also include a transcription terminator sequence. As used herein, a “transcription terminator region” is a sequence that signals transcription termination. The entire transcription terminator may be obtained from a protein-encoding gene, which may be the same or different from the inserted binding domain-immunoglobulin fusion encoding gene or the source of the promoter. Transcription terminators are optional components of the expression systems herein, but are employed in preferred embodiments.

The plasmids or other vectors used herein include a promoter in operative association with the DNA encoding the protein or polypeptide of interest and are designed for expression of proteins in a suitable host as described above (e.g., bacterial, murine, or human) depending upon the desired use of the plasmid (e.g., administration of a vaccine containing binding domain-immunoglobulin fusion encoding sequences). Suitable promoters for expression of proteins and polypeptides herein are widely available and are well known in the art. Inducible promoters or constitutive promoters that are linked to regulatory regions are preferred. Such promoters include, for example, but are not limited to, the T7 phage promoter and other T7-like phage promoters, such as the T3, T5 and SP6 promoters, the trp, lpp, and lac promoters, such as the lacUV5, from E. coli; the P10 or polyhedrin gene promoter of baculovirus/insect cell expression systems (see, e.g., U.S. Pat. Nos. 5,243,041, 5,242,687, 5,266,317, 4,745,051, and 5,169,784) and inducible promoters from other eukaryotic expression systems. For expression of the proteins such promoters are inserted in a plasmid in operative linkage with a control region such as the lac operon.

Preferred promoter regions are those that are inducible and functional in mammalian cells, for example. Examples of suitable inducible promoters and promoter regions for bacterial expression include, but are not limited to: the E. coli lac operator responsive to isopropyl β-D-thiogalactopyranoside (IPTG; see Nakamura et al., 1979 Cell 18:1109-1117); the metallothionein promoter metal-regulatory-elements responsive to heavy-metal (e.g., zinc) induction (see, e.g., U.S. Pat. No. 4,870,009); the phage T7lac promoter responsive to IPTG (see, e.g., U.S. Pat. No. 4,952,496; and Studier et al., 1990 Meth. Enzymol. 185:60-89) and the TAC promoter. Depending on the expression host system to be used, plasmids may optionally include a selectable marker gene or genes that are functional in the host. Thus, for example, a selectable marker gene includes any gene that confers a phenotype on bacteria that allows transformed bacterial cells to be identified and selectively grown from among a vast majority of untransformed cells. Suitable selectable marker genes for bacterial hosts, for example, include the ampicillin resistance gene (Amp^{r}), tetracycline resistance gene (Tc^{r}) and the kanamycin resistance gene (Kan^{r}). The kanamycin resistance gene is presently preferred for bacterial expression.

In various expression systems, plasmids or other vectors may also include DNA encoding a signal for secretion of the operably linked protein. Secretion signals suitable for use are widely available and are well known in the art. Prokaryotic and eukaryotic secretion signals functional in E. coli may be employed. Depending on the expression systems, presently preferred secretion signals may include, but are not limited to, those encoded by the following E. coli genes: ompA, ompT, ompF, ompC, beta-lactamase, and alkaline phosphatase, and the like (von Heijne, J. Mol. Biol. 184:99-105, 1985). In addition, the bacterial pelB gene secretion signal (Lei et al., J. Bacteriol. 169:4379, 1987), the phoA secretion signal, and the cek2 functional in insect cell may be employed. The most preferred secretion signal for certain expression systems is the E. coli ompA secretion signal. Other prokaryotic and eukaryotic secretion signals known to those of skill in the art may also be employed (see, e.g., von Heijne, J. Mol. Biol. 184:99-105, 1985). Using the methods described herein, one of skill in the art can substitute secretion signals that are functional in either yeast, insect or mammalian cells to secrete proteins from those cells.

Preferred plasmids for transformation of E. coli cells include the pET expression vectors (e.g., pET-11a, pET-12a-c, pET-15b; see U.S. Pat. No. 4,952,496; available from Novagen, Madison, Wis.). Other preferred plasmids include the pKK plasmids, particularly pKK 223-3, which contains the tac promoter (Brosius et al., 1984 Proc. Natl. Acad. Sci. 81:6929; Ausubel et al., Current Protocols in Molecular Biology; U.S. Pat. Nos. 5,122,463, 5,173,403, 5,187,153, 5,204,254, 5,212,058, 5,212,286, 5,215,907, 5,220,013, 5,223,483, and 5,229,279). Plasmid pKK has been modified by replacement of the ampicillin resistance gene with a kanamycin resistance gene. (Available from Pharmacia; obtained from pUC4K, see, e.g., Vieira et al. (1982 Gene 19: 259-268; and U.S. Pat. No. 4,719,179.) Baculovirus vectors, such as pBlueBac (also called pJVETL and derivatives thereof), particularly pBlueBac III (see, e.g., U.S. Pat. Nos. 5,278,050, 5,244,805, 5,243,041, 5,242,687, 5,266,317, 4,745,051, and 5,169,784; available from Invitrogen, San Diego) may also be used for expression of the polypeptides in insect cells. Other plasmids include the pIN-IIIompA plasmids (see U.S. Pat. No. 4,575,013; see also Duffaud et al., 1987 Meth. Enz. 153: 492-507), such as pIN-IIIompA2.

Preferably, if one or more DNA molecules is replicated in bacterial cells, the preferred host is E. coli. The preferred DNA molecule is such a system also includes a bacterial origin of replication, to ensure the maintenance of the DNA molecule from generation to generation of the bacteria. In this way, large quantities of the DNA molecule can be produced by replication in bacteria. In such expression systems, preferred bacterial origins of replication include, but are not limited to, the f1-ori and col E1 origins of replication. Preferred hosts for such systems contain chromosomal copies of DNA encoding T7 RNA polymerase operably linked to an inducible promoter, such as the lacUV promoter (see U.S. Pat. No. 4,952,496). Such hosts include, but are not limited to, lysogens E. coli strains HMS174(DE3)pLysS, BL21(DE3)pLysS, HMS174(DE3) and BL21(DE3). Strain BL21(DE3) is preferred. The pLys strains provide low levels of T7 lysozyme, a natural inhibitor of T7 RNA polymerase.

The DNA molecules provided may also contain a gene coding for a repressor protein. The repressor protein is capable of repressing the transcription of a promoter that contains sequences of nucleotides to which the repressor protein binds. The promoter can be derepressed by altering the physiological conditions of the cell. For example, the alteration can be accomplished by adding to the growth medium a molecule that inhibits the ability to interact with the operator or with regulatory proteins or other regions of the DNA or by altering the temperature of the growth media. Preferred repressor proteins include, but are not limited to the E. coli lacI repressor responsive to IPTG induction, the temperature sensitive λ cI857 repressor, and the like. The E. coli lacI repressor is preferred.

In general, recombinant constructs of the subject invention will also contain elements necessary for transcription and translation. In particular, such elements are preferred where the recombinant expression construct containing nucleic acid sequences encoding binding domain-immunoglobulin fusion proteins is intended for expression in a host cell or organism. In certain embodiments of the present invention, cell type preferred or cell type specific expression of a cell binding domain-immunoglobulin fusion encoding gene may be achieved by placing the gene under regulation of a promoter. The choice of the promoter will depend upon the cell type to be transformed and the degree or type of control desired. Promoters can be constitutive or active and may further be cell type specific, tissue specific, individual cell specific, event specific, temporally specific or inducible. Cell-type specific promoters and event type specific promoters are preferred. Examples of constitutive or nonspecific promoters include the SV40 early promoter (U.S. Pat. No. 5,118,627), the SV40 late promoter (U.S. Pat. No. 5,118,627), CMV early gene promoter (U.S. Pat. No. 5,168,062), and adenovirus promoter. In addition to viral promoters, cellular promoters are also amenable within the context of this invention. In particular, cellular promoters for the so-called housekeeping genes are useful. Viral promoters are preferred, because generally they are stronger promoters than cellular promoters. Promoter regions have been identified in the genes of many eukaryotes including higher eukaryotes, such that suitable promoters for use in a particular host can be readily selected by those skilled in the art.

Inducible promoters may also be used. These promoters include MMTV LTR (PCT WO 91/13160), inducible by dexamethasone; metallothionein promoter, inducible by heavy metals; and promoters with cAMP response elements, inducible by cAMP. By using an inducible promoter, the nucleic acid sequence encoding a binding domain-immunoglobulin fusion protein may be delivered to a cell by the subject invention expression construct and will remain quiescent until the addition of the inducer. This allows further control on the timing of production of the gene product.

Event-type specific promoters are active or up-regulated only upon the occurrence of an event, such as tumorigenicity or viral infection. The HIV LTR is a well-known example of an event-specific promoter. The promoter is inactive unless the tat gene product is present, which occurs upon viral infection. Some event-type promoters are also tissue-specific.

Additionally, promoters that are coordinately regulated with a particular cellular gene may be used. For example, promoters of genes that are coordinately expressed may be used when expression of a particular binding construct of the invention, for example, a binding domain-immunoglobulin fusion protein-encoding gene is desired in concert with expression of one or more additional endogenous or exogenously introduced genes. This type of promoter is especially useful when one knows the pattern of gene expression relevant to induction of an immune response in a particular tissue of the immune system, so that specific immunocompetent cells within that tissue may be activated or otherwise recruited to participate in the immune response.

In addition to the promoter, repressor sequences, negative regulators, or tissue-specific silencers may be inserted to reduce non-specific expression of binding domain-immunoglobulin fusion protein encoding genes in certain situations, such as, for example, a host that is transiently immunocompromised as part of a therapeutic strategy. Multiple repressor elements may be inserted in the promoter region. Repression of transcription is independent on the orientation of repressor elements or distance from the promoter. One type of repressor sequence is an insulator sequence. Such sequences inhibit transcription (Dunaway et al., 1997 Mol Cell Biol 17: 182-9; Gdula et al., 1996 Proc Natl Acad Sci USA 93: 9378-83, Chan et al., 1996 J Virol 70: 5312-28; Scott and Geyer, 1995 EMBO J 14: 6258-67; Kalos and Fournier, 1995 Mol Cell Biol 15: 198-207; Chung et al., 1993 Cell 74: 505-14) and will silence undesired background transcription.

Repressor elements have also been identified in the promoter regions of the genes for type II (cartilage) collagen, choline acetyltransferase, albumin (Hu et al., 1992 J. Cell Growth Differ. 3(9): 577-588), phosphoglycerate kinase (PGK-2) (Misuno et al., 1992 Gene 119(2): 293-297), and in the 6-phosphofructo-2-kinase/fructose-2,6-bisphosphatase gene. (Lemaigre et al., Mol. Cell Biol. 11(2):1099-1106). Furthermore, the negative regulatory element Tse-1 has been identified in a number of liver specific genes, and has been shown to block cAMP response element(CRE)-mediated induction of gene activation in hepatocytes. (Boshart et al., 1990 Cell 61(5):905-916,).

In preferred embodiments, elements that increase the expression of the desired product are incorporated into the construct. Such elements include internal ribosome binding sites (IRES; Wang and Siddiqui, 1995 Curr. Top. Microbiol. Immunol 203: 99; Ehrenfeld and Semler, 1995 Curr. Top. Microbiol. Immunol. 203: 65; Rees et al., 1996 Biotechniques 20: 102; Sugimoto et al.,: 1994 Biotechnology 12 :694). IRES increase translation efficiency. As well, other sequences may enhance expression. For some genes, sequences especially at the 5′ end inhibit transcription and/or translation. These sequences are usually palindromes that can form hairpin structures. Any such sequences in the nucleic acid to be delivered are generally deleted. Expression levels of the transcript or translated product are assayed to confirm or ascertain which sequences affect expression. Transcript levels may be assayed by any known method, including Northern blot hybridization, RNase probe protection and the like. Protein levels may be assayed by any known method, including ELISA, western blot, immunocytochemistry or other well known techniques.

Other elements may be incorporated into the constructs of the invention, for example, into binding domain-immunoglobulin fusion protein encoding constructs of the present invention. In preferred embodiments, the construct includes a transcription terminator sequence, including a polyadenylation sequence, splice donor and acceptor sites, and an enhancer. Other elements useful for expression and maintenance of the construct in mammalian cells or other eukaryotic cells may also be incorporated (e.g., origin of replication). Because the constructs are conveniently produced in bacterial cells, elements that are necessary for, or that enhance, propagation in bacteria are incorporated. Such elements include an origin of replication, a selectable marker and the like.

As provided herein, an additional level of controlling the expression of nucleic acids encoding constructs of the invention, for example, binding domain-immunoglobulin fusion proteins, delivered to cells for gene therapy, for example, may be provided by simultaneously delivering two or more differentially regulated nucleic acid constructs. The use of such a multiple nucleic acid construct approach may permit coordinated regulation of an immune response such as, for example, spatiotemporal coordination that depends on the cell type and/or presence of another expressed encoded component. Those familiar with the art will appreciate that multiple levels of regulated gene expression may be achieved in a similar manner by selection of suitable regulatory sequences, including but not limited to promoters, enhancers and other well known gene regulatory elements.

The present invention also relates to vectors, and to constructs prepared from known vectors that include nucleic acids of the present invention, and in particular to “recombinant expression constructs”, including any of various known constructs, including delivery constructs, useful for gene therapy, that include any nucleic acids encoding, for example, binding domain-immunoglobulin fusion proteins and polypeptides according to the invention as provided herein; to host cells which are genetically engineered with vectors and/or other constructs of the invention and to methods of administering expression or other constructs comprising nucleic acid sequences encoding, for example, binding domain-immunoglobulin fusion polypeptides and fusion proteins of the invention, or fragments or variants thereof, by recombinant techniques.

Various constructs of the invention, including for example, binding domain-immunoglobulin fusion proteins, can be expressed in virtually any host cell, including in vivo host cells in the case of use for gene therapy, under the control of appropriate promoters, depending on the nature of the construct (e.g., type of promoter, as described above), and on the nature of the desired host cell (e.g., whether postmitotic terminally differentiated or actively dividing; e.g., whether the expression construct occurs in host cell as an episome or is integrated into host cell genome).

Appropriate cloning and expression vectors for use with prokaryotic and eukaryotic hosts are described, for example, by Sambrook, et al., Molecular Cloning: A Laboratory Manual, Second Edition, Cold Spring Harbor, N.Y., (1989); as noted herein, in particularly preferred embodiments of the invention, recombinant expression is conducted in mammalian cells that have been transfected or transformed with the subject invention recombinant expression construct. See also, for example, Machida, Calif., “Viral Vectors for Gene Therapy: Methods and Protocols”; Wolff, J A, “Gene Therapeutics: Methods and Applications of Direct Gene Transfer” (Birkhauser 1994); Stein, U and Walther, W (eds.P, “Gene Therapy of Cancer: Methods and Protocols” (Humana Press 2000); Robbins, P D (ed.), “Gene Therapy Protocols” (Humana Press 1997); Morgan, J R (ed.), “Gene Therapy Protocols” (Humana Press 2002); Meager, A (ed.), “Gene Therapy Technologies, Applications and Regulations: From Laboratory to Clinic” (John Wiley & Sons Inc. 1999); MacHida, C A and Constant, J G, “Viral Vectors for Gene Therapy: Methods and Protocols” (Humana Press 2002); “New Methods Of Gene Therapy For Genetic Metabolic Diseases NIH Guide,” Volume 22, Number 35, Oct. 1, 1993. See also recent U.S. patents relating to gene therapy, including vaccines, which include U.S. Pat. No. 6,384,210 (“Solvent for biopolymer synthesis, solvent microdroplets and methods of use”); U.S. Pat. No. 6,384,203 (“Family of immunoregulators designated leukocyte immunoglobulin-like receptors (LIR)”); U.S. Pat. No. 6,384,202 (“Cell-specific active compounds regulated by the cell cycle”); U.S. Pat. No. 6,384,018 (“Polynucleotide tuberculosis vaccine”); U.S. Pat. No. 6,383,814 (“Cationic amphiphiles for intracellular delivery of therapeutic molecules”); U.S. Pat. No. 6,383,811 (“Polyampholytes for delivering polyions to a cell”); U.S. Pat. No. 6,383,795 (“Efficient purification of adenovirus”); U.S. Pat. No. 6,383,794 (“Methods of producing high titer recombinant adeno-associated virus”); U.S. Pat. No. 6,383,785 (“Self-enhancing, pharmacologically controllable expression systems”); U.S. Pat. No. 6,383,753 (“Yeast mammalian regulators of cell proliferation”); U.S. Pat. No. 6,383,746 (“Functional promoter for CCR5”); U.S. Pat. No. 6,383,743 (“Method for serial analysis of gene expression”); U.S. Pat. No. 6,383,738 (“Herpes simplex virus ORF P is a repressor of viral protein synthesis”); U.S. Pat. No. 6,383,737 (“Human oxalyl-CoA Decarboxylase”); U.S. Pat. No. 6,383,733 (“Methods of screening for pharmacologically active compounds for the treatment of tumour diseases”); U.S. Pat. No. 6,383,522 (“Toxicity reduced composition containing an anti-neoplastic agent and a shark cartilage extract”); U.S. Pat. No. 6,383,512 (“Vesicular complexes and methods of making and using the same”); U.S. Pat. No. 6,383,481 (“Method for transplantation of hemopoietic stem cells”); U.S. Pat. No. 6,383,478 (“Polymeric encapsulation system promoting angiogenesis”); U.S. Pat. No. 6,383,138 (“Method for transdermal sampling of analytes”); U.S. Pat. No. 6,380,382 (“Gene encoding a protein having diagnostic, preventive, therapeutic, and other uses”); U.S. Pat. No. 6,380,371 (“Endoglycan: a novel protein having selectin ligand and chemokine presentation activity”); U.S. Pat. No. 6,380,369 (“Human DNA mismatch repair proteins”); U.S. Pat. No. 6,380,362 (“Polynucleotides, polypeptides expressed by the polynucleotides and methods for their use”); U.S. Pat. No. 6,380,170 (“Nucleic acid construct for the cell cycle regulated expression of structural genes”); U.S. Pat. No. 6,380,169 (“Metal complex containing oligonucleoside cleavage compounds and therapies”); U.S. Pat. No. 6,379,967 (“Herpesvirus saimiri as viral vector”); U.S. Pat. No. 6,379,966 (“Intravascular delivery of non-viral nucleic acid protease proteins, and uses thereof”).

Typically, for example, expression constructs are derived from plasmid vectors. One preferred construct is a modified pNASS vector (Clontech, Palo Alto, Calif.), which has nucleic acid sequences encoding an ampicillin resistance gene, a polyadenylation signal and a T7 promoter site. Other suitable mammalian expression vectors are well known (see, e.g., Ausubel et al., 1995; Sambrook et al., supra; see also, e.g., catalogues from Invitrogen, San Diego, Calif.; Novagen, Madison, Wis.; Pharmacia, Piscataway, N.J.; and others). Presently preferred constructs may be prepared that include a dihydrofolate reductase (DHFR) encoding sequence under suitable regulatory control, for promoting enhanced production levels of the binding domain-immunoglobulin fusion protein, which levels result from gene amplification following application of an appropriate selection agent (e.g., methotrexate).

Generally, recombinant expression vectors will include origins of replication and selectable markers permitting transformation of the host cell, and a promoter derived from a highly-expressed gene to direct transcription of a downstream structural sequence, as described above. The heterologous structural sequence is assembled in appropriate phase with translation initiation and termination sequences. Thus, for example, the binding domain-immunoglobulin fusion protein encoding nucleic acids as provided herein may be included in any one of a variety of expression vector constructs as a recombinant expression construct for expressing a binding domain-immunoglobulin fusion polypeptide in a host cell. In certain preferred embodiments the constructs are included in formulations that are administered in vivo. Such vectors and constructs include, chromosomal, nonchromosomal and synthetic DNA sequences, e.g., derivatives of SV40; bacterial plasmids; phage DNA; yeast plasmids; vectors derived from combinations of plasmids and phage DNA, viral DNA, such as vaccinia, adenovirus, fowl pox virus, and pseudorabies, or replication deficient retroviruses as described below. However, any other vector may be used for preparation of a recombinant expression construct, and in preferred embodiments such a vector will be replicable and viable in the host.

The appropriate DNA sequence(s) may be inserted into a vector, for example, by a variety of procedures. In general, a DNA sequence is inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described, for example, in Ausubel et al. (1993 Current Protocols in Molecular Biology, Greene Publ. Assoc. Inc. & John Wiley & Sons, Inc., Boston, Mass.); Sambrook et al. (1989 Molecular Cloning, Second Ed., Cold Spring Harbor Laboratory, Plainview, N.Y.); Maniatis et al. (1982 Molecular Cloning, Cold Spring Harbor Laboratory, Plainview, N.Y.); Glover (Ed.) (1985 DNA Cloning Vol. I and II, IRL Press, Oxford, UK); Hames and Higgins (Eds.), (1985 Nucleic Acid Hybridization, IRL Press, Oxford, UK); and elsewhere.

The DNA sequence in the expression vector is operatively linked to at least one appropriate expression control sequence(s) (e.g., a constitutive promoter or a regulated promoter) to direct mRNA synthesis. Representative examples of such expression control sequences include promoters of eukaryotic cells or their viruses, as described above. Promoter regions can be selected from any desired gene using CAT (chloramphenicol transferase) vectors or other vectors with selectable markers. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a nucleic acid encoding an binding domain-immunoglobulin fusion polypeptide is described herein.

Transcription of the DNA encoding proteins and polypeptides included within the present invention by higher eukaryotes may be increased by inserting an enhancer sequence into the vector. Enhancers are cis-acting elements of DNA, usually about from 10 to 300 bp that act on a promoter to increase its transcription. Examples including the SV40 enhancer on the late side of the replication origin bp 100 to 270, a cytomegalovirus early promoter enhancer, the polyoma enhancer on the late side of the replication origin, and adenovirus enhancers.

In other embodiments the invention provides an isolated polynucleotide encoding any of the constructs of the invention, for example, protein or polypeptide constructs of the invention including binding domain fusion proteins, and in related embodiments the invention provides a recombinant expression construct comprising such a polynucleotide, and in certain further embodiments the invention provides a host cell transformed or transfected with, or otherwise containing, such a recombinant expression construct. In another embodiment the invention provides a method of producing a construct of the invention, for example, a protein or polypeptide construct of the invention such as a binding domain fusion protein, comprising the steps of (a) culturing a host cell that has been transformed or transfected with, or otherwise made to contain, a polynucleotide construct of the invention under conditions that permit expression of the construct, for example, a construct encoding a binding domain fusion protein; and (b) isolating the construct, for example, the binding domain fusion protein, from the host cell culture.

The constructs, including polypeptide constructs, of the present invention include, for example, binding domain-immunoglobulin fusion polypeptides and fusion proteins having binding regions such as binding domain polypeptide amino acid sequences that are identical or similar to sequences known in the art, or fragments or portions thereof. For example by way of additional illustration and not limitation, a anti-CD28 scFv-trappin construct [SEQ ID NO:______] is contemplated for use according to the instant invention, as are portions of such polypeptides and/or polypeptides having at least about 70% similarity (preferably greater than a 70% identity) and more preferably about 90% similarity (more preferably greater than a 90% identity) to the reported polypeptide and still more preferably about 95% similarity (still more preferably greater than a 95% identity) to the reported polypeptides and to portions of such polypeptides, wherein such portions of a binding domain-immunoglobulin fusion polypeptide, for example, generally contain at least about 30 amino acids and more preferably at least about 50 amino acids. Extracellular domains include, for example, portions of a cell surface molecule, and in particularly preferred embodiments cell surface molecules that are integral membrane proteins or that comprise a plasma membrane spanning transmembrane domain, that are constructed to extend beyond the outer leaflet of the plasma membrane phospholipid bilayer when the molecule is expressed at a cell surface, preferably in a manner that exposes the extracellular domain portion of such a molecule to the external environment of the cell, also known as the extracellular milieu. Methods for determining whether a portion of a cell surface molecule comprises an extracellular domain are well known to the art and include, for example, experimental determination (e.g.,direct or indirect labeling of the molecule, evaluation of whether the molecule can be structurally altered by agents to which the plasma membrane is not permeable such as proteolytic or lipolytic enzymes) or topological prediction based on the structure of the molecule (e.g., analysis of the amino acid sequence of a polypeptide) or other methodologies.

In other embodiments there is provided a host cell transformed or transfected with, or otherwise containing, any such recombinant cloning or expression construct. Host cells include the cells of a subject undergoing ex vivo cell therapy including, for example, ex vivo gene therapy.

In another aspect, the present invention relates to host cells containing the herein described nucleic acid constructs, such as, for example, recombinant binding domain-immunoglobulin fusion expression constructs. Host cells are genetically engineered (transduced, transformed or transfected) with the vectors and/or expression constructs of this invention which may be, for example, a cloning vector, a shuttle vector, or an expression construct. The vector or construct may be, for example, in the form of a plasmid, a viral particle, a phage, etc. The engineered host cells can be cultured in conventional nutrient media modified as appropriate for activating promoters, selecting transformants or amplifying particular genes such as genes encoding binding domain-immunoglobulin fusion polypeptides or binding domain-immunoglobulin fusion proteins. The culture conditions for particular host cells selected for expression, such as temperature, pH and the like, will be readily apparent to the ordinarily skilled artisan.

The host cell for production or expression of a construct of the invention, for example, can be a higher eukaryotic cell, such as a mammalian cell, or a lower eukaryotic cell, such as a yeast cell, or the host cell can be a prokaryotic cell, such as a bacterial cell. Representative examples of appropriate host cells according to the present invention include, but need not be limited to, bacterial cells, such as E. coli, Streptomyces, Salmonella tvphimurium; fungal cells, such as yeast; insect cells, such as Drosophila S2 and Spodoptera Sf9; animal cells, such as CHO, COS or 293 cells; adenoviruses; plant cells, or any suitable cell already adapted to in vitro propagation or so established de novo. The selection of an appropriate host is deemed to be within the scope of those skilled in the art from the teachings herein.

Various mammalian cell culture systems can also be employed to express recombinant protein. Examples of mammalian expression systems include the COS-7 lines of monkey kidney fibroblasts, described by Gluzman, 1981 Cell 23:175, and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences, for example as described herein regarding the preparation of binding domain-immunoglobulin fusion expression constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including but not limited to, for example, calcium phosphate transfection, DEAE-Dextran mediated transfection, or electroporation (Davis et al., 1986 Basic Methods in Molecular Biology).

In a related embodiment there is provided a method of producing a polypeptide or protein or other construct of the invention, for example, including a binding domain fusion protein, comprising the steps of (a) culturing a host cell as described or provided for herein under conditions that permit expression of the construct, for example, a binding domain fusion protein; and (b) isolating the construct, for example, the binding domain fusion protein from the host cell or host cell culture.

Embodiments of the binding domain fusion protein include a strep tag, which can be used for purification. Strep tag is a sequence of 8 to 9 amino acids that reversibly binds streptavidin and includes without limitation AWRHPQFGG, AQRHPQFGG, WSHPQFEK, and SWSHPQFEK.

The inventions described and claimed herein include novel molecules useful, for example, as therapeutics and other purposes including diagnostic and research purposes. Such molecules have, for example, antigen binding or other binding function(s) and one or more effector functions. DNA constructs of the invention are useful in, for example, gene therapies, including in vivo and ex vivo gene therapies.

Gene therapy is the use of genetic material to treat disease. It comprises strategies to replace defective genes or add new genes to cells and/or tissues, and is being developed for application in the treatment of cancer, the correction of metabolic disorders and in the field of immunotherapy. Gene therapies of the invention include the use of various constructs of the invention, with or without a separate carrier or delivery vehicle or constructs, for treatment of the diseases, disorders, and/or conditions noted herein. Such constructs may also be used as vaccines for treatment or prevention of the diseases, disorders, and/or conditions noted herein. DNA vaccines, for example, make use of polynucleotides encoding immunogenic protein and nucleic acid determinants to stimulate the immune system against pathogens or tumor cells. Such strategies can stimulate either acquired or innate immunity or can involve the modification of immune function through cytokine expression. In vivo gene therapy involves the direct injection of genetic material into a patient or animal model of human disease. Vaccines and immune modulation are systemic therapies. With tissue-specific in vivo therapies, such as those that aim to treat cancer, localized gene delivery and/or expression/targeting systems are preferred. Diverse gene therapy vectors have been designed to target specific tissues, and procedures have seen developed to physically target specific tissues, for example, using catheter-based technologies, all of which are contemplated herein. Ex vivo approaches to gene therapy are also contemplated herein and involve the removal, genetic modification, expansion and re-administration of a patient's own cells. Examples include bone marrow transplantation for cancer treatment or the genetic modification of lymphoid progenitor cells. Ex vivo gene therapy is preferably applied to the treatment of cells that are easily accessible and can survive in culture during the gene transfer process (such as blood or skin cells).

Useful gene therapy vectors include adenoviral vectors, lentiviral vectors, Adeno-associated virus (AAV) vectors, Herpes Simplex Virus (Hsv) vectors, and retroviral vectors. Gene therapies may also be carried out using “naked DNA,” lipsome-based delivery, lipid-based delivery (including DNA attached to positively charged lipids), and electroporation.

As provided herein, in certain embodiments, including but not limited to gene therapy embodiments, the vector may be a viral vector such as, for example, a retroviral vector. Miller et al., 1989 BioTechniques 7:980; Coffin and Varmus, 1996 Retroviruses, Cold Spring Harbor Laboratory Press, NY. For example, retroviruses from which the retroviral plasmid vectors may be derived include, but are not limited to, Moloney Murine Leukemia Virus, spleen necrosis virus, retroviruses such as Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus.

Retroviruses are RNA viruses that can replicate and integrate into the genome of a host cell via a DNA intermediate. This DNA intermediate, or provirus, may be stably integrated into the host cell DNA. According to certain embodiments of the present invention, an expression construct may comprise a retrovirus into which a foreign gene that encodes a foreign protein is incorporated in place of normal retroviral RNA. When retroviral RNA enters a host cell coincident with infection, the foreign gene is also introduced into the cell, and may then be integrated into host cell DNA as if it were part of the retroviral genome. Expression of this foreign gene within the host results in expression of the foreign protein.

Most retroviral vector systems that have been developed for gene therapy are based on murine retroviruses. Such retroviruses exist in two forms, as free viral particles referred to as virions, or as proviruses integrated into host cell DNA. The virion form of the virus contains the structural and enzymatic proteins of the retrovirus (including the enzyme reverse transcriptase), two RNA copies of the viral genome, and portions of the source cell plasma membrane containing viral envelope glycoprotein. The retroviral genome is organized into four main regions: the Long Terminal Repeat (LTR), which contains cis-acting elements necessary for the initiation and termination of transcription and is situated both 5′ and 3′ of the coding genes, and the three coding genes gag, pol, and env. These three genes gag, pol, and env encode, respectively, internal viral structures, enzymatic proteins (such as integrase), and the envelope glycoprotein (designated gp70 and p15e) which confers infectivity and host range specificity of the virus, as well as the “R” peptide of undetermined function.

Separate packaging cell lines and vector producing cell lines have been developed because of safety concerns regarding the uses of retroviruses, including their use in expression constructs as provided by the present invention. Briefly, this methodology employs the use of two components, a retroviral vector and a packaging cell line (PCL). The retroviral vector contains long terminal repeats (LTRs), the foreign DNA to be transferred and a packaging sequence (y). This retroviral vector will not reproduce by itself because the genes which encode structural and envelope proteins are not included within the vector genome. The PCL contains genes encoding the gag, pol, and env proteins, but does not contain the packaging signal “y”. Thus, a PCL can only form empty virion particles by itself. Within this general method, the retroviral vector is introduced into the PCL, thereby creating a vector-producing cell line (VCL). This VCL manufactures virion particles containing only the retroviral vector's (foreign) genome, and therefore has previously been considered to be a safe retrovirus vector for therapeutic use.

“Retroviral vector construct” refers to an assembly that is, within preferred embodiments of the invention, capable of directing the expression of a sequence(s) or gene(s) of interest, such as binding domain-immunoglobulin fusion encoding nucleic acid sequences. Briefly, the retroviral vector construct must include a 5′ LTR, a tRNA binding site, a packaging signal, an origin of second strand DNA synthesis and a 3′ LTR. A wide variety of heterologous sequences may be included within the vector construct, including for example, sequences which encode a protein (e.g., cytotoxic protein, disease-associated antigen, immune accessory molecule, or replacement gene), or which are useful as a molecule itself (e.g., as a ribozyme or antisense sequence).

Retroviral vector constructs of the present invention may be readily constructed from a wide variety of retroviruses, including for example, B, C, and D type retroviruses as well as spumaviruses and lentiviruses (see, e.g., RNA Tumor Viruses, Second Edition, Cold Spring Harbor Laboratory, 1985). Such retroviruses may be readily obtained from depositories or collections such as the American Type Culture Collection (“ATCC”; Rockville, Md.), or isolated from known sources using commonly available techniques. Any of the above retroviruses may be readily utilized in order to assemble or construct retroviral vector constructs, packaging cells, or producer cells of the present invention given the disclosure provided herein, and standard recombinant techniques (e.g., Sambrook et al, Molecular Cloning: A Laboratory Manual, 2d ed., Cold Spring Harbor Laboratory Press, 1989; Kunkle, 1985 PNAS 82:488).

Suitable promoters for use in viral vectors generally may include, but are not limited to, the retroviral LTR; the SV40 promoter; and the human cytomegalovirus (CMV) promoter described in Miller, et al., 1989 Biotechniques 7:980-990, or any other promoter (e.g., cellular promoters such as eukaryotic cellular promoters including, but not limited to, the histone, pol III, and β-actin promoters). Other viral promoters that may be employed include, but are not limited to, adenovirus promoters, thymidine kinase (TK) promoters, and B19 parvovirus promoters. The selection of a suitable promoter will be apparent to those skilled in the art from the teachings contained herein, and may be from among either regulated promoters or promoters as described above.

As described above, the retroviral plasmid vector is employed to transduce packaging cell lines to form producer cell lines. Examples of packaging cells which may be transfected include, but are not limited to, the PE501, PA317, ψ-2, ψ-AM, PA12, T19-14X, VT-19-17-H2, ψCRE, ψCRIP, GP+E-86, GP+envAm12, and DAN cell lines as described in Miller, Human Gene Therapy, 1:5-14 (1990). The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the binding domain-immunoglobulin fusion polypeptides or fusion proteins. Such retroviral vector particles then may be employed, to transduce eukaryotic cells, either in vitro or in vivo. The transduced eukaryotic cells will express the nucleic acid sequence(s) encoding the binding domain-immunoglobulin fusion polypeptide or fusion protein. Eukaryotic cells which may be transduced include, but are not limited to, embryonic stem cells, as well as hematopoietic stem cells, hepatocytes, fibroblasts, circulating peripheral blood mononuclear and polymorphonuclear cells including myelomonocytic cells, lymphocytes, myoblasts, tissue macrophages, dendritic cells, Kupffer cells, lymphoid and reticuloendothelia cells of the lymph nodes and spleen, keratinocytes, endothelial cells, and bronchial epithelial cells.

As another example of an embodiment of the invention in which a viral vector is used to prepare, for example, a recombinant binding domain-immunoglobulin fusion expression construct, in one preferred embodiment, host cells transduced by a recombinant viral construct directing the expression of binding domain-immunoglobulin fusion polypeptides or fusion proteins may produce viral particles containing expressed binding domain-immunoglobulin fusion polypeptides or fusion proteins that are derived from portions of a host cell membrane incorporated by the viral particles during viral budding.

In another embodiment there is provided a pharmaceutical composition comprising any one of the above or herein described polypeptide or protein or other constructs of the invention, for example (including, for example, binding domain fusion proteins), in combination with a physiologically acceptable carrier.

In another embodiment the invention provides a pharmaceutical composition comprising, for example, an isolated, purified, or pure polynucleotide encoding any one of the polypeptide or protein constructs of the invention, for example (including, for example, binding domain fusion proteins), in combination with a physiologically acceptable carrier, or for example, in combination with, or in, a gene therapy delivery vehicle or vector.

Constructs of the invention, for example, binding domain-immunoglobulin fusion proteins, or compositions comprising one or more polynucleotides encoding same as described herein (for example, to be administered under conditions and for a time sufficient to permit expression of a binding domain-immunoglobulin fusion protein in a host cell in vivo or in vitro, for gene therapy, for example, among other things), may be formulated into pharmaceutical compositions for administration according to well known methodologies. Pharmaceutical compositions generally comprise one or more recombinant expression constructs, and/or expression products of such constructs, in combination with a pharmaceutically acceptable carrier, excipient or diluent. Such carriers will be nontoxic to recipients at the dosages and concentrations employed. For nucleic acid-based formulations, or for formulations comprising expression products of the subject invention recombinant constructs, about 0.01 μg/kg to about 100 mg/kg body weight will be administered, for example, typically by the intradermal, subcutaneous, intramuscular or intravenous route, or by other routes. A preferred dosage, for example, is about 1 μg/kg to about 1 mg/kg, with about 5 μg/kg to about 200 μg/kg particularly preferred. It will be evident to those skilled in the art that the number and frequency of administration will be dependent upon the response of the host. “Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

“Pharmaceutically acceptable salt” refers to salts of the compounds of the present invention derived from the combination of such compounds and an organic or inorganic acid (acid addition salts) or an organic or inorganic base (base addition salts). The compounds of the present invention may be used in either the free base or salt forms, with both forms being considered as being within the scope of the present invention.

The pharmaceutical compositions that contain one or more nucleic acid constructs of the invention, for example, binding domain-immunoglobulin fusion protein encoding constructs (or their expressed products) may be in any form which allows for the composition to be administered to a patient. For example, the composition may be in the form of a solid, liquid or gas (aerosol). Typical routes of administration include, without limitation, oral, topical, parenteral (e.g., sublingually or buccally), sublingual, rectal, vaginal, and intranasal. The term parenteral as used herein includes subcutaneous injections, intravenous, intramuscular, intrasternal, intracavemous, intrathecal, intrameatal, intraurethral injection or infusion techniques. The pharmaceutical composition is formulated so as to allow the active ingredients contained therein to be bioavailable upon administration of the composition to a patient. Compositions that will be administered to a patient take the form of one or more dosage units, where for example, a tablet may be a single dosage unit, and a container of one or more compounds of the invention in aerosol form may hold a plurality of dosage units.

For oral administration, an excipient and/or binder may be present. Examples are sucrose, kaolin, glycerin, starch dextrins, sodium alginate, carboxymethylcellulose and ethyl cellulose. Coloring and/or flavoring agents may be present. A coating shell may be employed.

The composition may be in the form of a liquid, e.g., an elixir, syrup, solution, emulsion or suspension. The liquid may be for oral administration or for delivery by injection, as two examples. When intended for oral administration, preferred compositions contain, in addition to one or more binding domain-immunoglobulin fusion construct or expressed product, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

A liquid pharmaceutical composition as used herein, whether in the form of a solution, suspension or other like form, may include one or more of the following adjuvants: sterile diluents such as water for injection, saline solution, preferably physiological saline, Ringer's solution, isotonic sodium chloride, fixed oils such as synthetic mono or digylcerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

It may also be desirable to include other components in the preparation, such as delivery vehicles including but not limited to aluminum salts, water-in-oil emulsions, biodegradable oil vehicles, oil-in-water emulsions, biodegradable microcapsules, and liposomes. Examples of immunostimulatory substances (adjuvants) for use in such vehicles include N-acetylmuramyl-L-alanine-D-isoglutamine (MDP), lipopoly-saccharides (LPS), glucan, IL-12, GM-CSF, gamma interferon and IL-15.

While any suitable carrier known to those of ordinary skill in the art may be employed in the pharmaceutical compositions of this invention, the type of carrier will vary depending on the mode of administration and whether a sustained release is desired. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109. In this regard, it is preferable that the microsphere be larger than approximately 25 microns.

Pharmaceutical compositions may also contain diluents such as buffers, antioxidants such as ascorbic acid, low molecular weight (less than about 10 residues) polypeptides, proteins, amino acids, carbohydrates including glucose, sucrose or dextrins, chelating agents such as EDTA, glutathione and other stabilizers and excipients. Neutral buffered saline or saline mixed with nonspecific serum albumin are exemplary appropriate diluents. Preferably, product is formulated as a lyophilizate using appropriate excipient solutions (e.g., sucrose) as diluents.

As described above, the subject invention includes compositions capable of delivering nucleic acid molecules encoding binding domain-immunoglobulin fusion proteins. Such compositions include recombinant viral vectors (e.g., retroviruses (see WO 90/07936, WO 91/02805, WO 93/25234, WO 93/25698, and WO 94/03622), adenovirus (see Berkner, 1988 Biotechniques 6: 616-627; Li et al., 1993 Hum. Gene Ther. 4: 403-409; Vincent et al., Nat. Genet. 5: 130-134; and Kolls et al., 1994 Proc. Natl. Acad. Sci. USA 91: 215-219), pox virus (see U.S. Pat. No. 4,769,330; U.S. Pat. No. 5,017,487; and WO 89/01973)), recombinant expression construct nucleic acid molecules complexed to a polycationic molecule (see WO 93/03709), and nucleic acids associated with liposomes (see Wang et al., 1987 Proc. Natl. Acad. Sci. USA 84: 7851). In certain embodiments, the DNA may be linked to killed or inactivated adenovirus (see Curiel et al., 1992 Hum. Gene Ther. 3: 147-154; Cotton et al., 1992 Proc. Natl. Acad. Sci. USA 89:6094). Other suitable compositions include DNA-ligand (see Wu et al., 1989 J Biol. Chem. 264: 16985-16987) and lipid-DNA combinations (see Felgner et al., 1989 Proc. Natl. Acad. Sci; USA 84: 7413-7417).

In addition to direct in vivo procedures, ex vivo procedures may be used in which cells are removed from a host, modified, and placed into the same or another host animal. It will be evident that one can utilize any of the compositions noted above for introduction of constructs of the invention, for example, binding domain-immunoglobulin fusion proteins or of binding domain-immunoglobulin fusion protein encoding nucleic acid molecules into tissue cells in an ex vivo context. Protocols for viral, physical and chemical methods of uptake are well known in the art.

Accordingly, the present invention is useful for treating a patient having a B cell disorder or a malignant condition, or for treating a cell culture derived from such a patient. As used herein, the term “patient” refers to any warm-blooded animal, preferably a human. A patient may be afflicted with cancer or a malignant condition, such as B cell lymphoma, or may be normal (i.e., free of detectable disease and infection). A “cell culture” includes any preparation amenable to ex vivo treatment, for example a preparation containing immunocompetent cells or isolated cells of the immune system (including, but not limited to, T cells, macrophages, monocytes, B cells and dendritic cells). Such cells may be isolated by any of a variety of techniques well known to those of ordinary skill in the art (e.g., Ficoll-hypaque density centrifugation). The cells may (but need not) have been isolated from a patient afflicted with a B cell disorder or a malignant condition, and may be reintroduced into a patient after treatment.

A liquid composition intended for either parenteral or oral administration should contain an amount of a construct of the invention, for example, a binding domain-immunoglobulin fusion protein encoding construct or expressed product, such that a suitable dosage will be obtained. Typically, this amount is at least 0.01 wt % of a binding domain-immunoglobulin fusion construct or expressed product in the composition. When intended for oral administration, this amount may be varied to be between 0.1 and about 70% of the weight of the composition. Preferred oral compositions contain between about 4% and about 50% of binding domain-immunoglobulin fusion construct or expressed product(s). Preferred compositions and preparations are prepared so that, for example, a parenteral dosage unit contains between 0.01 to 1% by weight of active compound.

The pharmaceutical composition may be intended for topical administration, in which case the carrier may suitably comprise a solution, emulsion, ointment, or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of a construct of the invention, for example, a binding domain-immunoglobulin fusion construct or expressed product, of from about 0.1 to about 10% w/v (weight per unit volume).

The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol.

In the methods of the invention, a construct of the invention, for example, a binding domain-immunoglobulin fusion encoding constructs or expressed product(s), may be administered through use of insert(s), bead(s), timed-release formulation(s), patch(es) or fast-release formulation(s).

Constructs of the invention, for example, antigen-binding constructs of the invention, may be administered or co-administered to an animal or patient in combination with, or at the same or about the same time, as other compounds. In one aspect, one or more constructs, including for example one or more antigen-binding constructs, are administered to an animal or patient in conjunction with one or more chemotheraputic compounds such as alkylating agents, nucleoside analogues, and the like. The administration or co-administration of one or more constructs, including one or more antigen-binding constructs, of the invention and one or more chemotheraputic agents can be used for the treatment of tumors or cancer in an animal or patient. Exemplary cancers include, but are not limited to, head and neck cancer, breast cancer, colorectal cancer, gastric cancer, hepatic cancer, bladder cancer, cervical cancer, endometrial cancer, lung cancer (non-small cell), ovarian cancer, pancreatic cancer, prostate cancer; choriocarcinoma (lung cancer);hairy cell leukemia, chronic lymphotic leukemia, acute lymphocytic leukemia (breast & bladder), acute myelogenous leukemia, meningeal leukemia, chronic myelogenous leukemia, erythroleukemia. More commonly the cancers treated include non-Hodgkin's lymphoma (osteogenic sarcoma, adult soft tissue sarcoma), T-cell lymphoma, chronic lymphocytic leukaemia, slowly growing non-Hodgkin's lymphomas, Hodgkin's lymphoma and ovarian cancer.

Examples of an alkylating agents that can be co-administered with one or more constructs, including one or more antigen-binding constructs, of the invention include mechlorethamine, chlorambucil, ifosfamide, melphalan, busulfan, carmustine, lomustine, procarbazine, dacardazine, cisplatin, carboplatin, mitomycin C, cyclophosphamide, isosfamide, hexamethylmelamine, thiotepa, and dacarbazine, and analogues thereof. See for example U.S. Pat. No. 3,046,301 describing the synthesis of chlorambucil, U.S. Pat. No. 3,732,340 describing the synthesis of ifosfamide, U.S. Pat. No. 3,018,302 for the synthesis of cyclophosphamide, U.S. Pat. No. 3,032,584 describing the synthesis of melphalan, and Braunwald et al., “Harrison's Principles of Internal Medicine,” 15th Ed., McGraw-Hill, New York, N.Y., pp. 536-544 (2001) for clinical aspects of cyclophosphamide, chlorambucil, melphalan, ifosfamide, procarbazine, hexamethylmelamine, cisplatin, and carboplatin. Examples of nucleoside analogues, include, but are not limited to, fludarabine pentostatin, methotrexate, fluorouracil, fluorodeoxyuridine, CB3717, azacitidine, cytarabine, floxuridine, mercaptopurine, 6-thioguanine, cladribine and analogues thereof. One example is the combination of constructs, including antigen-binding constructs, that bind CD20. This construct acts as a chemosensitising agent and works together with chemotherapeutic agents, such that less chemotherapeutic agents are necessary to achieve anti-tumor or anti-cancer effects. For example, U.S. Pat. No. 3,923,785 describing the synthesis of pentostatin, U.S. Pat. No. 4,080,325 describing the synthesis of methotrexate, U.S. Pat. No. 2,802,005 describing the synthesis of fluorouracil, and Braunwald et al., “Harrison's Principles of Internal Medicine,” 15th Ed., McGraw-Hill, New York, N.Y., pp. 536-544 (2001) for clinical aspects of methotrexate, 5-fluorouracil, cytosine arabinoside, 6-mercaptopurine, 6-thioguanine, and fludarabine phosphate.

In another aspect, one or more constructs, including one or more antigen-binding constructs, of the invention can be administered or co-administered compounds that inhibit topoisomerase II or compounds that otherwise interact with nucleic acids in cells. Such compounds include, for example, doxorubicin, epirubicin, etoposide, teniposide, mitoxantrone, and analogues thereof. In one example, this combination is used in treatment to reduce tumor cell contamination of peripheral blood progenitor cells (PBSC) in conjunction with high-dose chemotherapy and autologous stem cell support (HDC-ASCT). See U.S. Pat. No. 6,586,428 to Geroni et al.

In anther aspect, one or more constructs, including one or more antigen-binding constructs, of the invention can be administered or co-administered with therapeutic drugs. For example, Virulizin (Lorus Therapeutics), which is believed to stimulate the release of tumour necrosis factor, TNF-alpha, by tumour cells in vitro and stimulate activation of macrophage cells. This can be used in combination with one or more constructs, including one ore more antigen-binding constructs, of the invention to increase cancer cell apoptosis and treat various types of cancers including Pancreatic Cancer, Malignant Melanoma, Kaposi's Sarcoma (KS), Lung Cancer, Breast Cancer, Uterine, Ovarian and Cervical Cancer. Another example is CpG 7909 (Coley Pharmaceutical Group), which is believed to activate NK cells and monocytes and enhance ADCC. This drug can be used in combination with cancer or tumor specific constructs, including antigen-binding constructs, of the invention, such as an anti-CD20 construct, to treat non-Hodgkin's lymphoma and other cancers.

One or more constructs, including one or more antigen-binding constructs, of the invention can also be combined with angiogensis inhibitors to increase anti-tumor effects. Angiogenisis is the growth of new blood vessels. This process allows tumors to grow and metastasize. Inhibiting angiogeneisis can help prevent metastasis, and stop the spread of tumors cells. Angiogenisis inhibitors include, but are not limited to, angiostatin, endostatin, thrombospondin, platelet factor 4, Cartilage-derived inhibitor (CDI), retinoids, Interleukin-12, tissue inhibitor of metalloproteinase 1, 2 and 3 (TIMP-1, TIMP-2, and TIMP-3) and proteins that block the angiogensis signaling cascade, such as anti-VEGF (Vascular Endothelial Growth Factor) and IFN-alpha. Angiogenesis inhibitors can be administered or co-administered with tumor specific constructs, including antigen-binding constructs capable of mediating, for example, ADCC and/or complement fixation or chemotherapy-conjugated antigen-binding of the invention to combat various types of cancers, for example, solid tumor cancers such as lung and breast cancer.

In another aspect, one or more constructs, including one or more antigen-binding constructs, of the invention can be administered or co-administered with disease modifying anti-rheumatic agents (DMAR agents) for the treatment of rheumatoid arthritis, psoriasis, ulcerative colitus, systemic lupus erythematosus (SLE), Crohn's disease, ankylosing spondylitis, and various inflammatory disease processes. In such treatment, the constructs, for example, antigen-binding constructs, of the invention are commonly administered in conjunction with compounds such as azathioprine, cyclosporin, gold, hydroxychloroquine, methotrexate, penicallamine, sulphasalazine, and the like.

In another aspect, one or more constructs, including one or more antigen-binding constructs, of the invention can be administered or co-administered with agents or compounds that counteract the biological effects of interleukin-1, including for example interleukin-1 inhibitors and interleukin-1 receptor antagonist. It is thought that interleukin-I has a role in the generation of rheumatoid arthritis (RA), inflammation, and the destruction of joints. IL-1 inhibitors can also be used in conjunction with the constructs, including antigen-binding constructs, of the invention to treat arthritis, inflammatory bowel disease, sepsis and septic shock, ischemic injury, reperfusion, ischemic brain injury such as cerebral palsy and multiple sclerosis. See U.S. Pat. No. 6,159,460 to Thompson et al. In another aspect, for example, one or more constructs, including one or more antigen-binding constructs, of the invention can be administered or co-administered to an animal or patient in conjunction with one or more glucocorticoids for example, methylprednisilone, dexamethasone, hydrocortisone, and the like. Glucocorticoids have been used to induce apoptosis and inhibit growth, independent of ADCC and CDC. These compounds can be combined with constructs, including antigen-binding constructs, of the invention capable of inducing apoptosis in cancer cells. In one example is the anti-CD20, and anti-CD40 antigen-binding constructs, which can be used to induce apoptosis in B-cells, are combined with glutcocorticoids to treat B-cell non-Hodgkin's lymphoma (NHL).

In another aspect, one or more constructs, including one or more antigen-binding constructs, of the invention can be administered or co-administered with p38 inhibitors or antagonists. The p38 mitogen-activated protein kinase pathway is involved in a number of cellular processes instrumental to the development of rheumatoid arthritis. For example, the activation and infiltration of leukocytes as well as the production of inflammatory cytokines are p38-dependent processes.

In another aspect, one or more constructs, including one or more antigen-binding constructs, of the invention are administered or co-administered with compounds that promote the differentiation and proliferation of B-cells. Cytokines such as \ interleukin-4 (IL-4) and interleukin-6 (IL-6), in additional to other biological activities, have been shown to stimulate antibody synthesis and secretion by activated B lympocytes. In a particular aspect of the invention, constructs, including antigen-binding constructs that recognize and bind CD20 are co-administered with one or more of interleukin-4 (IL-4) and interleukin-6 (IL-6).

In another aspect one or more constructs, including one or more antigen-binding constructs, of the invention can be administered or co-administered with Interleukin-2 (IL-2). Interleukin 2 (IL-2) is a lymphokine that increases production of effector cells, such as CD4+T-helper cells, CD8 cytotoxic cells, antibody producing B cells, natural killer cells (NK), and monocytes/macrophages. IL-2 helps produce T-cells, which in turn secrete more of the IL-2 (an “autocrine loop”). IL-2 can be used to augment antibody-dependent cell-mediated cytotoxicity (ADCC) and immunotherapies associated with constructs of the invention. In one example, an anti-CD20 construct of the invention and IL-2 are used to treat patients with relapsed or refractory follicular non-Hodgkin's lymphoma. In another example IL-2 is administered or co-administered with HIV immunotherapies to help with T cell recovery.

In another aspect one or more constructs, including one or more antigen-binding constructs, of the invention can be administered or co-administered with Interleukin-12 (IL-12). IL-12 is know to enhance cytolytic T-cell responses, promote the development of helper T cells, enhance the activity of natural killer (NK) cells, and induces the secretion of IFN-γ in T and NK cells. IL-12 also increases many helper and effector cells that mediate apoptosis. In another aspect of the invention, one or more constructs, including one or more antigen-binding constructs, are administered or co-administered with IL-12 in the treatment of an animal or patient with a tumor or cancer. For example, a construct, including an antigen-binding construct, of the invention that binds CD20 combined with IL-2 for the treatment of a patient with B-cell non-Hodgkin's lymphoma (NHL).

One or more constructs, including one or more antigen-binding constructs, of the invention can also be combined with immunomodulators to boost the efficacy of the antigen-binding constructs of the invention. Immunomodulators include, but are not limited to, Colony Stimulating Factors (CSF), Tumor necrosis Factors (TNF), and Interferons (IFN).

CSFs can include granulocyte-macrophage CSF (GM-CSF), granulocyte-CSF (G-CSF), and macrophage CSF (M-CSF). GM-CSF is thought to regulate the development of neutrophils, macrophages, monocytes and eosinophils. G-CSF has been shown to induce neutrophil production, and M-CSF production. M-CSF has been shown to stimulate macrophages and monocytes. The use of CSFs to treat neutropenia in cancer patients has been long established. In one example, constructs, including antigen-binding constructs, of the invention can be combined with GM-CSF, G-CSF or combinations thereof in order to accelerate recovery from neutropenia in patients after bone marrow trans-plantation and chemotherapy. Neutrophils play a major role in fighting microbes such as bacterial, fungi and parasites. Patients with neutropenia are particularly susceptible to bacterial and wide spread fungal infections. In another example, a construct, including an antigen binding construct, of the invention can be combined with GM-CSF-treated neutrophils, monocytes and macrophages to increase activity against bacteria, fungi, etc, including the dreaded Pneumocystis carinii.

An example of an IFN is interferon alpha (IFN-α). IFN-α is made naturally by some types of white blood cell as part of the immune response when the body reacts to cancers or viral infections. It has two main modes of attack, interfering with growth and proliferation of cancer cells and it boosting the production of killer T cells and other cells that attack cancer cells. Interferon is also thought to facilitate cancer cells to put out chemical signals that make them better targets for the immune system, and has been used in recent years for several different types of cancer, particularly kidney cancer, melanoma, multiple myeloma, and some types of leukemia. It is also used to treat viral infections such as hepatitis. Interferon-alpha2a, for example, enhances ADCC and can be combined with one or more constructs, including antigen-binding constructs, of the invention to increase the efficiency of ADCC activity associated with the construct. In another example, one or more constructs, including one or more antigen-binding constructs of the invention are administered or co-administered to an animal or patient with interferon-gamma (IFN-γ), which has been show to increase the number of anti-CD20 antigens on B cells and bone marrow plasma cells (BMPC). This is particularly useful for the treatment of patients with multiple myelomas, which have a reduced expression of CD20 in their B cells and bone marrow plasma cells (BMPC). Accordingly, the treatment of multiple myeloma patients with constructs, including antigen-binding constructs of the invention, in particular constructs that bind CD20, may be usefully co-administered in conjunction with IFN-γ

TNF is a class of natural chemicals with anticancer properties. One example of a TNF is TNF-alpha. TNF-alpha has also been shown to have synergistic effects with IFN-gamma and IL-12. In another example, TNF can be administered or co-administered with one or more tumor specific constructs, including one or more antigen-binding constructs, of the invention, and include chemotherapy-conjugated antigen binding constructs of the invention, together with IFN-gamma, IL-12 or various combinations thereof. TNF is also known to be an inflammatory regulation molecule. TNF-alpha antibodies or antagonist(s) can be combined with anti-T cell constructs, including antigen-binding constructs, of the invention to treat patients with rheumatoid arthritis, psoriasis, ulcerative colitus, systemic lupus erythematosus (SLE), Crohn's disease, ankylosing spondylitis, and various inflammatory disease processes.

In another aspect, one or more constructs, including one or more antigen-binding constructs, of the invention can be administered or co-administered with another antibody or antigen-binding construct of the invention. One example is a construct, for example, an antigen-binding construct of the invention capable of binding CD20 combined with a construct capable of binding CD22, CD19 or combinations thereof. This combination is effective as a treatment for indolent and aggressive forms of B-cell lymphomas, and acute and chronic forms of lymphatic leukemias. See U.S. Pat. No. 6,306,393 to Goldberg. In another example, constructs, including antigen-binding constructs, of the invention are co-administered with other constructs such as antigen-binding constructs of the invention that aid in mediating apoptosis. For example, a combination of one or more constructs, including one or more antigen-binding constructs of the invention capable of binding CD28, CD3, CD20 or a combination thereof. The combination of anti-CD28 and CD3 provides a method for prolonged proliferation of T-cells. See U.S. Pat. No. 6,352,694 to June et al. This prolonged T-cell proliferation increases the efficiency immune dependent cytotoxicity, particularly those associated with anti-CD20.

In another aspect, constructs, including antigen-binding constructs, of the invention can be administered or co-administered with one or more T-cell regulatory molecules. One example is a combination with interleukin-12 (IL-12). The IL-12 cytokine stimulates cell-mediated immunity, has angiostatic activity, and possesses significant anti-tumor effects in a variety of tumor models. IL-12 has also been shown to stimulate the production of interferon-gamma (IFN-γ). Accordingly, the treatment of multiple myeloma patients with one or more constructs, including one or more antigen-binding constructs, of the invention, in particular those that bind CD20, is expected to be more efficacious when co-administered in conjunction with IL-12. In another example, one or more constructs, including one or more antigen-binding constructs, of the invention can be administered or co-administered with a binding-domain construct of the invention other protein capable of binding CTLA-4 to enhance the anti-tumor immune response, by inhibiting the downregulation of T-cell activation.

In another aspect, one or more constructs, including one or more antigen-binding constructs, of the invention can be combined with gene therapies. In one example, a chemotherapy-conjugated construct of the invention is administered or co-administered with the Bcl-2 antisense oligonucleotide. Bcl-2 is associated with tumor resistance to anti-cancer therapies, and is believed to block chemotherapy-induced cell death. In another example one or more constructs, including one or more antigen-binding constructs, of the invention is administered or co-administered with an adenovirus for delivery of a “suicide gene.” The adenovirus inserts the gene directly into the tumor cells, which makes these cells sensitive to an otherwise ineffective drug. Drug treatment then destroys the tumor cells, while leaving healthy cells untouched. However, once therapy is complete stray cancer cells that escaped therapy can reestablish and metastasize. Combining gene therapy with one or more constructs, including one or more antigen-binding constructs, will help kill stray cancer cells and minimize cancer reoccurrence.

A similar combination can be used with palliative (non-radical) operations to surgically remove tumors. In this example one or more constructs, including one or more antigen-binding constructs, of the invention can be administered before and after surgical extractions of tumors in order to increase the immune response and reduce the likelihood of reoccurrence by killing any cancer cells that were not removed during the surgery.

Another aspect combines a cancer or antigen vaccine and T-cell regulator molecules. For example, the binding portion, for example, an antigen-binding portion, of a construct can be specific for a cancer cell or antigen, or a protein fragment from a cancer cell or antigen. This can help mediate an immune response against a particular tumor or antigen. Such constructs can be combined with T-cell regulators to increase the efficiency of the immune response.

In another example, one or more constructs, including one or more antigen-binding constructs, of the invention is administered or co-administered with retinoids. Retinoids include Vitamin A and its derivatives, which have the ability to stop cells from dividing and cause them to differentiate. Vitamin A is combined with an anti-cancer construct(s), including antigen-binding construct(s), of the invention to combat various forms of cancer.

The terms “binding construct” and “antigen-binding construct” as used herein may refer to, for example, engineered polypeptides, recombinant polypeptides, synthetic, semi-synthetic or other fusion proteins that are capable of binding a target, for example, an antigen. Antigen-binding constructs of the invention may be used in various applications, including those within the variety of uses to which antibodies or related immunoglobulin-type constructs may be put. Constructs, including antigen-binding constructs of the invention can be used in in vivo and in vitro experiments for therapeutic, diagnostic, research, and other purposes. Such uses include, for example, the following.

Constructs, including antigen-binding constructs of the invention may be used for immunohistochemistry applications. For example, they may be used for immunolocalization of a particular antigen or group of antigens in a tissue. Tissue can be fixed and incubated with antigen-binding constructs of interest. These constructs can then be localized using a secondary antibody or binding construct of the invention coupled to a label, for example, to a gold particle or an enzyme that gives a chemical reaction, like horseradish peroxidase or beta-galactosidase. A secondary antibody or binding construct is frequently made that is reactive against, for example, a portion of the primary binding construct. Thus, for example, if the primary binding construct has a human tail portion, the secondary antibody or binding construct could be, for example, a rabbit anti-mouse antibody or antigen-binding construct that has been linked to beta-galactosidase. Alternatively the antibody or binding construct of the invention can be purified and then conjugated to another molecule to produce a fluorescent antibody or binding construct.

Constructs, including antigen-binding constructs of the invention can also be used to detect the location of an antigen or antigens on the surface of cells or to detect the location of intracellular materials using, for example, Immunoelectron Microscopy. Electron dense materials such as ferritin or colloidal gold, for example, can be conjugated to an antigen-binding construct. Scanning electron microscopy can be used to detect the localization of the antigen/binding construct complex.

Constructs, including antigen-binding constructs of the invention may also be used to quantitate the presence of an antigen or antigens using one of a variety of immunoassay formats, for example, a radioimmunoassay (RIA) format or an enzyme-linked immunosorbent assay (ELISA) format. There are many variants of these approaches, but those are based on a similar idea. For example, if an antigen can be bound to a solid support or surface, or is in solution, it can be detected by reacting it with a specific antigen-binding construct of the invention. The presence or amount of the construct can then be detected or quantitated by reacting it with, for example, either a secondary antibody or a second antigen-binding construct of the invention by incorporating a label directly into the primary antibody. Alternatively, for example, an antigen-binding polypeptide of the invention can be bound to a solid surface and the antigen added. A second antibody or antigen-binding polypeptide(s) of the invention that recognizes a distinct epitope on the antigen can then be added and detected. This technique is commonly referred to as a “sandwich assay”, which is frequently used to avoid problems of high background or non-specific reactions, among other reasons.

Because the binding constructs of the invention can have high affinity/affinities and/or selectivity/selectivities for a particular epitope or epitopes, they can also be used as affinity reagents, for example, in protein or antigen purification. In one example of such a process, antigen-binding constructs of the invention are immobilized on a suitable support, for example, Sephadex resin or filter paper. The immobilized construct is exposed to a sample containing, or suspected of containing, a target protein(s) or antigen(s). The support is rinsed with a suitable buffer that will remove unwanted materials. The support is washed with another buffer that will release the bound protein(s) or antigen(s).

Because particular binding constructs of the invention can bind to proteins or other antigens with high affinity and selectivity they can also be used as a criterion for the importance of a particular enzyme or other macromolecule in a particular reaction. If an antigen-binding construct of the invention can interfere with a reaction in a solution, this will indicate that the construct may be binding specifically to a protein or other antigenic material involved in that reaction.

Constructs, including antigen-binding constructs of the invention can also be used as receptor blockers or inhibitors or antagonists.

Constructs, including antigen-binding constructs of the invention can also be used in identifying and studying the function(s) of proteins. If an antigen-binding construct of the invention reacts with a specific protein, for example, that protein can subsequently be precipitated from solution, for example. Precipitation is typically performed by using a secondary antibody or antigen-binding construct of the invention that links primary complexes together. Alternatively, the complex can be removed by reacting the solution with either protein A or, for example, depending on the construct, an anti-Fc antibody, for example, which has been attached to beads, for example, so that can be easily removed form the solution.

Constructs, including antigen-binding constructs of the invention can also be used in conjunction with gel-shift experiments to identify specific nucleic acid-binding proteins such as DNA-binding proteins. For example, DNA-binding proteins can be assayed by their ability to bind with high affinity to a particular oligonucleotide. The mobility of an oligonucleotide associated with the protein is far different than the mobility of a free oligonucleotide and results in a gel migration pattern and signal that is commonly referred to as a gel shift. The addition of the construct to the binding assay can have either of two effects. If the construct binds to a region of a protein not involved in DNA binding it can result in a complex that has even a slower mobility and is detected as a greater shift in mobility (a super-shift). Alternatively, if the construct binds to a region of the protein involved in recognizing the DNA then it can disrupt the binding and eliminate the shift. In either case, the data from these experiments can serve as a criterion to identify a DNA-binding protein, for example.

It is also possible to use constructs, including antigen-binding constructs of the invention to detect a protein by western blotting after fractionation by SDS-PAGE, for example. Once fractionated proteins are transferred to a membrane such as a nitrocellulose sheet, they are exposed to a particular antigen-binding construct of the invention that specifically recognizes, or recognizes to a desired degree of selectivity, proteins immobilized to the blot. This allows particular proteins to be identified. This approach is particularly useful if the mobility of the protein changes during an experiment. For example, incorporation of a phosphate or a carbohydrate, or cleavage of the protein, results in a change in mobility that can be followed in straightforward manner by western analysis. With appropriate controls, this approach can be used to measure the abundance of a protein in response to experimental manipulations.

The combination of SDS gels and immunoprecipitation can also be extremely effective. If a particular protein can be immunoprecipitated in a solution, both supernatant and precipitated fractions can be separated on an SDS gel and studied using an antigen-binding constructs of the invention.

Sometimes a binding construct of the invention directed against one protein will also precipitate a second protein that interacts with the first protein. The second protein, as well as the first, can then be seen by staining the gel or by autoradiography. This relationship is frequently the first indication that a protein functions as part of a complex and it can also be used to demonstrate a physical interaction of two proteins that are hypothesized to interact on the basis of other evidence (e.g., a two hybrid screen or a supressor mutation). This approach can be combined with western blotting analysis in several extremely effective ways.

Thus, for example, antigen-binding constructs of the invention can be used in a combination of immunoprecipitation and western analysis in the study, for example, of signal transduction and protein processing. For example, an immunoprecipitated protein can be subsequently studied by western analysis using a different antibody or antigen-binding construct of the invention that binds to the protein. The most useful of are those that are directed against particular structural determinants that may be present in a protein. Thus, an antibody or antigen-binding construct of the invention directed against a region of the protein that undergoes proteolytic processing can be useful to follow proteolytic processing. Additionally, a construct of the invention or a mixture of antigen-binding constructs of the invention that recognize phophorylated peptides (e.g., anti PY (phosphorylated tyrosine) can be used to follow the extent of phosphorylation of a protein (using western analysis) after it is precipitated, or visa versa. Glycosylation reactions can also be followed by antigen-binding constructs of the invention directed against a carbohydrate epitope (or by lectins, i.e., proteins that recognize carbohydrates). Likewise, some antigen-binding constructs of the invention can be made that specifically recognize a phosphorylated epitope, for example, that will recognize a tyrosine or a serine residue after phosphorylation, but will not bind (or detectably bind) the epitope in the absence of phosphate. This approach can be used to determine the phosphorylation state of a particular protein. For example, the phosphorylation of CREB (the cAMP response element binding protein) can be followed by an antibody that specifically recognizes an epitope in a way that is dependent on the phosphorylation of serine 133.

Constructs, including antigen-binding constructs of the invention can also be used to screen expression libraries to isolate candidate polynucleotides that express or present a particular epitope, or that have a particular affinity or expression characteristic.

Constructs, including antigen-binding constructs of the invention that bind to a cell surface can also be used as a marker to quantitate the fraction of cells expressing that marker using flow cytometry. If different antigen binding constructs of the invention/fluorescent dye combinations are used, for example, the fraction of cells expressing several antigens can be determined.

Constructs, including antigen-binding constructs of the invention that function like anti-idiotype antibodies, i.e., antibodies against the binding domain of another antibody, can be used in any of a number of methods in which is would be desirable or useful to mimic the structure of an antigen. Such uses include, for example, uses as cancer vaccines (including antigen-binding constructs of the invention that incorporate a molecular adjuvant), as probes for receptors, as receptor agonists, as receptor antagonists, as receptor blockers or inhibitors, and so on.

In another aspect, constructs, including antigen-binding constructs of the invention may bispecific and thus capable of binding to two distinct epitopes, which may be present on the same or different cell types.

In vivo uses of constructs of the invention, including antigen-binding constructs, include therapy, alone or in combination with one or more other therapies, for various diseases including cancers as well as B-cell disorders including autoimmune diseases. In some cases the constructs of the invention are administered to a patient. In other cases, the construct may be coupled to another molecule by techniques known in the art, for example, a fluorescent molecule to aid in imaging a target, or a therapeutic drug and/or a toxin or an isotope, chemotherapeutic drug, or other organic or non-organic enzyme regulator to aid in killing a target.

For example, a labeling molecule or atom can be conjugated or otherwise linked to the antigen-binding construct of the invention to aid in imaging or as a diagnostic agent. These include, but are not limited to enzymatic labels, radioisotopes or radioactive compounds or elements, fluorescent compounds or metals, chemiluminescent compounds and bioluminescent compounds. Thus, binding constructs or antigen-binding constructs of the invention can be conjugated to a drug, which allows specific drug targeting and increased efficiency once the drug reaches the target. This facilitates drug therapy while reducing systemic toxicity and side effects. This allows use of drugs that would otherwise be unacceptable when administered systemically. Dosage will depend on the potency of the drug and the efficiency of the carrier construct. Other examples of in vivo uses include the use of binding constructs or antigen-binding constructs of the invention in which a toxin is chemically linked or conjugated to an polypeptide of the invention to form, for example, molecules that may be termed “immunoconjugates” or “immunotoxins.” Typically, for example, such a toxin may include one ore more radioisotopes (for example, Iodine-131, Yttrium-90, Rhenium-186, Copper-67, and/or Bishmuth-212), natural toxins, chemotherapy agents, biological response modifiers, or any other substance that is capable of assisting in damaging or killing a target cell, inhibiting target cell replication, or is effective in disrupting a desired cellular function in a target cell.

The toxin portion of the immunotoxin can be derived form various sources. Toxins are commonly derived from plants or bacteria, but toxins of human origin or synthetic toxins can be used as well, for example. Examples of toxins derived from bacteria or plants include, but are not limited to, abrin, α-sarcin, diptheria toxin, ricin, saporin, and pseudomonas exotoxin. Examples of mammalian enzymes include, but are not limited to, ribonucleases (RNAse) and deoxyribonucleases. Numerous immunotoxins that may be used with one or more constructs of the invention have been described in the art. See, for example, U.S. Pat. No. 4,753,894 to Frankel et al.; U.S. Pat. No. 6,099,842 to Pastan et al.; Nevelle, et al., 1982 Immunol Rev. 62: 75-91; Pastan et al., 1992 Ann Rev Biochem 61: 331-354; Chaudary et al., 1989 Nature 339: 394; and Batra et al., 1991 Mol. Cell. Biol. 11: 2200. Modified toxins described herein and those described in the various publications are also within the scope of the instant invention.

Generally, the immunotoxins and other therapeutic agents of this invention are administered at a concentration that is therapeutically effective to treat or prevent a particular disease, disorder, or condition, such as for the treatment of tumors and malignancies, the treatment of autoimmune diseases, allergies and inflammation, etc. This effective dosage and mode of administration will depend on the animal or patient being treated, the disease or condition being treated, the strength of the immunoconjugates or immunotoxins and the efficiency of the conjugate. To accomplish this goal, the immunotoxins may be formulated using a variety of acceptable formulations and excipients known in the art. Typically, for example, the immunotoxins are administered by injection, either intravenously or intraperitoneally. Methods to accomplish this administration are known to those of ordinary skill in the art. It another aspect, the invention includes topically or orally administered compositions such as an aerosol or cream or patch that may be capable of transmission across mucous membranes.

Formulants may be added to an immunoconjugates or immunotoxins of the invention before administration to a patients being treated. A liquid formulation is most common, but other formulations are within the scope of the invention. The formulants may include for example oils, polymers, vitamins, carbohydrates, amino acids, salts, buffers, albumin, surfactants, or bulking agents. Carbohydrates can include sugar or sugar alcohols such as mono, di, or polysaccharides, or water-soluble glucans. The saccharides or glucans can include for example fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” may be defined as a C₄ to C₈ hydrocarbon having an —OH group and includes, for example, galactitol, inositol, mannitol, xylitol, sorbitol, glycerol, and arabitol. These sugars or sugar alcohols mentioned above may be used individually or in combination. There is no fixed limit to the amount used as long as the sugar or sugar alcohol is soluble in the aqueous preparation. In one aspect, the sugar or sugar alcohol concentration is between 0.5 w/v % and 15 w/v %, typically between 1.0 w/v % and 7.0 w/v %, more typically between 2.0 and 6.0 w/v %.

Exemplary amino acids include levorotary (L) forms of camitine, arginine, and betaine; however, other amino acids may be added. Commonly used polymers include polyvinylpyrrolidone (PVP) with an average molecular weight between 2,000 and 3,000, for example, or polyethylene glycol (PEG) with an average molecular weight between 3,000 and 5,000, for example. A buffer can be used in the composition to minimize pH changes in the solution before lyophilization or after reconstitution. Any physiological buffer may be used, but citrate, phosphate, succinate, and glutamate buffers or mixtures thereof are more commonly utilized. The concentration can be, for example, from 0.01 to 0.3 molar. Higher or lower concentrations may be used.

Immunotoxins of the invention can be chemically modified by covalent conjugation to a polymer to increase their circulating half-life, for example. Exemplary polymers and methods to attach them to peptides are referenced in U.S. Pat. No. 4,766,106 to Katre et al.; U.S. Pat. No. 4,179,337 to Davis et al.; U.S. Pat. No. 4,495,285 to Shimizu et al.; and U.S. Pat. No. 4,609,546 to Hiratani.

In another aspect, methods of treating, preventing, or suppressing diseases, disorders and conditions relating to the activity or activation of proteases are provided. Such disorders and conditions include that would be benefited or ameliorated by anti-proteinase action. In another aspect, the invention provides a method of treating a subject having or suspected of having a malignant condition or immune system disorder (e.g. a T cell disorder), comprising administering to a patient a therapeutically effective amount of any of the pharmaceutical compositions described or claimed herein.

Some examples of diseases and disorders that may be treated or ameliorated by methods utilizing therapeutic agents and compositions provided herein include diseases and disorders of the immune system (e.g. those that can be improved by modulating immune system functions); infections (e.g. bacterial, viral, fungal, and infections by other parasitic organisms); proliferating diseases (e.g., tumors and cancers); respiratory diseases, disorders and conditions, including ARDS; vascular diseases, disorders and conditions; inflammation; and inflammatory diseases, disorders and conditions.

Lung disorders or conditions can be treated, for example, by modulating immune system function, by promoting lung cell proliferation, or by promoting lung cell regeneration. Representative lung cell disorders include the treatment of asthma (e.g. as determined by an inhibition of leukocyte influx into airways after chronic allergan exposure, prevention of antigen-induced decrease of tracheal mucus velocity, or an inhibition of late-phase bronchoconstriction and development of hyper-responsiveness), the treatment of acute respiratory distress syndrome (ARDS), the treatment of cystic fibrosis, and the treatment of pneumonia.

A number of other diseases, disorders, and conditions may be treated in embodiments of the invention, including but are not limited to, Grave's disease, Hashimoto's disease, rheumatoid arthritis, systemic lupus erythematosus, Sjogrens Syndrome Immune Thrombocytopenic purpura, multiple sclerosis, myasthenia gravis, scleroderma, psoriasis, Inflammatory Bowel Disease including Crohn's disease and ulcerative colitis, Inflammatory Bowel Disease including Crohn's disease and Ulcerative colitis, are autoimmune diseases of the digestive system.

One embodiment is directed to a method of treating a patient having cancer or another proliferative disorder comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L} (constant region of a light chain).

Another embodiment is directed to a method of treating a patient having an inflammatory disorder comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L}.

Another embodiment is directed to a method of treating a patient having rheumatoid arthritis comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L}.

Another embodiment is directed to a method for the treatment of an HIV infection in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L}.

Another embodiment is directed to a method for the treatment of treatment of a pulmonary or lung disorder in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L} (constant region of a light chain).

Another embodiment is directed to a method for the treatment of treatment of a pulmonary or lung disorder in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L} (constant region of a light chain).

Another embodiment is directed to a method for the treatment of a pulmonary or lung inflammation in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a C_{H}2 C_{H}3, a C_{H}3, a hinge-CH2CH3, a hinge-C_{H}3, a CH1-hinge-C_{H}2C_{H}3, a C_{H}1-hinge-CH3, and C_{L}.

Another embodiment is directed to a method for the treatment of asthma in a patient comprising administering an effective anti-inflammatory amount of i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound or composition described herein, for example a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L} (constant region of a light chain).

Another embodiment is directed to a method for the treatment of asthma in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L} (constant region of a light chain).

Another embodiment is directed to a method for the treatment of acute respiratory distress syndrome (ARDS) in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L} (constant region of a light chain).

Another embodiment is directed to a method for the treatment of cystic fibrosis in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L} (constant region of a light chain).

Another embodiment is directed to a method for the treatment of pneumonia in a patient comprising administering an effective anti-inflammatory amount of i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound or composition described herein, for example a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L}.

Another embodiment is directed to a method for the treatment of a vascular disorder in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L}.

Another embodiment is directed to a method for the treatment of an ophthalmic disease or disorder in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L}.

Another embodiment is directed to a method for the treatment of an age related macular degenerative disease in a patient comprising administering an effective anti-inflammatory amount of a compound or composition described herein, for example i) a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain; or ii) a compound comprising a protease inhibitor molecule connected to an immunoglobulin domain, said immunoglobulin domain selected from the group consisting of a CH2CH3, a CH3, a hinge-CH2CH3, a hinge-CH3, a CH1-hinge-CH2CH3, a CH1-hinge-CH3, and C_{L}.

In certain embodiments, methods of treatment provided herein utilize the binding domain that is capable of binding CD28. In certain preferred embodiments, the binding domain is capable of inhibiting T cell activation, or treating one or more condition or disorder selected from inflammation, a proliferative disorder (e.g. cancer), or an infection (e.g. bacterial, fungal, viral infection). CD28 is member of the immunoglobulin super gene family and is a transmembrane adhesion receptor expressed as a 44-kD dimer on the surface of a major subset of human T cells. It is a homodimeric type I transmembrane glycoprotein expressed as a 220 amino acid precursor with an amino terminal signal sequence of 27 amino acids. The mature protein contains 134 amino acids in the extracellular domain and 27 amino acids in the transmembrane region with a 41 amino acid cytoplasm tail. CD28 is a member of a heterophilic cell adhesion complex, and is the receptor for the B-cell-restricted B7/BB-1 antigen. CD28 serves as a surface component of a signal transduction pathway that modulates T-cell lymphokine production and increases the resistance of T-cell responses to various immunosuppressive agents. CD28 is expressed at high levels all mature CD3³⁰ thymocytes, plasma cells, and most peripheral T lymphocytes. For reviews of CD28 structure and function, see June, C. H., et al., Immunol. Today 15: 321, 1994; June, C. H., et al., Immunol Today 11: 211, 1990; and Linsley, P. S., and Ledbetter, J. A., 1993 Annu. Rev. Immunol. 11: 191. Fusion proteins of a scFv immunoglobulin region that binds CD28 and the protease inhibitor α₁-antitrypsin, which are not binding domain fusion proteins according to the present invention, have been reported See Vanhove, B., “Selective blockade of CD28 and not CTLA-4 with a single-chain Fv-α₁-antitrypsin fusion antibody”, 2003 Blood 102(2).

In certain embodiments, methods of treatment provided herein utilize the binding domain that is capable of biding to a VEGF or VEGF precursor to modulate (e.g. inhibit) VEGF activity, expression, or the like. In certain preferred embodiments, the binding domain is capable of inhibiting T cell activation, or treating one or more condition or disorder selected from a proliferative disorder, including cancers that involve neovascularization and vascularization.

One embodiment of the binding domain fusion protein comprises a binding domain having a variable L chain (amino acid residues 1-112), linker (113-117), and variable H chain (118-238) chain that recognizes CD28 (2E12), a dimerization domain (249-265) containing an IgG1 hinge with a serine substitution for one of its three cysteine residues, a WAP domain (266-374) contained in SLPI, and a WSHPQFEK Strep tag (residues 375-382) (SEQ ID NO:1). The gene sequence is reported with a signal peptide (nucleic acid bases 7-75).

One embodiment of the binding domain fusion protein comprises a binding domain containing a variable H chain (amino acid residues 1-120), linker (residues 121-137), and variable L chain (residues 138-243) chain that recognizes VEGF, a dimerization domain (residues 244-260) containing an IgG1 hinge with a serine substitution for one of its cysteine residues, a WAP domain (residues 261-360) contained in SLPI, and a WSHPQFEK Strep tag (residues 370-377) (SEQ ID NO:4). The gene sequence is reported with a signal peptide (nucleic acid bases 19-75).

One embodiment of the binding domain fusion protein comprises a binding domain containing a variable L chain (amino acid residues 1-106), linker (107-122), and variable H chain (123-242) chain that recognizes VEGF, a dimerization domain (243-259) containing an IgG1 hinge with a serine substitution for one of its three cysteine residues, and a WAP domain (260-368) contained in SLPI, and a WSHPQFED Step Tag (residues 369-376). (SEQ ID NO:5). The gene sequence is reported with a signal peptide (nucleic acid bases 21-83).

One embodiment of the binding domain fusion protein comprises a binding domain containing a variable L chain (amino acid residues 1-112), linker (113-127), and variable H chain (128-248) chain that recognizes CD28 (2E12), a dimerization domain (239-255) containing an IgG1 hinge with a serine substitution for one of its three cysteine residues, a WAP domain (256-364) contained in SLPI, and a WSHPQFEK Strep tag (residues 365-372) (SEQ ID NO:2). The gene sequence is reported with a signal peptide (nucleic acid bases 7-75).

One embodiment of the binding domain fusion protein comprises a binding domain containing a variable H chain (amino acid residues 1-120), linker (121-127), and variable L chain (128-233) chain that recognizes VEGF, a dimerization domain (234-250) containing an IgG1 hinge with a serine substitution for all three cysteine residues, and a WAP domain (251-359) contained in SLPI, and a WSHPQFED Step Tag (residues 360-367). (SEQ ID NO:6). The gene sequence is reported with a signal peptide (nucleic acid bases 19-75).

One embodiment of the binding domain fusion protein comprises a binding domain containing a variable L chain (amino acid residues 1-106), linker (107-112), and variable H chain (113-232) chain that recognizes VEGF, a dimerization domain (233-249) containing an IgG1 hinge with a serine substitution for all three cysteine residues, and a WAP domain (250-358) contained in SLPI, and a WSHPQFED Step Tag (residues 359-366). (SEQ ID NO:7). The gene sequence is reported with a signal peptide (nucleic acid bases 21-83).

One embodiment of the binding domain fusion protein comprises a binding domain containing a variable L chain (amino acid residues 1-112), linker (residues 113-127), and variable H chain (residues 128-248) chain that recognizes CD28 (2E12), a dimerization domain (residues 249-265) containing an IgG1 hinge with a serine substitution for all three of its cysteine residues, a WAP domain (residues 266-372) contained in SLPI, a spacer (373-388), a C_{H}3 domain (residues 389-497), and a WSHPQFEK Strep tag (residues 498-505) (SEQ ID NO:3). The gene sequence is reported with a signal peptide (nucleic acid bases 7-75).

One embodiment of the binding domain fusion protein comprises a binding domain containing a variable H chain (amino acid residues 1-120), linker (residues 121-137), and variable L chain (residues 138-243) chain that recognizes VEGF, a dimerization domain (residues 244-260) containing an IgG1 hinge with a serine substitution for all three cysteine residues, a WAP domain (residues 261-367) contained in SLPI, a spacer (368-382), a C_{H}3 domain (residues 383-494), and a WSHPQFEK Strep tag (residues 493-500) (SEQ ID NO:8). The gene sequence is reported with a signal peptide (nucleic acid bases 19-75).

One embodiment of the binding domain fusion protein comprises a binding domain containing a variable L chain (amino acid residues 1-106), linker (residues 107-122), and variable H chain (residues 123-242) chain that recognizes VEGF, a dimerization domain (residues 243-259) containing an IgG1 hinge with a serine substitution for all three cysteine residues, a WAP domain (residues 260-366) contained in SLPI, a spacer (367-381), a C_{H}3 domain (residues 382-491), and a WSHPQFEK Strep tag (residues 492-499) (SEQ ID NO:9). The gene sequence is reported with a signal peptide (nucleic acid bases 21-83).

Within the scope of the present invention is a binding domain fusion protein comprised of, for example, protein inhibition domains incorporating the following exemplary proteinase inhibition domains.

P03973a Residues 31-76:

(SEQ ID NO: 10)

KAGVCPPKKSAQCLRYKKPECQSDWQCPGKKRCCPDTCGIKCLDPV
P03973b Residues 85-130:

(SEQ ID NO: 11)

KPGKCPVTYGQCLMLNPPNFCEMDGQCKRDLKCCMGMCGKSCVSPV
P19957 Residues 72-117:

(SEQ ID NO: 12)

KPGSCPIILIRCAMLNPPNRCLKDTDCPGIKKCCEGSCGMACFVPQ
O95925 Residues 29-73:

(SEQ ID NO: 13)

FPRRCPKIREECEFQERDVCTKDRQCQDNKKCCVFSCGKKCLDLK
Q9H1F0a Residues 22-79:

(SEQ ID NO: 14)

GYRDKKRMQKTQLSPEIKVCQQQPKLYLCKHLCESHRDCQANNICCSTY
CGNVCMSIL
Q9H1F0b Residues 37-75:

(SEQ ID NO: 15)

EIKVCQQQPKLYLCKHLCESHRDCQANNICCSTYCGNV
Q8IUB3 Residues 22-73:

(SEQ ID NO: 16)

GYRDKMRMQRIKVCEKRPSIDLCIHHCSYFQKCETNKICCSAFCGNICMS
IL
Q9HC57 Residues 62-108:

(SEQ ID NO: 17)

RADRCPPPPTLPPGACQAARCQADSECPRHRRCCYNGCAYACLEAV
>Q14508a Residues 32-74:

(SEQ ID NO: 18)

KTGVCPELQADQNCTQECVSDSECADNLKCCSAGCATFCSLPN
Q14508b Residues 76-124:

(SEQ ID NO: 19)

SLPNDKEGSCPQVNINFPQLGLCRDQCQVDSQCPGQMKCCRNGCGKVSC
V TPNF
Q8IUB2a Residues 29-69:

(SEQ ID NO: 20)

KEGECPPHKNPCKELCQGDELCPAEQKCCTTGCGRICRDIP
Q8IUB2b Residues 72-114:

(SEQ ID NO: 21)

RKRDCPRVIRKQSCLKRCITDETCPGVKKCCTLGCNKSCVVPI S
Q8IUB2c Residues 122-162:

(SEQ ID NO: 22)

FGGECPADPLPCEELCDGDASCPQGHKCCSTGCGRTCLG DI
Q8IUB2d Residues 166-207

(SEQ ID NO: 23)

DIEGGRGGDCPKVLVGLCLVGCVMDENCQAGEKCCKSGCGRFCVPPV
Q8TCV5a Residues 30-74:

(SEQ ID NO: 24)

KSGGCPPDDGPCLLSVPDQCVEDSQCPLTRKCCYRACFRQCVPRV
Q8TCV5b Residues 77-121:

(SEQ ID NO: 25)

KLGSCPEDQLRCLSPMNHLCHKDSDCSGKKRCCHSACGRDCRDPA
Q9BQY9 Residues 31-69:

(SEQ ID NO: 26)

KPCPKIKVECEVEEIDQCTKPRDCPENMKCCPFSRGKKC
Q8IUB0a Residues 47-90:

(SEQ ID NO: 27)

KPGLCPKERLTCTTELPDSCNTDFDCKEYQKCCFFACQKKCMDP
Q8IUB0b Residues 150-193:

(SEQ ID NO: 28)

CRTACMLIVKDGQCPLFPFTERKECPPSCHSDIDCPQTDKCCESRCGFVC
ARA
Q8IUB0c Residues 197-239:

(SEQ ID NO: 29)

KKGFCPRKPLLCTKIDKPKCLQDEECPLVEKCCSHCGLKCMDP
Q8NEX5 Residue 24-89:

(SEQ ID NO: 30)

SFWNKDPFLDMIRETECWVQPPYKYCEKRCTKIMTCVRPNHTCCWTYCG
NICLDNE EPLKSMLNP
Q8NEX6 Residues 26-87:

(SEQ ID NO: 31)

EMRKKRYDRKELLLEECWGKPNVKECTNKCSKAFRCKDKNYTCCWTYC
GNICWINVETSGDY
Q8WWY7 Residues 30-74:

(SEQ ID NO: 32)

KAGVCPADNVRCFKSDPPQCHTDQDCLGERKCCYLHCGFKCVIPV
Q8IUB5 Residues 23-93:

(SEQ ID NO: 33)

SPKQRVLKYILEPPPCISAPENCTHLCTMQEDCEKGFQCCSSFCGIVCSS
ETFQKRNR IKHKGSEVIMPAN

The WAP domain region of a binding domain fusion protein is comprised of any domain or portion of trappin. Preferred WAP components are:

(SEQ ID NO: 34)

trappin-1 (two WAP domains)
MKSSGLFPFLVLLALGTLAPWAVEGSGKSFKAGVCPPKKSAQCLRY
KKPECQSDWQCPGKKRCCPDTCGIKCLDPVDTPNPTRRKPGKCPVTYG
QCLMLNPPNFCEMDGQCKRDLKCCMGMCGKSCVSPVKA
VEGSGKSFKAGVCPPKKSAQCLRY
KKPECQSDWQCPGKKRCCPDTCGIKCLDPVDTPNPTRRKPGKCPVTYG
QCLMLNPPNFCEMDGQCKRDLKCCMGMCGKSCVSPVKA

SEQ ID NO: 35

amino terminal domain of trappin-1
MKSSGLFPFLVLLALGTLAPWAVEGSGKSFKAGVCPPKKSAQCLRY
KKPECQSDWQCPGKKRCCPDTCGIKCLDPV
MVEGSGKSFKAGVCPPKKSAQCLRYKKPECQSDWQCPGKKRCCPDTCGI
KCLDPV

SEQ ID NO: 36

carboxyl terminal domain of trappin-1
DTPNPTRRKPGKCPVTYGQCLMLNPPNFCEMDGQCKRDLKCCMGMCGK
SCVSPVKA

SEQ ID NO: 37

WAP domain 5
MRTQSLLLLGALLAVGSQLPAVFGRKKGEKSGGCPPDDGPCLLSVPDQC
V
EDSQCPLTRKCCYRACFRQCVPRVSVKLGSCPEDQLRCLSPMNHLCHKDS
DCSGKKRCCHSACGRDCRDPARG

SEQ ID NO: 38

SWAM1
MWPNSILVLMTLLISSTLVTGGGVKGEEKRVCPPDYVRCIRQDDPQCYSD
NDCGDQEICCFWQCGFKCVLPVKDNSEEQIPQSKVGGVKGEEKRVCPPDY
VRCIRQDDPQCYSDNDCGDQEICCFWQCGFKCVLPVKDNSEEQIPQSKV

SEQ ID NO: 39

SWAM2
MKLLGLSLLAVTILLCCNMARPEIKKKNVFSKPGYCPEYRVPCPFVLIPK
CRRDKGCKDALKCCFFYCQMRCVDPWESPEARPEIKKKNVFSKPGYCPEY
RVPCPFVLIPKCRRDKGCKDALKCCFFYCQM RCVDPWESPE

The following Examples are offered by way of illustration and not by way of limitation.

### EXAMPLE 1

### Preparation of Synthetic Constructs of Binding Domain V_{L}—V_{H} Regions

This Example describes the making of a polynucleotide construct that encodes a binding domain fusion protein that recognize CD28 as a target-associated molecule and a binding domain fusion protein that recognizes VEGF as a target-associated molecule.

In making the constructs, the variable region of the H and L chains can be cloned from a mAb that reacts specifically with a desired target. The mAb from which the variable regions are cloned can be, for example, murine or human in origin, and is preferably human. If the mAb is murine in origin, the variable regions are typically humanized by placing the complementarity determining regions (CDRs) into the framework regions (FR) of a human variable region. A peptide linker can be placed between the variable regions. A exemplary linker contains amino acid sequences rich in glycine and serine, for example, a (G₄S)ₓ linker where x is an integer from 2 to 5, or 3-4. The variable regions can be oriented in either way:

NH₃⁺—V_{L}—V_{H}—COO⁻ or NH₃⁺—V_{H}—V_{L}—COO⁻.

The amino acid sequence of the desired binding domain fusion protein can be back translated into nucleic acid sequence using codon optimization matched to the expression host cell. The portions of genes of interest are synthesized synthetically by chemical synthesis. Restriction sites are inserted at the ends of all or a portion of the domains to facilitate substituting various domains into various binding domain fusion proteins.

Synthetic construction of 2E12 V_{L}—V_{H} single chain Fv (scFv) wt. The DNA fragments of 2E12 VL and 2E12 VH were separately generated by overlap extension of a set of 8 oligonucleotides of length about 66 to 69 bases long using the polymerase enzyme. These oligonucleotides were cloned into the TA cloning vector (Invitrogen). The fragments were assembled into scFv with V_{L} in front of V_{H} and a linker of either 15 amino acid ([G₄S]₃)or 5 amino acid (G₄S) were used to link the two fragments. The sequence of the constructs were confirmed by DNA sequencing.

The polynucleotide was constructed using the overlapping PCR extension method involving 8 oligonucleotides each for V_{L} and V_{H} domain of the scFv followed by gene amplification using two short end primers. The list of 16 oligonucleotides are shown in Table 1. For each V_{L} and V_{H} construction, a set of 8 oligonucleotides were mixed with the two short end oligonucleotides and PCR reactions were set up using TAQ polymerase employing the following conditions: initial 94° C. melting for 1 minute followed by 30 cycles of the following: 94° C. for 1 minute, 50 ° C. for 2 minutes and 72° C. for 3 minutes.

TABLE 1

Oligonucleotides for VL construction:

| | | |
|---|---|---|
| 2E12 VLF 1A | aagcttatgg attttcaagt gcagattttc agcttcctgc taatcagtgc ttcagtcata atgtccaga | (SEQ ID NO:) |
| 2E12 VLF 1B | tctttggctg tgtctctagg tcagagagcc accatctcct gcagagccag tgaaagtgtt gaatattat | (SEQ ID NO:) |
| 2E12 VLF 1C | ccaggacagc cacccaaact cctcatctct gctgctagca acgtagaatc tggggtccct gccaggttt | (SEQ ID NO:) |
| 2E12 VLF 1D | aacatccatc ctgtggagga ggatgatatt gcaatgtatt tctgtcagca aagtaggaag gttccatgg | (SEQ ID NO:) |
| 2E12 VLR 1A | tagagacaca gccaaagaag ctggagattg ggtgagcaca atgtcgactc ctctggacat tatgactga | (SEQ ID NO:) |
| 2E12 VLR 1B | tttgggtggc tgtcctggtt tctgttggta ccactgcatt aaacttgtga cataatattc aacactttc | (SEQ ID NO:) |
| 2E12 VLR 1C | ctccacagga tggatgttga ggctgaagtc tgtcccagac ccactgccac taaacctggc agggacccc | (SEQ ID NO:) |
| 2e12 VLR 1D | ggatccaccg ccaccccgtt tgatttccag cttggtgcct ccaccgaacg tccatggaac cttcctact | (SEQ ID NO:) |

Oligonucleotides for VH construction:

| | | |
|---|---|---|
| 2E12 VHF 1A | ggatccggcg gaggtgggtc gggtggcggc ggatctcagg tgcagctgaa ggagtcagga cctggc | (SEQ ID NO:) |
| 2E12 VHF 1B | acatgcaccg tctcagggtt ctcattaacc ggctatggtg taaactgggt tcgccagcct ccagga | (SEQ ID NO:) |
| 2E12 VHF 1C | ggtgatggaa gcacagacta taattcagct ctcaaatcca gactatcgat caccaaggac aactcc | (SEQ ID NO:) |
| 2E12 VHF 1D | ctgcaaactg atgacacagc cagatactac tgtgctcgag atggttatag taactttcat tactatg | (SEQ ID NO:) |
| 2E12 VHR 1A | ccctgagacg gtgcatgtga tggacaggct ctgtgagggc gccaccaggc caggtcctga ctcctt | (SEQ ID NO:) |
| 2E12 VHR 1B | gtctgtgctt ccatcacccc atatcattcc cagccactct agaccctttc ctggaggctg gcgaac | (SEQ ID NO:) |
| 2E12 VHR 1C | tgtgtcatca gtttgcagac tgttcatttt taagaaaact tggctcttgg agttgtcctt ggtgat | (SEQ ID NO:) |
| 2e12 VHR 1D | tgatcagaggagacggtgactgaggttccttgaccccagtagtccataacatagtaatgaaagttac | (SEQ ID NO:) |

The V_{L} and V_{H} fragments were gel isolated based on size and ligated into the TOPO cloning vector (Invitrogen) and sequenced to verify their sequences. The V_{L} and V_{H} fragments were then digested with the appropriate restriction enzymes and assembled by ligation into the pUC19 vector to generate the V_{L}—V_{H} 2E12 wt scFv. The sequence was then verified by DNA sequencing. The DNA and protein sequence of 2E12 scFv wt is shown in Table 2. The scFv fragment was then ligated into the PD18 vector bearing the SSS or SCC hinge plus the CH2/CH3 domains at the 3′ end of the scFv gene for expression as 2E12 SMIPs in COS cells.

TABLE 2

DNA sequence of 2E12 VL-VH:

| | |
|---|---|
| aagcttatgg attttcaagt gcagattttc agcttcctgc taatcagtgc ttcagtcata | 60 |
| atgtccagag gagtcgacat tgtgctcacc caatctccag cttctttggc tgtgtctcta | 120 |
| ggtcagagag ccaccatctc ctgcagagcc agtgaaagtg ttgaatatta tgtcacaagt | 180 |
| ttaatgcagt ggtaccaaca gaaaccagga cagccaccca aactcctcat ctctgctgct | 240 |
| agcaacgtag aatctggggt ccctgccagg tttagtggca gtgggtctgg gacagacttt | 300 |
| agcctcaaca tccatcctgt ggaggaggat gatattgcaa tgtatttctg tcagcaaagt | 360 |
| aggaaggttc catggacgtt cggtggaggc accaagctgg aaatcaaacg gggtggcggt | 420 |
| ggatacggcg gaggtgggtc gggtggcggc ggatctcagg tgcagctgaa ggagtcagga | 480 |
| cctggcctgg tggcgccctc acagagcctg tccatcacat gcaccgtctc agggttctca | 540 |
| ttaaccggct atggtgtaaa ctgggttcgc cagcctccag gaaagggtct agagtggctg | 600 |
| ggaatgatat ggggtgatgg aagcacagac tataattcag ctctcaaatc cagactatcg | 660 |
| atcaccaagg acaactccaa gagccaagtt ttcttaaaaa tgaacagtct gcaaactgat | 720 |
| gacacagaca gatactactg tgctcgagat ggttatagta actttcatta ctatgttatg | 780 |
| gactactggg gtcaaggaac ctcagtcacc gtctcctctg atca | 824 |
| signal peptide - 7-75 | |
| VL: 76-411 | |
| Linker: 412-456 | |
| VH: 457-819 | |
| Protein sequence of 2E12 VL-VH: | |

| | |
|---|---|
| 1 | divltqspas lavslgqrat iscrasesve yyvtslmqwy qqkpgqppkl lisaasnves |
| 61 | gvparfsgsg sgtdfslnih pveeddiamy fcqqsrkvpw tfgggtklei krggggsggg |
| 121 | gsgqggsqvq lkesgpglva psqslsitct vsgfsltgyg vnwvrqppgk glewlgmiwg |
| 181 | dgstdynsal ksrlsitkdn sksqvflkmn slqtddtary ycardgysnf hyyvmdywgq |
| 241 | gtsvtvss |

VL: 1-112
Linker: 113-127
VH: 128-248

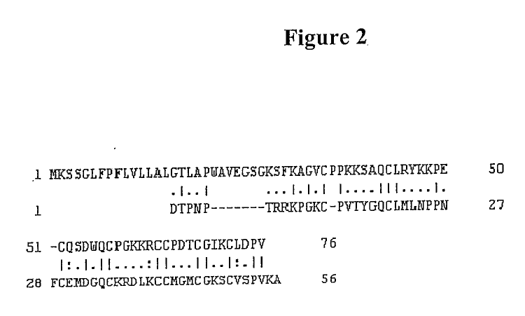

Synthetic construction of mouse anti-VEGF V_{L}—V_{H} single chain Fv (scFv) wt. The polynucleotide was constructed using the overlapping PCR extension method involving 8 oligonucleotides each for V_{L} and V_{H} domain of the scFv followed by gene amplification using two short end primers. The list of 16 oligonucleotides are shown in Table 3. For each V_{L} and V_{H} construction, a set of 8 oligonucleotides were mixed with the two short end oligonucleotides and PCR reactions were set up using high fidelity TAQ polymerase employing the following conditions: initial 94° C. melting for 1 minute followed by 30 cycles of the following: 94° C. for 1 minute, 50 ° C. for 2 minutes and 72° C. for 3 minutes. The final concentration of each of the eight long oligonucleotides were 10 nM whereas the short oligonucleotides were 1 uM. FIG. 2 shows the step involved in the generation of the full gene from the 8 oligonucleotides. The gene construction and amplification all happened in a single tube and in a single PCR reaction.

The V_{L} and V_{H} fragments were gel isolated based on size and ligated into the TOPO cloning vector (Invitrogen) and sequenced to verify their sequences. The V_{L} and V_{H} fragments were then digested with the appropriate restriction enzymes and assembled by ligation into the pUC19 vector to generate the V_{L}—V_{H} anti-VEGF wt scFv. The sequence was then verified by DNA sequencing. The DNA and protein sequence of mouse anti-human VEGF scFv wt is shown in Table 3.

TABLE 3

Sequence of the 20 oligonucleotides (16 long and 4
short). F indicates forward primer whereas R indicates reverse primer.

| | |
|---|---|
| a-mvegfhvlfr-F1 | ATAGTCTAGG TCGACATTGT GCTGACACAG TTTCCTGCTA GCCTTAGCGT ATTTTTGGGG CA |
| (SEQ ID NO:) | |
| a-mvegfhvlfr-F2 | GCCAAAGTGT CAGTACATAT GGCTATAGTT ATATGCACTG GAACCAACAG AAACCAGGAC AG |
| (SEQ ID NO:) | |
| a-mvegfhvlfr-F3 | ATCCAATCTA GAATTTGGGG TCCCTGCCAG GTTCAGTGGC AGTGGGTCTG GGACAGACTT CA |
| (SEQ ID NO:) | |
| a-mvegfvlfr-F4 | GAGGATGCTG CAACCTATTA TTGTCAGCAC ATTAGGGAGC TTCCTTACAC GTTCGGAGGG GG |
| (SEQ ID NO:) | |
| a-mvegfvlfr-R1 | ATGTACTGAC ACTTTGGCTG GCCCTGCATG AAATGGTGGC CCTCTGCCCC AAAAATACGC TA |
| (SEQ ID NO:) | |
| a-mvegfvlfr-R2 | CCAAATTCTA GATTGGATAC AAGATAAATG AGGAGTCTGG GTGGCTGTCC TGGTTTCTGT TG |
| (SEQ ID NO:) | |
| a-mvegfvlfr-R3 | ATAGGTTGCA GCATCCTCCT CCTCCACAGG ATGGATGTTG AGGGTGAAGT CTGTCCCAGA CC |
| (SEQ ID NO:) | |
| a-mvegfvlfr-R4 | ACCTCCGCCG GATCCACCGC CACCTTTGAT TTCCAGCTTG GTCCCCCCTC CGAACGTGTA A |
| (SEQ ID NO:) | |
| a-mvegfVLFrtsht-F | ATAGTCTAGG TCGACATTGT GCTG |
| (SEQ ID NO:) | |
| a-mvegfVLFrtsht-R | ACCTCCGCCG GATCCACCGC CAC |
| (SEQ ID NO:) | |
| a-mvegfhvhbk-F1 | GGTGGCGGTG GATCCGGCGG AGGTGGGTCG GGTGGCGGCGG ATCGGAGGTAC AGCTTCTGGA |
| (SEQ ID NO:) | GTCTGGG |
| a-mvegfhvhbkr-F2 | TTGTCCTGCA CAGCTTCTGG CTTCAACATT AAAGACACCTA TATGCACTGG GTGAAGCAGA |
| (SEQ ID NO:) | GGCCTGAA |
| a-mvegfhvhbk-F3 | GCGAATGGTA ATACTAAATA TGACCCGAAG TTCCAGGGCA AGGCCACTAT AACAGCAGAC |
| (SEQ ID NO:) | ACATCCTCC |
| a-mvegfvhbk-F4 | TCTGAGGACA CCGCGGTCTA TTACTGTGCT AGGCCATCTA TTTACTACGG TAGTAACCAC |
| (SEQ ID NO:) | TGGTACTTC |
| a-mvegfvhbk-R1 | AGAAGCTGTG CAGGACAACT TGACTGAGGC CCCTGGCTTCA CAAGCTCTGC CCCAGACTCC |
| (SEQ ID NO:) | AGAAGCTG |
| a-mvegfvhbk-R2 | TTTAGTATTA CCATTCGCAG GATCGATCCT TCCGATCCAC TCCAGGCCCT GTTCAGGCCT |
| (SEQ ID NO:) | CTGCTTCAC |
| a-mvegfvhbk-R3 | GACCGCGGTG TCCTCAGATG TCAGGCTGCT GAGCTGCAGG TAGGCTGTGT TGGAGGATGT |
| (SEQ ID NO:) | GTCTGCTGT |
| a-mvegfvhbkr-R4 | TGATACTATC AGATCTGAGG AGACGGTGAC TGAGGTTCCT GCGCCCCAGA CATCGAAGTA |
| (SEQ ID NO:) | CCAGTGGTTA CT |
| a-mvegfVHbksht-F | GGTGGCGGTG GATCCGGCGG AGGT |
| (SEQ ID NO:) | |
| a-mvegfVHbksht-R | TGATACTATC AGATCTGAGG AGA |
| (SEQ ID NO:) | |

TABLE 4
Nucleotide and Protein Sequence for mouse antihuman VEGF VL-VH.

DNA sequence of mouse anti-human VEGF VL-VH:

| | |
|---|---|
| aagcttgccg ccatggattt tcaagtgcag attttcagct tcctgctaat cagtgcttca | 60 |
| gtcataattg ccagaggagt cgactctgag ctgactcagg accctgctgt gtctgtggcc | 120 |
| ttgggacaga cagtcaggat cacatgccaa ggagacagcc tcagaagcta ttatgcaagc | 180 |
| tggtaccagc agaagccagg acaggcccct gtacttgtca tctatggtaa aaacaaccgg | 240 |
| ccctcaggga taccagaccg attctctggc tccagctcag gaaacacagc ttccttgacc | 300 |
| atcactgggg ctcaggcgga agatgaggct gactattact gtaactcccg ggacagcagt | 360 |
| ggtaaccatg tggtattcgg cggagggacc aagctgaccg tcctaggtgg cggtggctcg | 420 |
| ggcggtggtg ggtcgggtgg cggcgggagc tctcaggtgc agctggtgca gtctggggct | 480 |
| gagtcgaaga agcctggggc ctcagtgaag gtttcctgca aggcttctgg atacaccttc | 540 |
| actagctatg ctatgcattg ggtgcgccag gcccccggac aaaggcttga gtggatggga | 600 |
| tggatcaacg ctggcaatgg taacacaaaa tattcacaga agttccaggg cagagtcacc | 660 |
| attaccaggg acacatccgc gagcacagcc tacatggagc tgagcagcct gagatccgaa | 720 |
| gacacggccg tgtattactg tgcaaggttg acgcggaata agtttaagtc gcgtggtcat | 780 |
| tggggccaag gtaccctggt caccgtgtcg agagatctg | 824 |
| signal peptide - 13-81 | |
| VL: 82-405 | |
| Linker: 406-450 | |
| VH: 451-819 | |

Protein sequence of mouse anti-human VEGF VL-VH:

| | |
|---|---|
| DSELTQDPAV SVALGQTVRI TCQGDSLRSY YASWYQQKPG QAPVLVIYGK NNRPSGIPDR | 60 |
| FSGSSSGNTA SLTITGAQAE DEADYYCNSR DSSGNHVVFG GGTKLTVLGG GGSGGGGSGG | 120 |
| GGSSQVQLVQ SGAESKKPGA SVKVSCKASG YTFTSYAMHW VRQAPGQRLE WMGWINAGNG | 180 |
| NTKYSQKFQG RVTITRDTSA STAYMELSSL RSEDTAVYYC ARLTRNKFKS RGHWGQGTLV | 240 |
| TVSRDL | 246 |
| VL: 1-108 | |
| Linker: 109-123 | |
| VH: 124-246 | |

Synthetic Construction of Human Anti-VEGF V_{L}—V_{H} Single Chain Fv (scFv)

This method involves the use of overlapping oligonucleotide primers and PCR using a high fidelity DNA polymerase (Invitrogen PCR HIFI mix) to synthesize an immunoglobulin V-region. Starting at the middle of the V-region sequence, 45-70 base primers are designed such that the growing chain is extended by 40-50 bases in either direction and contiguous primers overlap by a minimum of 20 bases. Each PCR step requires two primers, one priming on the anti-sense strand (forward or sense primer) and one priming on the sense strand (reverse or anti-sense primer) to create a growing double-stranded PCR product as shown by the example below. During primer design, changes can be made in the nucleotide sequence of the final product to create restriction enzyme sites, destroy existing restriction enzyme sites, add flexible linkers, change, delete or insert bases that alter the amino acid sequence, optimize the overall DNA sequence to enhance primer synthesis and maintain codon usage rules for the organism used to express the synthetic gene.

The heavy and light chain variable regions are synthesized separately and each V-region is synthesized in a two step process (PCR 1 and 2). In the following example, a VH is synthesized, but the process is the same for a VL synthesis. Primers are numbered sequentially followed by its directional designation ( F=forward, R=reverse ) and the concentration (μM) of each primer in the PCR.
Step 1—94 C, 4 m—3 cycles 94° C., 30 s/65° C., 30 s/70° C., 30 s—one 72 C, 6 m extension 5106F 10 μM; 5107R 10 μM
Step 2—27 cycles after addition of the following primers:
5102F 10 uM; 5103R 10 uM; 5104F 10 uM; 5105R 10 uM
Step 3—isolated the PCR product (by gel purification) from PCR 1 and diluted 1:20
PCR 2 Step 1—94 C, 4 m—30 cycles 94 C, 1 m/55 C, 1 m/72 C, 1 m—one 72 C, 6 m extension 1 uL of diluted PCR 1; 5098F 10 uM; 5099R 10 uM; 5100F 10 uM; 5101R 10 uM; 5108F 10 uM; 5109R 10 uM; 5100F 10 uM; and 5101R 10 uM
PCR product purification (by gel purification) to remove excess primers
TA TOPO cloning (Invitrogen PCR 2.1 topo vector) to sequence
Restriction digest and three-way ligation to the Human anti VEGF vL and to the css hinge IgG in the PD18 vector

TABLE 5

Primers used for synthesis of Human anti VEGFscFv VH + VL
Light Chain primers = #5086-5097
Heavy Chain primers = #5098-5109

| | | |
|---|---|---|
| Seq# | Seq Name | Seq 5′ to 3′ |

| | | |
|---|---|---|
| 5086F | vlavegfvlvh5-1 | ACGCGTAAGC TTGCCGCCCC ATGGCCTGGA CCCCTCTCTG GCTCACT |
| | (SEQ ID NO:) | |
| 5087R | vlavegfvlvh3-1 | AGAGCTCCCG CCGCCACCCG ACCCACCACC GCCCGAGCCA CCGCCA |
| | (SEQ ID NO:) | |
| 5088F | vlavegfvlvh5-2 | GGACCCCTCT CTGGCTCACT CTCCTCACTC TTTGCATAGG TTCCGTGGTT TCTTCTGAGC |
| | (SEQ ID NO:) | TGACTCAGGA C |
| 5089R | vlavegfvlvh3-2 | CACCGCCCGA GCCACCGCCA CCTAGGACGG TCAGCTTGGT CCCTCCGCCG AATACCACAT |
| | (SEQ ID NO:) | GGTTACCACT |
| 5090F | vlavegfvlVh5-3 | TCTTCTGAGC TGACTCAGGA CCCTGCTGTG TCTGTGGCCT TGGGACAGAC AGTCAGGATC ACATG |
| | (SEQ ID NO:) | |
| 5091R | vlavegfvlvh3-3 | AATACCACAT GGTTACCACT GCTGTCCCGG GAGTTACAGT AATAGTCAGC CTCATCTTCC GCCTG |
| | (SEQ 10 NO:) | |
| 5092F | vlavegfvlvh5-4 | CAGACAGTCA GGATCACATG CCAAGGAGAC AGCCTCAGAA GCTATTATGC AAGCTGGTAC CAGCAG |
| | (SEQ ID NO:) | |
| 5093R | vlavegfvlvh3-4 | TCAGCCTCAT CTTCCGCCTG AGCCCCAGTG ATGGTCAAGG AAGCTGTGTT TCCTGAGCTG GAGCC |
| | (SEQ ID NO:) | |
| 5094F | vlavegfvlvh5-5 | TATGCAAGCT GGTACCAGCA GAAGCCAGGA CAGGCCCCTG TACTTGTCAT CTATGGTAAA AACAACCG |
| | (SEQ ID NO:) | |
| 5095R | vlavegfvlvh3-5 | GTGTTTCCTG AGCTGGAGCC AGAGAATCGG TCTGGTATCC CTGAGGGCCG GTTGTTTTTA CCATAGAT |
| | (SEQ ID NO:) | |
| 5096F | vlavegfvhvl5-1a | GGGAGCTCTT CTGAGCTGAC TCAGGACCCT |
| | (SEQ ID NO:) | |
| 5097R | vlavegfvhvl3-1a | CAGATCTAGG ACGGTCAGCT TGGTCCCTCC GCCGAATACC ACATGGTTAC CA |
| | (SEQ ID NO:) | |
| 5098F | vhavegfvhvl5-1 | ACGCGTAAGC TTGCCGCCAT GGACTGGACC TGGAGAATCC TCTTCTTGGT GGCAGCAGCC ACAGG |
| | (SEQ ID NO:) | |
| 5099R | vhavegfvhvl3-1 | AGAGCTCCCG CCGCCACCCG ACCCACCACC GCCCGAGCCA CCG |
| | (SEQ ID NO:) | |
| 5100F | vhavegfvhvl5-2 | TGGTGGCAGC AGCCACAGGA GCCCACTCCC AGGTGCAGCT GGTGCAGTCT GGGGCTGAGT |
| | (SEQ ID NO:) | CGAAGAAGCC |
| 5101R | vhavegfvhvl3-2 | CACCACCGCC CGAGCCACCG CCACCTCTCG ACACGGTGAC CAGGGTAC |
| | (SEQ ID NO:) | |
| 5102F | vhavegfvhvl5-3 | GGCTGAGTCG AAGAAGCCTG GGGCCTCAGT GAAGGTTTCC TGCAAGGCTT CTGGATACAC CTTCACTA |
| | (SEQ ID NO:) | |
| 5103R | vhavegfvhvl3-3 | CTTGGCCCCA ATGACCACGC GACTTAAACT TATTCCGCGT CAACCTTGCA CAGTAATACA CGGCCGTGTC |
| | (SEQ ID NO:) | |
| 5104F | vhavegfvhvl5-4 | TTCTGGATAC ACCTTCACTA GCTATGCTAT GCATTGGGTG CGCCAGGCCC CCGGACAAAG GCTTGAGTGG |
| | (SEQ ID NO:) | |
| 5105R | vhavegfvhvl3-4 | ACAGTAATAC ACGGCCGTGT CTTCGGATCT CAGGCTGCTC AGCTCCATGT AGGCTGTGCT CGCGGATGTG |
| | (SEQ ID NO:) | |
| 5106F | vhavegfvhvl5-5 | CCGGACAAAG GCTTGAGTGG ATGGGATGGA TCAACGCTGG CAATGGTAAC ACAAAATATT CACAGAAG |
| | (SEQ ID NO:) | |
| 5107R | vhavegfvhvl3-5 | GGCTGTGCTC GCGGATGTGT CCCTGGTAAT GGTGACTCTG CCCTGGAACT TCTGTGAATA TTTTGTGT |
| | (SEQ ID NO:) | |
| 5108F | vhavegfvlvh5-1a | GGGAGCTCTC AGGTGCAGCT GGTGCAGTCT GGGGCTGAGT CGAAGAAGCC |
| | (SEQ ID NO:) | |
| 5109R | vhavegfvlvh3-1a | CAGATCTCTC GACACGGTGA CCAGGGTACC TTGGCCCCAA TGACCACGC |

Final Human Anti VEGF (vL+vH orientation) nucleotide and amino acid sequence after ligation (SEQ ID NO:)

| | M A W T |
|---|---|
| 1 | ACGCGTAAGC TTGCCGCCCC ATGGCCTGGA |
| | P L W L T L |
| | CCCCTCTCTG GCTCACTCTC |
| | L T L C I G S V V S |
| 51 | CTCACTCTTT GCATAGGTTC CGTGGTTTCT |
| | S E L T Q D P |
| | TCTGAGCTGA CTCAGGACCC |
| | A V S V A L G Q T V |
| 101 | TGCTGTGTCT GTGGCCTTGG GACAGACAGT |
| | R I T C Q G D |
| | CAGGATCACA TGCCAAGGAG |
| | S L R S Y Y A S W Y |
| 151 | ACAGCCTCAG AAGCTATTAT GCAAGCTGGT |
| | Q Q K P G Q |
| | ACCAGCAGAA GCCAGGACAG |
| | A P V L V I Y G K N |
| 201 | GCCCCTGTAC TTGTCATCTA TGGTAAAAAC |
| | N R P S G I P |
| | AACCGGCCCT CAGGGATACC |
| | D R F S G S S S G N |
| 251 | AGACCGATTC TCTGGCTCCA GCTCAGGAAA |
| | T A S L T I T |
| | CACAGCTTCC TTGACCATCA |
| | G A Q A E D E A D Y |
| 301 | CTGGGGCTCA GGCGGAAGAT GAGGCTGACT |
| | Y C N S R D |
| | ATTACTGTAA CTCCCGGGAC |
| | S S G N H V V F G G |
| 351 | AGCAGTGGTA ACCATGTGGT ATTCGGCGGA |
| | G T K L T V L |
| | GGGACCAAGC TGACCGTCCT |
| | G G G G S G G G G S |
| 401 | AGGTGGCGGT GGCTCGGGCG GTGGTGGGTC |
| | G G G G S S Q |
| | GGGTGGCGGC GGGAGCTCTC |
| | V Q L V Q S G A E S |
| 451 | AGGTGCAGCT GGTGCAGTCT GGGGCTGAGT |
| | K K P G A S |
| | CGAAGAAGCC TGGGGCCTCA |
| | V K V S C K A S G Y |
| 501 | GTGAAGGTTT CCTGCAAGGC TTCTGGATAC |
| | T F T S Y A M |
| | ACCTTCACTA GCTATGCTAT |
| | H W V R Q A P G Q R |
| 551 | GCATTGGGTG CGCCAGGCCC CCGGACAAAG |
| | L E W M G W I |
| | GCTTGAGTGG ATGGGATGGA |
| | N A G N G N T K Y S |
| 601 | TCAACGCTGG CAATGGTAAC ACAAAATATT |
| | Q K F Q G R |
| | CACAGAAGTT CCAGGGCAGA |
| | V T I T R D T S A S |
| 651 | GTCACCATTA CCAGGGACAC ATCCGCGAGC |
| | T A Y M E L S |
| | ACAGCCTACA TGGAGCTGAG |
| | S L R S E D T A V Y |
| 701 | CAGCCTGAGA TCCGAAGACA CGGCCGTGTA |
| | Y C A R L T R |
| | TTACTGTGCA AGGTTGACGC |
| | N K F K S R G H W G |
| 751 | GGAATAAGTT TAAGTCGCGT GGTCATTGGG |
| | Q G T L V T |
| | GCCAAGGTAC CCTGGTCACC |
| | V S R D L |
| 801 | GTGTCGAGAG ATCTG |

Final Human Anti VEGF (vL+vH Orientation) amino acid sequence (SEQ ID NO:)

MAWTPLWLTLLTLCIGSVVSSELTQDPAVSVALGQTVRITCQGDSLRSYY
ASWYQQKPGQAPVLVIYGKNNRPSGIPDRFSGSSSGNTASLTITGAQAED
EADYYCNSRDSSGNHVVFGGGTKLTVLGGGGSGGGGSGGGGSSQVQLVQS
GAESKKPGASVKVSCKASGYTFTSYAMHWVRQAPGQRLEWMGWINAGNGN
TKYSQKFQGRVTITRDTSASTAYMELSSLRSEDTAVYYCARLTRNKFKSR
GHWGQGTLVTVSRD

Final Human Anti VEGF (vH+vL orientation) Nucleotide Sequence After Ligation (SEQ ID NO:)

| | |
|---|---|
| 1 | ACGCGTAAGC TTGCCGCCAT GGACTGGACC TGGAGAATCC |
| | TCTTCTTGGT |
| 51 | GGCAGCAGCC ACAGGAGCCC ACTCCCAGGT GCAGCTGGTG |
| | CAGTCTGGGG |
| 101 | CTGAGTCGAA GAAGCCTGGG GCCTCAGTGA AGGTTTCCTG |
| | CAAGGCTTCT |
| 151 | GGATACACCT TCACTAGCTA TGCTATGCAT TGGGTGCGCC |
| | AGGCCCCCGG |
| 201 | ACAAAGGCTT GAGTGGATGG GATGGATCAA CGCTGGCAAT |
| | GGTAACACAA |
| 251 | AATATTCACA GAAGTTCCAG GGCAGAGTCA CCATTACCAG |
| | GGACACATCC |
| 301 | GCGAGCACAG CCTACATGGA GCTGAGCAGC CTGAGATCCG |
| | AAGACACGGC |
| 351 | CGTGTATTAC TGTGCAAGGT TGACGCGGAA TAAGTTTAAG |
| | TCGCGTGGTC |
| 401 | ATTGGGGCCAAGGTACCCTGGTCACCGTGTCGAGAGGTGGCGG |
| | TGGCTCG |
| 451 | GGCGGTGGTG GGTCGGGTGG CGGCGGGAGC TCTTCTGAGC |
| | TGACTCAGGA |
| 501 | CCCTGCTGTG TCTGTGGCCT TGGGACAGAC AGTCAGGATC |
| | ACATGCCAAG |
| 551 | GAGACAGCCT CAGAAGCTAT TATGCAAGCT GGTACCAGCA |
| | GAAGCCAGGA |
| 601 | CAGGCCCCTG TACTTGTCAT CTATGGTAAA AACAACCGGC |
| | CCTCAGGGAT |
| 651 | ACCAGACCGA TTCTCTGGCT CCAGCTCAGG AAACAGAGCT |
| | TCCTTGACCA |
| 701 | TCACTGGGGC TCAGGCGGAA GATGAGGCTG ACTATTACTG |
| | TAACTCCCGG |
| 751 | GACAGCAGTG GTAACCATGT GGTATTCGGC GGAGGGACCA |
| | AGCTGACCGT |
| 801 | CCTAGATCTG |

Final Human Anti VEGF (vH+vL Orientation) Amino Acid Sequence After Ligation

MDWTWRLLFLVAAATGAHSQVQLVQSGAESKKPGASVKVSCKASGYTFTS
YAMHWVRQAPGQRLEWMGWINAGNGNTKYSQKFQGRVTITRDTSASTAYM
ELSSLRSEDTAVYYCARLTRNKFKSRGHWGQGTLVTVSRGGGGSGGGGSG
GGGSSSELTQDPAVSVALGQTVRITCQGDSLRSYYASWYQQKPGQAPVLV
IYGKNNRPSGIPDRFSGSSSGNTASLTITGAQAEDEADYYCNSRDSSGNH
VVFGGGTKLTVLDL

### EXAMPLE 2

### Construction and Characterization of 2E12-SLPI Conjugates

The SLPI DNA was originally cloned by PCR from an Ovarian cell line. Three different fragments were created. First, the SCC hinge SLPI strep tag was generated by adding the SCC hinge N-terminal to the SLPI gene and the strep tag C-terminal to the SLPI gene using PCR. Second, the SSS hinge SLPI strep tag was generated by adding the SSS hinge N-terminal to the SLPI gene and the strep tag C-terminal to the SLPI gene using PCR. And finally, the SSS hinge SLPI CH3 strep tag was generated by creating SSS hinge SPLI and CH3 strep tag fragments through PCR and fusing them by overlap extension of the two fragments. All the sequences were confirmed by DNA sequencing.

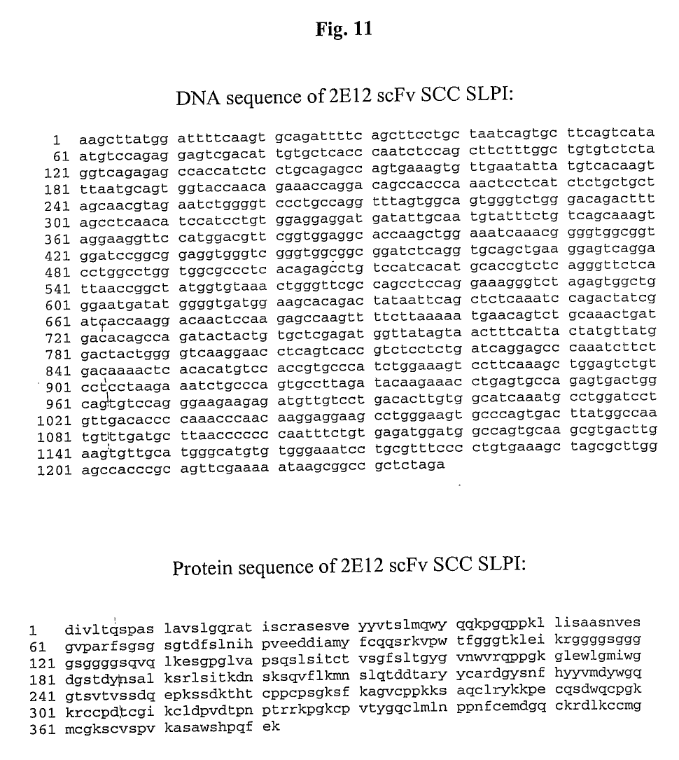

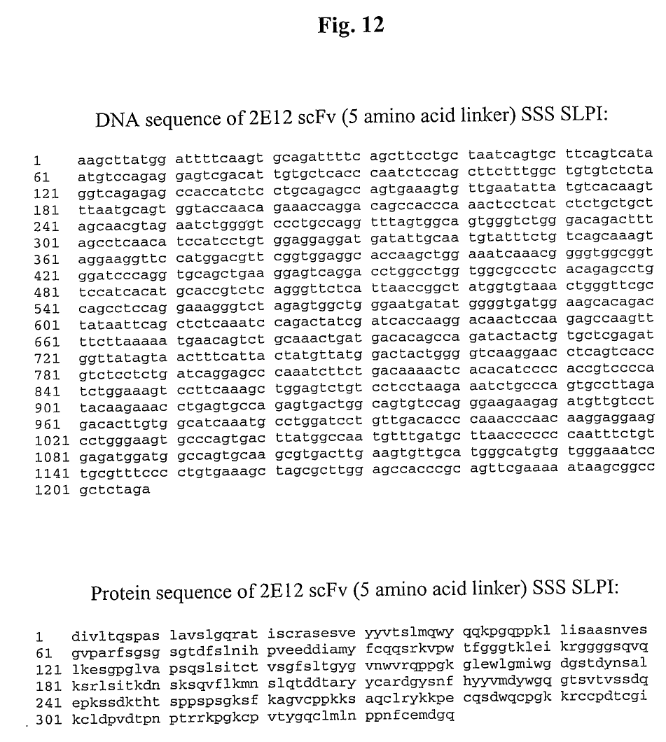

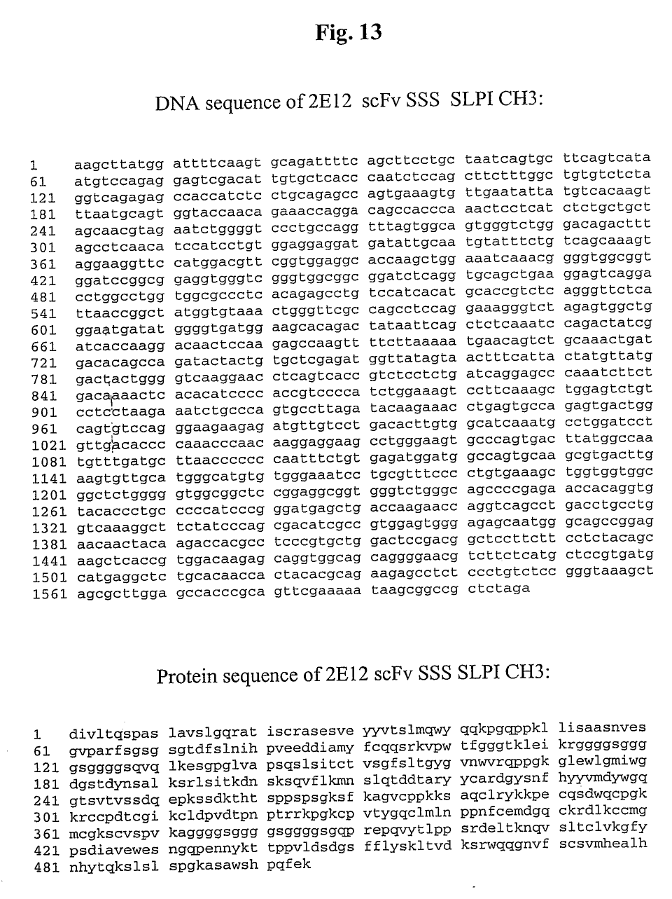

Three final molecules were generated by combining different fragments described above. First, the 2E12 scFv with 15 amino acid linker was assembled with the SCC hinge SPLI tag using the BciI restriction site to give the 2E12 scFv-SCC-SPLI tag (FIG. 11). Second, the 2E12 scFv with 5 amino acid linker was assembled with the SSS hinge SPLI tag to give the 2E12 scFv(5aa linker)-SSS-SLPI tag (FIG. 12). Finally, the 2E12 scFv with 15 amino acid linker was assembled with SSS SLPI CH3 to give 2E12 scFv-SSS-SLPI-CH3 tag (FIG. 13).

Protein Expression and Purification. All the constructs were transfected into COS7 cell lines using lipofectmine2000 (Invitrogen) according to the manufacturer's protocol in 24-well plate with each well containing 0.5 ml of media. The serum media was replaced with serum free media a day following transfection. The supernatants were collected three days later and tested for activity. For a larger scale expression, dishes of 150 mm were used and the supernatants were collected every three days three times, pooled and clarified by passing through a filter. The supernatants were then passed through a protein A immobilized column that had been pre-equilibrated with 100 mM Tris pH 8 buffer. The column was exhaustively washed with PBS and the protein eluted with 100 mM Citric acid, pH 2.5. The eluted protein was then dialyzed against PBS and concentrated. Concentrations of each protein were determined by the absorbance at 280 nm using extinction coefficient and molecular mass calculated from their amino acid sequence.

The three 2E12-SLPI constructs were transfected into COS7 cells as described above, but media containing serum was used instead of serum free media following transfection. The supernatants were also collected as above. For purification, the supernatants were passed through a strep-tactin column that had been equilibrated with the binding buffer (100 mM Tris-Cl pH 8.0, 150 mM NaCl, 1 mM EDTA). After exhaustive washing of the column with binding buffer, the protein was eluted with same buffer plus 2.5 mM desthiobiotin. The eluted protein was dialyzed against PBS, concentrated and quantitated using OD280 as described above.

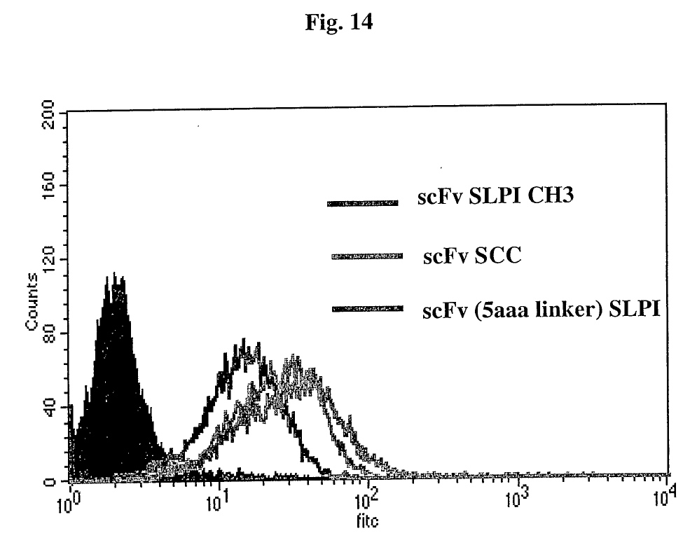

Binding activity of 2E12-SLPI conjugate. The mammalian vectors harboring different conjugate genes (scFv-SCC-SLPI, scFv(5aa linker)-SSS-SLPI and scFv-SSS-SLPI-CH3) were transfected into COS cells and the supernatants collected. The supernatants were then incubated with CD28-CHO cells washed, and then probed with goat anti-SLPI followed by rabbit anti-goat Fitc. The cells were then analysed using FACS assay. The data is shown in FIG. 14. We have shown that we can make functional 2E12-SLPI conjugates via 2E12 scFv-SSC-SLPI and 2E12 scFv-SSS-SLPI-CH3 as these molecules bind to CD28-CHO and can be detected by anti-SLPI antibody in the FACS assay.

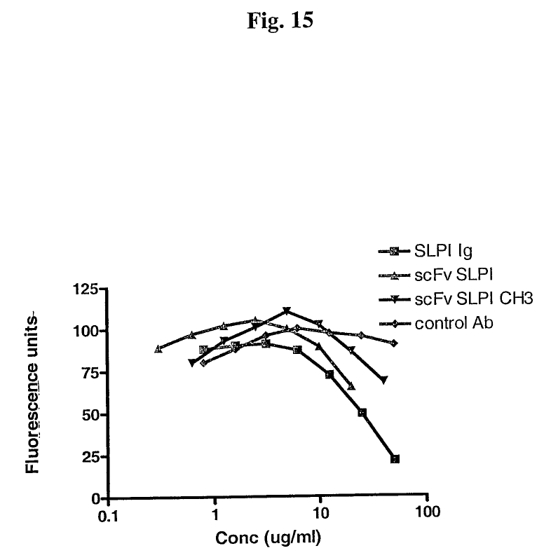

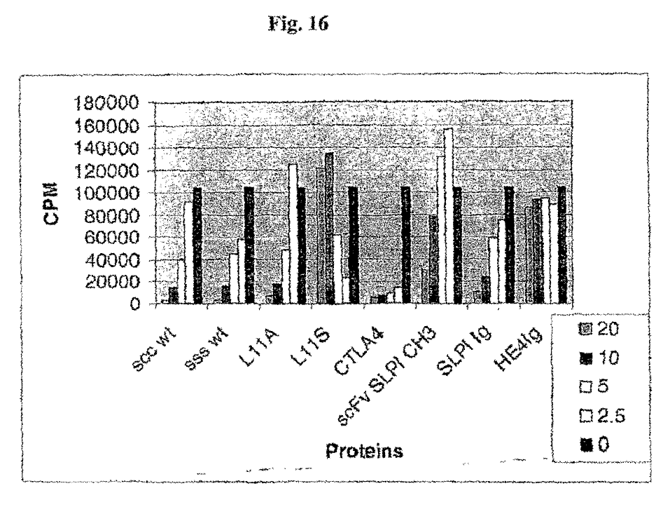

Protease inhibition activity of 2E12-SLPI conjugates and SLPI Ig.Purified samples of SLPI Ig (question—is this construct previously called SLPI Ig?), 2E12 scFv-SCC-SLPI, scFv-SSSI-SLPI-CH3 were further examined for their ability to inhibit protease activity of elastase. FIG. 15 shows the data. Two of the 2E12-SLPI conjugates (scFv-SLPI and scFv-SLPI-CH3) and SLPI Ig inhibited elastase proteolytic activity in a dose dependent fashion. Also, these molecules displayed protease inhibition activity by blocking elastase activity, implying that both the front and back end of the conjugates are functional. Effect of 2E12-SLPI conjugate and SLPI IG on PBMC proliferation. The effect of SLPI Ig and selected 2E12-SLPI conjugates on CD3 and PHA blast PBMC were also examined. See FIG. 16. We have seen that 2E12-SLPI conjugates (scFv-SLPI-CH3 and scFV-SLPI) has mild inhibitory effect on PMBC proliferation. SLPI Ig alone also has inhibitory effect on PMBC proliferation.

### EXAMPLE 3

### Purification of Binding Domain Fusion Protein

In the Example, a binding domain fusion protein is purified that comprises a binding domain that recognizes CD28 and a WAP domain-type protease inhibitor that comprises the sequence of 58-107 of human SLPI. CHO DHFR cells are transfected with the synthetic genes using the general method described by Morris et al. (Morris, A. E., Jiang, Y. J., McChesney, R. E., Jackson, A. E., Bancroft, C., and Chasin, L. A, Gene 94, 289-294, 1990). Chinese hamster dihydrofolate reductase-encoding gene (DHFR) is used as an effective dominant selectable reporter to construct stably transfected DHFR− CHO cells as a recipient cell for transfected genes. Transfected cells are selected on the basis of DHFR+ phenotype.

Chinese hamster ovary cells (CHO DG44) that have the endogenous DHFR gene deleted are used for expression of the binding domain fusion proteins. The DHFR⁻ cells are grown in medium containing HT (hypoxanthine, thymidine) so that all surviving cells must retain the plasmid. The plasmid (PD18) containing the gene for binding domain fusion protein expression is replicated extrachromosomally and is amplified in methotrexate.

The concentration of SLPI or SLPI-containing binding domain fusion protein is measured by an ELISA using a kit purchased from HyCult Biotechnology (HK316, human SLPI ELISA). The concentration of elafin or elafin-containing binding domain fusion protein is measured by ELISA using a test kit (HK318, human Elafin/SKALP ELISA) also from HyCult Biotechnology.

CHO cells containing a plasmid the specifies a binding domain fusion protein comprised of a binding domain that recognizes CD28, a WAP domain having the sequence of 58-107 of human SLPI, and a dimerization domain having the sequence of human CH3 is grown in Excell 302 (JRH Biosciences) medium supplemented with 4 mM glutamine, pen/strep, sodium pyruvate, and MEM nonessential amino acids (all from Invitrogen stock solutions) under serum free conditions with recombinant insule (ZN full chain). The cells are grown in a fermentor and the density of the cells is monitored. The concentration of the binding domain fusion protein is monitored using an ELISA kit that measures the concentration of SLPI. At a time corresponding to peak concentrations of binding domain fusion protein, the fermentation is stopped and the cell medium is separated from cells and cell debris by filtration. The filtered medium is passed over a column of immobilized Protein A and the column washed with buffer. The absorbance at 280 nm is monitored. The concentration of binding domain fusion protein is monitored in selected fractions by ELISA specific for human SLPI. When the absorbance at 280 nM has reached a low level; i.e. 0.1 to 0.3 absorbance units, the column is eluted with pH 3 glycine-citrate buffer to remove binding domain fusion protein. The fractions containing the eluted protein are immediately neutralized with Tris buffer and dialyzed into an appropriate buffer (0.1 M Hepes, pH 7.2, containing 150 mM NaCl or 0.1 M sodiuim phosphate, pH 7.2, containing 150 mM NaCl) compatible with stability of the binding domain fusion protein. The binding domain fusion protein is sterile filtered and stored under conditions that preserve its biological activity.

### EXAMPLE 4

### Analysis of Binding Domain Fusion Protein for Antibacterial Effect

The purified binding domain fusion proteins or samples at an intermediate stage of purification are transferred to a buffer that is compatible with antimicrobial assay. Although many commonly used buffers normally used for soluble proteins may be used, suitable buffers are phosphate buffer saline (PBS); 50 mM potassium phosphate, pH 7.2; 50 mM sodium phosphate, pH 70; 50 mM potassium phosphate, pH 7.2, containing 150 mM NaCl; and 50 mM sodium phosphate, pH 7.0, containing 150 mM NaCl.

Luminescence quantitative assay: A suitable bacteria is E. coli DH5α containing the luminescence plasmid pCGLS1 (Frackman, S., et al., 1998 Proc. Natl. Acad. Sci. 95, 14961-14966,). The bacteria are grown at 30 C to 37 C in LB (Luria-Bertani) media untillog phase is reached, and then collected by centrifugation and resuspended in 10 mM potassium phosphate, pH 7.2, containing 1% growth medium. The bacteria (10⁶) are incubated in a 96-well dish with various concentrations of binding domain fusion proteins for 4 hrs. The NaCl concentration is also varied between 0 and 200 mM. After 4 hrs incubation, luminescence is quantitated with a luminometer. Luminescence is an indication of the presence of a live cell or a cell that contains a luminescence plasmid. A graph of luminescence units versus the concentration of the added binding domain fusion proteins indicates the concentration at which 50% of the luminescence is inhibited. Each binding domain fusion protein is compared to an authentic example of highly purified trappin protein (SLPI, elafin, etc.) to quantitatively determine their relative antibacterial effects.

Colony forming unit (CFU) assay: Binding domain fusion proteins are assayed against bacteria samples which are then counted for live cells. Those skilled in the art recognize that many different bacteria may be used to determine biological activity of a binding domain fusion protein. A suitable bacteria is Pseudomonas aeruginosa. Bacteria are grown in a suitable medium, such as trypticase soy broth had 37° C. until log phase is reached. The cells are then collected by centrifugation and resuspended at 2×10⁵ CFU/ml in an appropriate buffer. A suitable buffer is 10 mM potassium phosphate, pH 7.2. The bacteria are incubated with various concentrations of the binding domain fusion protein in incubated for 4-6 hrs at 37° C. At the end of the incubation time, the bacteria are spread on the surface of agar plates and allowed to grow for 24 hrs had 37° C. The number of colonies is determined and compared to CFU results using authentic purified samples of trappins.

Other suitable bacteria include E. coli ML or Staphylococcus aureus, for example grown in tryptic soy broth and used in mid-logarithmic phase. The bacteria are collected by centrifugation and washed with 10 mM sodium phosphate, pH 7.4, and resuspended 10 mM sodium phosphate, pH 7.4, containing 1% tryptic soy broth. The bacteria concentration is estimated by spectrophotometry (0.2 A620=5×10⁷ bacteria/ml). To 450:1 of a bacterial cell culture containing 5×10⁴ bacteria/ml is added 50:1 of various concentrations of binding domain fusion protein. The cultures are incubated at 37 C. At the time of addition to the binding domain fusion protein, a sample of 100:1 is removed and is serially diluted to determine colony forming units. After 2 hr incubation, another 100:1 is removed for CFU determination. The CFU of controls is determined at 0 and 2 hr. The percent inhibition is determined by 100−(100× CFU sample/CFU control) and the percent of control is 100× CFU sample/CFU control. Bacteriocidal activity is exhibited by partial or complete inhibition of bacterial growth after treatment with a binding domain fusion protein.

The specificity of inhibition is tested by adding antibodies that react specifically with various trappins to the binding domain fusion protein before addition and incubation with bacteria. Various concentrations of antibodies are added to the binding domain fusion protein in order to achieve maximal inhibition of the antibacterial activity. Antibodies against SLPI (HP9024 rabbit polyclonal anti-human SLPI; HM2037 mouse Mab against human SLPI HyCult Biotechnology); elafin (HP9026 rabbit polyclonal anti-human elafin/SKALP, H2062 mouse Mab against human elafin/SKALP, SLPI HyCult Biotechnology) are used to block the antibacterial activity of the binding domain fusion proteins.

### EXAMPLE 5

### Inhibition of Elastase Activity by Binding Fusion Proteins

In this Example, the protease inhibition activity of a binding domain fusion protein comprising a protease inhibitor is evaluated. Several proteinases can be used to determine inhibition and specificity of inhibition of proteinases by binding domain fusion proteins. The protease inhibition activity of binding domain fusion protein, SLPI , elafin, and the parent proteinase inhibitor contained in a binding domain fusion protein is compared. In one assay, binding domain fusion protein, SLPI, elafin, or other proteinase inhibitor is used to inhibit one concentration of elastase, chymotrypsin, and cathepsin G in the presence and absence of various concentrations of binding domain fusion protein, SLPI, elafin, or parent proteinase inhibitor contained in the binding domain fusion protein. The assay buffer is compatible with proteinase activity. A suitable buffer is 50 mM Hepes, pH 7.4, containing 100 mM NaCl. Proteinase concentration is from 1 to 100 nM, more preferably from 10 to 60 nM, and depends on the particular proteinase used. Chymotrypsin is assayed in the presence of 10 mM calcium chloride. The concentration of binding domain fusion protein and other proteinase inhibitors used for comparison is from 0.01 to 100 nM and depends on the biological activity of the inhibitor. Binding domain fusion protein and other proteinase inhibitors and proteinase target is incubated for 30 min at the 25 C before the proteinase activity is determined. A suitable substrate for chymotrypsin and cathepsin G is N-succinyl-Ala-Ala-Pro-Phe-p-nitroanilide. Suitable substrates for elastase is N-methoxysuccinyl-Ala-Ala-Pro-Val-p-nitroanilide. Both p-nitroanilide substrates are dissolved in dimethyl sulfoxide. When the substrates are diluted into buffer, the concentration of dimethyl sulfoxide is kept as low as possible, preferably below 10% and more preferably below 5%. The mixture of binding domain fusion protein and proteinase is added to the substrates and the absorbance at 405 nm is recorded overtime in a 96-well format. The ratio of the reaction rates in the presence of inhibitor to that in the absence of inhibitor is equal to 1−([Eo]+[Io]+Kᵢ^{app})−{([Eo]+{Io]+Kᵢ^{app})²−4[Eo][Io]}^{1/2} /2(2[Eo]), where [Eo] and [Io] or the total concentrations of proteinase and binding domain fusion protein, respectively, and Kᵢ^{app} is the apparent inhibition constant. The inhibition constant, Kᵢ′ is calculated by correcting the Kᵢ^{app} values for the effect of substrate concentration using Kᵢ^{app}=Kᵢ(1+[So]/Kₘ), where [So] is a substrate concentration and Kₘ is the Michaelis-Menten constant.

In another assay, elastase (human neutrophil elastase, Calbiochem) is incubated with various concentrations of inhibitor (i.e., binding domain fusion protein, elafin, SLPI, etc.)in a buffer that is compatible for both proteins. Because elastase is somewhat labile and loses activity with time, it is preferred that fresh solutions of elastase are used. A suitable buffer is a 0.1 M Tris HCl, pH 7.8, containing 0.2 M NaCl and 0.05% Triton X-100. The concentration of elastase is between 5 and 10 nM, and preferably 8 nM. Elastase is incubated with various concentrations of a binding domain fusion protein for 10 to 20 min, and preferably 15 min, at 25 C. Elastase activity is determined by adding a sample (10 to 25:1) of the incubated mixture to 150:1 of 0.33 mM methoxysuccinyl-Ala-Ala-Pro-Val-p-nitroanilide (Sigma Aldrich M4765) in a 96-well dish and reading the absorbance at 405 nm as a function of time use in a 96-well plate reader. Similarly, the ability of a binding domain fusion protein to inhibit trypsin is tested using N-benzoyl-DL-arginine-p-nitroanilide (Sigma Aldrich, B4875). Kinetic constants are determined from the time course (Bieth, J. G., Biochem. Med. 32, 387-397, 1984).

In a another assay, N-methoxysuccinyl-Ala-Ala-Pro-Val-7-amido-4-methylcoumarin (Sigma Aldrich), a fluorogenic substrate for elastase, is dissolved in 0.1 M Hepes, pH 7.5, containing 0.5 M NaCl and 1 to 10% dimethylsulfoxide To 390:1 of substrate is added to 10:1 of elastase or elastase premixed with binding domain fusion protein. A fluorometer is used to measure fluorescence emission at 455 nm with an excitation wavelength of 383 nm. Measurements are recorded as a function of time for 10 to 20 minutes at 25 C. A plot of fluorescence intensity vs. time is made and the initial velocity is calculated and recorded. The assay is also performed using a 96-well dish and plate reader to record fluorescence intensity overtime. A specific activity is calculated based on a molar concentrations of the proteinases and inhibitors (i.e. binding domain fusion protein) added. A standard of human leukocytes elastase is used for comparison. The percent inhibition of a standard amount of binding domain fusion protein and a standard amount of elastase is used to compare the relative inhibitory capability of different binding domain fusion proteins.

### EXAMPLE 6

### Synergistic Effects Between Binding Domain Fusion Proteins and Inhibitory Proteins Found in Airways

Synergy is tested using a checkerboard assay format (Krogstad, D., and Moellering, R. C., in Antibiotics in Laboratory Medicine, 2^{nd} ed., edited by Lorian V. Baltimore, Williams & Wilkins, 1986, p. 557-578). Serial solutions of a binding domain fusion protein and a component of airway surface fluid, such as lysozyme, lactoferrin, etc.) are made so that each row and each column contain a fixed amount of binding domain fusion protein and an increasing amount of lysozyme, lactoferrin, etc. Within the scope of the invention is the use and testing of molecules not specifically known to be in ASL. The concentration of each of the components is adjusted in 2-fold serial dilutions so that they cover the range from 2- to 4-fold above the EC50 for each agent to 100- to 200-fold below. The binding domain fusion proteins and airway agents tested for synergy are all tested by themselves for antibacterial activity. E. coli DH5α containing the luminescence plasmid pCGLS1 (5×10⁶ CFU) are added to each well and incubated for 4 hrs. with proteins and mixtures of proteins. The luminescence is quantitated using a luminometer. Each row or column of the matrix represents a combination experiment, the fractional inhibition concentration (FIC) of each agent is calculated from each combination. The concentration that kills when used in combination with another agent divided by the concentration that has the same effect when used alone is equal to the FIC (Hall, M. J., Middleton, R. F., and Westmacott, D., J. Antimicrobiol. Chemother. 11, 427-433, 1983). To obtain the FIC index, the FIC values for a combination of two proteins is added. Combinations that act in an additive fashion have an FIC index of approximately one. Combinations that act synergistically have an FIC index of less than one. Combinations that act antagonistically have an FIC index of greater than one. Synergistic or antagonistic combinations that deviate substantially from one are more effective than combinations that are closer to one. Isobolograms are made with these data for easy interpretation. A concave isobologram signifies synergistic activity of the two components in the assay. binding domain fusion proteins that contain trappins are compared to the trappins incubated with lysozyme, lactoferrin, etc.

Samples of recombinant human lysozyme, human lactoferrin, cathelicidin LL-37, tobramycin, human defensins, human-β-defensin-1 (HBD-1), human β-defensin-2 (HBD-2), HNP-1 defensin, or secretory IgA (airway agents) are examples of components in airway fluids. These and other poteintial synergistic molecules are mixed with binding domain fusion proteins in various combinations and ratios. Elafin/SKALP (HC4011), Elafin/SKALP ELISA (HK318), human SLPI ELISA (HC316) human elastase ELISA (HK319), human lactoferrin (HP9034), human LLC peptide (HC4013), human lysozyme (H8035), human neutrophil defensins 1-3 (HK317) are obtained from Hycult Biotechnology, The Netherlands.

### EXAMPLE 6

### Analysis of Binding Domain Fusion Protein By a Time-Kill Method

The ability of subinhibitory concentrations of one agent to improve the killing ability of another agent overtime is tested using binding domain fusion proteins and components of ASL, such as lysozyme, lactoferrin, etc. (Krogstad, D., and Moellering, R. C., in Antibiotics in Laboratory Medicine, 2^{nd} ed., edited by Lorian V. Baltimore, Williams & Wilkins, 1986, p. 557-578). E. coli DH5∀ containing the luminescence plasmid pCGLS1 (5×10⁶ cfu) are incubated with (i) ASL component (such as lysozyme), which is present at a concentration equal to its EC50, (ii) half the concentration of a binding domain fusion protein that produced no decrease in luminescence, (iii) a combination of ASL component and binding domain fusion protein, and (iv) buffer alone. A time course of luminescence is determined and various incubation times are used ranging from 30 min to 6.5 hrs at hourly intervals. A plot of luminescence units versus time for each of the three reactions containing protein(s) (i, ii, and iii) is made and compared to buffer (iv). If the presence of subinhibitory concentration of binding domain fusion protein is effective in enhancing killing by a ASL component, this will it be observed in the plot. Within the scope of the invention is the use and testing of molecules not specifically known to be in ASL.

### EXAMPLE 7

### Augmentation of the Production of Hepatocyte Growth Factor by Binding Domain Fusion Proteins

Fibroblast cell cultures are assayed for the production of heptocyte growth factor (HGF) in the presence and absence of various concentrations of binding domain fusion proteins. Although several types of fibroblast cell cultures may be used, suitable fibroblasts are human lung fibroblasts CCD-11Lu, CCD-25Lu, CCD-32Lu, and CCD-33Lu, all of which are purchased from the American Type Culture Collection (Rockville, Md.). Cells are grown in Dulbecco's modified Eagle's medium (DMEM) containing 10% fetal calf serum in 5% carbon dioxide at 37° C. Cells are plated on 6- or 12-well dishes at the 5×10⁴ cells/cm² for 24 hours before they are washed three times with PBS (Ca and Mg free) and then incubated with various concentrations of binding domain fusion proteins in the presence of serum free DMEM. After incubation for 16 to 24 hours with binding domain fusion protein, the cell medium is collected and clarified by centrifugation. An ELISA (enzyme linked immunosorbent assay) is used to quantitate HGF. An ELISA kit is used for this purpose and is obtained from commercial sources, such as DHG00 Human HGF Quantikine ELISA kit (R&D Systems, Minneapolis, Minn.). A standard curve using a wide concentration range of HGF is used to determine the concentration of HGF in the cell medium. A concentration range of binding domain fusion protein is used to determine the maximal effect on the increase of HGF production. The parent trappin is used for comparison.

### EXAMPLE 8

### Analysis of Binding Domain Fusion Protein for Anti-Inflammation Effect in Vivo

Mice are given by intracheal instillation a complex of Pseudomonas aeruginosa 508 and agar microbeads that are smaller than 200 μm diameter (Gosselin, D., DeSanctis, J., Boulé, M., Skamene, E., Matouck, C., and Radzioch, D., Infect. Immun. 63, 3272-3278, 1995). The method of Jin et al. (Jin, F. Y., Nathan, C., Radzioch, D., and Ding, A., Cell 88, 417-426, 1997) is used to determine the effect of binding domain fusion protein on the inflammatory response of a bacterial challenge to the pulmonary system. P. aeruginosa is grown until log phase is reached and then a sample of the bacteria are vigorously stirred in Trypticase soy agar and heavy oil on ice. The live bacterial titer of the microbeads is determined by homogenizing the beads and plating serial dilutions on Trypticase soy agar medium. To instill the microbeads, a small incision is made at the ventral medline over the trachea of an anesthetized mouse and a 50:1 volume of the microbeads containing 5×10⁴ live bacteria is injected into the trachea. An additional 50:1 of air is introduced through a 22-gauge IV catheter extended into the trachea. The incision is closed with surgical clips. Sham controls are made by instilling microbeads made with buffer in place of bacteria. Total RNA is prepared from lungs removed at various times (0; 3, 6, and 24 hr; and 3, 5, 7, and 14 day) after instillation of the bacteria sample. A Northern blot is made by electrophoresis of 25 :g of RNA/lane on a 1% agarose gel using a buffer of 20 mM 3-(N-morpholino)propanesuflonic acid (MOPS), pH 7.0, containing 50 mM sodium acetate, 1 mM EDTA, and 2% formaldehyde. After transferring the RNA to a nylon membrane (NEN Research Products, Boston, Mass.), a Prime-a-Gene kit (Promega, Madison, Wis.) is used to probe label (10⁶ cpm/ml) the hybridized sample for 18 hr at 42 C. cDNA for SLPI and cDNA for β-actin control to normalize the Northern blots is hybridized. The hybridization buffer is 5×SSC, 5× Denhardt solution, 50% formamide, and 1% SDS containing 100 :g of sperm DNA per ml. An autoradiograph is developed for the Northern blot and the image density of the SLPI and ∃-actin bands are determined. The SLPI image intensity as a function of time is corrected for differences in loading using the image density of the β-actin band. Binding domain fusion protein is given by tail vein injection to the mice on days 0, 1, 2, 3, 4, and 7. A sham injection of buffer is given to the control mice on the same schedule. The dose of binding domain fusion protein is initially 1 mg/kg and is adjusted in additional experiments depending on the results. A dose response is obtained using doses of binding domain fusion protein that produce little or no effect to a dose that produces a maximal effect. Using the 0 time lane as the denominator, the —fold increase in SLPI cDNA content as a function of time is graphed. Jin et al. have demonstrated that SLPI cDNA increases in response to instillation of P. aeruginosa. When compared to the sham treated control samples, a positive effect against inflammation caused by P. aeruginosa is reduced production of SLPI mRNA in the treated mice.

### EXAMPLE 9

### Analysis of binding Domain Fusion Protein for Inhibiting in Vitro Effects of LPS

In response to stimulation by LPS, B cells secrete SLPI and simultaneously begin to proliferate and produce immunoglobulins. A preferred method uses various concentrations of binding domain fusion protein and other proteinase inhibitors to inhibit the effects of LPS in a culture of mouse spleen cells in the presence of LPS. Mouse spleen cells and macrophages that are deficient in SLPI (Nakamura, A., Mori, Y., Hagiwara, K., Suzuki, T., Sakakibara, T., Kikuchi, T., Igarashi, T., Ebina, M., Abe, T., Miyazaki, J., Takai, T., and Nukiwa, T., J. Exp. Med. 5, 669-674, 2003) are compared to mouse spleen cells and macrophages from wild type mice. Macrophages are collected from the intraperitoneal cavity of SLPI^{-/-} and SLPI^{+/+} mice have been treated with 2 ml of 4% thioglycollate medium (Sigma). The cells are washed with ice cold PBS and then cultured for one hour to allow adherent cells to attach to the culture dish. The nonadherent cells are removed by washing. The adherent peritoneal macrophages are incubated with 100 ng of LPS/ml or LPS and 100 U of gamma interferon (IFN( ) overnight in DMEM containing 10% fetal bovine serum in the presence and absence of various concentrations of binding domain fusion protein and other proteinase inhibitors. The concentrations of IL-6, IL-1ε, and TNF-α in the culture supernatants are determined by an ELISA. NO₂⁻ is determined by Nitrate/Nitrite colorometric kit (Cayman Chemical). The concentrations of the cytokines and NO₂⁻ are compared for the SLPI deficient and wild type macrophages as a function of the concentration of binding domain fusion protein.

SLPI^{-/-} are known to be sensitive to LPS-induced endotoxin shock. SLPI^{-/-} and wild type mice are injected with an intraperitoneal LPS challenge (1 mg/mouse) and are then observed for survival. At the time of LPS challenge, mice are given various doses binding domain fusion protein and proteinase inhibitors (SLPI, elafin, etc.) for comparison. All injections of binding domain fusion protein and proteinase inhibitors are into the tail vein. The number of mice living on each day is plotted for binding domain fusion protein and the protein inhibitors to determine if binding domain fusion protein protects against LPS challenge. A delay in the death of mice given a binding domain fusion protein compared to a buffer control indicates that binding domain fusion protein protected against LPS challenge.

### EXAMPLE 10

### Analysis of Binding Domain Fusion Protein for Anti-Inflammation Effect in Vitro

The procedures of Jin et al. (Jin, F. Y., Nathan, C., Radzioch, D., and Ding, A., Cell 88, 417-426, 1997 are used to determine the effect of a binding domain fusion protein on macrophage cells in vitro Activated primary mouse macrophages are prepared by intraperitoneal injection of 2 ml of 4% Brewer's thioglycollate broth (Difco, Detroit, Mich.) into a group of five mice. After 4 days, the peritoneal cavity is washed and primary macrophages are collected. A preferred macrophage cell line is RAW 264.7 macrophage cell line obtained from ATCC, Manassas, Va. RAW 264.7 is grown in RPMI 1640 medium containing 10% heat inactivated fetal bovine serum. The medium is assayed for LPS contamination and not used if the LPS concentration is above 25 pg/ml. Other macrophage cell lines are also suitable to use in this assay.

Monolayers of the cells are prepared in 24-well or 96-well culture dishes. For 96-well dishes, 10⁵ cells/well is used in 150:1 of medium. Cells are incubated with LPS, IFN(, or both for 48 hrs. The culture medium (100:1) is collected and placed in 96-well dishes. One hundred microliters of Griess' reagent (1% sulfanilamide, 0.1% napththylethylenediamine dihydrochloride, and 2.5% phosphoric acid) is added and the absorbance at 550 nm is determined. The concentration of nitrite in the buffer is subtracted from all determinations. A standard curve of sodium nitrite concentration against absorbance at 550 nm is used to determine the nitrite concentrations of the cultured samples. The nitrite concentrations of cells incubated with various concentrations of binding domain fusion protein are determined. A positive effect of binding domain fusion protein is a relative reduction of nitrite production compared to the absence of binding domain fusion protein.

### EXAMPLE 11

### Biacore Analysis of Binding Domain Fusion Protein for Binding to Elastase

The analysis of binding kinetics, which can be used to calculate thermodynamic binding constants, is performed by covalently immobilizing a soluble binding domain fusion protein on BIAcore chips and passing a solution of proteases over the chip surface. Proteases are also immobilized on the BIAcore chip and a solution of a binding domain fusion protein is passed over the chip. The instrument detects surface plasmon resonance changes over time. Surface plasmon resonance is sensitive to the mass of material that is adsorbed on a thin hydrophilic film on the surface of the gold-coated chip. As the mass of the SMIP builds up on the chip surface, surface plasmon resonance is increased and the data is captured continuously on a computer for further analysis. Automated software is used to calculate the microscopic kinetic constants. From the rate of change in resonance units at various concentrations of the SMIP, the microscopic rate of association is calculated. When the resonance signal has reached a plateau at a single concentration of the solute (protease or binding domain fusion protein), the flow of the solute solution is discontinued and buffer is used to wash off the bound solute. From the rate at which resonance units decrease (mass on the chip is lost as the bound protein dissociates), the rate of dissociation of the binding domain fusion protein:protease complex can be calculated. The shape of the curves is often diagnostic for the presence of aggregates in the solute solution. The ratio of the microscopic association and dissociation rate constants is equal to the thermodynamic binding constant (Ka). The inverse of Ka is the thermodynamic dissociation constant (Kd). The binding domain fusion proteins will be compared to the parent trappins.

To test the specificity of binding of binding domain fusion proteins to elastase, elastase is treated with N-methoxysuccinyl-Ala-Ala-Pro-Val-chloromethyl ketone (Sigma Aldrich M4814) in order to inactivate the active site. The adduct is passed over the binding domain fusion protein that is immobilized on the BIAcore chip. The adduct fails to bind to the binding domain fusion protein if elastase is inactivated. Inactivation of elastase is demonstrated by its lack of activity (inability to release p-nitroaniline) against N-methoxysuccinyl-Ala-Ala-Pro-Val-p-nitroanilide substrate.

To demonstrate specificity of binding, an immobilized binding domain fusion protein is blocked by reaction with antibodies directed against the trappin portion of the binding domain fusion proteins. After the binding domain fusion protein is immobilized on the surface of the BIAcore chip, the chip is further blocked by passing a solution of antibodies, such as rabbit polyclonal anti-human SLPI or rabbit anti-human elafin/SKALP (Hycult Biotechnology), over the chip. After the chip is washed free of antibody, a solution of elastase or elastase inactivated with N-methoxysuccinyl-Ala-Ala-Pro-Val-chloromethyl ketone is passed over the BIAcore chip and the resonance units recorded. A chip that contains binding domain fusion protein:antibody complex has reduced capability of binding elastase.

### EXAMPLE 12

### Analysis of Binding Domain Fusion Protein for Inhibition of Chymotrypsin, Trypsin, and Other Proteases

The ability of binding domain fusion protein, SLPI, elafin, and the parent proteinase inhibitor contained in a binding domain fusion protein are compared for their inhibitory effect (a characteristic of biological activity) on various proteases. Binding domain fusion protein, SLPI, elafin, or proteinase inhibitor is used to inhibit one concentration of elastase, chymotrypsin, or cathepsin G in the presence and absence of various concentrations of binding domain fusion protein, SLPI, elafin, or parent proteinase inhibitor contained in the binding domain fusion protein. The assay buffer is compatible with proteinase activity. A preferred buffer is 50 mM Hepes, pH 7.4, containing 100 mm NaCl. Proteinase concentration is from 1 to 100 nM, and more preferably from 10 to 60 nM. The proteinase concentration will depend on the particular proteinase used. Chymotrypsin is assayed in the presence of 10 mM calcium chloride. The concentration of binding domain fusion protein and other proteinase inhibitor used for comparison is from 0.01 to 100 nM depends on the biological activity of the inhibitor. binding domain fusion protein and other proteinase inhibitors and proteinase target is incubated for 30 min at the 25 C before the proteinase activity is determined.

### EXAMPLE 13

### Analysis of Binding Domain Fusion Protein for Inhibition of HIV-1

Several methods are known to those skilled in the art to quantitate inhibition of HIV replication and to assay cells for HIV infection that are acutely, chronically, and latently infected with HIV-1. In a preferred method (Shine, N. R., Wang, S. C., Konopka, K., Burks, E. A., Duzgunes, N., and Whitman, C. P., Bioorg. Chem. 30, 249-263,2002), the ability of a binding domain fusion protein to inhibit HIV-1 replication in PMA-treated THP-1 monocytic cells (American Type Culture Collection TIB-202). The cells are grown at 37 C in RMPI1640 medium supplemented with 10% heat inactivated fetal bovine serum. A single cell suspension of THP-1 cells is treated with phorbol 12-myristate 13-acetate (PMA) to stimulate differentiaton. A monocytotropic strain of HIV, HIV-1_{Ba-L} (Advanced Biotechnologies, Columbia, Mo.), is propogated in macrophages (Pretzer, E., Flasher, D., and Duzgunes, 1997 Antiviral. Res. 34: 1-15). PMA-treated cells adhere to culture dishes and undergo morphological change to a flat, amoeboid shape. The cells are grown for approximately seven days. The cells are preincubated with various concentrations of binding domain fusion protein, SLPI, or elafin two hrs at 37 C. The cells are then infected with HIV-1_{Ba-L}. After 2 hr. at 37 C, the cells are washed three times and then cultured in RPMI medium. The amount of infection by HIV is quantitated by determining the load of viral p24 present in culture supernatants using in ELISA specific for p24 (AIDS Vaccine Program, National Cancer Institute, Frederick, Md.). binding domain fusion protein is also preincubated with HIV for 2 hr. before HIV is added to the cells. A binding domain fusion protein is biologically active if it reduces the amount of p24 observed in a culture supernatant compared to a control using buffer.

Some protease inhibitors are thought to affect very late events (posttranscriptional and translational) in the postintegrative phase of virus replication. In a second preferred method (Turpin, J. A., Buckheit, R. W., Jr., Derse, D., Hollingshead, M., Williamson, K., Palamone, C., Osterline, M. C., Hill, S. A., Graham, L., Schaeffer, C. A., Bu, M., Huang, M., Cholody, W. M., Michejda, C. J., and Rice, W. G., Antimicrobiol. Agents Chemother. 42, 487-494), chronically infected H9/HLV-III_{B} NIH 1983 cells (5×10⁴ cells/ml) are cultured for five days in the presence and absence of different concentrations of binding domain fusion proteins or trappins. The supernatants of the cultures after five days are used to determine virion-associated reverse transcriptase activity. Cell viability is determined by XTT in a reduction assay. A binding domain fusion protein is biologically active if it reduces virion-associated reverse transcriptase activity and/or increases cell viability in a culture supernatant compared to a control using buffer.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, the claims referred to above including but not limited to any original claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A compound comprising a binding domain polypeptide capable of binding to a proteinase-associated molecule, a polypeptide comprising a proteinase inhibitor domain, and optionally, a polypeptide comprising a connecting region that connects the binding domain polypeptide and the polypeptide comprising the proteinase inhibitor domain.

2. A compound of claim 1 wherein said binding domain polypeptide comprises an immunoglobulin or portion thereof.

3. A compound of claim 1 wherein said binding domain polypeptide comprises a monoclonal antibody or binding portion thereof.

4. A compound of claim 2 wherein said binding domain polypeptide is selected from Fab, Fab′, F(ab′)₂ and Fv fragments.

5. A compound of claim 1 wherein said binding domain polypeptide comprises a single chain protein.

6. A compound of claim 1 wherein said binding domain polypeptide comprises an immunoglobulin light chain variable region and an immunoglobulin heavy chain variable region polypeptide.

7. A compound of claim 1 wherein said binding domain polypeptide comprises two immunoglobulin heavy chain variable region polypeptides.

8. A compound of claim 1 wherein said binding domain polypeptide comprises a single chain Fv.

9. A compound of claim 1 wherein said binding domain polypeptide comprises a single chain Fv encoded by a synthetic polynucleotide.

10. A compound of claim 9 wherein said synthetic polynucleotide is synthesized by combining more than one oligonuclotide.

11. A compound of claim 9 wherein said synthetic polynucleotide is synthesized by combining more than one oligonuclotide by a PCR procedure.

12. A compound of claim 1 wherein said binding domain polypeptide comprises a single chain Fv encoded by a polynucleotide isolated from a library using phage display.

13. A compound of claim 1 wherein said binding domain polypeptide comprises a single chain Fv encoded by a polynucleotide isolated from hybridoma.

14. A compound of claim 1 wherein said binding domain polypeptide binds to an antigen on a leukocyte.

15. A compound of claim 1 wherein said binding domain polypeptide binds to an antigen on a T lymphocyte.

16. A compound of claim 1 wherein said binding domain polypeptide is an scFv that binds to an antigen on a T lymphocyte.

17. A compound of claim 16 wherein said T lymphocyte antigen is selected from CD2, CD3, CD4, CD5, CD6, CD7, CD8, CD25, CD28, CD69, CD154, CD152 (CTLA-4), and ICOS.

18. A compound of claim 1 wherein said binding domain polypeptide binds to an antigen on a helper T cell.

19. A compound of claim 1 wherein said binding domain polypeptide binds to an antigen on a monocyte.

20. A compound of claim 1 wherein said binding domain polypeptide binds to an antigen on a dendritic cell.

21. A compound of claim 1 wherein said binding domain polypeptide binds to an antigen on an immune effector cell.

22. A compound of claim 1 wherein said binding domain polypeptide binds to an antigen on a B cell.

23. A compound of claim 1 wherein said binding domain polypeptide binds to a target that is an antigen selected from CD2, CD3, CD4, CD5, CD6, CD7, CD8, CD19, CD20, CD21, CD22, CD23, CD25, CD28, CD37, CD40, CD45, CD45 RA, CD45 RO, CD69, CD154, CD152 (CTLA-4), ICOS, MHC class II, VEGF.

24. A compound of claim 1 wherein said binding domain polypeptide binds to VEGF.

25. A compound of claim 1 wherein said binding domain polypeptide binds to CD28.

26. A compound of claim 1 wherein said proteinase inhibitor domain comprises a trappin polypeptide having proteinase inhibitor activity.

27. A compound of claim 26 wherein said trappin polypeptide comprises a naturally occurring SLPI polypeptide.

28. A compound of claim 26 wherein said trappin polypeptide comprises a analog of a naturally occurring SLPI polypeptide.

29. A compound of claim 26 wherein said trappin polypeptide is an analog of a naturally occurring trappin polypeptide.

30. A compound of claim 1 wherein said proteinase inhibitor domain comprises a WAP domain having proteinase inhibitor activity.

31. A compound of claim 30 wherein said WAP domain is an analog of a naturally occurring WAP motif polypeptide.

32. A compound of claim 1 wherein said proteinase inhibitor domain comprises a TIMP having proteinase inhibitor activity.

33. A compound of claim 32 wherein said TIMP polypeptide is an analog of a naturally occurring TIMP polypeptide.

34. A compound of claim 1 wherein said proteinase inhibitor domain comprises a cystatin having proteinase inhibitor activity.

35. A compound of claim 34 wherein said cystatin is an analog of a naturally occurring cystatin polypeptide.

36. A compound of claim 1 wherein said proteinase inhibitor domain comprises a defensin.

37. A compound of claim 36 wherein said defensin is a non-naturally occurring analog of a naturally occurring defensin polypeptide.

38. A compound of claim 1 wherein said proteinase inhibitor inhibits a proteinase selected from elastase, alpha₁-proteinase, proteinase 3, chymotrypsin, trypsin, human mast cell chymase, stratum corneum chymotryptic enzyme, human leukocyte elastase, human cathepsin G, bovine chymotrypsin, pig chymotrypsin, tryptase, human leukocyte elastase, pig pancreatic elastase, stratum corneum chymotryptic enzyme.

39. A compound of claim 1 wherein said proteinase inhibitor domain inhibits a proteinase having a substrate selected from elastin, proteoglycans, collagen, VEGF precursor.

40. A compound of claim 1 wherein said proteinase inhibitor domain is mutated to have an increased or reduced proteinase inhibition activity relative to a non-mutated proteinase inhibitor domain.

41. A compound of claim 1 that is modified to effect the specificity for a target proteinase that is inhibited.

42. A compound of claim 1 wherein said proteinase inhibitor has at least one biological activity selected from i) modulates the activity of an endogenous protease, ii) modulates the expression of an endogenous proteinase, iii) modulates signal transduction, iv) modulates the activity of a signal transduction molecule, and v) modulates the expression of a signal transduction molecule.

43. A compound of claim 1 comprising a proteinase inhibitor analog having at least one biological activity selected from i) modulates the activity of an endogenous protease, ii) modulates the expression of an endogenous proteinase, iii) modulates signal transduction, iv) modulates the activity of a signal transduction molecule, and v) modulates the expression of a signal transduction molecule.

44. A compound of claim 1 comprising a TGase motif.

45. A compound of claim 1 comprising two or more TGase motifs.

46. A compound of claim 44 or 45 wherein said TGase motif comprises the amino acid sequence Gly-Gln-Asp-Pro-Val-Lys.

47. A compound of claim 1 further comprising a dimerization domain.

48. A compound of claim 47 wherein said dimerization domain comprises an immunoglobulin CH3 domain or portion thereof.

49. A compound of claim 1 wherein said connecting region comprises a dimerization domain.

50. A compound of claim 1 wherein said connecting region comprises a naturally occurring hinge region selected from a human hinge or portion thereof, human IgG hinge or a portion thereof, human IgA hinge or a portion thereof, human IgE hinge or a portion thereof, camelid hinge region or a portion thereof, IgG1 llama hinge region or portion thereof, nurse shark hinge region or portion thereof, and spotted ratfish hinge region or a portion thereof.

51. A compound of claim 1 wherein said connecting region comprises a naturally occurring hinge region selected from a human hinge or portion thereof, human IgG hinge or a portion thereof, human IgA hinge or a portion thereof, human IgE hinge or a portion thereof, camelid hinge region or a portion thereof, IgG1 llama hinge region or portion thereof, nurse shark hinge region or portion thereof, and spotted ratfish hinge region or a portion thereof.

52. A compound of claim 1 wherein said connecting region comprises a human IgE hinge or a portion thereof.

53. A compound of claim 1 wherein said connecting region comprises a human IgG1, IgG2, IgG3 or IgG4 hinge region having either zero or one cysteine residue.

54. A compound of claim 1 wherein said connecting region comprises a human IgG hinge region having between zero and two cysteine residues.

55. A compound of claim 1 wherein said connecting region comprises a wild type human IgG1 immunoglobulin hinge region.

56. A compound of claim 1 wherein said connecting region comprises a glycosylation site.

57. A compound of claim 1 further comprising a synthetic proteinase inhibitor conjugated to the connecting region.

58. A compound of claim 1 wherein said connecting region has no cysteine residues capable of forming disulfide bonds.

59. A compound of claim 1 wherein said connecting region has one cysteine residue.

60. A compound of claim 1 wherein said connecting region comprises a mutated wild-type immunoglobulin hinge region polypeptide comprising no more than one cysteine residue.

61. A compound of claim 1 wherein said connecting region is altered so that said compound has a reduced ability to dimerize.

62. A compound of claim 1 wherein said connecting region comprises a mutated wild-type immunoglobulin hinge region polypeptide or portion thereof comprising first, second, and third cysteine residues, where said first cysteine reside is N-terminal to said second cysteine and said second cysteine is N-terminal to said third cysteine, wherein said first cysteine residue is substituted or deleted.

63. A compound of claim 1 wherein said connecting region is from about 15 to about 115 amino acids in length.

64. A compound of claim 1 wherein said connecting region is from about 10 to about 50 amino acids in length.

65. A compound of claim 1 wherein said connecting region is from about 15 to about 35 amino acids in length.

66. A compound of claim 1 wherein said connecting region is from about 18 to about 32 amino acids in length.

67. A compound of claim 1 wherein said connecting region is from about 5 to about 15 amino acids in length.

68. A compound of claim 1 further comprising an immunoglobulin constant region domain.

69. A compound of claim 68 comprising an immunoglobulin CH3 constant region domain.

70. A compound of claim 68 comprising an immunoglobulin CH2CH3 constant region domains.

71. A compound comprising: i) a first polypeptide having a binding domain polypeptide capable of binding to a target molecule; ii) a second polypeptide comprising a connecting region attached to said first polypeptide; iii) a third polypeptide comprising a proteinase inhibitor domain or a domain that regulates a proteinase inhibitor; and iv) a fourth polypeptide comprising a immunoglobulin constant region or portion thereof.

72. A compound of claim 68 wherein said immunoglobulin constant region domain is capable of mediating an effector function selected from complement dependent cytotoxicity, antibody dependent cellular cytotoxicity, FcR binding, protein A binding, and decreasing a number of target cells.

73. A compound of claim 1 further comprising one or more dimerization domain and one or more TGase domain, wherein said proteinase inhibitor domain comprises one or more WAP domain.

74. A compound of claim 1 further comprising one or more dimerization domain, wherein said first polypeptide is N-terminal to said second polypeptide and said second polypeptide is N-terminal to said proteinase inhibitor domain, wherein said proteinase inhibitor domain comprises one or more WAP domain, and wherein said one or more WAP domain is N-terminal to said one or more dimerization domain.

75. A compound of claim 1 further comprising one or more dimerization domain, wherein said first polypeptide is N-terminal to said second polypeptide and said second polypeptide is N-terminal to said one or more dimerization domain, and wherein said dimerization domain is N-terminal to said proteinase inhibitor domain, and wherein said proteinase inhibitor domain comprises one or more WAP domain.

76. A compound of claim 1 further comprising one or more dimerization domain and one or more TGase domain, wherein said first polypeptide is N-terminal to said second polypeptide and said second polypeptide is N-terminal to said proteinase inhibitor domain, wherein said proteinase inhibitor domain comprises one or more WAP domain, and wherein said one or more WAP domain is N-terminal to said one or more dimerization domain, and wherein said dimerization domain is N-terminal to said one or more TGase domain.

77. A compound of claim 1 further comprising one or more dimerization domain and one or more TGase domain, wherein said first polypeptide is N-terminal to said second polypeptide and said second polypeptide is N-terminal to said proteinase inhibitor domain, wherein said proteinase inhibitor domain comprises one or more WAP domain, and wherein said one or more WAP domain is N-terminal to said one or more TGase domain, and wherein said TGase domain is N-terminal to said one or more dimerization domain.

78. A compound of any one of claims 73 to 77 wherein said one or more WAP domain is a non-naturally occurring analog of a naturally occurring WAP motif.

79. A compound of any one of claims 73 to 78 comprising two to five WAP domains.

80. A compound of any one of claims 73 to 78 comprising one or two WAP domains.

81. A compound of claim 1 having antibacterial activity.

82. A method of treating a patient having a bacterial infection comprising administering an effective antibacterial amount of a compound according to claim 1.

83. A compound of claim 1 having anti-inflammatory activity.

84. A compound comprising a protease inhibitor molecule connected to an immunoglobulin domain, said immunoglobulin domain selected from the group consisting of a CH2CH3, a hinge-CH2CH3, a hinge-CH3, a CH1-hinge-CH2CH3, a CH1-hinge-CH3, and C_{L}.

85. The compound of claim 1 wherein said immunoglobulin domain is a primate immunoglobulin domain.

86. The compound of claim 1 wherein said immunoglobulin domain is a human immunoglobulin domain.

87. The compound of claim 1 wherein said protease inhibitor is a protein.

88. A compound of claim 1 wherein one or more of said CH2 or CH3 domains comprises an amino acid substitution or deletion.

89. A compound of claim 5 comprising more than one amino acid substitution or deletion.

90. A method of treating a patient having an inflammatory disorder comprising administering an effective anti-inflammatory amount of a compound according to claim 1.

91. A method of treating a patient having rheumatoid arthritis comprising administering an effective anti-inflammatory amount of a compound according to claim 1.

92. A compound of claim 1 that is effective for the treatment of an HIV infection in a patient.

93. A method of treating a patient having an HIV infection comprising administering an effective amount of a compound according to claim 1.

94. A compound of claim 1 that is effective for the treatment of a pulmonary or lung disorder in a patient.

95. A method of treating a pulmonary or lung disorder in a patient comprising administering to the patient an effective amount of a compound according to claim 1.

96. A compound of claim 1 that is effective for the treatment of pulmonary or lung inflammation in a patient.

97. A method of treating pulmonary or lung inflammation in a patient comprising administering to the patient an effective anti-inflammatory amount of a compound according to claim 1.

98. A compound of claim 1 that is effective for the treatment of a vascular disorder in a patient.

99. A method of treating a vascular disorder in a patient comprising administering to the patient an effective amount of a compound according to claim 1.

100. A compound of claim 1 that is effective for the treatment of an ophthalmic disease or disorder.

101. A method of treating a ophthalmic disorder or disease in a patient comprising administering to the patient an effective amount of a compound according to claim 1.

102. A compound of claim 1 that is effective for the treatment of age related macular degenerative disease.

103. A method of treating a of age related macular degenerative disease in a patient comprising administering to the patient an effective amount of a compound according to claim 1.

104. A polynucleotide encoding any of the compounds of claims 1-81.

105. A polynucleotide of claim 104 that is operably linked to a promoter or other sequence that enhances expression of the polynucleotide in a cell.

106. A cell containing a polynucleotide of claim 104 or 105.

107. A recombinant vector capable of expressing a protein according to any one of claims 1-81.

108. A method of expressing a protein according to any one of claims 1-81 under conditions in which the protein is expressed.
